(19) United States
(12) Patent Application Publication
(10) Pub. No.: US 2010/0160424 A1
(43) Pub. Date: Jun. 24, 2010

(54) RENIN INHIBITORS

(76) Inventors: John J. Baldwin, Gwynedd Valley, PA (US); Salvacion Cacatian, Blue Bell, PA (US); David A. Claremon, Maple Glen, PA (US); Lawrence W. Dillard, Yardley, PA (US); Patrick T. Flaherty, Pittsburgh, PA (US); Alexey V. Ishchenko, Somerville, MA (US); Gerard McGeehan, Garnet Valley, PA (US); Robert D. Simpson, Wilmington, DE (US); Suresh B. Singh, Kendall Park, NJ (US); Colin M. Tice, Ambler, PA (US); Zhenrong Xu, Horsham, PA (US); Jing yuan, Lansdale, PA (US); Wei Zhao, Eagleville, PA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/665,213

(22) PCT Filed: Jun. 20, 2008
(86) PCT No.: PCT/US08/07662

§ 371 Date: Feb. 19, 2010

Related U.S. Application Data
(60) Provisional application No. 60/936,400, filed on Jun. 20, 2007.

(51) Int. Cl. A61K 31/335; C07D 309/04; C07C 271/10; C07D 313/04; A61K 31/351; A61K 31/24; A61P 9/10

(52) U.S. Cl. 514/450; 549/426; 560/27; 549/346; 514/459; 514/539

(57) ABSTRACT

Described are compounds that bind to aspartic proteases to inhibit their activity. They are useful in the treatment or amelioration of diseases associated with aspartic protease activity. Also described are methods of use of the compounds described herein in ameliorating or treating aspartic protease related disorders in a subject in need thereof.

### RELATED APPLICATION

This application claims the benefit of U.S. Provisional Application No. 60/936,400, filed on Jun. 20, 2007. The entire teachings of the above application are incorporated herein by reference.

### BACKGROUND OF THE INVENTION

Aspartic proteases, including renin, β-secretase (BACE), Candida albicans secreted aspartyl proteases, HIV protease, HTLV protease and plasmepsins I and II, are implicated in a number of disease states. In hypertension elevated levels of angiotensin I, the product of renin catalyzed cleavage of angiotensinogen are present. Elevated levels of β-amyloid, the product of BACE activity on amyloid precursor protein, are widely believed to be responsible for the amyloid plaques present in the brains of Alzheimer's disease patients. Secreted aspartyl proteases play a role in the virulence of the pathogen Candida albicans. The viruses HIV and HTLV depend on their respective aspartic proteases for viral maturation. Plasmodium falciparum uses plasmepsins I and II to degrade hemoglobin.

In the renin-angiotensin-aldosterone system (RAAS) the biologically active peptide angiotensin II (Ang II) is generated by a two-step mechanism. The highly specific aspartic protease renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAAS represents a major advance in the treatment of cardiovascular diseases (Zaman, M. A. et al Nature Reviews Drug Discovery 2002, 1, 621-636). ACE inhibitors and AT₁ blockers have been accepted as treatments of hypertension (Waeber B. et al., “The renin-angiotensin system: role in experimental and human hypertension”, in Berkenhager W. H., Reid J. L. (eds): Hypertension, Amsterdam, Elsevier Science Publishing Co, 1996, 489-519; Weber M. A., Am. J Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N Engl. J. Med, 1992, 327, 669).

Interest in the development of renin inhibitors stems from the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be bypassed by chymase, a serine protease (Husain A., J. Hypertens., 1993, 11, 1155). In patients, inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the ATI receptor (e.g., by losartan) on the other hand overexposes other AT-receptor subtypes to Ang II, whose concentration is dramatically increased by the blockade of AT1 receptors. In summary, renin inhibitors are not only expected to be superior to ACE inhibitors and AT₁ blockers with regard to safety, but more importantly also with regard to their efficacy in blocking the RAAS.

Only limited clinical experience (Azizi M. et al., J. Hypertens., 1994, 12, 419; Neutel J. M. et al., Am. Heart, 1991, 122, 1094) has been generated with renin inhibitors because their peptidomimetic character imparts insufficient oral activity (Kleinert H. D., Cardiovasc. Drugs, 1995, 9, 645). The clinical development of several compounds has been stopped because of this problem together with the high cost of goods. It appears as though only one compound has entered clinical trials (Rahuel J. et al., Chem. Biol., 2000, 7, 493; Mealy N. E., Drugs of the Future, 2001, 26, 1139). Thus, metabolically stable, orally bioavailable and sufficiently soluble renin inhibitors that can be prepared on a large scale are not available. Recently, the first non-peptide renin inhibitors were described which show high in vitro activity (Oefner C. et al., Chem. Biol., 1999, 6, 127; Patent Application WO 97/09311; Maerki H. P. et al., Il Farmaco, 2001, 56, 21). The present invention relates to the unexpected identification of renin inhibitors of a non-peptidic nature and of low molecular weight. Orally active renin inhibitors which are active in indications beyond blood pressure regulation where the tissular renin-chymase system may be activated leading to pathophysiologically altered local functions such as renal, cardiac and vascular remodeling, atherosclerosis, and restenosis, are described.

All documents cited herein are incorporated by reference.

### SUMMARY OF THE INVENTION

Compounds have now been found which are bind to aspartic proteases to inhibit their activity. They are useful in the treatment or amelioration of diseases associated with aspartic protease activity.

One embodiment of the invention is compound represented by Structural Formula (I):

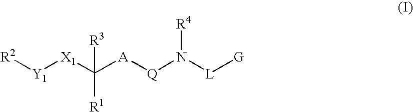

wherein:

X₁ is a covalent bond, —O—, —S—, —S(O)—, —S(O)₂—;

Yᵢ is a covalent bond or C₁-C₁₀ alkylene, alkenylene or C₁-C₁₀ alkynylene, each optionally substituted at one or more substitutable carbon atom with halogen, cyano, nitro, hydroxy, (C₁-C₃)alkyl, (C₁-C₃)alkoxy or halo(C₁-C₃)alkoxy, provided that Y₁ is a covalent bond only when X₁ is a covalent bond;

A is a saturated or unsaturated 4-, 5-, 6-, or 7-membered ring which is optionally bridged by (CH₂)ₚ via bonds to two members of said ring, wherein said ring is composed of carbon atoms and 0-2 hetero atoms selected from the group consisting of 0, 1, or 2 nitrogen atoms, 0 or 1 oxygen atoms, and 0 or 1 sulfur atoms, said ring being optionally and independently substituted with zero to four halogen atoms, (C₁-C₆)alkyl groups, halo(C₁-C₆)alkyl groups or oxo groups such that when there is substitution with one oxo group on a carbon atom it forms a carbonyl group, and when there is substitution of one or two oxo groups on sulfur it forms sulfoxide or sulfone groups, respectively;

p is 1 to 3;

R¹ is (C₃-C₇) cycloalkyl, phenyl, heteroaryl, or bicyclic heteroaryl each optionally substituted with 1 to 3 groups independently selected from: fluorine, chlorine, bromine, cyano, nitro, hydroxy, (C₁-C₆)alkyl, (C₃-C₆)cycloalkyl, (C₄-C₇)cycloalkylalkyl, (C₂-C₆)alkenyl, (C₅-C₇)cycloalkylalkenyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₅-C₇)cycloalkylalkynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy and (C₁-C₆)alkanesulfonyl; and phenyl, heteroaryl, phenoxy, heteroaryloxy, phenylthio, heteroarylthio, benzyl, heteroarylmethyl, benzyloxy and heteroarylmethoxy, each optionally substituted with 1 to 3 groups independently selected from: fluorine, chlorine, bromine, cyano, nitro, hydroxy, (C₁-C₃)alkyl, halo(C₁-C₃)alkyl, (C₁-C₃) alkoxy, and halo(C₁-C₃)alkoxy, and aminocarbonyl;
R² is —NHC(═NR¹²)(NH₂), —NHC(═NR¹²)(NHR⁹),

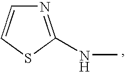

—OC(O)(NH₂), —OC(S)(NH₂), —SC(S)(NH₂), —SC(O)(NH₂), —OC(O)(NHR⁹), —OC(S)(NHR⁹), —SC(S)(NHR⁹), —SC(O)(NHR⁹), —NHC(O)OR⁹, —NHC(S)SR⁹, —NHC(S)OR⁹, —NHC(O)SR⁹, —C(O)R⁹, —C(S)R⁹, —C(O)(NH₂), —C(S)(NH₂), —C(O)(NHR⁹), —C(S)(NHR⁹) or —NHC(O)H, wherein R⁹ is a straight or branched C₁-C₅ alkyl, straight or branched C₁-C₅ haloalkyl, (C₃-C₄)cycloalkyl or straight or branched C₁-C₅ alkoxyalkyl and R¹² is H, (C₁-C₆)alkyl, phenyl, heteroaryl, cyano, nitro, —S(O)R^{9,} —S(O₂)R⁹, —S(O₂)NHR⁹, —S(O₂)NR⁹R⁹, —C(O)R⁹, —C(S)R⁹, —C(O)OR⁹, —C(S)OR⁹, —C(O)(NH₂), —C(O)(NHR⁹);

R³ is —H, —F, C₁-C₅ alkyl, —NHC(O)R¹⁰, —OH or —OR¹⁰, wherein R¹⁰ is C₁-C₃ alkyl, provided that when R³ is —F or —OH, then X₁ is not —O—, —S—, —S(O)—, —S(O)₂— and R²—Y₁—X₁ is not —OC(O)(NH₂), —OC(S)(NH₂), —SC(S)(NH₂), —SC(O)(NH₂), —OC(O)(NHR⁹), —OC(S)(NHR⁹), —SC(S)(NHR⁹), —SC(O)(NHR⁹), —NHC(O)OR⁹, —NHC(S)OR⁹, —NHC(S)SR⁹, —NHC(O)SR⁹ or —NHC(O)H;

Q is Q1, Q2, Q3, Q4, Q5, or Q6:

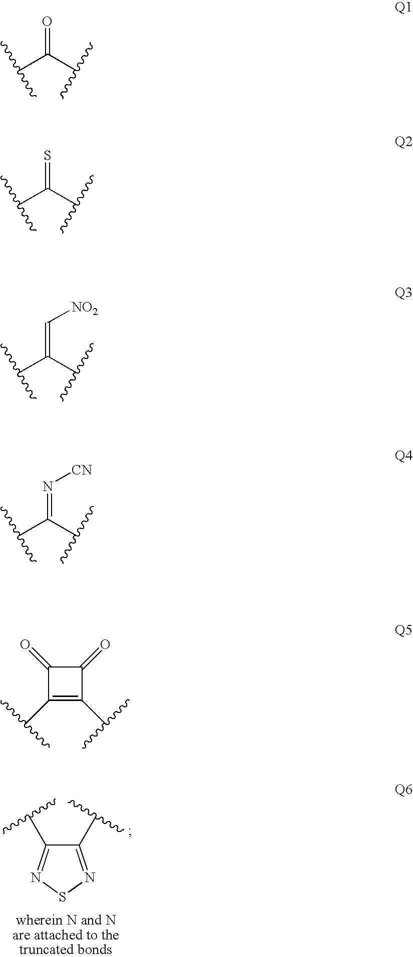

R⁴ is H, (C₁-C₆)alkyl, halo(C₁-C₆)alkyl, (C₁-C₃)alkoxy(C₁-C₃)alkyl, or cyano(C₁-C₆)alkyl;

G is OH, OR^{e}, NH₂, NHR^{e}, NR^{e}R^{f}, C(═NH)NH₂, C(═NH)NHR^{e}, NHC(═NH)NH₂, or NHC(=NH)NHR^{e};

L is 1) a linear (C₂-C₄)alkyl chain when G is OH, OR^{e}, NH₂, NHR^{e}, NR^{e}R^{f}, NHC(═NH)NH₂, or NHC(═NH)NHR^{e}, or 2) a linear (C₁-C₃)alkyl chain when G is C(═NH)NH₂ or C(═NH)NHR^{e};

L is optionally substituted by 1-4 groups independently selected from R⁵, R^{5a}, R⁶, and R^{6a}; one or more of the carbon atoms of L may be part of a 3-, 4-, 5-, 6-, or 7-membered saturated ring composed of carbon atoms, and 0-2 hetero atoms selected from 0 or 1 nitrogen atoms, 0 or 1 oxygen atoms, and 0 or 1 sulfur atoms; said saturated ring being optionally substituted with up to four groups selected from halogen, (C₁-C₆)alkyl, halo(C₁-C₆)alkyl, (C₃-C₆)cycloalkyl, halo(C₃-C₆)cycloalkyl, (C₄-C₇)cycloalkylalkyl, halo(C₄-C₇)cycloalkylalkyl, and oxo, such that when there is substitution with one oxo group on a carbon atom it forms a carbonyl group and when there is substitution of one or two oxo groups on sulfur it forms sulfoxide or sulfone groups, respectively;

R⁵, R^{5a}, R⁶, and R^{6a} is each independently selected from 1) H, (C₁-C₁₂)alkyl, halo(C₁-C₁₂)alkyl, hydroxy(C₁-C₁₂)alkyl, (C₃-C₁₀)cycloalkyl, (C₃-C₁₀)cycloalkyl, (C₃-C₁₀)cycloalkylalkyl, halo(C₃-C₁₀)cycloalkylalkyl, hydroxy(C₃-C₁₀)cycloalkylalkyl, (C₁-C₂)alkyl(C₃-C₁₀)cycloalkylalkyl, halo(C₁-C₂)alkyl(C₃-C₁₀)cycloalkylalkyl, di(C₁-C₂)alkyl(C₃-C₁₀)cycloalkylalkyl, hydroxy(C₁-C₂)alkyl(C₃-C₁₀)cycloalkylalkyl, hydroxy di(C₁-C₂)alkyl(C₃-C₁₀)cycloalkylalkyl, (C₂-C₁₂)alkenyl, (C₅-C₈)cycloalkyl(C₁-C₃)alkenyl, (C₂-C₁₂)alkynyl, (C₃-C₈)cycloalkyl(C₁-C₃)alkynyl, (C₄-C₁₂)bicycloalkyl(C₁-C₃)alkyl, (C₈-C₁₄)tricycloalkyl(C₁-C₃)alkyl, (C₁-C₆)alkoxy(C₁-C₆)alkyl, halo(C₁-C₆)alkoxy(C₁-C₆)alkyl, (C₃-C₈)cycloalkoxy(C₁-C₃)alkyl, (C₁-C₆)alkylthio(C₁-C₆)alkyl, halo(C₁-C₆)alkylthio(C₁-C₆)alkyl, (C₃-C₈)cycloalkylthio(C₁-C₃)alkyl, saturated heterocyclyl, and saturated heterocyclyl(C₁-C₃)alkyl wherein (a) hydrogen atoms in these groups are optionally substituted by 1 to 6 groups independently selected from halogen, cyano, nitro, hydroxy, (C₁-C₆)alkyl, (C₁-C₆)alkoxy, (C₃-C₆)cycloalkyl, (C₃-C₇)cycloalkylalkyl, halo(C₃-C₇)cycloalkylalkyl, (C₂-C₆)alkenyl, halo(C₂-C₆)alkenyl, (C₃-C₇)cycloalkylalkenyl, (C₂-C₆)alkynyl, halo(C₂-C₆)alkynyl, (C₃-C₇)cycloalkylalkoxy, halo(C₃-C₇)cycloalkylalkoxy, (C₃-C₇)cycloalkoxy, halo(C₁-C₆)alkyl, (C₃-C₇)cycloalkylalkynyl, halo(C₃-C₇)cycloalkylalkynyl, halo(C₁-C₆)alkoxy, halo(C₃-C₇)cycloalkyl, halo(C₃-C₇)cycloalkoxy, (C₁-C₆)alkylsulfonyl, aminocarbonyl and wherein (b) divalent sulfur atoms are optionally oxidized to sulfoxide or sulfone;

or 2) phenyl, naphthyl, heteroaryl, phenyl(C₁-C₃)alkyl, phenoxymethyl, naphthyl(C₁-C₃)alkyl, and heteroaryl(C₁-C₃)alkyl, each optionally substituted with 1 to 3 groups independently selected from: halogen (fluorine, chlorine, bromine, and iodine), cyano, nitro, amino, hydroxy, carboxy, (C₁-C₆)alkyl, (C₃-C₆)cycloalkyl, (C₄-C₇)cycloalkylalkyl, (C₂-C₆)alkenyl, halo(C₂-C₆)alkenyl, (C₃-C₆)cycloalkylalkenyl, (C₂-C₆)alkynyl, halo(C₂-C₆)alkynyl, (C₃-C₆)cycloalkyl-(C₂-C₄)alkynyl, halo(C₃-C₇)cycloalkylalkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkythio, (C₄-C₇)cycloalkylalkylthio, halo(C₁-C₆)alkylthio, halo(C₃-C₆)cycloalkythio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)-cycloalkylalkanesulfonyl, (C₁-C₆)alkylamino, di(C₁-C₆)alkylamino, (C₁-C₆)-alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkythio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkyl-alkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkane-sulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylamino-carbonyl(C₁-C₆)alkyl, di(C₁-C₈)alkylaminocarbonyl(C₁-C₆)alkyl and (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylamino-carboxy(C₁-C₆)alkyl and di(C₁-C₈)alkylaminocarboxy(C₁-C₆)alkyl, phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl, wherein the aromatic and heteroaromatic groups are optionally substituted with 1 to 3 groups independently selected from fluorine, chlorine, cyano, (C₁-C₃)alkyl, halo(C₁-C₃)alkyl, (C₁-C₃)alkoxy, halo(C₁-C₃)-alkoxy, (C₁-C₃)alkanesulfonyl, and (C₁-C₃)alkoxycarbonyl;

R^{e} is a) (C₁-C₁₂)alkyl, (C₄-C₁₂)cycloalkylalkyl, halo(C₁-C₁₂)alkyl, halo(C₄-C₁₂)cycloalkylalkyl, (C₂-C₁₂)alkenyl, (C₅-C₁₂)cycloalkylalkenyl, halo(C₂-C₁₂)alkenyl, halo(C₅-C₁₂)cycloalkylalkenyl, (C₂-C₁₂)alkynyl, (C₅-C₁₂)cycloalkylalkynyl, halo(C₂-C₁₂)alkynyl, halo(C₅-C₁₂)cycloalkylalkynyl, (C₁-C₆)alkoxy(C₁-C₆)alkyl, halo(C₁-C₆)alkoxy(C₁-C₆)alkyl, (C₁-C₆)alkylthio(C₁-C₆)alkyl, halo(C₁-C₆)alkylthio(C₁-C₆)alkyl, (C₁-C₆)alkanesulfinyl(C₁-C₆)alkyl, halo(C₁-C₆)alkane-sulfinyl(C₁-C₆)alkyl, (C₁-C₆)alkanesulfonyl(C₁-C₆)alkyl, halo(C₁-C₆)alkanesulfonyl(C₁-C₆)alkyl, aminocarbonyl(C₁-C₆)alkyl, (C₁-C₆)alkylaminocarbonyl(C₁-C₆)alkyl, di(C₁-C₆)alkylamino-carbonyl(C₁-C₆)alkyl, cyano(C₁-C₆)alkyl, carboxy(C₁-C₆)alkyl, (C₁-C₆)alkoxycarbonyl(C₁-C₆)alkyl, saturated heterocyclyl, or saturated heterocyclyl(C₁-C₆)alkyl or b) phenyl, naphthyl, heteroaryl, phenyl(C₁-C₃)alkyl, naphthyl(C₁-C₃)alkyl, or heteroaryl(C₁-C₃)alkyl, each of a) and b) is optionally substituted by 1 to 3 groups independently selected from: 1) fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl-(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkythio, (C₄-C₇)cycloalkylalkylthio, halo(C₁-C₆)alkylthio, halo(C₃-C₆)cycloalkythio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkythio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkyl-alkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkane-sulfonyl(C₁-C₆)alkyl, (C₃-C₈)cycloalkanesulfonyl(C₁-C₆)alkyl, (C₄-C₈) cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylamino-carbonyl(C₁-C₆)alkyl, di(C₁-C₈)alkylaminocarbonyl(C₁-C₆)alkyl (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylamino-carboxy(C₁-C₆)alkyl and di(C₁-C₈)alkylaminocarboxy(C₁-C₆)alkyl; or 2) phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl, each optionally substituted with 1 to 3 groups independently selected from fluorine, chlorine, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)-alkoxycarbonyl; or

b) R^{e} is a saturated divalent radical composed of carbon atoms, and 0, 1 or 2 hetero atoms selected from 0 or 1 nitrogen atoms, 0 or 1 oxygen atoms, and 0 or 1 sulfur atoms that is attached to any core carbon atom on L to form a saturated 3-, 4-, 5-, 6-, or 7-membered L-G ring; said L-G ring being optionally substituted with 1 to 4 groups selected from halogen, fluorine, (C₁-C₈)alkyl, halo(C₁-C₈)alkyl, (C₃-C₈)cycloalkyl, halo(C₃-C₈)cycloalkyl, hydroxy(C₃-C₈)cycloalkyl, (C₃-C₈)cycloalkyl(C₁-C₃)alkyl, halo(C₃-C₈)cycloalkyl(C₁-C₃)alkyl, hydroxy(C₃-C₈)cycloalkyl(C₁-C₃)alkyl, (C₁-C₈)alkoxy, halo(C₁-C₈)alkoxy, (C₃-C₈)cycloalkoxy, halo(C₃-C₈)cycloalkoxy, hydroxy(C₃-C₈)cycloalkoxy, (C₁-C₈)alkoxy(C₁-C₃)alkyl, halo(C₁-C₈)alkoxy(C₁-C₃)alkyl, (C₃-C₈)cycloalkoxy(C₁-C₃)alkyl, halo(C₃-C₈)cycloalkoxy(C₁-C₃)alkyl, hydroxy(C₃-C₈)cycloalkoxy(C₁-C₃)alkyl, (C₃-C₈)cycloalkyl(C₁-C₃)alkoxy(C₁-C₃)alkyl, halo(C₃-C₈)cycloalkyl(C₁-C₃)alkoxy(C₁-C₃)alkyl, hydroxy(C₃-C₈)cycloalkyl(C₁-C₃)alkoxy(C₁-C₃)alkyl, (C₁-C₈)alkylthio, halo(C₁-C₈)alkylthio, (C₃-C₈)cycloalkylthio, halo(C₃-C₈)cycloalkylthio, hydroxy(C₃-C₈)cycloalkylthio, (C₃-C₈)cycloalkyl(C₁-C₃)alkylthio, halo(C₃-C₈)cycloalkyl(C₁-C₃)alkylthio, hydroxy(C₃-C₈)cycloalkyl(C₁-C₃)alkylthio, (C₁-C₈)alkylthio(C₁-C₃)alkyl, halo(C₁-C₈)alkylthio(C₁-C₃)alkyl, (C₃-C₈)cycloalkylthio(C₁-C₃)alkyl, halo(C₃-C₈)cycloalkylthio(C₁-C₃)alkyl, hydroxy(C₃-C₈)cycloalkylthio(C₁-C₃)alkyl, (C₃-C₈)cycloalkyl(C₁-C₃)alkylthio(C₁-C₃)alkyl, halo(C₃-C₈)cycloalkyl(C₁-C₃)alkylthio(C₁-C₃)alkyl, hydroxy(C₃-C₈)cycloalkyl(C₁-C₃)alkylthio(C₁-C₃)alkyl, heterocyclyl, and oxo;

R^{f} is (C₁-C₆)alkyl or halo(C₁-C₆)alkyl;

or an enantiomer, diastereomer, or a pharmaceutically acceptable salt thereof;
provided that:

A is not 2,4-morpholine or 1,3-piperidine

when

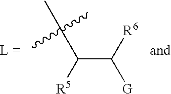

R² is —NHC(═NR¹²)(NH₂), —NHC(═NR¹²)(NHR⁹),

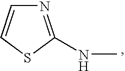

—OC(O)(NH₂), —OC(S)(NH₂), —SC(S)(NH₂), —SC(O)(NH₂), —OC(O)(NHR⁹), —OC(S)(NHR⁹), —SC(S)(NHR⁹), —SC(O)(NHR⁹), —NHC(O)OR⁹, —NHC(S)SR⁹, —NHC(S)OR⁹, —NHC(O)SR⁹, —C(O)R⁹, —C(S)R⁹, —C(O)(NH₂), —C(S)(NH₂), —C(O)(NHR⁹), —C(S)(NHR⁹) or —NHC(O)H, wherein R⁹ is a straight or branched C₁-C₅ alkyl, straight or branched C₁-C₅ haloalkyl, (C₃-C₄)cycloalkyl or straight or branched C₁-C₅alkoxyalkyl and R¹² is H, (C₁-C₆)alkyl, phenyl, heteroaryl, cyano, nitro, —S(O)R^{9,} —S(O₂)R⁹, —S(O₂)NHR⁹, —S(O₂)NR⁹R⁹, —C(O)R⁹, —C(S)R⁹, —C(O)OR⁹, —C(S)OR⁹, —C(O)(NH₂), —C(O)(NHR⁹).

In another embodiment, the present invention is directed to pharmaceutical compositions comprising a compound described herein or enantiomers, diastereomers, or salts thereof and a pharmaceutically acceptable carrier or excipient.

In another embodiment, the present invention is directed to a method of antagonizing aspartic protease inhibitors in a subject in need thereof comprising administering to the subject an effective amount of a compound described herein or an enantiomer, diastereomer, or salt thereof.

In another embodiment, the present invention is directed to method for treating or ameliorating an aspartic protease mediated disorder in a subject in need thereof comprising administering to said subject an effective amount of a compound described herein or an enantiomer, diastereomer, or salt thereof.

In another embodiment, the present invention is directed to a method for treating or ameliorating a renin mediated disorder in a subject in need thereof comprising administering to the subject an effective amount of a compound described herein or an enantiomer, diastereomer, or salt thereof.

In another embodiment, the present invention is directed to a method for the treatment of hypertension in a subject in need thereof comprising administering to the subject a compound described herein in combination therapy with one or more additional agents said additional agent selected from the group consisting of α-blockers, β-blockers, calcium channel blockers, diuretics, angiotensin converting enzyme (ACE) inhibitors, dual ACE and neutral endopeptidase (NEP) inhibitors, angiotensin-receptor blockers (ARBs), aldosterone synthase inhibitors, aldosterone-receptor antagonists, and endothelin receptor antagonists.

### DETAILED DESCRIPTION OF THE INVENTION

The invention is directed to aspartic protease inhibitor compounds represented by Structural Formula I or enantiomers, diastereomers or a pharmaceutically acceptable salts thereof (i.e., pharmaceutically acceptable salts of the compounds, enantiomers or diastereomers). Values and particular values for the variables in Structural Formula I are provided in the following paragraphs. It is understood that the invention encompasses all combinations of the substituent variables (i.e., R¹, R², R³, etc.) defined herein. For Structural Formula I:

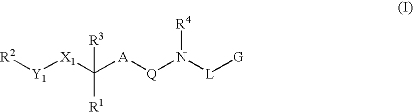

or an enantiomer, diastereomer or a pharmaceutically acceptable salt thereof:

In one embodiment, X₁ is a covalent bond, —O—, —S—, —S(O)—, —S(O)₂—. In a particular embodiment, X₁ is a covalent bond or —O—. In another particular embodiment, X₁ is —O—.

In one embodiment, Y₁ is a covalent bond or C₁-C₁₀ alkylene, C₁-C₁₀ alkenylene or C₁-C₁₀ alkynylene, each optionally substituted at one or more substitutable carbon atom with halogen, cyano, nitro, hydroxy, (C₁-C₃)alkyl, (C₁-C₃)alkoxy or halo(C₁-C₃)alkoxy, provided that Y₁ is a covalent bond only when X₁ is a covalent bond.

In a particular embodiment, Y₁ is a covalent bond. In another particular embodiment, Y₁ is a C₁-C₅ alkylene optionally substituted as described above. In another particular embodiment, Y₁ is a C₂-C₃ alkylene (e.g., —(CH₂)—ₘ; m=2 or 3) optionally substituted as described above. More particularly Y₁ is a C₂-C₃ alkylene when X₁ is O.

In one embodiment, A is a saturated or unsaturated 4-, 5-, 6-, or 7-membered ring which is optionally bridged by (CH₂)ₚ via bonds to two members of said ring, wherein said ring is composed of carbon atoms and 0-2 hetero atoms selected from the group consisting of 0, 1, or 2 nitrogen atoms, 0 or 1 oxygen atoms, and 0 or 1 sulfur atoms, said ring being optionally and independently substituted with zero to four halogen atoms, (C₁-C₆)alkyl groups, halo(C₁-C₆)alkyl groups or oxo groups such that when there is substitution with one oxo group on a carbon atom it forms a carbonyl group, and when there is substitution of one or two oxo groups on sulfur it forms sulfoxide or sulfone groups, respectively and p is 1 to 3, provided that A is not 2,4-morpholine or 1,3-piperidine

when

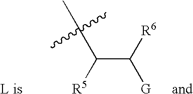

R² is —NHC(═NR¹²)(NH₂), —NHC(═NR¹²)(NHR⁹),

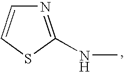

—OC(O)(NH₂), —OC(S)(NH₂), —SC(S)(NH₂), —SC(O)(NH₂), —OC(O)(NHR⁹), —OC(S)(NHR⁹), —SC(S)(NHR⁹), —SC(O)(NHR⁹), —NHC(O)OR⁹, —NHC(S)SR⁹, —NHC(S)OR⁹, —NHC(O)SR⁹, —C(O)R⁹, —C(S)R⁹, —C(O)(NH₂), —C(S)(NH₂), —C(O)(NHR⁹), —C(S)(NHR⁹) or —NHC(O)H, wherein R⁹ is a straight or branched C₁-C₅ alkyl, straight or branched C₁-C₅ haloalkyl, (C₃-C₄)cycloalkyl or straight or branched C₁-C₅alkoxyalkyl and R¹² is H, (C₁-C₆)alkyl, phenyl, heteroaryl, cyano, nitro, —S(O)R^{9,} —S(O₂)R⁹, —S(O₂)NHR⁹, —S(O₂)NR⁹R⁹, —C(O)R⁹, —C(S)R⁹, —C(O)OR⁹, —C(S)OR⁹, —C(O)(NH₂), —C(O)(NHR⁹).

In a particular embodiment, A is a saturated or unsaturated 4-, 5-, 6-, or 7-membered ring which is optionally bridged by (CH₂)ₚ via bonds to two members of said ring, wherein said ring is composed of carbon atoms, said ring being optionally and independently substituted with zero to four halogen atoms, (C₁-C₆)alkyl groups, halo(C₁-C₆)alkyl groups or oxo groups such that when there is substitution with one oxo group on a carbon atom it forms a carbonyl group and p is 1 to 3. In another particular embodiment of this invention, A is an optionally substituted phenyl or optionally substituted cyclohexyl. More particularly, A is phenyl or cyclohexyl. Most particularly, A is phenyl.

In one embodiment, R¹ is (C₃-C₇)cycloalkyl, phenyl, heteroaryl, or bicyclic heteroaryl each optionally substituted with 1 to 3 groups independently selected from: fluorine, chlorine, bromine, cyano, nitro, hydroxy, (C₁-C₆)alkyl, (C₃-C₆)cycloalkyl, (C₄-C₇)cycloalkylalkyl, (C₂-C₆)alkenyl, (C₅-C₇)cycloalkylalkenyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₅-C₇)-cycloalkylalkynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy and (C₁-C₆)alkanesulfonyl; and phenyl, heteroaryl, phenoxy, heteroaryloxy, phenylthio, heteroarylthio, benzyl, heteroarylmethyl, benzyloxy and heteroarylmethoxy, each optionally substituted with 1 to 3 groups independently selected from: fluorine, chlorine, bromine, cyano, nitro, hydroxy, (C₁-C₃)alkyl, halo(C₁-C₃)alkyl, (C₁-C₃)-alkoxy, and halo(C₁-C₃)alkoxy, and aminocarbonyl.

In a particular embodiment, R¹ is a phenyl, optionally substituted with (R¹¹)ₙ, wherein n is 0-3 and R¹¹ is independently selected from: fluorine, chlorine, bromine, cyano, nitro, hydroxy, (C₁-C₆)alkyl, (C₃-C₆)cycloalkyl, (C₄-C₇)cycloalkylalkyl, (C₂-C₆)alkenyl, (C₅-C₇)cycloalkylalkenyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₅-C₇)-cycloalkylalkynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy and (C₁-C₆)alkanesulfonyl; and phenyl, heteroaryl, phenoxy, heteroaryloxy, phenylthio, heteroarylthio, benzyl, heteroarylmethyl, benzyloxy and heteroarylmethoxy, each optionally substituted with 1 to 3 groups independently selected from: fluorine, chlorine, bromine, cyano, nitro, hydroxy, (C₁-C₃)alkyl, halo(C₁-C₃)alkyl, (C₁-C₃)-alkoxy, and halo(C₁-C₃)alkoxy, and aminocarbonyl.

In another particular embodiment, R¹ is phenyl optionally substituted with 1-3 groups independently selected from chloro, fluoro or methyl. In another particular embodiment, R¹ is phenyl substituted with chloro. In a most particular embodiment, R¹ is phenyl substituted with chloro at the carbon atom that is meta to the carbon atom that links phenyl to the rest of the molecule.

In one embodiment, R² is —NHC(═NR¹²)(NH₂), —NHC(═NR¹²)(NHR⁹),

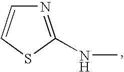

—OC(O)(NH₂), —OC(S)(NH₂), —SC(S)(NH₂), —SC(O)(NH₂), —OC(O)(NHR⁹), —OC(S)(NHR⁹), —SC(S)(NHR⁹), —SC(O)(NHR⁹), —NHC(O)OR⁹, —NHC(S)SR⁹, —NHC(S)OR⁹, —NHC(O)SR⁹, —C(O)R⁹, —C(S)R⁹, —C(O)(NH₂), —C(S)(NH₂), —C(O)(NHR⁹), —C(S)(NHR⁹) or —NHC(O)H.

In a particular embodiment, R² is —NHC(═NR¹²)(NH₂), —NHC(═NR¹²)(NHR⁹),

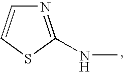

—OC(O)(NH₂), —OC(S)(NH₂), —OC(O)(NHR⁹), —OC(S)(NHR⁹), —NHC(O)OR⁹, —NHC(S)SR⁹, —NHC(S)OR⁹, —NHC(O)SR⁹, —C(O)R⁹, —C(S)R⁹, —C(O)(NH₂), —C(S)(NH₂), —C(O)(NHR⁹), —C(S)(NHR⁹) or —NHC(O)H.

In another particular embodiment, R² is —OC(O)(NHR⁹), —NHC(O)OR⁹, —C(O)R⁹, —C(O)(NHR⁹), or —NHC(O)H. In a more particular embodiment, R² is —OC(O)(NHR⁹), —NHC(O)OR⁹, —C(O)R⁹, —C(O)(NHR⁹), or —NHC(O)H and R⁹ is methyl or ethyl. In a more particular embodiment of this invention, R² is —NHC(O)OR⁹ and R⁹ is methyl or ethyl. In a most particular embodiment, R² is —NHC(O)OCH₃.

In one embodiment, R⁹ is a straight or branched C₁-C₅ alkyl, straight or branched C₁-C₅ haloalkyl, (C₃-C₄)cycloalkyl or straight or branched C₁-C₅ alkoxyalkyl and R¹² is H, (C₁-C₆)alkyl, phenyl, heteroaryl, cyano, nitro, —S(O)R^{9,} —S(O)₂R⁹, —S(O)₂NHR⁹, —S(O)₂NR⁹R⁹, —C(O)R⁹, —C(S)R⁹, —C(O)OR⁹, —C(S)OR⁹, —C(O)(NH₂), —C(O)(NHR⁹). In a particular embodiment of this invention, R⁹ is methyl or ethyl. In a most particular embodiment, R⁹ is methyl.

In one embodiment, R³ is —H, —F, C₁-C₅ alkyl, —NHC(O)R¹⁰, —OH or —OR¹⁰, wherein R¹⁰ is C₁-C₃ alkyl, provided that when R³ is —F or —OH, then X₁ is not —O—, —S—, —S(O)—, —S(O)₂— and R²—Y₁—X₁ is not —OC(O)(NH₂), —OC(S)(NH₂), —SC(S)(NH₂), —SC(O)(NH₂), —OC(O)(NHR⁹), —OC(S)(NHR⁹), —SC(S)(NHR⁹), —SC(O)(NHR⁹), —NHC(O)OR⁹, —NHC(S)OR⁹, —NHC(S)SR⁹, —NHC(O)SR⁹ or —NHC(O)H. In a particular embodiment of the invention, R³ is H.

In one embodiment, Q is Q1, Q2, Q3, Q4, Q5, or Q6:

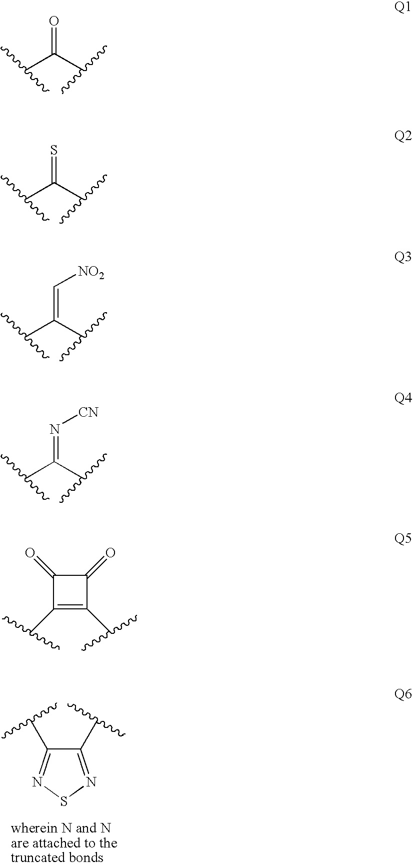

In a particular embodiment of the invention, Q is Q1: —C(O)—.

In one embodiment, R⁴ is H, (C₁-C₆)alkyl, halo(C₁-C₆)alkyl, (C₁-C₃)alkoxy(C₁-C₃)alkyl, or cyano(C₁-C₆)alkyl. In a particular embodiment of the invention, R⁴ is H.

In one embodiment, G is OH, OR^{e}, NH₂, NHR^{e}, NR^{e}R^{f}, C(═NH)NH₂, C(═NH)NHR^{e}, NHC(═NH)NH₂, or NHC(═NH)NHR^{e} and R^{e} and R^{f} are described below.

In a particular embodiment of this invention, G is OH, NH₂ or NHR^{e}. In a more particular embodiment, G is OH, NH₂ or NHR^{e} and R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl.

In another particular embodiment, G is NH₂ or NHR^{e}. In another particular embodiment of this invention, G is NHR^{e} and R^{e} is methyl or R⁵ and R^{e} together are —(CH₂)₃— optionally substituted with C₁-Cₐ alkyl or cyclohexyl. In a more particular embodiment, G is NH₂ or NHR^{e} and R^{e} is methyl.

In one embodiment of this invention, L is 1) a linear (C₂-C₄)alkyl chain when G is OH, OR^{e}, NH₂, NHR^{e}, NR^{e}R^{f}, NHC(═NH)NH₂, or NHC(═NH)NHR^{e}, or 2) a linear (C₁-C₃)alkyl chain when G is C(═NH)NH₂ or C(═NH)NHR^{e} and L is optionally substituted by 1-4 groups independently selected from R⁵, R^{5a}, R⁶, and R^{6a}; one or more of the carbon atoms of L may be part of a 3-, 4-, 5-, 6-, or 7-membered saturated ring composed of carbon atoms, and 0-2 hetero atoms selected from 0 or 1 nitrogen atoms, 0 or 1 oxygen atoms, and 0 or 1 sulfur atoms; said saturated ring being optionally substituted with up to four groups selected from halogen, (C₁-C₆)alkyl, halo(C₁-C₆)alkyl, (C₃-C₆)cycloalkyl, halo(C₃-C₆)cycloalkyl, (C₄-C₇)cycloalkylalkyl, halo(C₄-C₇)cycloalkylalkyl, and oxo, such that when there is substitution with one oxo group on a carbon atom it forms a carbonyl group and when there is substitution of one or two oxo groups on sulfur it forms sulfoxide or sulfone groups, respectively.

In another embodiment of this invention L is a C₂ alkyl chain, optionally substituted with R⁵ and R⁶.

In one embodiment of this invention, R⁵, R^{5a}, R⁶, and R^{6a} is each independently 1) H, (C₁-C₁₂)alkyl, halo(C₁-C₁₂)alkyl, hydroxy(C₁-C₁₂)alkyl, (C₃-C₁₀)cycloalkyl, (C₃-C₁₀)cycloalkyl, (C₃-C₁₀)cycloalkylalkyl, halo(C₃-C₁₀)cycloalkylalkyl, hydroxy(C₃-C₁₀)cycloalkylalkyl, (C₁-C₂)alkyl(C₃-C₁₀)cycloalkylalkyl, halo(C₁-C₂)alkyl(C₃-C₁₀)cycloalkylalkyl, di(C₁-C₂)alkyl (C₃-C₁₀)cycloalkylalkyl, hydroxy(C₁-C₂)alkyl(C₃-C₁₀)cycloalkylalkyl, hydroxy di(C₁-C₂)alkyl(C₃-C₁₀)cycloalkylalkyl, (C₂-C₁₂)alkenyl, (C₅-C₈)cycloalkyl(C₁-C₃)alkenyl, (C₂-C₁₂)alkynyl, (C₃-C₈)cycloalkyl(C₁-C₃)alkynyl, (C₄-C₁₂)bicycloalkyl(C₁-C₃)alkyl, (C₈-C₁₄)tricycloalkyl(C₁-C₃)alkyl, (C₁-C₆)alkoxy(C₁-C₆)alkyl, halo(C₁-C₆)alkoxy(C₁-C₆)alkyl, (C₃-C₈)cycloalkoxy(C₁-C₃)alkyl, (C₁-C₆)alkylthio(C₁-C₆)alkyl, halo(C₁-C₆)alkylthio(C₁-C₆)alkyl, (C₃-C₈)cycloalkylthio(C₁-C₃)alkyl, saturated heterocyclyl, and saturated heterocyclyl(C₁-C₃)alkyl wherein (a) hydrogen atoms in these groups are optionally substituted by 1 to 6 groups independently selected from halogen, cyano, nitro, hydroxy, (C₁-C₆)alkyl, (C₁-C₆)alkoxy, (C₃-C₆)cycloalkyl, (C₃-C₇)cycloalkylalkyl, halo(C₃-C₇)cycloalkylalkyl, (C₂-C₆)alkenyl, halo(C₂-C₆)alkenyl, (C₃-C₇)cycloalkylalkenyl, (C₂-C₆)alkynyl, halo(C₂-C₆)alkynyl, (C₃-C₇)cycloalkylalkoxy, halo(C₃-C₇)cycloalkylalkoxy, (C₃-C₇)cycloalkoxy, halo(C₁-C₆)alkyl, (C₃-C₇)cycloalkylalkynyl, halo(C₃-C₇)cycloalkylalkynyl, halo(C₁-C₆)alkoxy, halo(C₃-C₇)cycloalkyl, halo(C₃-C₇)cycloalkoxy, (C₁-C₆)alkylsulfonyl, aminocarbonyl and wherein (b) divalent sulfur atoms are optionally oxidized to sulfoxide or sulfone; or 2) phenyl, naphthyl, heteroaryl, phenyl(C₁-C₃)alkyl, phenoxymethyl, naphthyl(C₁-C₃)alkyl, and heteroaryl(C₁-C₃)alkyl, each optionally substituted with 1 to 3 groups independently selected from: halogen (fluorine, chlorine, bromine, and iodine), cyano, nitro, amino, hydroxy, carboxy, (C₁-C₆)alkyl, (C₃-C₆)cycloalkyl, (C₄-C₇)cycloalkyl alkyl, (C₂-C₆)alkenyl, halo(C₂-C₆)alkenyl, (C₃-C₆)cycloalkylalkenyl, (C₂-C₆)alkynyl, halo(C₂-C₆)alkynyl, (C₃-C₆)cycloalkyl-(C₂-C₄)alkynyl, halo(C₃-C₇)cycloalkylalkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkythio, (C₄-C₇)cycloalkylalkylthio, halo(C₁-C₆)alkylthio, halo(C₃-C₆)cycloalkythio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)-cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkythio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkyl-alkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkane-sulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylamino-carbonyl(C₁-C₆)alkyl, di(C₁-C₈)alkylaminocarbonyl(C₁-C₆)alkyl and (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylamino-carboxy(C₁-C₆)alkyl and di(C₁-C₈)alkylaminocarboxy(C₁-C₆)alkyl, phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl, wherein the aromatic and heteroaromatic groups are optionally substituted with 1 to 3 groups independently selected from fluorine, chlorine, cyano, (C₁-C₃)alkyl, halo(C₁-C₃)alkyl, (C₁-C₃)alkoxy, halo(C₁-C₃)-alkoxy, (C₁-C₃)alkanesulfonyl, and (C₁-C₃)alkoxycarbonyl.

In another particular embodiment, one of R⁵ and R⁶ is —H or methyl and the other is a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy.

In a more particular embodiment, R⁶ is —H or methyl and R⁵ is a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy.

In another more particular embodiment, R⁵ is —H or methyl and R⁶ is a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy.

In another particular embodiment, R⁵ is (C₁-C₇)alkyl, halo(C₁-C₇)alkyl, hydroxy(C₁-C₇)alkyl, cyclohexylmethyl, halocyclohexylmethyl, hydroxy cyclohexylmethyl, 2-(cyclohexyl)ethyl, (C₁-C₂)alkyl cyclohexylmethyl, di(C₁-C₂)alkyl cyclohexylmethyl, hydroxy(C₁-C₂)alkyl cyclohexylmethyl, hydroxy di(C₁-C₂)alkylcyclohexylmethyl, (3-noradamantyl)methyl, (tetrahydropyranyl)methyl, or oxepanyl methyl and R⁶ is —H or methyl.

In another particular embodiment, R⁶ is (C₁-C₇)alkyl, halo(C₁-C₇)alkyl, hydroxy(C₁-C₇)alkyl, cyclohexylmethyl, halocyclohexylmethyl, hydroxy cyclohexylmethyl, 2-(cyclohexyl)ethyl, (C₁-C₂)alkyl cyclohexylmethyl, di(C₁-C₂)alkyl cyclohexylmethyl, hydroxy(C₁-C₂)alkyl cyclohexylmethyl, hydroxy di(C₁-C₂)alkylcyclohexylmethyl, (3-noradamantyl)methyl, (tetrahydropyranyl)methyl, or oxepanyl methyl and R⁵ is —H or methyl.

In a more particular embodiment, R⁵ is cyclohexylmethyl, (tetrahydropyranyl)methyl, or oxepanyl methyl and R⁶ is —H. In another more particular embodiment of this invention, R⁶ is cyclohexylmethyl, (tetrahydropyranyl)methyl, or oxepanyl methyl and R⁵ is —H.

In one embodiment, R^{e} is a) (C₁-C₁₂)alkyl, (C₄-C₁₂)cycloalkylalkyl, halo(C₁-C₁₂)alkyl, halo(C₄-C₁₂)cycloalkylalkyl, (C₂-C₁₂)alkenyl, (C₅-C₁₂)cycloalkylalkenyl, halo(C₂-C₁₂)alkenyl, halo(C₅-C₁₂)cycloalkylalkenyl, (C₂-C₁₂)alkynyl, (C₅-C₁₂)cycloalkylalkynyl, halo(C₂-C₁₂)alkynyl, halo(C₅-C₁₂)cycloalkylalkynyl, (C₁-C₆)alkoxy(C₁-C₆)alkyl, halo(C₁-C₆)alkoxy(C₁-C₆)alkyl, (C₁-C₆)alkylthio(C₁-C₆)alkyl, halo(C₁-C₆)alkylthio(C₁-C₆)alkyl, (C₁-C₆)alkanesulfinyl(C₁-C₆)alkyl, halo(C₁-C₆)alkane-sulfinyl(C₁-C₆)alkyl, (C₁-C₆)alkanesulfonyl(C₁-C₆)alkyl, halo(C₁-C₆)alkanesulfonyl(C₁-C₆)alkyl, aminocarbonyl(C₁-C₆)alkyl, (C₁-C₆)alkylaminocarbonyl(C₁-C₆)alkyl, di(C₁-C₆)alkylamino-carbonyl(C₁-C₆)alkyl, cyano(C₁-C₆)alkyl, carboxy(C₁-C₆)alkyl, (C₁-C₆)alkoxycarbonyl(C₁-C₆)alkyl, saturated heterocyclyl, or saturated heterocyclyl(C₁-C₆)alkyl or b) phenyl, naphthyl, heteroaryl, phenyl(C₁-C₃)alkyl, naphthyl(C₁-C₃)alkyl, or heteroaryl(C₁-C₃)alkyl, each optionally substituted by 1 to 3 groups independently selected from: 1) fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl-(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkythio, (C₄-C₇)cycloalkylalkylthio, halo(C₁-C₆)alkylthio, halo(C₃-C₆)cycloalkythio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkane-sulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)-cycloalkylalkanesulfonyl, (C₁-C₆)alkylamino, di(C₁-C₆)alkylamino, (C₁-C₆)alkoxy-(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl and di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C -C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio(C₁-C₆)alkyl, (C₃-C₈)cycloalkythio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkylalkylthio(C₁-C₆)alkyl, (C₁-C₈)alkanesulfinyl(C₁-C₆)alkyl, (C₃-C₈)-cycloalkanesulfinyl(C₁-C₆)alkyl, (C₄-C₈)cycloalkyl-alkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkane-sulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkane-sulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl(C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylamino-carboxy(C₁-C₆)alkyl and di(C₁-C₈)alkylaminocarboxy(C₁-C₆)alkyl; or 2) phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl, each optionally substituted with 1 to 3 groups independently selected from fluorine, chlorine, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)-alkoxycarbonyl; or b) R^{e} is a saturated divalent radical composed of carbon atoms, and 0, 1 or 2 hetero atoms selected from 0 or 1 nitrogen atoms, 0 or 1 oxygen atoms, and 0 or 1 sulfur atoms that is attached to any core carbon atom on L to form a saturated 3-, 4-, 5-, 6-, or 7-membered L-G ring; said L-G ring being optionally substituted with 1 to 4 groups selected from halogen, fluorine, (C₁-C₈)alkyl, halo(C₁-C₈)alkyl, (C₃-C₈)cycloalkyl, halo(C₃-C₈)cycloalkyl, hydroxy(C₃-C₈)cycloalkyl, (C₃-C₈)cycloalkyl(C₁-C₃)alkyl, halo(C₃-C₈)cycloalkyl(C₁-C₃)alkyl, hydroxy(C₃-C₈)cycloalkyl(C₁-C₃)alkyl, (C₁-C₈)alkoxy, halo(C₁-C₈)alkoxy, (C₃-C₈)cycloalkoxy, halo(C₃-C₈)cycloalkoxy, hydroxy(C₃-C₈)cycloalkoxy, (C₁-C₈)alkoxy(C₁-C₃)alkyl, halo(C₁-C₈)alkoxy(C₁-C₃)alkyl, (C₃-C₈)cycloalkoxy(C₁-C₃)alkyl, halo(C₃-C₈)cycloalkoxy(C₁-C₃)alkyl, hydroxy(C₃-C₈)cycloalkoxy(C₁-C₃)alkyl, (C₃-C₈)cycloalkyl(C₁-C₃)alkoxy(C₁-C₃)alkyl, halo(C₃-C₈)cycloalkyl(C₁-C₃)alkoxy(C₁-C₃)alkyl, hydroxy(C₃-C₈)cycloalkyl(C₁-C₃)alkoxy(C₁-C₃)alkyl, (C₁-C₈)alkylthio, halo(C₁-C₈)alkylthio, (C₃-C₈)cycloalkylthio, halo(C₃-C₈)cycloalkylthio, hydroxy(C₃-C₈)cycloalkylthio, (C₃-C₈)cycloalkyl(C₁-C₃)alkylthio, halo(C₃-C₈)cycloalkyl(C₁-C₃)alkylthio, hydroxy(C₃-C₈)cycloalkyl(C₁-C₃)alkylthio, (C₁-C₈)alkylthio(C₁-C₃)alkyl, halo(C₁-C₈)alkylthio(C₁-C₃)alkyl, (C₃-C₈)cycloalkylthio(C₁-C₃)alkyl, halo(C₃-C₈)cycloalkylthio(C₁-C₃)alkyl, hydroxy(C₃-C₈)cycloalkylthio(C₁-C₃)alkyl, (C₃-C₈)cycloalkyl(C₁-C₃)alkylthio(C₁-C₃)alkyl, halo(C₃-C₈)cycloalkyl(C₁-C₃)alkylthio(C₁-C₃)alkyl, hydroxy(C₃-C₈)cycloalkyl(C₁-C₃)alkylthio(C₁-C₃)alkyl, heterocyclyl, and oxo.

In another particular embodiment of this invention, R^{e} is a) (C₁-C₆)alkyl, halo(C₁-C₆)alkyl, (Cₐ-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl.

In another particular embodiment, R^{e} is methyl or R⁵ and R^{e} together are —(CH₂)₃— optionally substituted with C₁-C₄ alkyl or cyclohexyl. In a more particular embodiment of this invention, R^{e} is methyl or R⁶ and R^{e} together are —(CH₂)₃— optionally substituted with C₁-C₄ alkyl or cyclohexyl. In another more particular embodiment of this invention, R^{e} is methyl or R⁵ and R^{e} together are —(CH₂)₃— optionally substituted with C₁-C₄ alkyl or cyclohexyl. In a most particular embodiment of this invention, R^{e} is methyl.

In one embodiment of this invention, R^{f} is (C₁-C₆)alkyl or halo(C₁-C₆)alkyl.

In a first specific embodiment, the aspartic protease inhibitor of the invention is represented by Structural Formulae (Ia) or (Ib) or a pharmaceutically acceptable salt of the aspartic protease inhibitor represented by Structural Formula (Ia) or Structural Formula (Ib):

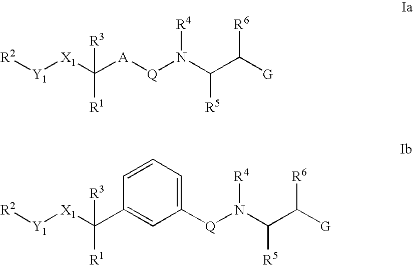

Values and particular values for the variables in Structural Formulas (Ia) and (Ib) are as provided for Structural Formula (I) above.

A first set of values for Structural Formulas (Ia) and (Ib) is as provided in the following paragraphs:

R² is —NHC(═NR¹²)(NH₂), —NHC(═NR¹²)(NHR⁹),

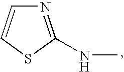

—OC(O)(NH₂), —OC(S)(NH₂), —OC(O)(NHR⁹), —OC(S)(NHR⁹), —NHC(O)OR⁹, —NHC(S)SR⁹, —NHC(S)OR⁹, —NHC(O)SR⁹, —C(O)R⁹, —C(S)R⁹, —C(O)(NH₂), —C(S)(NH₂), —C(O)(NHR⁹), —C(S)(NHR⁹) or —NHC(O)H, and R⁹ is a straight or branched C₁-C₅ alkyl, straight or branched C₁-C₅ haloalkyl, (C₃-C₄)cycloalkyl or straight or branched C₁-C₅ alkoxyalkyl and R¹² is H, (C₁-C₆)alkyl, phenyl, heteroaryl, cyano, nitro, —S(O)R^{9,} —S(O₂)R⁹, —S(O₂)NHR⁹, —S(O₂)NR⁹R⁹, —C(O)R⁹, —C(S)R⁹, —C(O)OR⁹, —C(S)OR⁹, —C(O)(NH₂), —C(O)(NHR⁹); and

the remainder of the values and particular values for Structural Formulas (Ia) and (Ib) are as described for Structural Formula (I).

A second set of values for Structural Formulas (Ia) and (Ib) are as provided in the following paragraphs:

R² is —OC(O)(NHR⁹), —NHC(O)OR⁹, —C(O)R⁹, —C(O)(NHR⁹), or —NHC(O)H;

R⁹ is methyl or ethyl; and

the remainder of the values and particular values for Structural Formulas (Ia) and (Ib) are as described for Structural Formula (I).

A third set of values for Structural Formulas (Ia) and (Ib) are as provided in the following paragraphs:

R² is —NHC(O)OR⁹;

R⁹ is methyl or ethyl; and

the remainder of the values and particular values for Structural Formulas (Ia) and (Ib) are as described for Structural Formula (I).

A fourth set of values for Structural Formulas (Ia) and (Ib) are as provided in the following paragraphs:

R² is —NHC(O)OCH₃; and

the remainder of the values and particular values for Structural Formulas (Ia) and (Ib) are as described for Structural Formula (I).

A fifth set of values for Structural Formulas (Ia) and (Ib) are as provided in the following paragraphs:

R² is —NHC(═NR¹²)(NH₂), —NHC(═NR¹²)(NHR⁹),

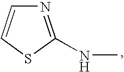

—OC(O)(NH₂), —OC(S)(NH₂), —OC(O)(NHR⁹), —OC(S)(NHR⁹), —NHC(O)OR⁹, —NHC(S)SR⁹, —NHC(S)OR⁹, —NHC(O)SR⁹, —C(O)R⁹, —C(S)R⁹, —C(O)(NH₂), —C(S)(NH₂), —C(O)(NHR⁹), —C(S)(NHR⁹) or —NHC(O)H and R⁹ is a straight or branched C₁-C₅ alkyl, straight or branched C₁-C₅ haloalkyl, (C₃-C₄)cycloalkyl or straight or branched C₁-C₅ alkoxyalkyl and R¹² is H, (C₁-C₆)alkyl, phenyl, heteroaryl, cyano, nitro, —S(O)R^{9,} —S(O₂)R⁹, —S(O₂)NHR⁹, —S(O₂)NR⁹R⁹, —C(O)R⁹, —C(S)R⁹, —C(O)OR⁹, —C(S)OR⁹, —C(O)(NH₂), —C(O)(NHR⁹);

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl;

and the remainder of the values and particular values for Structural Formulas (Ia) and (Ib) are as described for Structural Formula (I).

A sixth set of values for Structural Formulas (Ia) are as provided in the following paragraphs:

A is a saturated or unsaturated 4-, 5-, 6-, or 7-membered ring which is optionally bridged by (CH₂)ₚ via bonds to two members of said ring, wherein said ring is composed of carbon atoms, said ring being optionally and independently substituted with zero to four halogen atoms, (C₁-C₆)alkyl groups, halo(C₁-C₆)alkyl groups or oxo groups such that when there is substitution with one oxo group on a carbon atom it forms a carbonyl group;

p is 1 to 3;

R² is —NHC(═NR¹²)(NH₂), —NHC(═NR¹²)(NHR⁹),

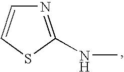

—OC(O)(NH₂), —OC(S)(NH₂), —OC(O)(NHR⁹), —OC(S)(NHR⁹), —NHC(O)OR⁹, —NHC(S)SR⁹, —NHC(S)OR⁹, —NHC(O)SR⁹, —C(O)R⁹, —C(S)R⁹, —C(O)(NH₂), —C(S)(NH₂), —C(O)(NHR⁹), —C(S)(NHR⁹) or —NHC(O)H, and R⁹ is a straight or branched C₁-C₅ alkyl, straight or branched C₁-C₅ haloalkyl, (C₃-C₄)cycloalkyl or straight or branched C₁-C₅ alkoxyalkyl and R¹² is H, (C₁-C₆)alkyl, phenyl, heteroaryl, cyano, nitro, —S(O)R^{9,} —S(O₂)R⁹, —S(O₂)NHR⁹, —S(O₂)NR⁹R⁹, —C(O)R⁹, —C(S)R⁹, —C(O)OR⁹, —C(S)OR⁹, —C(O)(NH₂), —C(O)(NHR⁹); and

the remainder of the values and particular values for Structural Formulas (Ia) and (Ib) are as described for Structural Formula (I).

In a second specific embodiment, the aspartic protease inhibitor of the invention is represented by Structural Formula II or Structural Formula (IIa), or a pharmaceutically acceptable salt of the aspartic protease inhibitor represented by Structural Formula (II) or (IIa):

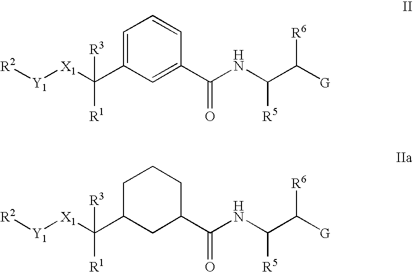

Values and particular values for the variables in Structural Formula (II) and Structural Formula (IIa) are as provided for Structural Formula (I) above.

A first set of values for Structural Formulas (II) and (IIa) is as provided in the following paragraphs:

one of R⁵ and R⁶ is —H or methyl and the other is as described for Structural Formula (I); and

the remainder of the values and particular values for Structural Formula (II) and (IIa) are as described for Structural Formula (I).

A second set of values for Structural Formulas (II) and (IIa) is as provided in the following paragraphs:

R⁶ is —H or methyl;

and the remainder of the values and particular values for Structural Formulas (II) and (IIa) are as described for Structural Formula (I).

A third set of values for Structural Formulas (II) and (IIa) is as provided in the following paragraphs:

R⁵ is —H or methyl; and

the remainder of the values and particular values for Structural Formulas (II) and (IIa) are as described for Structural Formula (I).

A fourth set of values for Structural Formulas (II) and (IIa) is as provided in the following paragraphs:

one of R⁵ and R⁶ is H or methyl and the other is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy;

and the remainder of the values and particular values for Structural Formulas (II) and (IIa) are as described for Structural Formula (I).

A fifth set of values for Structural Formulas (II) and (IIa) is as provided in the following paragraphs:

R⁶ is H or methyl and R⁵ is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl , (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy;

and the remainder of the values and particular values for Structural Formulas (II) and (IIa) are as described for Structural Formula (I).

A sixth set of values for Structural Formulas (II) and (IIa) is as provided in the following paragraphs:

R⁵ is H or methyl and R⁶ is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy;

and the remainder of the values and particular values for Structural Formulas (II) and (IIa) are as described for Structural Formula (I).

A seventh set of values for Structural Formulas (II) and (IIa) is as provided in the following paragraphs:

R² is —NHC(═NR¹²)(NH₂), —NHC(═NR¹²)(NHR⁹),

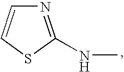

—OC(O)(NH₂), —OC(S)(NH₂), —OC(O)(NHR⁹), —OC(S)(NHR⁹), —NHC(O)OR⁹, —NHC(S)SR⁹, —NHC(S)OR⁹, —NHC(O)SR⁹, —C(O)R⁹, —C(S)R⁹, —C(O)(NH₂), —C(S)(NH₂), —C(O)(NHR⁹), —C(S)(NHR⁹) or —NHC(O)H and R⁹ is a straight or branched C₁-C₅ alkyl, straight or branched C₁-C₅ haloalkyl, (C₃-C₄)cycloalkyl or straight or branched C₁-C₅alkoxyalkyl and R¹² is H, (C₁-C₆)alkyl, phenyl, heteroaryl, cyano, nitro, —S(O)R^{9,} —S(O₂)R⁹, —S(O₂)NHR⁹, —S(O₂)NR⁹R⁹, —C(O)R⁹, —C(S)R⁹, —C(O)OR⁹, —C(S)OR⁹, —C(O)(NH₂), —C(O)(NHR⁹);

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl;

and the remainder of the values and particular values for Structural Formulas (II) and (IIa) are as described for Structural Formula (I).

A eighth set of values for Structural Formulas (II) and (IIa) is as provided in the following paragraphs:

one of R⁵ and R⁶ is —H or methyl and the other is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy;and

the remainder of the values and particular values for Structural Formulas (II) and (IIa) are as described for seventh set of values for Structural Formulas (II) and (IIa).

A ninth set of values for Structural Formulas (II) and (IIa) is as provided in the following paragraphs:

R⁵ is —H or methyl and R⁶ is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy; and

the remainder of the values and particular values for Structural Formulas (II) and (IIa) are as described for seventh set of values for Structural Formulas (II) and (IIa).

A tenth set of values for Structural Formulas (II) and (IIa) is as provided in the following paragraphs:

R⁶ is —H or methyl and R⁵ is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy;and

the remainder of the values and particular values for Structural Formulas (II) and (IIa) are as described for seventh set of values for Structural Formulas (II) and (IIa).

In a third specific embodiment, the aspartic protease inhibitor of the invention is represented Structural Formulas (III)-(VII), or an enantiomer, diastereomer or a pharmaceutically acceptable salt thereof:

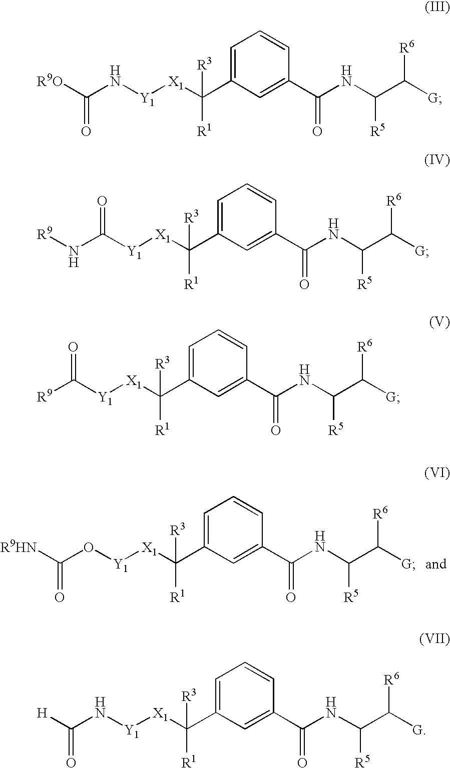

Values and particular values for the variables in Structural Formula (III)-(VII) are as provided for Structural Formula (I) above.

A first set of values for Structural Formulas (III)-(VII) is described in the following paragraphs:

one of R⁵ and R⁶ is —H or methyl and the other is as described for Structural Formula (I); and

the remainder of the values and particular values for Structural Formula (III)-(VII) are as described for Structural Formula (I).

A second set of values for Structural Formulas (III)-(VII) is described in the following paragraphs:

R⁶ is —H or methyl; and

the remainder of the values and particular values for Structural Formulas (III)-(VII) are as described for Structural Formula (I).

A third set of values for Structural Formulas (III)-(VII) is described in the following paragraphs:

R⁵ is —H or methyl; and

the remainder of the values and particular values for Structural Formulas (III)-(VII) are as described for Structural Formula (I).

A fourth set of values for Structural Formulas (III)-(VII) is described in the following paragraphs:

one of R⁵ and R⁶ is H or methyl and the other is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy; and

the remainder of the values and particular values for Structural Formulas (III)-(VII) are as described for Structural Formula (I).

A fifth set of values for Structural Formulas (III)-(VII) is described in the following paragraphs:

R⁶ is H or methyl and R⁵ is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy; and

the remainder of the values and particular values for Structural Formulas (III)-(VII) are as described for Structural Formula (I).

A sixth set of values for Structural Formulas (III)-(VII) is described in the following paragraphs:

R⁵ is H or methyl and R⁶ is selected from a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₁-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy; and

the remainder of the values and particular values for Structural Formulas (III)-(VII) are as described for Structural Formula (I).

A seventh set of values for Structural Formulas (III)-(VII) is described in the following paragraphs:

R² is —NHC(═NR¹²)(NH₂), —NHC(═NR¹²)(NHR⁹),

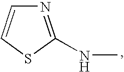

—OC(O)(NH₂), —OC(S)(NH₂), —OC(O)(NHR⁹), —OC(S)(NHR⁹), —NHC(O)OR⁹, —NHC(S)SR⁹, —NHC(S)OR⁹, —NHC(O)SR⁹, —C(O)R⁹, —C(S)R⁹, —C(O)(NH₂), —C(S)(NH₂), —C(O)(NHR⁹), —C(S)(NHR⁹) or —NHC(O)H and R⁹ is a straight or branched C₁-C₅ alkyl, straight or branched C₁-C₅ haloalkyl, (C₃-C₄)cycloalkyl or straight or branched C₁-C₅ alkoxyalkyl and R¹² is H, (C₁-C₆)alkyl, phenyl, heteroaryl, cyano, nitro, —S(O)R^{9,} —S(O₂)R⁹, —S(O₂)NHR⁹, —S(O₂)NR⁹R⁹, —C(O)R⁹, —C(S)R⁹, —C(O)OR⁹, —C(S)OR⁹, —C(O)(NH₂), —C(O)(NHR⁹);

G is OH, NH₂ or NHR^{e};

R^{e} is selected from a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl; and

the remainder of the values and particular values for Structural Formulas (III)-(VII) are as described for the fourth set of values for Structural Formulas (III)-(VII) and for Structural Formula (I).

A eighth set of values for Structural Formulas (III)-(VII) is described in the following paragraphs:

R² is —NHC(═NR¹²)(NH₂), —NHC(═NR¹²)(NHR⁹),

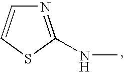

—OC(O)(NH₂), —OC(S)(NH₂), —OC(O)(NHR⁹), —OC(S)(NHR⁹), —NHC(O)OR⁹, —NHC(S)SR⁹, —NHC(S)OR⁹, —NHC(O)SR⁹, —C(O)R⁹, —C(S)R⁹, —C(O)(NH₂), —C(S)(NH₂), —C(O)(NHR⁹), —C(S)(NHR⁹) or —NHC(O)H and R⁹ is a straight or branched C₁-C₅ alkyl, straight or branched C₁-C₅ haloalkyl, (C₃-C₄)cycloalkyl or straight or branched C₁-C₅ alkoxyalkyl and R¹² is H, (C₁-C₆)alkyl, phenyl, heteroaryl, cyano, nitro, —S(O)R^{9,} —S(O₂)R⁹, —S(O₂)NHR⁹, —S(O₂)NR⁹R⁹, —C(O)R⁹, —C(S)R⁹, —C(O)OR⁹, —C(S)OR⁹, —C(O)(NH₂), —C(O)(NHR⁹);

G is OH, NH₂ or NHR^{e};

R^{e} is selected from a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C -C₂)alkyl, (C₁-C₈)alkoxy, halo(C₁-C₈)alkoxy, (C₃-C₆)cycloalkoxy, halo(C₃-C₆)cycloalkoxy, and heterocyclyl; and

the remainder of the values and particular values for Structural Formulas (III)-(VII) are as described for the fifth set of values for Structural Formulas (III)-(VII) and for Structural Formula (I).

A ninth set of values for Structural Formulas (III)-(VII) is described in the following paragraphs:

R² is —NHC(═NR¹²)(NH₂), —NHC(═NR¹²)(NHR⁹),

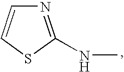

—OC(O)(NH₂), —OC(S)(NH₂), —OC(O)(NHR⁹), —OC(S)(NHR⁹), —NHC(O)OR⁹, —NHC(S)SR⁹, —NHC(S)OR⁹, —NHC(O)SR⁹, —C(O)R⁹, —C(S)R⁹, —C(O)(NH₂), —C(S)(NH₂), —C(O)(NHR⁹), —C(S)(NHR⁹) or —NHC(O)H and R⁹ is a straight or branched C₁-C₅ alkyl, straight or branched C₁-C₅ haloalkyl, (C₃-C₄)cycloalkyl or straight or branched C₁-C₅ alkoxyalkyl and R¹² is H, (C₁-C₆)alkyl, phenyl, heteroaryl, cyano, nitro, —S(O)R^{9,} —S(O₂)R⁹, —S(O₂)NHR⁹, —S(O₂)NR⁹R⁹, —C(O)R⁹, —C(S)R⁹, —C(O)OR⁹, —C(S)OR⁹, —C(O)(NH₂), —C(O)(NHR⁹);

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl; and

the remainder of the values and particular values for Structural Formulas (III)-(VII) are as described for the sixth set of values for Structural Formulas (III)-(VII) and for Structural Formula (I).

In a fourth specific embodiment, the aspartic protease inhibitor of the invention is represented by a structural formula selected from Structural Formulas (IIIa)-(VIIa), or an enantiomer, diastereomer or a pharmaceutically acceptable salt thereof:

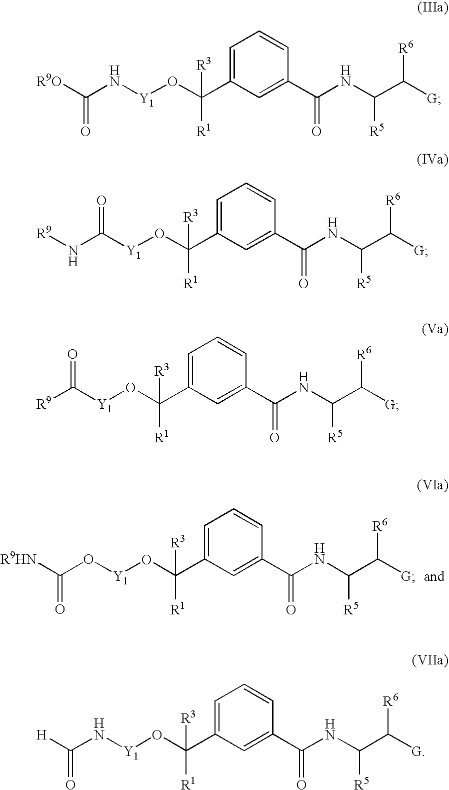

Values and particular values for the variables in Structural Formulas (IIIa)-(VIIa) are as provided for Structural Formula (I) above.

Alternatively, values and particular values for the variables in Structural Formulas (IIIa)-(VIIa) are as described for the first set of values for Structural Formulas (III)-(VII). In another alternative, values and particular values for the variables in Structural Formulas (IIIa)-(VIIa) are as described for the second set of values for Structural Formulas (III)-(VII). In another alternative, values and particular values for the variables in Structural Formulas (IIIa)-(VIIa) are as described for the third set of values for Structural Formulas (III)-(VII). In another alternative, values and particular values for the variables in Structural Formulas (IIIa)-(VIIa) are as described for the fourth set of values for Structural Formulas (III)-(VII). In another alternative, values and particular values for the variables in Structural Formulas (IIIa)-(VIIa) are as described for the fifth set of values for Structural Formulas (III)-(VII). In another alternative, values and particular values for the variables in Structural Formulas (IIIa)-(VIIa) are as described for the sixth set of values for Structural Formulas (III)-(VII). In another alternative, values and particular values for the variables in Structural Formulas (IIIa)-(VIIa) are as described for the seventh set of values for Structural Formulas (III)-(VII). In another alternative, values and particular values for the variables in Structural Formulas (IIIa)-(VIIa) are as described for the eighth set of values for Structural Formulas (III)-(VII). In another alternative, values and particular values for the variables in Structural Formulas (IIIa)-(VIIa) are as described for the ninth set of values for Structural Formulas (III)-(VII).

In a fifth specific embodiment, the aspartic protease inhibitor of the invention is represented by Structural Formulas (VIII)-(XII), or an enantiomer, diastereomer or a pharmaceutically acceptable salt thereof:

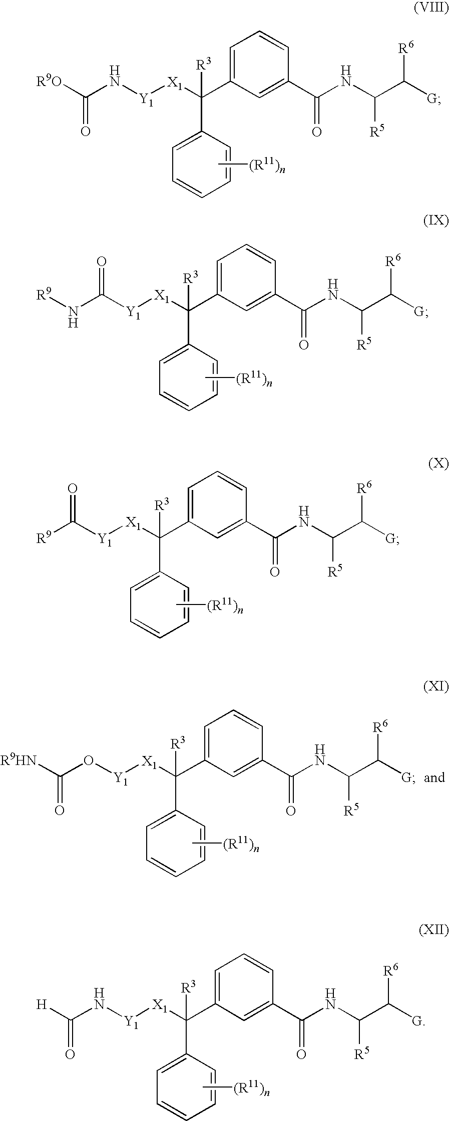

Values and particular values for the variables in Structural Formulas (VIII)-(XII) are as described for the first set of values for Structural Formulas (III)-(VII). Alternatively, values and particular values for the variables in Structural Formulas (VIII)-(XII) are as described for the second set of values for Structural Formulas (III)-(VII). In another alternative, values and particular values for the variables in Structural Formulas (VIII)-(XII) are as described for the third set of values for Structural Formulas (III)-(VII). In yet another alternative, values and particular values for the variables in Structural Formulas (VIII)-(XII) are as described for the fourth set of values for Structural Formulas (III)-(VII). In yet another alternative, values and particular values for the variables in Structural Formulas (VIII)-(XII) are as described for the fifth set of values for Structural Formulas (III)-(VII). In yet another alternative, values and particular values for the variables in Structural Formulas (VIII)-(XII) are as described for the sixth set of values for Structural Formulas (III)-(VII).

In a sixth specific embodiment, the aspartic protease inhibitor of the invention is represented by a structural formula selected from Structural Formulas (XIII)-(XVII), or an enantiomer, diastereomer or a pharmaceutically acceptable salt thereof:

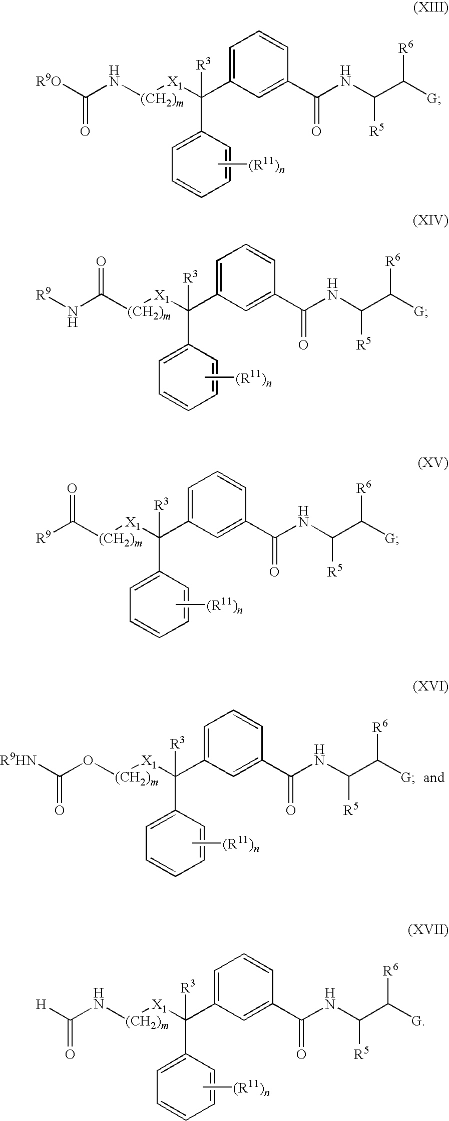

Values and particular values for the variables in Structural Formulas (XIII)-(XVII) are as provided for Structural Formula (I) above.

A first set of values for the aspartic protease inhibitor represented by Structural Formulas (XIII)-(XVII) is provided in the following paragraphs:

one of R⁵ and R⁶ is H or methyl and the other is a) H, (C₁-C₁₀)alkyl, (C₄-C₁₀)cycloalkylalkyl, halo(C₁-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy;

R¹¹ is fluorine, chlorine, bromine, cyano, nitro, (C₁-C₆)alkyl, (C₃-C₆)cycloalkyl, (C₄-C₇)cycloalkylalkyl, (C₂-C₆)alkenyl, (C₅-C₇)cycloalkylalkenyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₅-C₇)-cycloalkylalkynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy and (C₁-C₆)alkanesulfonyl; or 2) phenyl, heteroaryl, phenoxy, heteroaryloxy, phenylthio, heteroarylthio, benzyl, heteroarylmethyl, benzyloxy and heteroarylmethoxy, each optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy, and aminocarbonyl;

n is 0, 1, 2 or 3;

m is 2 or 3; and

values and particular values for the remainder of the variables in Structural Formulas (XIII)-(XVII) are as described for Structural Formula (I).

A second set of values for the aspartic protease inhibitor represented by Structural Formulas (XIII)-(XVII) is provided in the following paragraphs:
R² is —NHC(═NR¹²)(NH₂), —NHC(═NR¹²)(NHR⁹),

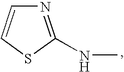

—OC(O)(NH₂), —OC(S)(NH₂), —OC(O)(NHR⁹), —OC(S)(NHR⁹), —NHC(S)SR⁹, —NHC(S)OR⁹, —NHC(O)SR⁹, —C(O)R⁹, —C(S)_{R}⁹, —C(O)(NH₂), —C(S)(NH₂), —C(O)(NHR⁹), —C(S)(NHR⁹) or —NHC(O)H and R⁹ is a straight or branched C₁-C₅ alkyl, straight or branched C₁-C₅ haloalkyl, (C₃-C₄)cycloalkyl or straight or branched C₁-C₅alkoxyalkyl and R¹² is H, (C₁-C₆)alkyl, phenyl, heteroaryl, cyano, nitro, —S(O)R^{9,} —S(O₂)R⁹, —S(O₂)NHR⁹, —S(O₂)NR⁹R⁹, —C(O)R⁹, —C(S)R⁹, —C(O)OR⁹, —C(S)OR⁹, —C(O)(NH₂), —C(O)(NHR⁹);

G is OH, NH₂ or NHR^{e};

R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl; and

the remainder of the values and particular values for Structural Formulas (XIII)-(XVII) are as described for the first set of values for Structural Formulas (XIII)-(XVII).

A third set of values for the aspartic protease inhibitor represented by Structural Formulas (XIII)-(XVII) is provided in the following paragraphs:

R⁵ is (C₁-C₇)alkyl, halo(C₁-C₇)alkyl, hydroxy(C₁-C₇)alkyl, cyclohexylmethyl, halocyclohexylmethyl, hydroxy cyclohexylmethyl, (C₁-C₂)alkyl cyclohexylmethyl, di(C₁-C₂)alkyl cyclohexylmethyl, hydroxy(C₁-C₂)alkyl cyclohexylmethyl, hydroxy di(C₁-C₂)alkylcyclohexylmethyl, (3-noradamantyl)methyl, (tetrahydropyranyl)methyl or oxepanylmethyl;

R⁶ is H or methyl

G is NH₂ or NHR^{e};

R^{e} is methyl or R⁵ and R^{e} together are —(CH₂)₃— optionally substituted with C₁-C₄ alkyl or cyclohexyl; and

values and particular values for the remainder of the variables are as described for the second set of values for Structural Formulas (XIII)-(XVII).

A fourth set of values for the aspartic protease inhibitor represented by Structural Formulas (XIII)-(XVII) is provided in the following paragraphs:

R⁶ is (C₁-C₇)alkyl, halo(C₁-C₇)alkyl, hydroxy(C₁-C₇)alkyl, cyclohexylmethyl, halocyclohexylmethyl, hydroxy cyclohexylmethyl, (C₁-C₂)alkyl cyclohexylmethyl, di(C₁-C₂)alkyl cyclohexylmethyl, hydroxy(C₁-C₂)alkyl cyclohexylmethyl, hydroxy di(C₁-C₂)alkylcyclohexylmethyl, (3-noradamantyl)methyl, (tetrahydropyranyl)methyl or oxepanylmethyl;

R⁵ is H or methyl

G is NH₂ or NHR^{e};

R^{e} is methyl or R⁶ and R^{e} together are —(CH₂)₃— optionally substituted with C₁-C₄ alkyl or cyclohexyl; and

values and particular values for the remainder of the variables are as described for the second set of values for Structural Formulas (XIII)-(XVII).

A fifth set of values for the aspartic protease inhibitor represented by Structural Formula (XIII)-(XVII) is provided in the following paragraphs:

R⁹ is methyl or ethyl;

R¹¹ is chloro, fluoro or methyl; and

values and particular values for the remainder of the variables are as described for the third set of values for Structural Formulas (XIII)-(XVII).

A sixth set of values for the aspartic protease inhibitor represented by Structural Formula (XIII)-(XVII) is provided in the following paragraphs:

R⁹ is methyl or ethyl;

R¹¹ is chloro, fluoro or methyl; and

values and particular values for the remainder of the variables are as described for the fourth set of values for Structural Formulas (XIII)-(XVII).

In seventh specific embodiment, the aspartic protease inhibitor of the invention is represented by a structural formula selected from Structural Formulas (XVIII)-(XXII), or an enantiomer, diastereomer or a pharmaceutically acceptable salt thereof:

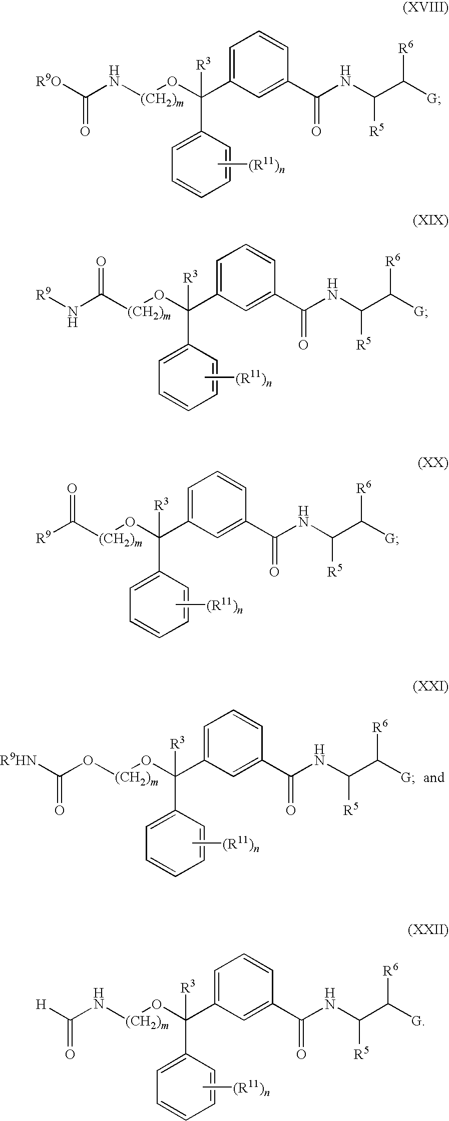

Values and particular values for the variables in Structural Formulas (XVIII)-(XXII) are as provided for Structural Formula (I) above.

Values and particular values for the variables in Structural Formulas (XVIII)-(XXII) are as described for the first set of values for Structural Formulas (XIII)-(XVII). Alternatively, values and particular values for the variables in Structural Formulas (XVIII)-(XXII) are as described for the second set of values for Structural Formulas (XIII)-(XVII). In another alternative, values and particular values for the variables in Structural Formulas (XVIII)-(XXII) are as described for the third set of values for Structural Formulas (XIII)-(XVII). In yet another alternative, values and particular values for the variables in Structural Formulas (XVIII)-(XXII) are as described for the fourth set of values for Structural Formulas (XIII)-(XVII). In yet another alternative, values and particular values for the variables in Structural Formulas (XVIII)-(XXII) are as described for the fifth set of values for Structural Formulas (XIII)-(XVII). In yet another alternative, values and particular values for the variables in Structural Formulas (XVIII)-(XXII) are as described for the sixth set of values for Structural Formulas (XIII)-(XVII).

In an eighth specific embodiment, the aspartic protease inhibitor of the invention is represented by a structural formula selected from Structural Formulas (XXIII)-(XXVII), or an enantiomer, diastereomer or a pharmaceutically acceptable salt thereof:

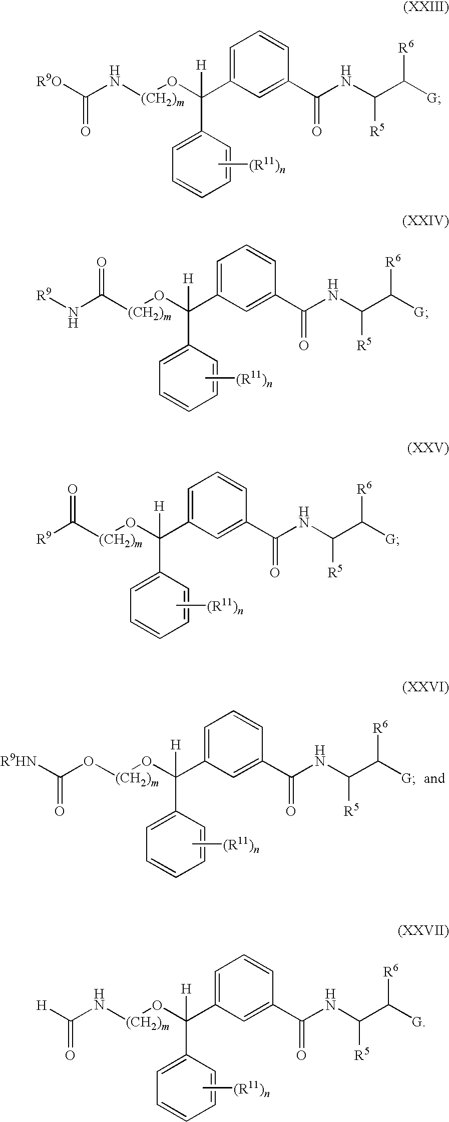

Values and particular values for the variables in Structural Formulas (XXIII)-(XXVII) are as provided for Structural Formula (I) above.

Values and particular values for the variables in Structural Formulas (XXIII)-(XXVII) are as described for the first set of values for Structural Formulas (XIII)-(XVII). Alternatively, values and particular values for the variables in Structural Formulas (XXIII)-(XXVII) are as described for the second set of values for Structural Formulas (XIII)-(XVII). In another alternative, values and particular values for the variables in Structural Formulas (XXIII)-(XXVII) are as described for the third set of values for Structural Formulas (XIII)-(XVII). In yet another alternative, values and particular values for the variables in Structural Formulas (XXIII)-(XXVII) are as described for the fourth set of values for Structural Formulas (XIII)-(XVII). In yet another alternative, values and particular values for the variables in Structural Formulas (XXIII)-(XXVII) are as described for the fifth set of values for Structural Formulas (XIII)-(XVII). In yet another alternative, values and particular values for the variables in Structural Formulas (XXIII)-(XXVII) are as described for the sixth set of values for Structural Formulas (XIII)-(XVII).

In a ninth specific embodiment, the aspartic protease inhibitor of the invention is represented by a structural formula selected from Structural Formula (XXVIII), or an enantiomer, diastereomer or a pharmaceutically acceptable salt thereof,

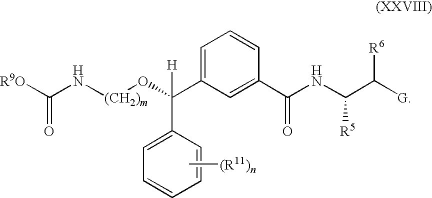

and at least one and preferably both stereocenters are as depicted.

Values and particular values for the variables in Structural Formula (XXVIII) are as provided for Structural Formula (I) above.

Values and particular values for the variables in Structural Formula (XXVIII) are as described for the first set of values for Structural Formulas (XIII)-(XVII). Alternatively, values and particular values for the variables in Structural Formula (XXVIII) are as described for the second set of values for Structural Formulas (XIII)-(XVII). In another alternative, values and particular values for the variables in Structural Formula (XXVIII) are as described for the third set of values for Structural Formulas (XIII)-(XVII). In yet another alternative, values and particular values for the variables in Structural Formula (XXVIII) are as described for the fourth set of values for Structural Formulas (XIII)-(XVII). In yet another alternative, values and particular values for the variables in Structural Formula (XXVIII) are as described for the fifth set of values for Structural Formulas (XIII)-(XVII). In yet another alternative, values and particular values for the variables in Structural Formula (XXVIII) are as described for the sixth set of values for Structural Formulas (XIII)-(XVII).

In a eleventh specific embodiment, the phenyl group (variable A of Formula (I)) in Formulas (Ib), (III), (IIIa), (IV), (IVa), (V), (Va), (VI), (VIa), (VII), (VIIa), (VIII-XXVIII) is a cyclohexyl group and the values and particular values are as defined for each of the Formulas (Ib), (III), (IIIa), (IV), (IVa), (V), (Va), (VI), (VIa), (VII), (VIIa), (VIII-XXVIII).

Another embodiment of the invention is each of the following compounds and their enantiomers, diastereomers, and salts:

TABLE 1

| No. | Name |
|---|---|
| I-1 | methyl 2-((3-chlorophenyl)(3-(1-(methylamino)-3-(tetrahydro-2H- |
| | pyran-4-yl)propan-2-ylcarbamoyl)phenyl)methoxy)ethylcarbamate |
| I-2 | methyl 2-((3-chlorophenyl)(3-(1-(methylamino)-3-(tetrahydro-2H- |
| | pyran-3-yl)propan-2-ylcarbamoyl)phenyl)methoxy)ethylcarbamate |
| I-3 | methyl 2-((3-chlorophenyl)(3-(1-cyclohexyl-3-(methylamino)- |
| | propan-2-ylcarbamoyl)phenyl)methoxy)ethylcarbamate |
| I-4 | methyl 2-((3-chlorophenyl)(3-(1-(methylamino)-3-(oxepan-3- |
| | yl)propan-2-ylcarbamoyl)phenyl)methoxy)ethylcarbamate |

or a diastereomer, enantiomer or salt thereof.

The following are compounds of the invention, especially their pharmaceutically acceptable salts:

TABLE 2

| No. | Structure | Name |
|---|---|---|
| I-1a | 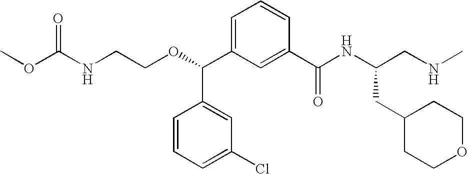 | methyl 2-((R)-(3-chlorophenyl)(3-((S)- 1-(methylamino)-3-(tetrahydro-2H- pyran-4-yl)propan-2- ylcarbamoyl)phenyl)methoxy)ethylcarba- mate |
| I-2a | 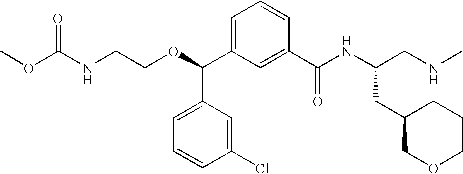 | methyl 2-((S)-(3-chlorophenyl)(3-((S)-1- (methylamino)-3-((R)-tetrahydro-2H- pyran-3-yl)propan-2- ylcarbamoyl)phenyl)methoxy)ethylcarba- mate |
| I-2b | 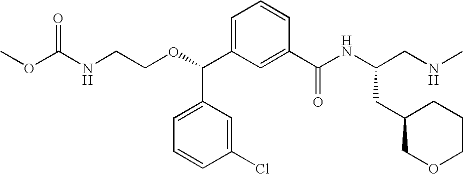 | methyl 2-((R)-(3-chlorophenyl)(3-((S)- 1-(methylamino)-3-((R)-tetrahydro-2H- pyran-3-yl)propan-2- ylcarbamoyl)phenyl)methoxy)ethylcarba- mate |
| I-3a | 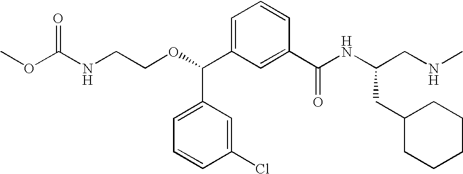 | methyl 2-((R)-(3-chlorophenyl)(3-((S)- 1-cyclohexyl-3-(methylamino)propan-2- ylcarbamoyl)phenyl)methoxy)ethylcarba- mate |
| I-3b | 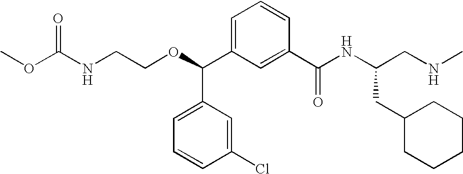 | methyl 2-((S)-(3-chlorophenyl)(3-((S)-1- cyclohexyl-3-(methylamino)propan-2- ylcarbamoyl)phenyl)methoxy)ethylcarba- mate |
| I-4a | 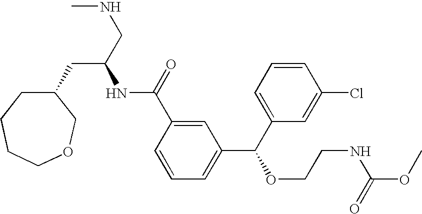 | methyl 2-((R)-(3-chlorophenyl)(3-((S)- 1-(methylamino)-3-((R)-oxepan-3- yl)propan-2- ylcarbamoyl)phenyl)methoxy)ethylcarba- mate |
| I-4b | 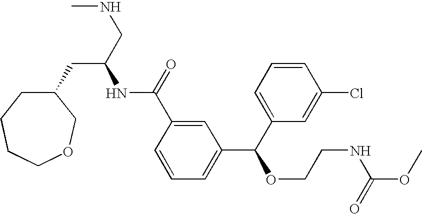 | methyl 2-((S)-(3-chlorophenyl)(3-((S)-1- (methylamino)-3-((R)-oxepan-3- yl)propan-2- ylcarbamoyl)phenyl)methoxy)ethylcarba- mate |
| I-5a | 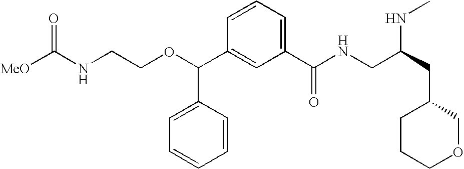 | methyl 2-((3-((S)-2-(methylamino)-3- ((R)-tetrahydro-2H-pyran-3- yl)propylcarbamoyl)phenyl)(phenyl)meth- oxy)ethylcarbamate |
| I-6a | 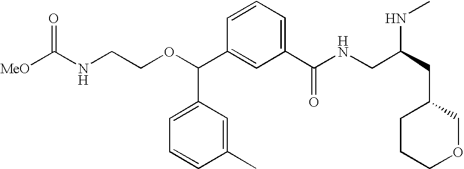 | methyl 2-((3-((S)-2-(methylamino)-3- ((R)-tetrahydro-2H-pyran-3- yl)propylcarbamoyl)phenyl)(m- tolyl)methoxy)ethylcarbamate |
| I-7a | 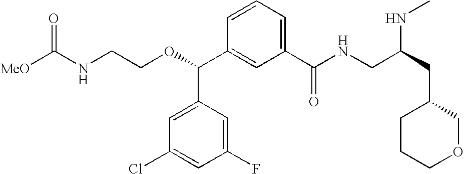 | methyl 2-((R)-(3-chloro-5- fluorophenyl)(3-((S)-2-(methylamino)-3- ((R)-tetrahydro-2H-pyran-3- yl)propylcarbamoyl)- phenyl)methoxy)ethylcarbamate |
| I-7b | 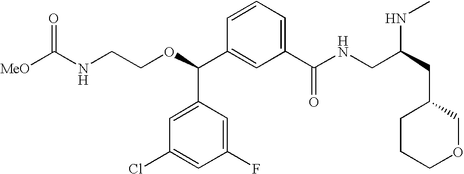 | methyl 2-((S)-(3-chloro-5- fluorophenyl)(3-((S)-2-(methylamino)-3- ((R)-tetrahydro-2H-pyran-3- yl)propylcarbmoyl)- phenyl)methoxy)ethylcarbamate |

or a diastereomer, enantiomer or salt thereof.

In another specific embodiment, the following are aspartic protease inhibitors of the present invention, or an enantiomer or diastereomer thereof Also included are pharmaceutically acceptable salts and solvates (e.g., hydrates) of the following compounds, or an enantiomer or diastereomer thereof.

TABLE 3

| Cpd | | |
|---|---|---|
| No. | Structure | Name |
| I*-1 | 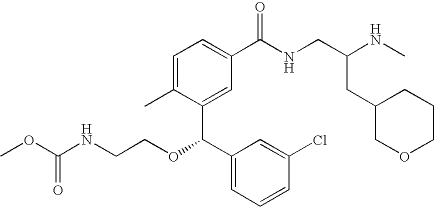 | methyl [2-({(3-chlorophenyl)[2- methyl-5-({[2-(methylamino)-3- (tetrahydro-2H-pyran-3- yl)propyl]amino}carbonyl)phenyl] methyl}oxy)ethyl]carbamate |
| I*-2 | 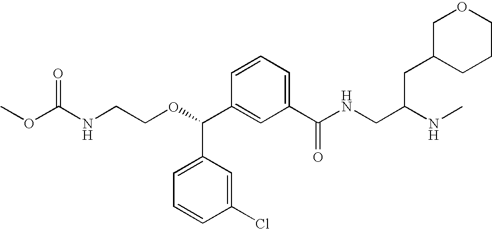 | methyl [2-({(3-chlorophenyl)[3-({[2- (methylamino)-3-(tetrahydro-2H- pyran-3-yl)propyl]amino} carbonyl)phenyl]methyl}oxy) ethyl]carbamate |
| I*-3 | 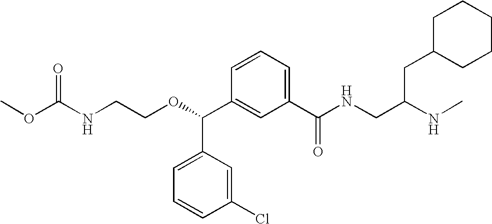 | methyl [2-({(3-chlorophenyl)[3-({[3- cyclohexyl-2- (methylamino)propyl]amino} carbonyl)phenyl]methyl}oxy) ethyl]carbamate |
| I*-4 | 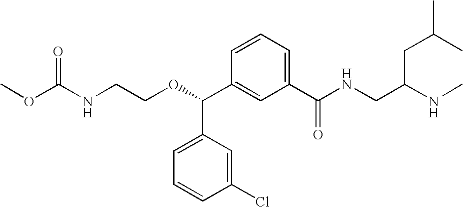 | methyl [2-({(3-chlorophenyl)[3-({[4- methyl-2-(methylamino) pentyl]amino}carbonyl)phenyl] methyl}oxy)ethyl]carbamate |
| I*-5 | 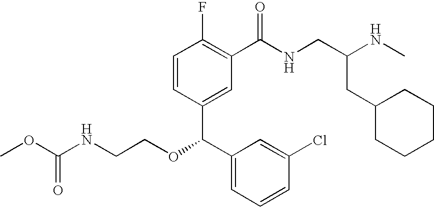 | methyl [2-({(3-chlorophenyl)[3-({[3- cyclohexyl-2-(methylamino)propyl] amino}carbonyl)-4-fluorophenyl] methyl}oxy)ethyl]carbamate |
| I*-6 | 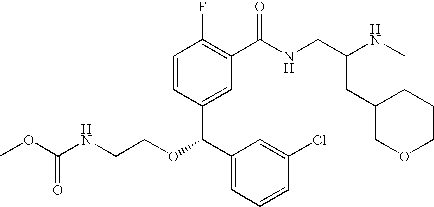 | methyl [2-({(3-chlorophenyl)[4- fluoro-3-({[2-(methylamino)-3- (tetrahydro-2H-pyran-3-yl)propyl] amino}carbonyl)phenyl]methyl}oxy) ethyl]carbamate |
| I*-7 | 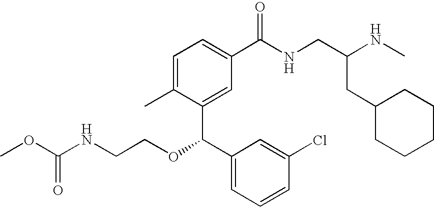 | methyl [2-({(3-chlorophenyl)[5-({[3- cyclohexyl-2- (methylamino)propyl]amino} carbonyl)-2-methylphenyl] methyl}oxy)ethyl]carbamate |
| I*-8 | 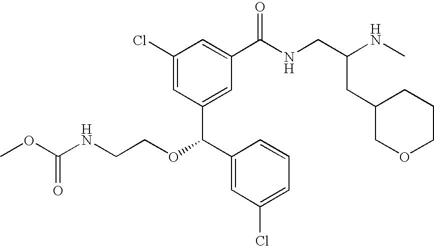 | methyl (2-{[[3-chloro-5-({[2- (methylamino)-3-(tetrahydro-2H- pyran-3-yl)propyl]amino} carbonyl)phenyl](3-chlorophenyl) methyl]oxy}ethyl)carbamate |
| I*-9 | 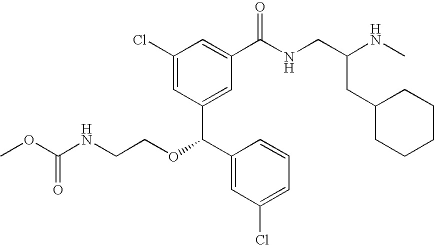 | methyl (2-{[[3-chloro-5-({[3- cyclohexyl-2-(methylamino) propyl]amino}carbonyl)phenyl](3- chlorophenyl)methyl]oxy} ethyl)carbamate |
| I*-10 | 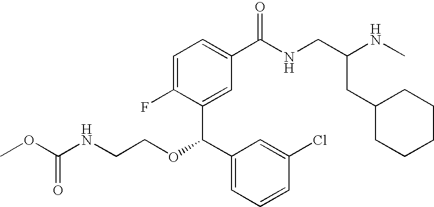 | methyl [2-({(3-chlorophenyl)[5-({[3- cyclohexyl-2-(methylamino)propyl] amino}carbonyl)-2-fluorophenyl] methyl}oxy)ethyl]carbamate |
| I*-11 | 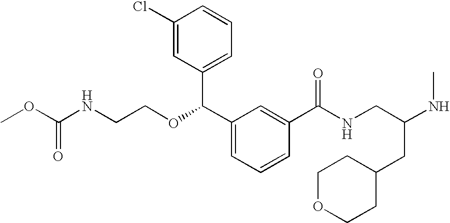 | methyl [2-({(3-chlorophenyl)[3-({[2- (methylamino)-3-(tetrahydro-2H- pyran-4-yl)propyl]amino} carbonyl)phenyl]methyl}oxy) ethyl]carbamate |
| I*-12 | 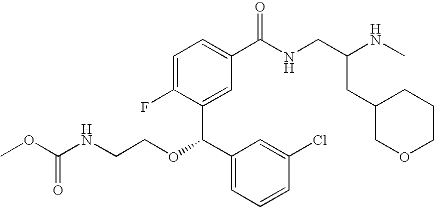 | methyl [2-({(3-chlorophenyl)[2- fluoro-5-({[2-(methylamino)-3- (tetrahydro-2H-pyran-3-yl)propyl] amino)carbonyl)phenyl]methyl}oxy) ethyl]carbamate |
| I*-13 | 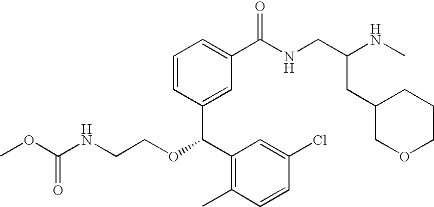 | methyl [2-({(5-chloro-2- methylphenyl)[3-({[2-(methylamino)- 3-(tetrahydro-2H-pyran-3- yl)propyl]amino} carbonyl)phenyl]methyl}oxy) ethyl]carbamate |

The following are selected aspartic protease inhibitors of this invention, and the pharmaceutically acceptable salts and solvates (e.g., hydrates) thereof.

TABLE 4

| Cpd | | |
|---|---|---|
| No.^{‡} | Structure | Name |
| I′-1a^{†} | 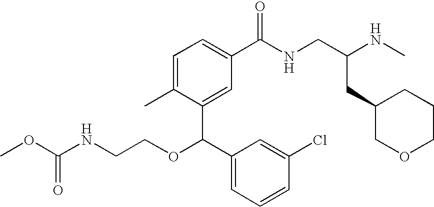 | methyl {2-[((3-chlorophenyl){2-methyl- 5-[({(2S)-2-(methylamino)-3-[(3R)- tetrahydro-2H-pyran-3-yl] propyl}amino)carbonyl]phenyl} methyl)oxy]ethyl}carbamate |
| I′-1b | 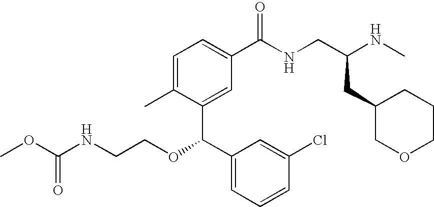 | methyl {2-[((S)-(3-chlorophenyl){2- methyl-5-[({(2S)-2-(methylamino)-3- [(3R)-tetrahydro-2H-pyran-3- yl]propyl}amino)carbonyl]phenyl} methyl)oxy]ethyl}carbamate |
| I′-2a^{†} | 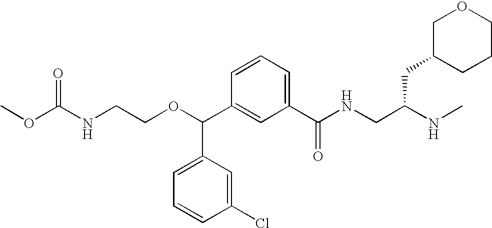 | methyl {2-[((3-chlorophenyl)(3-[({(2S)- 2-(methylamino)-3-[(3R)-tetrahydro- 2H-pyran-3-yl] propyl}amino)carbonyl]phenyl} methyl)oxy]ethyl}carbamate |
| I′-2b | 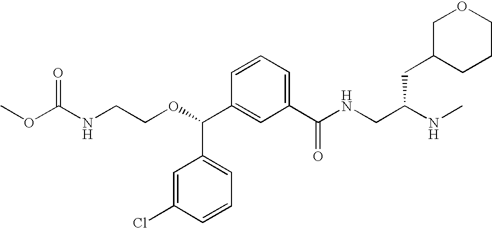 | methyl {2-[((R)-(3-chlorophenyl){3- [({(2S)-2-(methylamino)-3-[(3R)- tetrahydro-2H-pyran-3-yl]propyl} amino)carbonyl]phenyl} methyl)oxy]ethyl}carbamate |
| I′-2c | 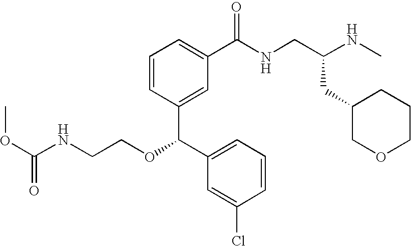 | methyl {2-[((R)-(3-chlorophenyl){3- [({(2R)-2-(methylamino)-3-[(3S)- tetrahydro-2H-pyran-3-yl] propyl}amino)carbonyl]phenyl} methyl)oxy]ethyl}carbamate |
| I′-2d | 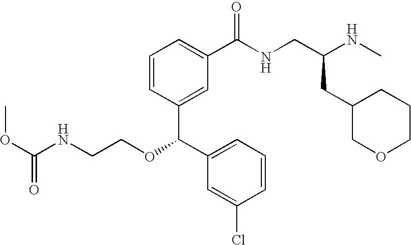 | methyl {2-[((R)-(3-chlorophenyl){3- [({(2S)-2-(methylamino)-3-[(3S)- tetrahydro-2H-pyran-3-yl] propyl}amino)carbonyl]phenyl} methyl)oxy]ethyl}carbamate |
| I′-2e | 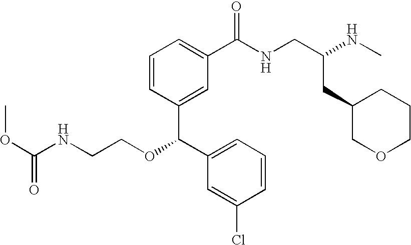 | methyl {2-[((R)-(3-chlorophenyl){3- [({(2R)-2-(methylamino)-3-[(3R)- tetrahydro-2H-pyran-3-yl] propyl}amino)carbonyl]phenyl} methyl)oxy]ethyl}carbamate |
| I′-3a | 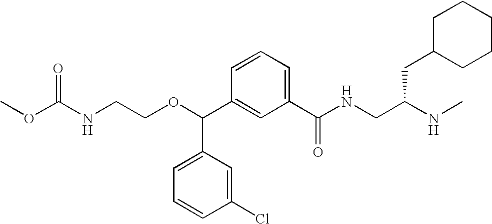 | methyl [2-({(3-chlorophenyl)[3-({[(2S)- 3-cyclohexyl-2- (methylamino)propyl]amino} carbonyl)phenyl]methyl}oxy)ethyl] carbamate |
| I′-4a | 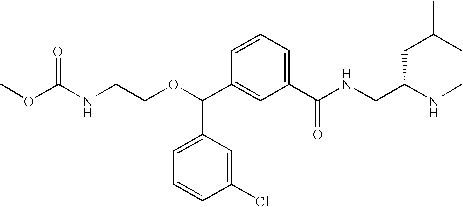 | methyl [2-({(3-chlorophenyl)[3-({[(2S)- 4-methyl-2-(methylamino) pentyl]amino}carbonyl)phenyl] methyl}oxy)ethyl]carbamate |
| I′-5a | 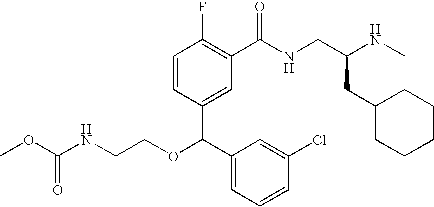 | methyl [2-({(3-chlorophenyl)[3-({[(2S)- 3-cyclohexyl-2- (methylamino)propyl]amino}carbonyl)- 4-fluorophenyl]methyl}oxy)ethyl] carbamate |
| I′-6a^{†} | 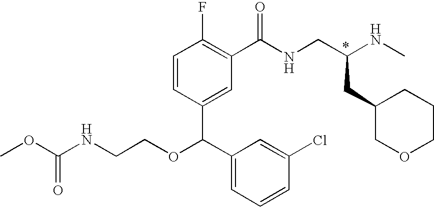 | methyl {2-[((3-chlorophenyl)(4-fluoro- 3-[({(2S)-2-(methylamino)-3-[(3R)- tetrahydro-2H-pyran-3-yl] propyl}amino)carbonyl]phenyl} methyl)oxy]ethyl}carbamate |
| I′-7a | 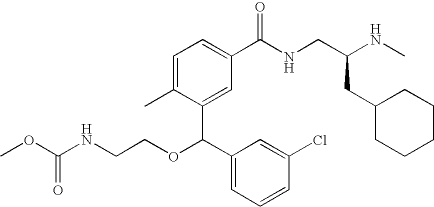 | methyl [2-({(3-chlorophenyl)[5-({[(2S)- 3-cyclohexyl-2- (methylamino)propyl]amino}carbonyl)- 2-methylphenyl]methyl}oxy)ethyl] carbamate |
| I′-8a | 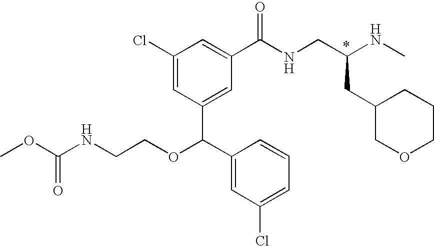 | methyl (2-[({3-chloro-5-[({(2S)-2- (methylamino)-3-[(3R)-tetrahydro-2H- pyran-3-yl]propyl}amino)carbonyl] phenyl}(3-chlorophenyl) methyl]oxy}ethyl)carbamate |
| I′-9a | 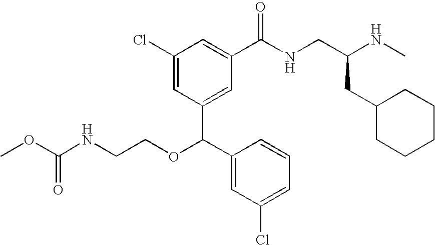 | methyl (2-{[[3-chloro-5-({[(2S)-3- cyclohexyl-2-(methylamino)propyl] amino}carbonyl)phenyl](3- chlorophenyl)methyl]oxy}ethyl) carbamate |
| I′-10a | 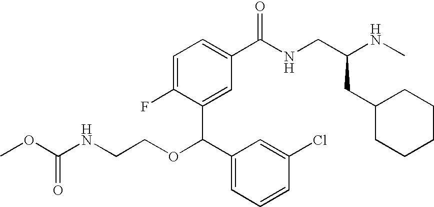 | methyl [2-({(3-chlorophenyl)[5-({[(2S)- 3-cyclohexyl-2- (methylamino)propyl]amino}carbonyl)- 2-fluorophenyl]methyl}oxy)ethyl] carbamate |
| I′-11a | 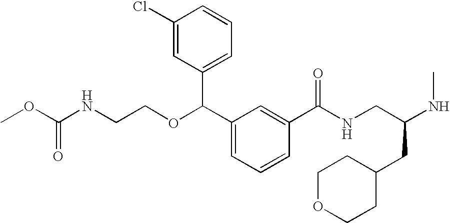 | methyl [2-({(3-chlorophenyl)[3-({[(2S)- 2-(methylamino)-3-(tetrahydro-2H- pyran-4- yl)propyl]amino}carbonyl)phenyl] methyl}oxy)ethyl]carbamate |
| I′-12a^{†} | 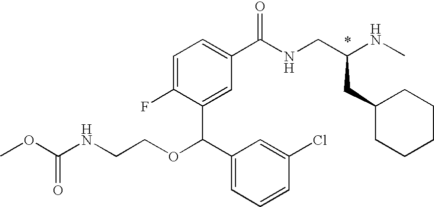 | methyl {2-[((3-chlorophenyl){2-fluoro- 5-[({(2S)-2-(methylamino)-3-[(3R)- tetrahydro-2H-pyran-3- yl]propyl}amino)carbonyl]phenyl} methyl)oxy]ethyl}carbamate |
| I′-13a | 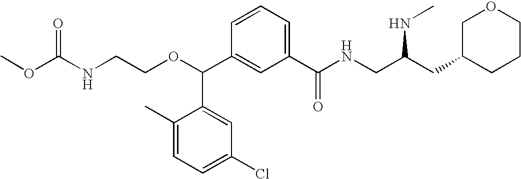 | methyl {2-[((5-chloro-2- methylphenyl){3-[({(2S)-2- (methylamino)-3-[(3R)-tetrahydro-2H- pyran-3-yl]propyl}amino)carbonyl] phenyl}methyl)oxy]ethyl}carbamate |
| I′-13b | 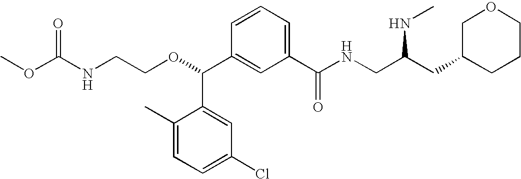 | methyl {2-[((R)-(5-chloro-2- methylphenyl){3-[({(2S)-2- (methylamino)-3-[(3R)-tetrahydro-2H- pyran-3-yl]propyl}amino) carbonyl]phenyl}methyl)oxy] ethyl}carbamate |

^{†}These compounds were prepared, isolated and evaluated as a 4:1 mixture of stereoisomers at the designated center (*).
^{‡}The above compounds were designated as “I′” as having substituent groups corresponding to compounds in the previous table (desiganted “I*”).

The following, including pharmaceutically acceptable salts thereof, are the preferred compounds: I-2b, I-3a, I-4a, and I-6a. The following, including pharmaceutically acceptable salts thereof are the more preferred compounds: I-4a and I-7a.

When any variable (e.g., aryl, heterocyclyl, R¹, R², etc.) occurs more than once in a compound, its definition on each occurrence is independent of any other occurrence.

“Alkyl” means a saturated aliphatic branched or straight-chain mono- or di-valent hydrocarbon radical having the specified number of carbon atoms. Thus, “(C₁-C₈)alkyl” means a radical having from 1-8 carbon atoms in a linear or branched arrangement. “(C₁-C₆)alkyl” includes methyl, ethyl, propyl, butyl, pentyl, and hexyl.

“Cycloalkyl” means a saturated aliphatic cyclic hydrocarbon radical having the specified number of carbon atoms. Thus, (C₃-C₇)cycloalkyl means a radical having from 3-7 carbon atoms arranged in a ring. (C₃-C₇)cycloalkyl includes cyclopropyl, cyclobutyl, cyclopentyl, cyclohexyl, and cycloheptyl.

Haloalkyl and halocycloalkyl include mono, poly, and perhaloalkyl groups where the halogens are independently selected from fluorine, chlorine, and bromine.

Saturated heterocyclic rings are 4-, 5-, 6-, and 7-membered heterocyclic rings containing 1 to 4 heteroatoms independently selected from N, O, and S, and include pyrrolidine, piperidine, tetrahydrofuran, tetrahydropyran, oxepane, tetrahydrothiophene, tetrahydrothiopyran, isoxazolidine, 1,3-dioxolane, 1,3-dithiolane, 1,3-dioxane, 1,4-dioxane, 1,3-dithiane, 1,4-dithiane, morpholine, thiomorpholine, thiomorpholine 1,1-dioxide, tetrahydro-2H-1,2-thiazine 1,1-dioxide, and isothiazolidine 1,1-dioxide. Oxo substituted saturated heterocyclic rings include tetrahydrothiophene 1-oxide, tetrahydrothiophene 1,1-dioxide, thiomorpholine 1-oxide, thiomorpholine 1,1-dioxide, tetrahydro-2H-1,2-thiazine 1,1-dioxide, and isothiazolidine 1,1-dioxide, pyrrolidin-2-one, piperidin-2-one, piperazin-2-one, and morpholin-2-one.

“Heteroaryl” means a monovalent heteroaromatic monocyclic or polycylic ring radical. Heteroaryl rings are 5- and 6-membered aromatic heterocyclic rings containing 1 to 4 heteroatoms independently selected from N, O, and S, and include furan, thiophene, pyrrole, imidazole, pyrazole, oxazole, isoxazole, thiazole, isothiazole, 1,2,3-triazole, 1,2,4-triazole, 1,3,4-oxadiazole, 1,2,5-thiadiazole, 1,2,5-thiadiazole 1-oxide, 1,2,5-thiadiazole 1,1-dioxide, 1,3,4-thiadiazole, pyridine, pyridine-N-oxide, pyrazine, pyrimidine, pyridazine, 1,2,4-triazine, 1,3,5-triazine, and tetrazole. Bicyclic heteroaryl rings are bicyclo[4.4.0] and bicyclo[4.3.0] fused ring systems containing 1 to 4 heteroatoms independently selected from N, O, and S, and include indolizine, indole, isoindole, benzo[b]furan, benzo[b]thiophene, indazole, benzimidazole, benzthiazole, purine, 4H-quinolizine, quinoline, isoquinoline, cinnoline, phthalazine, quinazoline, quinoxaline, 1,8-naphthyridine, and pteridine.

Bicycloalkyl rings are fused, bridged and Spiro ring systems and include bicyclo[1.1.0]butane, bicyclo[1.2.0]pentane, bicyclo[2.2.0]hexane, bicyclo[3.2.0]heptane, bicyclo[3.3.0]octane, bicyclo[4.2.0]octane, bicyclo[2.2.1]heptane, bicyclo[2.2.2]octane, bicyclo[3.2.1]octane, bicyclo[3.2.2]nonane, bicyclo[3.3.1]nonane, bicyclo[3.3.2]decane and bicyclo[3.3.3]undecane, spiro[2.2]pentane, spiro[2.3]hexane, spiro[3.3]heptane, spiro[2.4]heptane, spiro[3.4]octane, and spiro[2.5]octane.

Tricycloalkyl rings are fused, bridged and Spiro ring systems and include tricyclo[3.3.1.0^{3,7}]nonane (noradamantane) and tricyclo[3.3.1.1^{3,7}]decane (adamantane).

“Alkoxy” means an alkyl radical attached through an oxygen linking atom. “(C₁-C₄)-alkoxy” includes methoxy, ethoxy, propoxy, and butoxy.

“Aromatic” means an unsaturated cycloalkyl ring system.

“Aryl” means an aromatic monocyclic, or polycyclic ring system. Aryl systems include phenyl, naphthalenyl, fluorenyl, indenyl, azulenyl, and anthracenyl.

“Hetero” refers to the replacement of at least one carbon atom member in a ring system with at least one heteroatom selected from N, S, and O. A hetero ring may have 1, 2, 3, or 4 carbon atom members replaced by a heteroatom.

“Unsaturated ring” means a ring containing one or more double bonds and include cyclopentene, cyclohexene, cyclopheptene, cyclohexadiene, benzene, pyrroline, pyrazole, 4,5-dihydro-1H-imidazole, imidazole, 1,2,3,4-tetrahydropyridine, 1,2,3,6-tetrahydropyridine, pyridine and pyrimidine.

As used herein, 2,4-morpholine means:

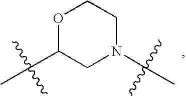

and 1,3-piperidine means

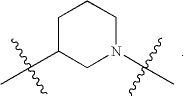

Certain compounds of Formula I may exist in various stereoisomeric or tautomeric forms. The invention encompasses all such forms, including active compounds in the form of essentially pure enantiomers, racemic mixtures, and tautomers, including forms those not depicted structurally.

The compounds of the invention may be present in the form of pharmaceutically acceptable salts. For use in medicines, the salts of the compounds of the invention refer to non-toxic “pharmaceutically acceptable salts.” Pharmaceutically acceptable salt forms include pharmaceutically acceptable acidic/anionic or basic/cationic salts.

Pharmaceutically acceptable acidic/anionic salts include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate, and triethiodide salts.

Salts of the disclosed compounds containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acid such as lysine and arginine.

When a disclosed compound or its pharmaceutically acceptable salt is named or depicted by structure, it is to be understood that solvates or hydrates of the compound or its pharmaceutically acceptable salts are also included. “Solvates” refer to crystalline forms wherein solvent molecules are incorporated into the crystal lattice during crystallization. Solvate may include water or nonaqueous solvents such as ethanol, isopropanol, DMSO, acetic acid, ethanolamine, and EtOAc. Solvates, wherein water is the solvent molecule incorporated into the crystal lattice, are typically referred to as “hydrates.” Hydrates include stoichiometric hydrates as well as compositions containing variable amounts of water.

When a disclosed compound or its pharmaceutically acceptable salt is named or depicted by structure, it is to be understood that the compound, including solvates thereof, may exist in crystalline forms, non-crystalline forms or a mixture thereof. The compound or its pharmaceutically acceptable salts or solvates may also exhibit polymorphism (i.e. the capacity to occur in different crystalline forms). These different crystalline forms are typically known as “polymorphs.” It is to be understood that when named or depicted by structure, the disclosed compound and its pharmaceutically acceptable salts, solvates or hydrates also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in solidifying the compound. For example, changes in temperature, pressure, or solvent may result in different polymorphs. In addition, one polymorph may spontaneously convert to another polymorph under certain conditions.

It may be necessary and/or desirable during synthesis to protect sensitive or reactive groups on any of the molecules concerned. Representative conventional protecting groups are described in T. W. Greene and P. G. M. Wuts “Protective Groups in Organic Synthesis” John Wiley & Sons, Inc., New York 1999. Protecting groups may be added and removed using methods well known in the art.

The invention also includes various isomers and mixtures thereof. “Isomer” refers to compounds that have the same composition and molecular weight but differ in physical and/or chemical properties. The structural difference may be in constitution (geometric isomers) or in the ability to rotate the plane of polarized light (stereoisomers).

Certain of the disclosed aspartic protease inhibitors may exist in various stereoisomeric forms. Stereoisomers are compounds which differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. The symbol “*” in a structural formula represents the presence of a chiral carbon center. “R” and “S” represent the configuration of substituents around one or more chiral carbon atoms. Thus, “R*” and “S*” denote the relative configurations of substituents around one or more chiral carbon atoms. When a chiral center is not defined as R or S, a mixture of both configurations is present.

“Racemate” or “racemic mixture” means a compound of equimolar quantities of two enantiomers, wherein such mixtures exhibit no optical activity; i.e., they do not rotate the plane of polarized light.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration.

Atoms (other than H) attached to a carbocyclic ring may be in a cis or trans configuration. In the “cis” configuration, the substituents are on the same side in relationship to the plane of the ring; in the “trans” configuration, the substituents are on opposite sides in relationship to the plane of the ring. A mixture of “cis” and “trans” species is designated “cis/trans”.

The point at which a group or moiety is attached to the remainder of the compound or another group or moiety can be indicated by [symbol] which represents [symbol] or “—”.

“R,” “S,” “S*,” “R*,” “E,” “Z,” “cis,” and “trans,” indicate configurations relative to the core molecule.

The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When a disclosed compound is named or depicted by structure without indicating the stereochemistry, and the inhibitor has at least one chiral center, it is to be understood that the name or structure encompasses one enantiomer of inhibitor free from the corresponding optical isomer, a racemic mixture of the inhibitor and mixtures enriched in one enantiomer relative to its corresponding optical isomer.

When a disclosed aspartic protease inhibitor is named or depicted by structure without indicating the stereochemistry and has at least two chiral centers, it is to be understood that the name or structure encompasses a diastereomer free of other diastereomers, a pair of diastereomers free from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s).

The compounds of the invention are useful for ameliorating or treating disorders or diseases in which decreasing the levels of aspartic protease products is effective in treating the disease state or in treating infections in which the infectious agent depends upon the activity of an aspartic protease. In hypertension elevated levels of angiotensin I, the product of renin catalyzed cleavage of angiotensinogen are present. Thus, the compounds of the invention can be used in the treatment of hypertension, heart failure such as (acute and chronic) congestive heart failure; left ventricular dysfunction; cardiac hypertrophy; cardiac fibrosis; cardiomyopathy (e.g., diabetic cardiac myopathy and post-infarction cardiac myopathy); supraventricular and ventricular arrhythmias; atrial fibrillation; atrial flutter; detrimental vascular remodeling; myocardial infarction and its sequelae; atherosclerosis; angina (whether unstable or stable); renal failure conditions, such as diabetic nephropathy; glomerulonephritis; renal fibrosis; scleroderma; glomerular sclerosis; microvascular complications, for example, diabetic retinopathy; renal vascular hypertension; vasculopathy; neuropathy; complications resulting from diabetes, including nephropathy, vasculopathy, retinopathy and neuropathy; diseases of the coronary vessels; proteinuria; albumenuria; post-surgical hypertension; metabolic syndrome; obesity; restenosis following angioplasty; eye diseases and associated abnormalities including raised intra-ocular pressure, glaucoma, retinopathy, abnormal vascular growth and remodelling; angiogenesis-related disorders, such as neovascular age related macular degeneration; hyperaldosteronism; anxiety states; and cognitive disorders (Fisher N. D.; Hollenberg N. K. Expert Opin. Investig. Drugs. 2001, 10, 417-26).

Elevated levels of βamyloid, the product of the activity of the well-characterized aspartic protease β-secretase (BACE) activity on amyloid precursor protein, are widely believed to be responsible for the development and progression of amyloid plaques in the brains of Alzheimer's disease patients. The secreted aspartic proteases of Candida albicans are associated with its pathogenic virulence (Naglik, J. R.; Challacombe, S. J.; Hube, B. Microbiology and Molecular Biology Reviews 2003, 67, 400-428). The viruses HIV and HTLV depend on their respective aspartic proteases for viral maturation. Plasmodium falciparum uses plasmepsins I and II to degrade hemoglobin.

A pharmaceutical composition of the invention may, alternatively or in addition to a compound of Formula I or any formula of the invention described herein, comprise a pharmaceutically acceptable salt of a compound of Formula I or a prodrug or pharmaceutically active metabolite of such a compound or salt and one or more pharmaceutically acceptable carriers therefor.

The compositions of the invention are aspartic protease inhibitors. Said compositions can contain compounds having a mean inhibition constant (IC₅₀) against aspartic proteases of between about 5,000 nM to about 0.01 nM; preferably between about 50 nM to about 0.01 nM; and more preferably between about 5 nM to about 0.01 nM.

The compositions of the invention can reduce blood pressure. Said compositions include compounds having an IC₅₀ for renin of between about 5,000 nM to about 0.01 nM; preferably between about 50 nM to about 0.01 nM; and more preferably between about 5 nM to about 0.01 nM.

The invention includes a therapeutic method for treating or ameliorating an aspartic protease mediated disorder in a subject in need thereof comprising administering to a subject in need thereof an effective amount of a compound of Formula I or any other formulas of the invention described herein, or the enantiomers, diastereomers, or salts thereof or composition thereof.

Administration methods include administering an effective amount (i.e., an effective amount) of a compound or composition of the invention at different times during the course of therapy or concurrently in a combination form. The methods of the invention include all known therapeutic treatment regimens.

“Prodrug” means a pharmaceutically acceptable form of an effective derivative of a compound (or a salt thereof) of the invention, wherein the prodrug may be: 1) a relatively active precursor which converts in vivo to a compound of the invention; 2) a relatively inactive precursor which converts in vivo to a compound of the invention; or 3) a relatively less active component of the compound that contributes to therapeutic activity after becoming available in vivo (i.e., as a metabolite). See “Design of Prodrugs”, ed. H. Bundgaard, Elsevier, 1985.

“Metabolite” means a pharmaceutically acceptable form of a metabolic derivative of a compound (or a salt thereof) of the invention, wherein the derivative is an active compound that contributes to therapeutic activity after becoming available in vivo.

“Effective amount” means that amount of active compound agent that elicits the desired biological response in a subject. Such response includes alleviation of the symptoms of the disease or disorder being treated. The effective amount of a compound of the invention in such a therapeutic method is from about 10 mg/kg/day to about 0.01 mg/kg/day, preferably from about 0.5 mg/kg/day to 5 mg/kg/day.

The invention includes the use of a compound of the invention for the preparation of a composition for treating or ameliorating an aspartic protease mediated chronic disorder or disease or infection in a subject in need thereof, wherein the composition comprises a mixture one or more compounds of the invention and an optional pharmaceutically acceptable carrier.

“Pharmaceutically acceptable carrier” means compounds and compositions that are of sufficient purity and quality for use in the formulation of a composition of the invention and that, when appropriately administered to an animal or human, do not produce an adverse reaction.

“Aspartic protease mediated disorder or disease” includes disorders or diseases associated with the elevated expression or overexpression of aspartic proteases and conditions that accompany such diseases.

An embodiment of the invention includes administering a renin inhibiting compound of Formula I or any formula of the invention described herein or composition thereof in a combination therapy (U.S. Pat. No. 5,821,232, U.S. Pat. No. 6,716,875, U.S. Pat. No. 5,663,188, Fossa, A. A.; DePasquale, M. J.; Ringer, L. J.; Winslow, R. L. “Synergistic effect on reduction in blood pressure with coadministration of a renin inhibitor or an angiotensin-converting enzyme inhibitor with an angiotensin II receptor antagonist” Drug Development Research 1994, 33(4), 422-8) with one or more additional agents for the treatment of hypertension including α-blockers, β-blockers, calcium channel blockers, diuretics, natriuretics, saluretics, centrally acting antiphypertensives, angiotensin converting enzyme (ACE) inhibitors, dual ACE and neutral endopeptidase (NEP) inhibitors, angiotensin-receptor blockers (ARBs), aldosterone synthase inhibitor, aldosterone-receptor antagonists, or endothelin receptor antagonist.

α-Blockers include doxazosin, prazosin, tamsulosin, and terazosin.

β-Blockers for combination therapy are selected from atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, carvedilol, and their pharmaceutically acceptable salts.

Calcium channel blockers include dihydropyridines (DHPs) and non-DHPs. The preferred DHPs are selected from the group consisting of amlodipine, felodipine, ryosidine, isradipine, lacidipine, nicardipine, nifedipine, nigulpidine, niludipine, nimodiphine, nisoldipine, nitrendipine, and nivaldipine and their pharmaceutically acceptable salts. Non-DHPs are selected from flunarizine, prenylamine, diltiazem, fendiline, gallopamil, mibefradil, anipamil, tiapamil, and verampimil and their pharmaceutically acceptable salts.

A diuretic is, for example, a thiazide derivative selected from amiloride, chlorothiazide, hydrochlorothiazide, methylchlorothiazide, and chlorothalidon.

Centrally acting antiphypertensives include clonidine, guanabenz, guanfacine and methyldopa.

ACE inhibitors include alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril. Preferred ACE inhibitors are benazepril, enalpril, lisinopril, and ramipril.

Dual ACE/NEP inhibitors are, for example, omapatrilat, fasidotril, and fasidotrilat.

Preferred ARBs include candesartan, eprosartan, irbesartan, losartan, olmesartan, tasosartan, telmisartan, and valsartan.

Preferred aldosterone synthase inhibitors are anastrozole, fadrozole, and exemestane.

Preferred aldosterone-receptor antagonists are spironolactone and eplerenone.

A preferred endothelin antagonist is, for example, bosentan, enrasentan, atrasentan, darusentan, sitaxentan, and tezosentan and their pharmaceutically acceptable salts.

An embodiment of the invention includes administering an HIV protease inhibiting compound of Formula I or any formula of the invention described herein or composition thereof in a combination therapy with one or more additional agents for the treatment of AIDS reverse transcriptase inhibitors, non-nucleoside reverse transcriptase inhibitors, other HIV protease inhibitors, HIV integrase inhibitors, entry inhibitors (including attachment, co-receptor and fusion inhibitors), antisense drugs, and immune stimulators.

Preferred reverse transcriptase inhibitors are zidovudine, didanosine, zalcitabine, stavudine, lamivudine, abacavir, tenofovir, and emtricitabine.

Preferred non-nucleoside reverse transcriptase inhibitors are nevirapine, delaviridine, and efavirenz.

Preferred HIV protease inhibitors are saquinavir, ritonavir, indinavir, nelfinavir, amprenavir, lopinavir, atazanavir, and fosamprenavir.

Preferred HIV integrase inhibitors are L-870,810 and S-1360.

Entry inhibitors include compounds that bind to the CD4 receptor, the CCR5 receptor or the CXCR4 receptor. Specific examples of entry inhibitors include enfuvirtide (a peptidomimetic of the HR2 domain in gp41) and sifurvitide.

A preferred attachment and fusion inhibitor is enfuvirtide.

An embodiment of the invention includes administering β-secretase inhibiting compound of Formula I or any formula of the invention described herein or composition thereof in a combination therapy with one or more additional agents for the treatment of Alzheimer's disease including tacrine, donepezil, rivastigmine, galantamine, and memantine.

An embodiment of the invention includes administering a plasmepsin inhibiting compound of Formula I or any formula of the invention described herein or composition thereof in a combination therapy with one or more additional agents for the treatment of malaria including artemisinin, chloroquine, halofantrine, hydroxychloroquine, mefloquine, primaquine, pyrimethamine, quinine, sulfadoxine.

Combination therapy includes co-administration of the compound of the invention and said other agent, sequential administration of the compound and the other agent, administration of a composition containing the compound and the other agent, or simultaneous administration of separate compositions containing of the compound and the other agent.

The invention further includes the process for making the composition comprising mixing one or more of the present compounds and an optional pharmaceutically acceptable carrier; and includes those compositions resulting from such a process, which process includes conventional pharmaceutical techniques.

The compositions of the invention include ocular, oral, nasal, transdermal, topical with or without occlusion, intravenous (both bolus and infusion), and injection (intraperitoneally, subcutaneously, intramuscularly, intratumorally, or parenterally). The composition may be in a dosage unit such as a tablet, pill, capsule, powder, granule, liposome, ion exchange resin, sterile ocular solution, or ocular delivery device (such as a contact lens and the like facilitating immediate release, timed release, or sustained release), parenteral solution or suspension, metered aerosol or liquid spray, drop, ampoule, auto-injector device, or suppository; for administration ocularly, orally, intranasally, sublingually, parenterally, or rectally, or by inhalation or insufflation.

Compositions of the invention suitable for oral administration include solid forms such as pills, tablets, caplets, capsules (each including immediate release, timed release, and sustained release formulations), granules and powders; and, liquid forms such as solutions, syrups, elixirs, emulsions, and suspensions. Forms useful for ocular administration include sterile solutions or ocular delivery devices. Forms useful for parenteral administration include sterile solutions, emulsions, and suspensions.

The compositions of the invention may be administered in a form suitable for once-weekly or once-monthly administration. For example, an insoluble salt of the active compound may be adapted to provide a depot preparation for intramuscular injection (e.g., a decanoate salt) or to provide a solution for ophthalmic administration.

The dosage form containing the composition of the invention contains an effective amount of the active ingredient necessary to provide a therapeutic effect. The composition may contain from about 5,000 mg to about 0.5 mg (preferably, from about 1,000 mg to about 0.5 mg) of a compound of the invention or salt form thereof and may be constituted into any form suitable for the selected mode of administration. The composition may be administered about 1 to about 5 times per day. Daily administration or post-periodic dosing may be employed.

For oral administration, the composition is preferably in the form of a tablet or capsule containing, e.g., 500 to 0.5 milligrams of the active compound. Dosages will vary depending on factors associated with the particular patient being treated (e.g., age, weight, diet, and time of administration), the severity of the condition being treated, the compound being employed, the mode of administration, and the strength of the preparation.

The oral composition is preferably formulated as a homogeneous composition, wherein the active ingredient is dispersed evenly throughout the mixture, which may be readily subdivided into dosage units containing equal amounts of a compound of the invention. Preferably, the compositions are prepared by mixing a compound of the invention (or pharmaceutically acceptable salt thereof) with one or more optionally present pharmaceutical carriers (such as a starch, sugar, diluent, granulating agent, lubricant, glidant, binding agent, and disintegrating agent), one or more optionally present inert pharmaceutical excipients (such as water, glycols, oils, alcohols, flavoring agents, preservatives, coloring agents, and syrup), one or more optionally present conventional tableting ingredients (such as corn starch, lactose, sucrose, sorbitol, talc, stearic acid, magnesium stearate, dicalcium phosphate, and any of a variety of gums), and an optional diluent (such as water).

Binder agents include starch, gelatin, natural sugars (e.g., glucose and beta-lactose), corn sweeteners and natural and synthetic gums (e.g., acacia and tragacanth). Disintegrating agents include starch, methyl cellulose, agar, and bentonite.

Tablets and capsules represent an advantageous oral dosage unit form. Tablets may be sugarcoated or filmcoated using standard techniques. Tablets may also be coated or otherwise compounded to provide a prolonged, control-release therapeutic effect. The dosage form may comprise an inner dosage and an outer dosage component, wherein the outer component is in the form of an envelope over the inner component. The two components may further be separated by a layer which resists disintegration in the stomach (such as an enteric layer) and permits the inner component to pass intact into the duodenum or a layer which delays or sustains release. A variety of enteric and non-enteric layer or coating materials (such as polymeric acids, shellacs, acetyl alcohol, and cellulose acetate or combinations thereof) may be used.

Compounds of the invention may also be administered via a slow release composition; wherein the composition includes a compound of the invention and a biodegradable slow release carrier (e.g., a polymeric carrier) or a pharmaceutically acceptable non-biodegradable slow release carrier (e.g., an ion exchange carrier).

Biodegradable and non-biodegradable slow release carriers are well known in the art. Biodegradable carriers are used to form particles or matrices which retain an active agent(s) and which slowly degrade/dissolve in a suitable environment (e.g., aqueous, acidic, basic and the like) to release the agent. Such particles degrade/dissolve in body fluids to release the active compound(s) therein. The particles are preferably nanoparticles (e.g., in the range of about 1 to 500 nm in diameter, preferably about 50-200 nm in diameter, and most preferably about 100 nm in diameter). In a process for preparing a slow release composition, a slow release carrier and a compound of the invention are first dissolved or dispersed in an organic solvent. The resulting mixture is added into an aqueous solution containing an optional surface-active agent(s) to produce an emulsion. The organic solvent is then evaporated from the emulsion to provide a colloidal suspension of particles containing the slow release carrier and the compound of the invention.

The compound of Formula I may be incorporated for administration orally or by injection in a liquid form such as aqueous solutions, suitably flavored syrups, aqueous or oil suspensions, flavored emulsions with edible oils such as cottonseed oil, sesame oil, coconut oil or peanut oil and the like, or in elixirs or similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone, and gelatin. The liquid forms in suitably flavored suspending or dispersing agents may also include synthetic and natural gums. For parenteral administration, sterile suspensions and solutions are desired. Isotonic preparations, which generally contain suitable preservatives, are employed when intravenous administration is desired.

The compounds may be administered parenterally via injection. A parenteral formulation may consist of the active ingredient dissolved in or mixed with an appropriate inert liquid carrier. Acceptable liquid carriers usually comprise aqueous solvents and other optional ingredients for aiding solubility or preservation. Such aqueous solvents include sterile water, Ringer's solution, or an isotonic aqueous saline solution. Other optional ingredients include vegetable oils (such as peanut oil, cottonseed oil, and sesame oil), and organic solvents (such as solketal, glycerol, and formyl). A sterile, non-volatile oil may be employed as a solvent or suspending agent. The parenteral formulation is prepared by dissolving or suspending the active ingredient in the liquid carrier whereby the final dosage unit contains from 0.005 to 10% by weight of the active ingredient. Other additives include preservatives, isotonizers, solubilizers, stabilizers, and pain-soothing agents. Injectable suspensions may also be prepared, in which case appropriate liquid carriers, suspending agents and the like may be employed.

Compounds of the invention may be administered intranasally using a suitable intranasal vehicle.

Compounds of the invention may also be administered topically using a suitable topical transdermal vehicle or a transdermal patch.

For ocular administration, the composition is preferably in the form of an ophthalmic composition. The ophthalmic compositions are preferably formulated as eye-drop formulations and filled in appropriate containers to facilitate administration to the eye, for example a dropper fitted with a suitable pipette. Preferably, the compositions are sterile and aqueous based, using purified water. In addition to the compound of the invention, an ophthalmic composition may contain one or more of: a) a surfactant such as a polyoxyethylene fatty acid ester; b) a thickening agents such as cellulose, cellulose derivatives, carboxyvinyl polymers, polyvinyl polymers, and polyvinylpyrrolidones, typically at a concentration n the range of about 0.05 to about 5.0% (wt/vol); c) (as an alternative to or in addition to storing the composition in a container containing nitrogen and optionally including a free oxygen absorber such as Fe), an anti-oxidant such as butylated hydroxyanisol, ascorbic acid, sodium thiosulfate, or butylated hydroxytoluene at a concentration of about 0.00005 to about 0.1% (wt/vol); d) ethanol at a concentration of about 0.01 to 0.5% (wt/vol); and e) other excipients such as an isotonic agent, buffer, preservative, and/or pH-controlling agent. The pH of the ophthalmic composition is desirably within the range of 4 to 8.

### Methods of Preparation

In the discussion below R¹, R², R³, Y1, X1, A, Q, R⁴, L, R^{a}, R^{b}, R^{c}, R^{d}, R^{e}, R^{f} and G are defined as described above for compounds of Formula I. In cases where the synthetic intermediates and final products of Formula I described below contain potentially reactive functional groups, for example amino, hydroxy, thiol and carboxylic acid groups, that may interfere with the desired reaction, it may be advantageous to employ protected forms of the intermediate. Methods for the selection, introduction and subsequent removal of protecting groups are well known to those skilled in the art. (T. W. Greene and P. G. M. Wuts “Protective Groups in Organic Synthesis” John Wiley & Sons, Inc., New York 1999). Such protecting group manipulations are assumed in the discussion below and not usually described explicitly. Generally, reagents in the reaction schemes are used in equimolar amounts; however, in certain cases it may be desirable to use an excess of one reagent to drive a reaction to completion. This is especially the case when the excess reagent can be readily removed by evaporation or extraction. Bases employed to neutralize HCl in reaction mixtures are generally used in slight to substantial excess (1.05-5 equivalents).

In the first process, a compound of Formula I wherein Q is Q1 is prepared by reaction of an intermediate of Formula II with an amine intermediate of Formula III:

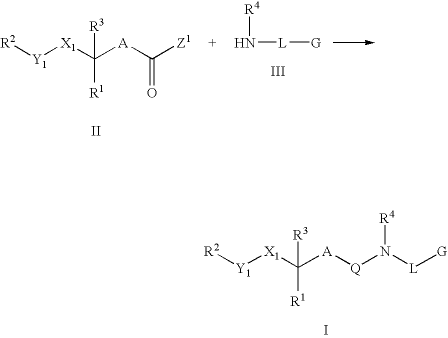

wherein Z¹ in II is a leaving group such as halide, alkanesulfonate, haloalkanesulfonate, arylsulfonate, aryloxide, heteroaryloxide, azole, azolium salt, or alkoxide.

Intermediates of Formula II wherein Z¹=chlorine and a carbon atom of A is attached to C(═O)Z¹ are prepared from carboxylic acid intermediates of formula IV:

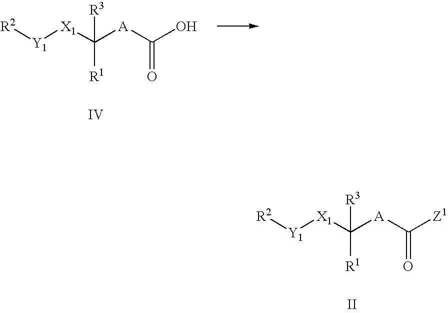

by reaction with, for example, thionyl chloride or oxalyl chloride.

Carboxylic acids of Formula IV wherein a carbon atom of A is a benzene ring can be prepared by palladium catalyzed carbonylation of halide intermediates of Formula V wherein Z² is chlorine, bromine or iodine in the presence of an alcohol such as methanol, followed by ester hydrolysis:

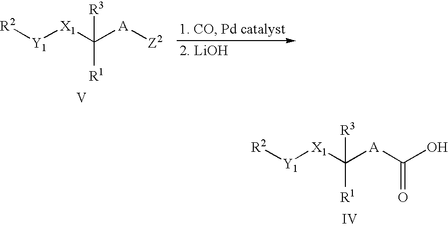

Suitable palladium catalysts include PdCl₂(PPh₃)₂.

Halide intermediates of Formula V wherein R² is NHC(═O)OR⁹ , X₁ is a covalent bond and Y₁ is alkylene, alkenylene or alkynylene, can be prepared from an amine of formula VI wherein Y₁ is alkylene, alkenylene or alkynylene, by reaction with a chloroformate R⁹OC(═O)Cl in the presence of a amine base such as pyridine or i-Pr₂NEt in an inert solvent such as CH₂Cl₂ or THF:

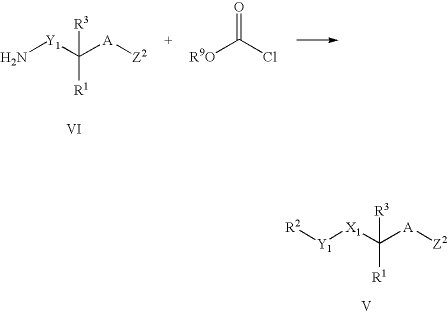

Amines of Formula VI, wherein R³ is OH, can be prepared by addition of organometallic reagents of Formula VII wherein M is Li, MgCl, MgBr or MgBr and the nitrogen is suitably protected to ketones of Formula VIII:

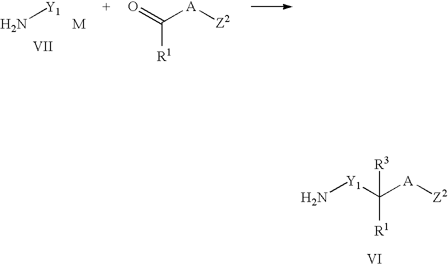

An example of an organometallic of Formula VII is (3-(2,2,5,5-tetramethyl-1,2,5-azadisilolidin-1-yl)propyl)magnesium bromide. Examples of ketones of Formula VIII, wherein A is a benzene ring and R¹ is optionally substituted phenyl, are benzophenones.

Halide intermediates of Formula V wherein R² is NHC(═O)OR⁹ , X₁ is O or S, Y₁ is alkylene, alkenylene or alkynylene and R³ is H can be prepared from an amine of Formula IX wherein X₁ is O or S, Y₁ is alkylene, alkenylene or alkynylene and R³ is H by reaction with a chloroformate R⁹OC(═O)Cl in the presence of a amine base such as pyridine or i-Pr₂NEt in an inert solvent such as CH₂Cl₂ or THF:

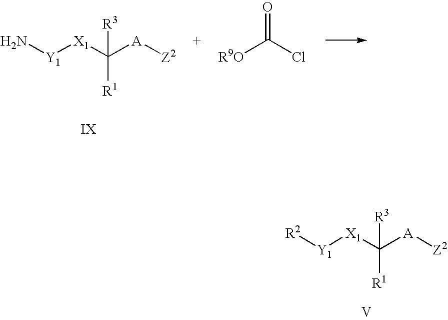

Amines of Formula IX can be prepared by reduction of nitriles of Formula X wherein Yₐ is an alkylene, alkenylene or alkynylene chain with one carbon fewer than in Y₁ using, for example, LiAlH₄ or BH₃.THF in an ethereal solvent such as THF.

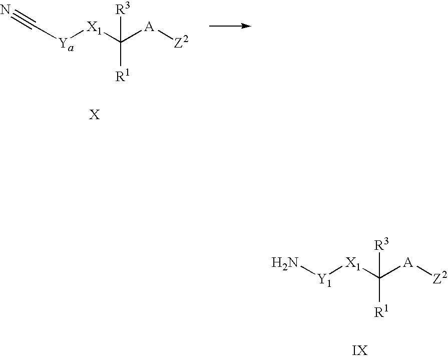

Amines of Formula IX can be prepared by reduction of carboxamides of Formula XI wherein Yₐ is an alkylene, alkenylene or alkynylene chain with one carbon fewer than in Y₁ using, for example, LiAlH₄ or BH₃.THF in an ethereal solvent such as THF.

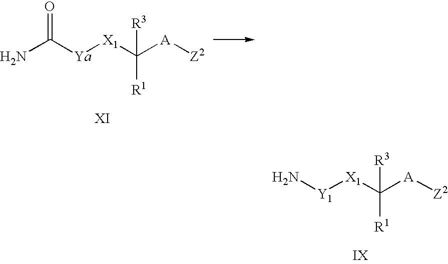

Intermediates of Formula III wherein L is a C₂ alkyl chain are prepared from natural and unnatural α-amino acids and by other methods (Lucet, D.; Le Gall, T.; Mioskowski, C. Angew. Chem. Int. Ed. 1998, 37, 2580-2617). Likewise, intermediates of Formula III wherein L is a C₃ or C₄ alkyl chain are prepared from β- and γ-amino acids, respectively.

In a second process, a compound of Formula I wherein Q is Q1 attached to a carbon atom of A is prepared by reaction of a carboxylic acid of Formula IV with an amine of Formula III in the presence of a peptide coupling reagent and a tertiary amine base such as Et₃N or i-Pr₂NEt:

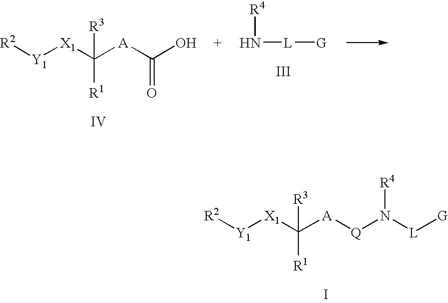

Standard peptide coupling agents well known to those skilled in the art include (i) carbodiimides such as DCC, DIC and EDC, which are optionally used in the presence of HOBt, (ii) HATU and HBTU, (iii) PyBOP and (iv) CDI.

In a third process, a compound of Formula I, wherein A is a benzene ring and X₁ is O, is prepared from an alcohol of Formula XII and an alcohol of Formula XIII in the presence of an acid such as p-toluenesulfonic acid:

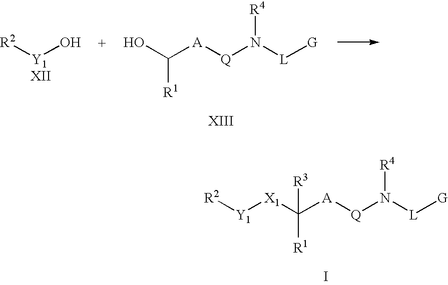

Alcohol intermediates of Formula XIII are prepared by reduction of ketones of Formula XIV using, for example NaBH₄ in MeOH or LiAlH₄ in THF or ether:

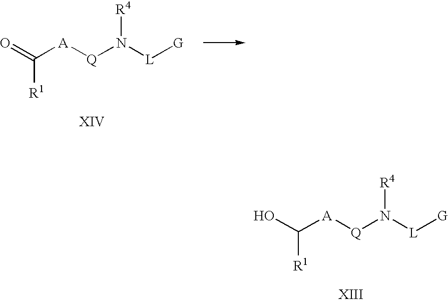

Ketone intermediates of Formula XIV are prepared by reaction of carboxylic acid derivatives of Formula XV, wherein Z⁴ is OH, OMe, NEt₂ or, preferably, NMeOMe, with organometallic reagents of Formula XVI, wherein M is Li, MgCl, MgBr or MgI:

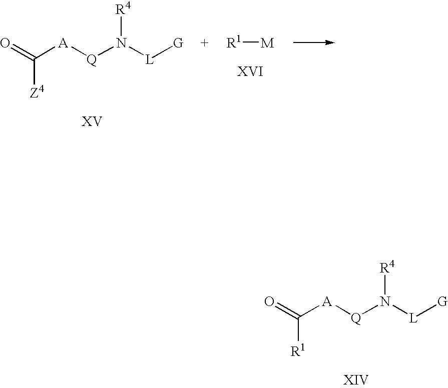

Ketone intermediates of Formula XIV, wherein Q is Q1, are also prepared by reaction of carboxylic acids of Formula XVII with amine intermediates of Formula III in the presence of a peptide coupling reagent and a tertiary amine base such as Et₃N or i-Pr₂NEt:

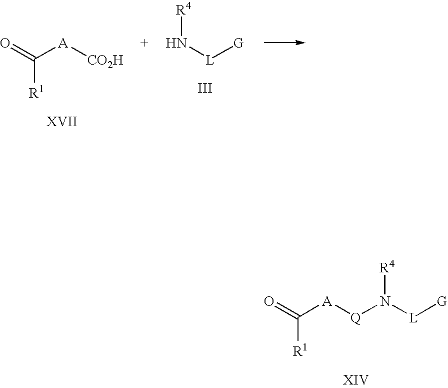

Carboxylic acids of Formula XVII, wherein A is a benzene ring and R¹ is an optionally substituted benzene ring, are benzoylbenzoic acids.

Carboxylic acids of Formula XVII, wherein A is cyclohexane and the R¹C(═O)— and —CO₂H substituents are attached in a 1,3-relationship, are prepared by reaction of anhydride XVIII with an organometallic reagent of Formula XVI, wherein M=Li, MgCl, MgBr or MgI, optionally in the presence of a copper(I) salt:

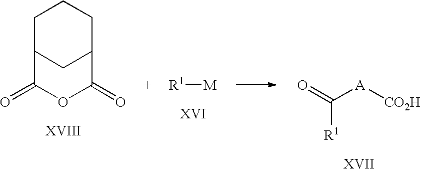

Alcohol intermediates of Formula XIII are prepared by reaction of aldehyde intermediates of Formula XIX with an organometallic reagent of Formula XVI, wherein M=Li, MgCl, MgBr or MgI:

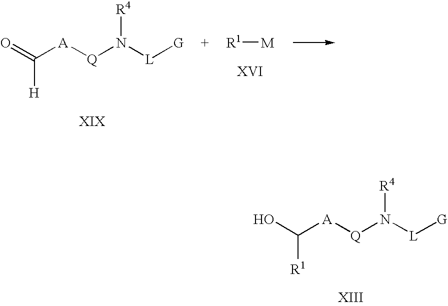

In a fourth process compounds of Formula I, wherein X₁ is O and R³ is H, are prepared by reaction of an alcohol of Formula XIII with an alkylating agent of Formula XX, wherein Z¹ is a leaving group such as bromide, iodide, methanesulfonate or trifluoromethanesulfonate:

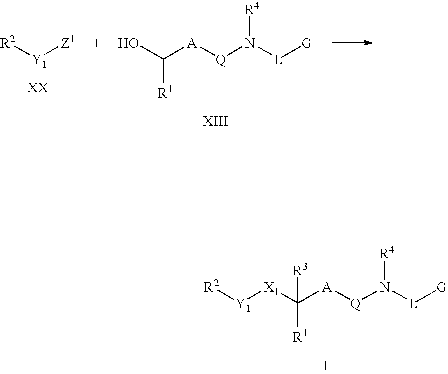

Alkylating agents of Formula XX are prepared from alcohols of Formula XII.

In a fifth process compounds of Formula I, wherein X₁ is S and R³ is H, are prepared by reaction of thiols of Formula XXI with compounds of Formula XXII, wherein Z¹ is a leaving group such as bromide or methanesulfonate:

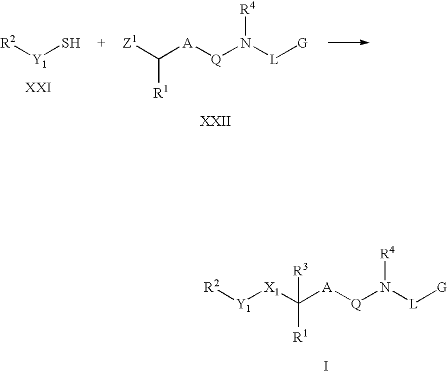

Compounds of Formula XXII are prepared from alcohols of Formula XIII.

In a sixth process compounds of Formula I wherein R² is R⁹OC(═O)NH are prepared by reaction of chloroformates having the formula R⁹OC(═O)Cl with amines of Formula XXIII in the presence of a amine base such as pyridine or i-Pr₂NEt in an inert solvent such as CH₂Cl₂ or THF:

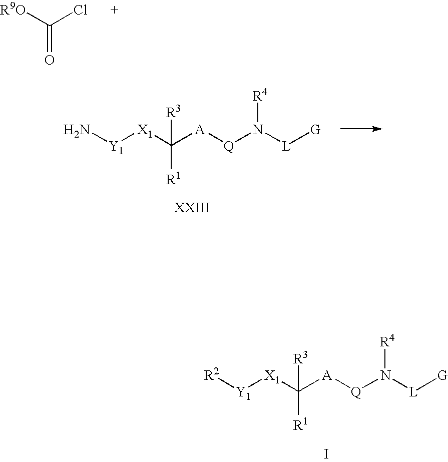

Amines of Formula XXIII wherein R³═OH, X₁ is a bond and Y₁ is not a bond, are prepared by addition of by addition of organometallic reagents of Formula VII, wherein M is Li, MgCl, MgBr or MgBr and the nitrogen is suitably protected, to ketones of Formula XV, followed by deprotection:

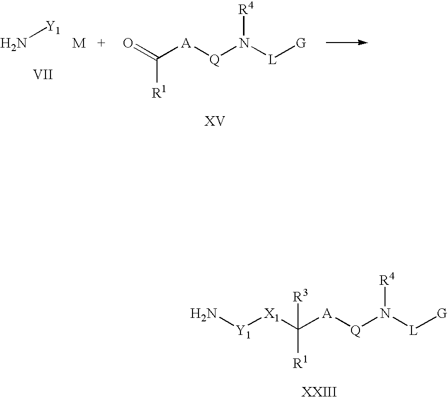

An example of an organometallic of Formula VII is (3-(2,2,5,5-tetramethyl-1,2,5-azadisilolidin-1-yl)propyl)magnesium bromide.

In a seventh process, compounds of Formula I are prepared from other compounds of Formula I:

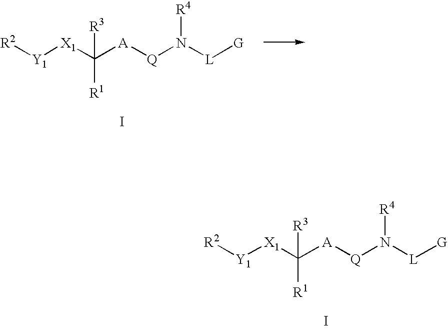

For example:
(1) compounds of Formula I, wherein Q is Q1, are converted to compounds of Formula I, wherein Q is Q2, by the action of P₂S₅ or Lawesson's reagent;
(2) compounds of Formula I, wherein R¹ is bromophenyl, are transformed to compounds of Formula I, wherein R¹ is biphenyl, using a Suzuki coupling;
(3) compounds of Formula I, wherein R¹ is bromophenyl, are transformed to compounds of Formula I, wherein R¹ is CN, using CuCN;
(4) compounds of Formula I, wherein R¹ is hydroxyphenyl, are transformed into compounds of Formula I, wherein R¹ is alkoxyphenyl, cycloalkoxyphenyl or cycloalkylalkoxyphenyl by treatment with a base such as NaH or KOH and an alkyl halide, cycloalkyl halide or cycloalkylalkyl halide;
(5) compounds of Formula I, wherein G is NH₂, are transformed into compounds of Formula I, wherein G is NHR^{e} and R^{e} is alkyl or benzyl, by reductive alkylation with an alkyl aldehyde or benzaldehyde respectively, using sodium cyanoborohydride or sodium triacetoxyborohydride.

The invention is further defined by reference to the examples, which are intended to be illustrative and not limiting.

Representative compounds of the invention can be synthesized in accordance with the general synthetic schemes described above and are illustrated in the examples that follow. The methods for preparing the various starting materials used in the schemes and examples are well within the knowledge of persons skilled in the art.

The following abbreviations have the indicated meanings:

| Abbreviation | Meaning |
|---|---|
| aq | aqueous |
| Boc | tert-butoxy carbonyl or t-butoxy carbonyl |
| (Boc)₂O | di-tert-butyl dicarbonate |
| brine | saturated aqueous NaCl |
| CH₂Cl₂ | methylene chloride |
| CH₃CN | acetonitrile |
| or MeCN | |
| Cpd | compound |
| d | day |
| DBU | 1,8-diazabicyclo[5.4.0]undec-7-ene |
| DIEA | N,N-diisopropylethylamine |
| DMAP | 4-(dimethylamino)pyridine |
| DMF | N,N-dimethylformamide |
| DMPU | 1,3-dimethyl-3,4,5,6-tetrahydro-2(1H)-pyrimidinone |
| EDC•HCl | 1-[3-(dimethylamino)propyl]-3-ethylcarbodiimide |
| | hydrochloride |
| equiv | equivalents |
| Et | ethyl |
| Et₂O | ethyl ether |
| EtOAc | ethyl acetate |
| Fmoc | 1-[[(9H-fluoren-9-ylmethoxy)carbonyl]oxy]- |
| Fmoc-OSu | 1-[[(9H-fluoren-9-ylmethoxy)carbonyl]oxy]-2,5- |
| | pyrrolidinedione |
| h, hr | hour |
| HOBt | 1-hydroxybenzotriazole |
| HATU | 2-(7-Aza-1H-benzotriazole-1-yl)-1,1,3,3- |
| | tetramethyluronium hexafluorophosphate |
| HBTU | 2-(1H-Benzotriazole-1-yl)-1,1,3,3-tetramethyluronium |
| | hexafluorophosphate |
| KHMDS | potassium hexamethyldisilazane |
| LAH or | lithium aluminum hydride |
| LiAlH₄ | |
| LC-MS | liquid chromatography-mass spectroscopy |
| LHMDS | lithium hexamethyldisilazane |
| Me | methyl |
| MeCN | acetonitrile |
| MeOH | methanol |
| MsCl | methanesulfonyl chloride |
| min | minute |
| MS | mass spectrum |
| NaH | sodium hydride |
| NaHCO₃ | sodium bicarbonate |
| NaN₃ | sodium azide |
| NaOH | sodium hydroxide |
| Na₂SO₄ | sodium sulfate |
| NMP | N-methylpyrrolidinone |
| Pd₂(dba)₃ | tris(dibenzylideneacetone)dipalladium(0) |
| Ph | phenyl |
| rt | room temperature |
| satd | saturated |
| SOCl₂ | thionyl chloride |
| TBAF | tetrabutylammonium fluoride |
| TEA | triethylamine or Et₃N |
| TEAF | tetraethylammonium fluoride |
| TEMPO | 2,2,6,6-tetramethyl-1-piperidinyloxy, free radical |
| Teoc | 1-[2-(trimethylsilyl)ethoxycarbonyloxy]- |
| Teoc-OSu | 1-[2-(trimethylsilyl)ethoxycarbonyloxy]pyrrolidin-2,5-dione |
| TFA | trifluoroacetic acid |
| THF | tetrahydrofuran |
| TMSCl | chlorotrimethylsilane or trimethylsilyl chloride |
| t_{R} | retention time |

### Purification Methods

Preparative HPLC refers to reverse phase HPLC on a C-18 column eluted with a water/acetonitrile gradient containing 0.01% TFA run on a Gilson 215 system.

Chromatography on silica gel refers to normal phase chromatography on a silica gel column or cartridge eluted with an hexanes/EtOAc gradient.

Preparative TLC refers to normal phase thin or thick layer chromatography on a silica gel plate eluted with an organic solvents or mixtures of organic solvents, such as hexanes/EtOAc mixtures.

Chiral HPLC refers to normal phase chromatography on a chiral column, such as chiralcel OD-H or AD-H, eluted with a mixture of organic solvents such as isopropanol in hexanes buffered with diethylamine

### Analytical Methods

LC-MS (3 min)
Column: Chromolith SpeedRod, RP-18e, 50×4.6 mm; Mobil phase: A: 0.01% TFA/water, B: 0.01% TFA/CH₃CN; Flow rate: 1 mL/min; Gradient:

| Time | | |
|---|---|---|
| (min) | A % | B % |
| 0.0 | 90 | 10 |
| 2.0 | 10 | 90 |
| 2.4 | 10 | 90 |
| 2.5 | 90 | 10 |
| 3.0 | 90 | 10 |

### Electrospray Ionization

The compounds of present invention can be synthesized by coupling a pyran intermediate represented by the following structure:

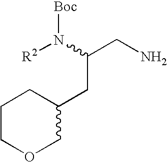

with a benzoic acid intermediate represented by the following structure:

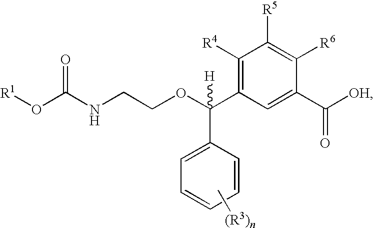

described in the following scheme:

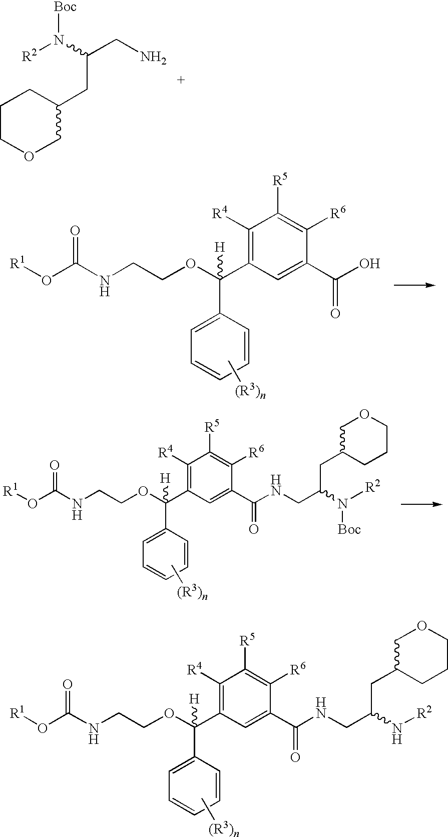

R⁴, R⁵ and R⁶ are optional substituents of the phenyl ring described above.

### Preparation of the Pyran Intermediate

The pyran intermediate may be prepared from pyroglutamic ester using the following synthetic scheme:

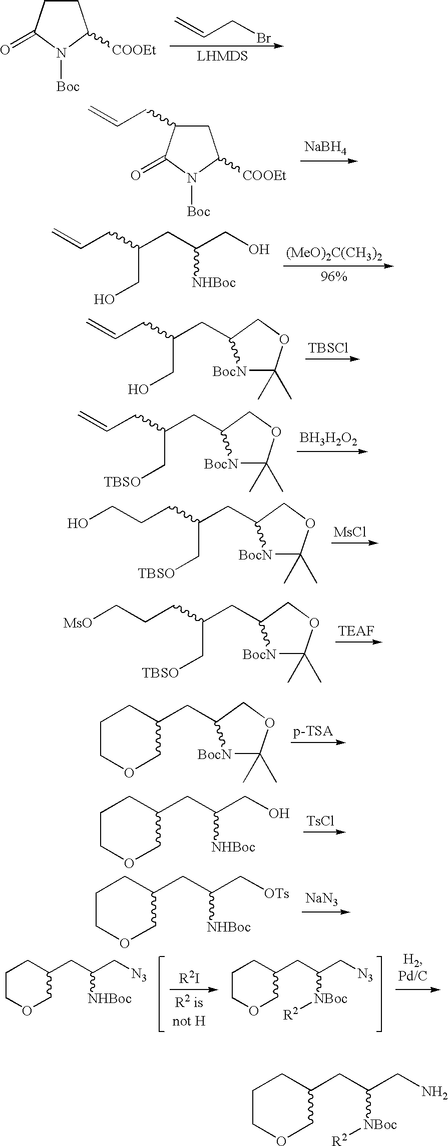

### Preparation of Diastereomerically Pure Pyran Intermediate

The chiral pyran intermediate may be obtained in diastereomerically pure form using the following synthetic scheme:

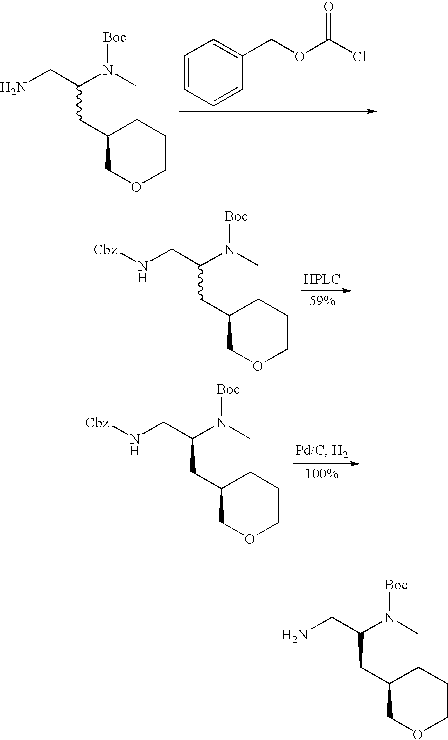

### Preparation of the Benzoic Acid Intermediate

An intermediate that is used in each of the methods for preparing the benzoic acid intermediate is a carbamate-protected amino-ethanol, which can be prepared using the following synthetic scheme.

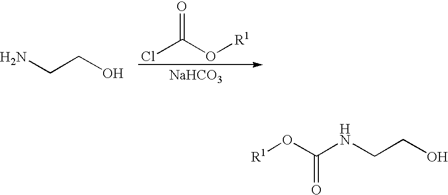

The benzoic acid intermediate can be prepared by using the following synthetic scheme.

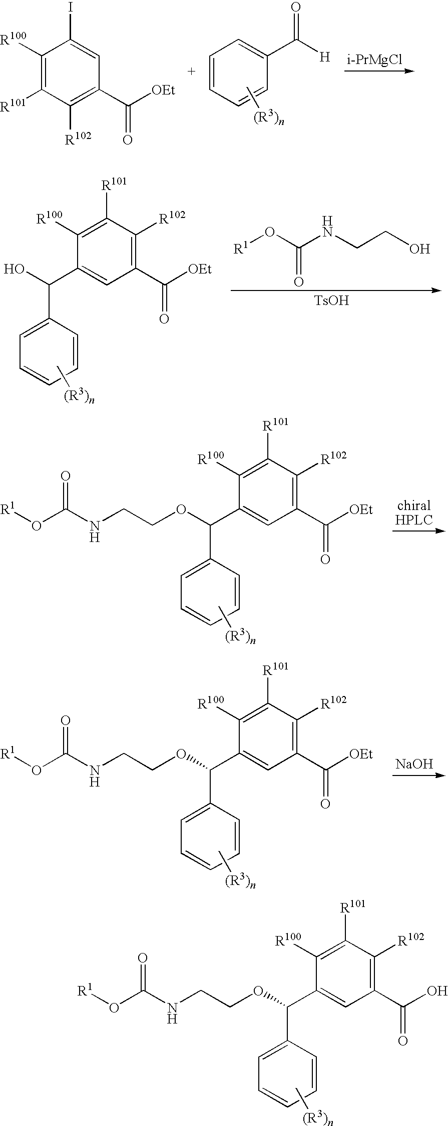

R¹⁰⁰, R¹⁰¹ and R¹⁰² are optional substituents of the phenyl ring as described above.

Alternatively, the benzoic acid intermediate can be prepared using the following synthetic scheme:

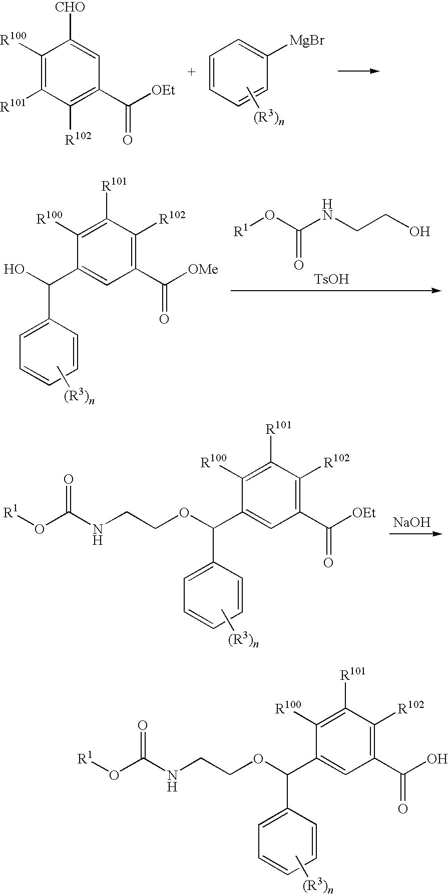

Alternatively, the benzoic acid intermediate can be prepared using the following synthetic scheme:

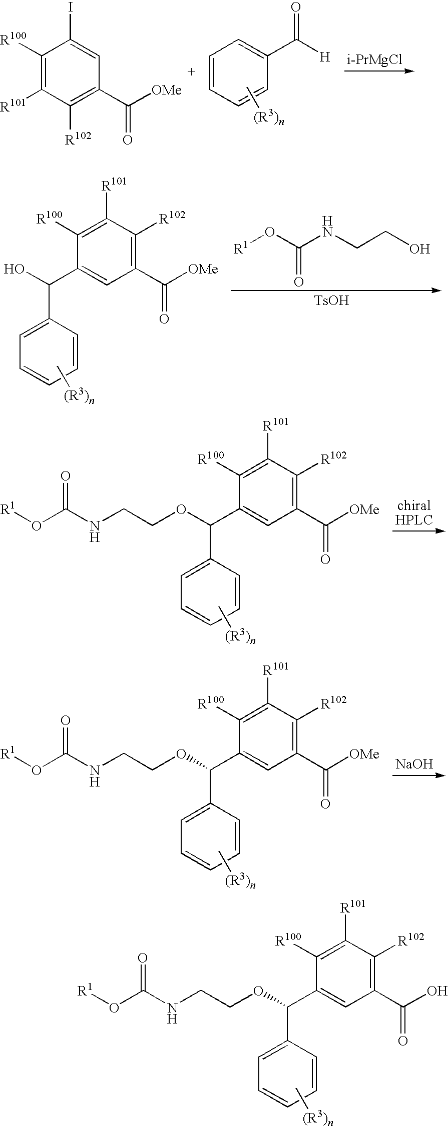

Alternatively, the benzoic acid intermediate can be prepared using the following synthetic scheme:

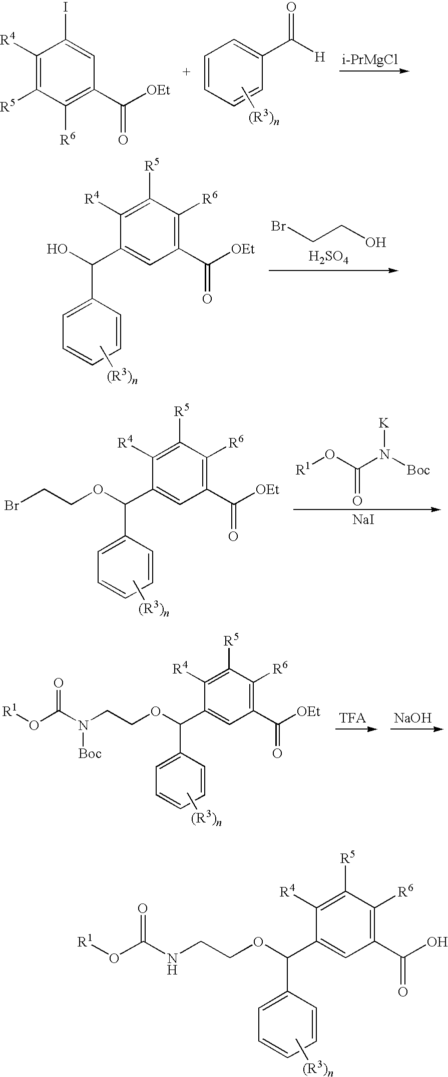

### Intermediate Preparation 1

### 2,2-dimethyl-4-(((R)-tetrahydro-2H-pyran-3-yl)methyl)oxazolidine

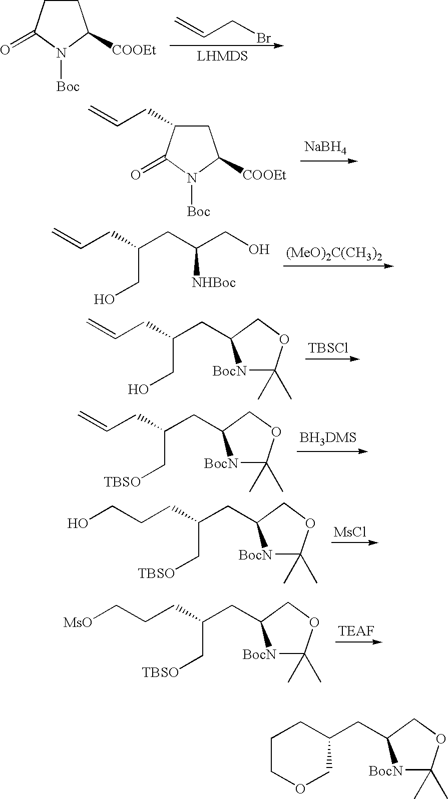

### Step 1. (2S,4R)-1-tert-butyl 2-ethyl 4-allyl-5-oxopyrrolidine-1,2-dicarboxylate

To a solution of HMDS in anhydrous THF (200 mL) was added dropwise 2.5 M n-BuLi in hexane (130 mL) and the mixture was stirred at −78° C. for 1 hr. To a solution of (S)-1-tert-butyl 2-ethyl 5-oxopyrrolidine-1,2-dicarboxylate (80 g, 0.311 mol) in anhydrous THF (1600 mL) stirred at −78° C. was added lithium hexamethyldisilazide in THF. After the reaction mixture was stirred at −78° C. for 1 hr, 3-bromopropene (38.47 g, 0.318 mol) in THF (200 mL) was added and stirring was continued for 2 hr. The reaction mixture was quenched with saturated ammonium chloride solution (600 mL) at −78° C. and extracted with EtOAc (3×500 mL). The combined organic layers were dried over Na₂SO₄, filtered and evaporated to dryness. The crude product was separated by column chromatography to afford (2S,4R)-1-tert-butyl 2-ethyl 4-allyl-5-oxopyrrolidine-1,2-dicarboxylate (15 g, 16%).

### Step 2. tert-butyl(2S,4R)-1-hydroxy-4-(hydroxymethyl)hept-6-en-2-ylcarbamate

To a solution of (2S,4R)-1-tert-butyl 2-ethyl 4-allyl-5-oxopyrrolidine-1,2-dicarboxylate (30 g, 0.1 mol) in MeOH/H₂O (700/70 mL) was added NaBH₄ (25 g, 0.66 mol), the result mixture was stirred 1 hr at rt and quenched with sat. aq. NH₄Cl (300 mL). The organic solvent was removed under vacuum and extracted with EtOAc (3×250 mL). The combined organic phases were washed with brine (250 mL) and dried over anhydrous Na₂SO₄, filtered and evaporated to afford crude tert-butyl(2S,4R)-1-hydroxy-4-(hydroxymethyl)hept-6-en-2-ylcarbamate (22 g, 85%). It was used in the next step without further purification.

### Step 3. (S)-tert-butyl 4-((R)-2-(hydroxymethyl)pent-4-enyl)-2,2-dimethyloxazolidine-3-carboxylate

To a solution of tert -butyl(2S,4R)-1-hydroxy-4-(hydroxymethyl)hept-6-en-2-ylcarbamate (6.8 g, 26.2 mmol) in acetone (150 mL), PTSA (0.45 g, 2.62 mmol) was added. The reaction mixture was cooled to −20° C. followed by the addition of 2,2-dimethoxypropane (4.1 g, 39.4 mmol). The resulting mixture was stirred and allowed to warm to rt for 1 hr. TEA (0.5 mL) was then added and stirred for another 5 min. The solvent was removed under reduced pressure. The residue was dissolved in Et₂O (300 mL), washed with 1 N HCl (80 mL), sat. aq. NaHCO₃ (80 mL), brine (80 mL) successively, and dried, filtered, and concentrated under vacuum to give crude (S)-tert-butyl 4-((R)-2-(hydroxymethyl)pent-4-enyl)-2,2-dimethyloxazolidine-3-carboxylate (7.5 g, 96%). It was used without further purification.

### Step 4. (S)-tert-butyl 4-((R)-2-((tert-butyldimethylsilyloxy)methyl)pent-4-enyl)-2,2-dimethyloxazolidine-3-carboxylate

To a solution of (S)-tert-butyl 4-((R)-2-(hydroxymethyl)pent-4-enyl)-2,2-dimethyloxazolidine-3-carboxylate (11.5 g, 38.4 mmol), imidazole (7.84 g, 115.2 mmol) and DMAP (234 mg, 1.92 mmol) in CH₂Cl₂ (200 mL) was added a solution of TBSCl (8.68 g, 57.6 mmol) in CH₂Cl₂ (100 mL) dropwise. The reaction mixture was stirred at rt for overnight. The reaction was washed with water (100 mL) and the aqueous layer was extracted with CH₂Cl₂ (3×100 mL), the combined organic layers was washed with brine (70 mL), then dried over Na₂SO₄, filtered and concentrated to give the crude product, which was purified by column chromatography to afford (S)-tert-butyl 4-((R)-2-((tert-butyldimethylsilyloxy)methyl)pent-4-enyl)-2,2-dimethyloxazolidine-3-carboxylate (9 g, 57%).

### Step 5. (5)-tert-butyl 4-((R)-2-((tert-butyldimethylsilyloxy)methyl)-5-hydroxypentyl)-2,2-dimethyloxazolidine-3-carboxylate

A solution of (S)-tert-butyl 4-((R)-2-((tert-butyldimethylsilyloxy)methyl)pent-4-enyl)-2,2-dimethyloxazolidine-3-carboxylate (26 g, 63 mmol) in THF (200 mL) was cooled in an ice-bath, followed by dropwise addition of 10 M BH₃.SMe₂ (6.3 mL). After stirring for 5 hr, 10% NaOH solution (32 mL) followed by 30% H₂O₂ (32 mL) were added carefully. The reaction mixture was stirred at rt for 16 hr. The reaction mixture was diluted with diethyl ether (500 mL) and the aqueous layer was extracted with diethyl ether (3×250 mL). The combined organic layers were washed with brine, dried over Na₂SO₄, filtered and concentrated to give the crude product, which was purified by column chromatography to afford (S)-tert-butyl 4-((R)-2-((tert-butyldimethylsilyloxy)methyl)-5-hydroxypentyl)-2,2-dimethyloxazolidine-3-carboxylate (19.6 g, 72%).

### Step 6. (S)-tert-butyl 4-((R)-2-((tert-butyldimethylsilyloxy)methyl)-5-(methylsulfonyloxy)pentyl)-2,2-dimethyloxazolidine-3-carboxylate

To a solution of (S)-tert-butyl 4-((R)-2-((tert-butyldimethylsilyloxy)methyl)-5-hydroxypentyl)-2,2-dimethyloxazolidine-3-carboxylate (32 g, 74.2 mmol) and Et₃N (22.5 g, 226 mmol) in CH₂Cl₂ (400 mL) was added a solution of MsCl (10.1 g, 89 mmol) in CH₂Cl₂ (50 mL) at 0-5° C. After addition, the reaction mixture was allowed to warm to rt and stir for 1 hr. The reaction was washed with water (200 mL) and the aqueous layer was extracted with CH₂Cl₂ (3×150 mL). The combined organic layers was washed with 10% citric acid (60 mL), sat. NaHCO₃ (60 mL) and brine (100 mL), then dried over Na₂SO₄, filtered and concentrated to give (S)-tert-butyl 4-((R)-2-((tert-butyldimethylsilyloxy)methyl)-5-(methylsulfonyloxy)pentyl)-2,2-dimethyloxazolidine-3-carboxylate (37.7 g, 100%), which was used in the next step without purification.

### Step 7. (S)-tert-butyl 2,2-dimethyl-4-(((R)-tetrahydro-2H-pyran-3-yl)methyl)oxazolidine-3-carboxylate

To a solution of (S)-tent-butyl 4-((R)-2-((tert-butyldimethylsilyloxy)methyl)-5-(methylsulfonyloxy)pentyl)-2,2-dimethyloxazolidine-3-carboxylate (37.7 g, 74.2 mmol) in THF (1000 mL) was added tetraethylammonium fluoride hydrate (41 g, 185.5 mmol) in portions. The reaction mixture was stirred under reflux overnight. The mixture was diluted with EtOAc (1000 mL), washed with water (300 mL) and brine (500 mL). The organic phase was dried over Na₂SO₄, filtered and concentrated in vacuo to give the crude product, which was purified by column chromatography to afford (5)-tert-butyl 2,2-dimethyl-4-(((R)-tetrahydro-2H-pyran-3-yl)methyl)oxazolidine-3-carboxylate (12.0 g, 54%).

### Intermediate Preparation 2

### tert-butyl (S)-1-amino-3-((R)-tetrahydro-2H-pyran-3-yl)propan-2-ylcarbamate

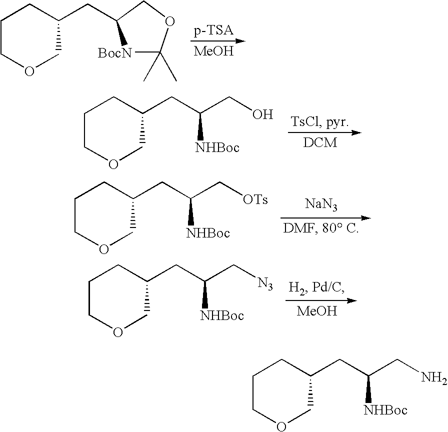

### Step 1. Preparation of tent-butyl (5)-1-hydroxy-3-((R)-tetrahydro-2H-pyran-3-yl)propan-2-ylcarbamate

To a solution of (S)-tert-butyl 2,2-dimethyl-4-(((R)-tetrahydro-2H-pyran-3-yl)methyl)oxazolidine-3-carboxylate (643 mg, 2.15 mmol) in MeOH (10 mL) was added p-TSA (37 mg, 0.22 mmol), then the solution was stirred at rt for 12 hr. TEA (2 mL) was added, followed by Boc₂O (46 mg, 0.21 mmol). After the addition the reaction solution was stirred for another 30 min. The organic solvent was removed under reduced pressure to give the crude product tert-butyl (S)-1-hydroxy-3-((R)-tetrahydro-2H-pyran-3-yl)propan-2-ylcarbamate. It was used in the next step without further purification. MS ESI +ve m/z 260 (M+1).

### Step 2. Preparation of (5)-2-(tert-butoxycarbonylamino)-3-((R)-tetrahydro-2H-pyran-3-yl)propyl 4-methylbenzenesulfonate

The above crude product tert-butyl (S)-1-hydroxy-3-((R)-tetrahydro-2H-pyran-3-yl)propan-2-ylcarbamate was dissolved in anhydrous DCM (22 mL). To this solution was added pyridine (2 mL) and TsCl (1.230 g, 6.45 mmol). After stirred at rt for 4 hr, another batch of pyridine (3 mL) and TsCl (0.700 g, 3.67 mmol) was added and stirred for another 12 hr. The reaction mixture was diluted with EtOAc (80 mL), washed with 1 N HCl (75 mL), followed by H₂O (2×30 mL), saturated aq. NaHCO₃, brine, and dried over anhydrous Na₂SO₄, and filtered, and concentrated under reduced pressure. The resulted slurry was purified through flash chromatography on silica gel (eluted with gradient system: 0-35% EtOAc in hexane) to afford (S)-2-(tert-butoxycarbonylamino)-3-((R)-tetrahydro-2H-pyran-3-yl)propyl 4-methylbenzenesulfonate, 670 mg, yield 75% for two steps. MS ESI +ve m/z 436 (M+Na).

### Step 3. tert-butyl (S)-1-azido-3-((R)-tetrahydro-2H-pyran-3-yl)propan-2-ylcarbamate

The solution of (S)-2-(tert-butoxycarbonylamino)-3-((R)-tetrahydro-2H-pyran-3-yl)propyl 4-methylbenzenesulfonate (132 mg, 0.32 mmol) and NaN₃ (62 mg, 0.95 mmol) in anhydrous DMF was heated to 80° C. under N₂ atmosphere for 1.5 hr, cooled to rt and diluted with EtOAc, and washed with H₂O (3×20 mL), followed by brine, and dried over anhydrous Na₂SO₄, and filtered, and concentrated under reduced pressure. The resulted slurry was purified through flash chromatography on silica gel (eluted with gradient system: 0-30% EtOAc in hexane) to afford tert-butyl (S)-1-azido-3-((R)-tetrahydro-2H-pyran-3-yl)propan-2-ylcarbamate 58 mg, yield 64%. MS ESI +ve m/z 307 (M+Na).

### Step 4: tert-butyl (S)-1-amino-3-((R)-tetrahydro-2H-pyran-3-yl)propan-2-ylcarbamate

Hydrogenation of tert-butyl (S)-1-azido-3-((R)-tetrahydro-2H-pyran-3-yl)propan-2-ylcarbamate (146 mg, 0.51 mmol) was carried out in MeOH (10 mL), 10% Pd/C (25 mg) under 40 psi of H₂ for 2 h. After filtration 114 mg of tert-butyl (S)-1-amino-3-((R)-tetrahydro-2H-pyran-3-yl)propan-2-ylcarbamate was obtained, yield 86%. MS ESI +ve m/z 259 (M+H).

### Intermediate Preparation 3

### tert-butyl (S)-1-amino-3-((R)-tetrahydro-2H-pyran-3-yl)propan-2-yl(methyl)carbamate

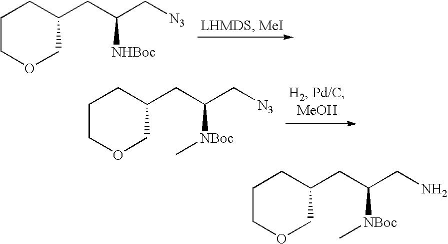

Step 1. tert-butyl (S)-1-azido-3-((R)-tetrahydro-2H-pyran-3-yl)propan-2-yl(methyl)carbamate

To a solution of tert-butyl (S)-1-azido-3-((R)-tetrahydro-2H-pyran-3-yl)propan-2-ylcarbamate (30 mg, 0.11 mmol) in anhydrous THF (4 mL) at −78° C. was added 1.0 M LHMDS solution in THF (253 μL, 0.25 mmol), then stirred at this temperature for 30 min. To this mixture was added MeI (125 μL, 0.22 mmol), then the temperature was allowed to warm to 0° C., and stand for 12 hr in the refrigerator. The reaction mixture was quenched with saturated aq. NH₄Cl, extracted with EtOAc (30 mL), the separated organic phase was washed with H₂O (2×10 mL), brine, and dried (Na₂SO₄), and filtered. The filtrate was concentrated, the resulting slurry was purified through flash chromatography on silica gel (eluted with gradient system, 0-30% EtOAc in hexane) to afford tert-butyl (S)-1-azido-3-((R)-tetrahydro-2H-pyran-3-yl)propan-2-yl(methyl)carbamate 31 mg, yield 100%. MS ESI +ve m/z 321 (M+Na).

### Step 2. tert-butyl (S)-1-amino-3-((R)-tetrahydro-2H-pyran-3-yl)propan-2-yl(methyl)carbamate

Hydrogenation of (S)-1-azido-3-((R)-tetrahydro-2H-pyran-3-yl)propan-2-yl(methyl)carbamate (62 mg, 0.51 mmol) was carried out in EtOAc (20 mL), 10% Pd/C (15 mg) under 40 psi of H₂ for 2 h. After filtration 52 mg of tert-butyl (S)-1-amino-3-((R)-tetrahydro-2H-pyran-3-yl)propan-2-ylcarbamate was obtained, yield 91%. MS ESI +ve m/z 273 (M+H).

### Intermediate Preparation 4

### tert-butyl (S)-1-amino-3-((R)-tetrahydro-2H-pyran-3-yl)propan-2-yl(methyl)carbamate

Alternatively, tert-butyl (S)-1-amino-3-((R)-tetrahydro-2H-pyran-3-yl)propan-2-yl(methyl)carbamate may be prepared by the following procedures:

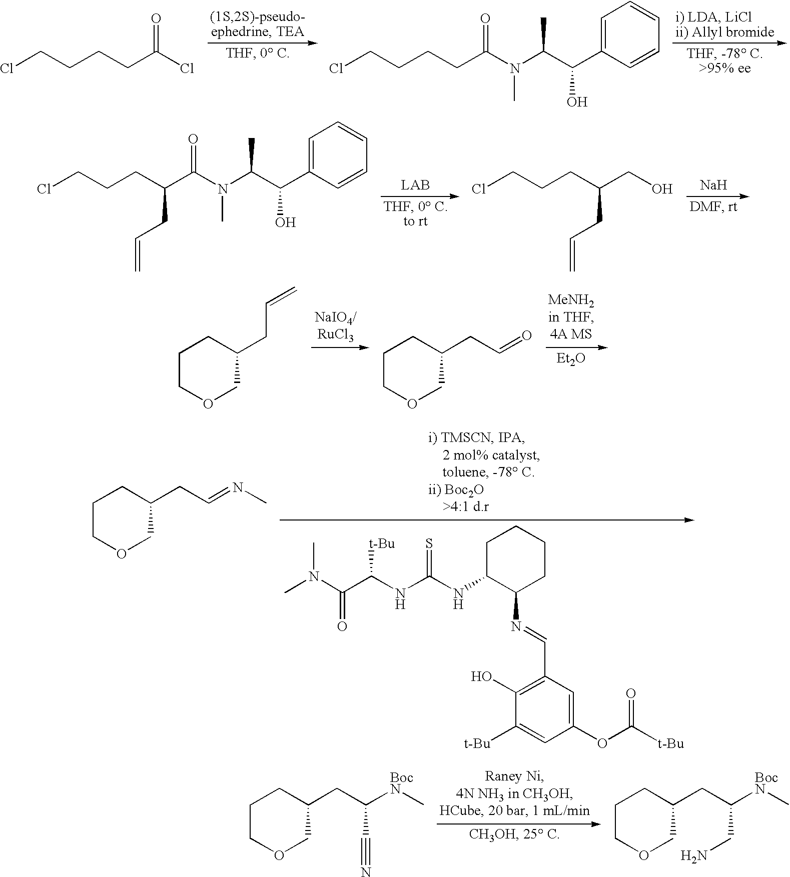

### Step 1. 5-Chloro-N-((1S,2S)-1-hydroxy-1-phenylpropan-2-yl)-N-methylpentanamide

To a magnetically stirred solution of (1S,2S)-pseudoephedrine (60 g, 363.1 mmol) in THF (600 mL) at room temperature was added triethylamine (65.4 mL, 472 mmol) in one portion. The resulting white suspension was cooled to 0° C. A solution of 5-chloropentanoyl chloride (49 mL, 381 mmol) in THF (130 mL) was added dropwise to the mixture over 45 min using an addition funnel. The mixture was then allowed to stir at 0° C. for 30 min. H₂O (40 mL) was added and the resulting mixture was concentrated to ˜10% of the original volume. The resulting solution was partitioned between H₂O/EtOAc and the layers were separated. The aqueous layer was extracted with EtOAc (600 mL). The combined organic layers were washed with saturated aqueous NaHCO₃, brine, dried over MgSO₄, filtered, and concentrated under reduced pressure to furnish the crude product as pale yellow oil. The crude amide was purified by flash chromatography (ISCO; 3×330 g column; CH₂Cl₂ to 5% MeOH/CH₂Cl₂) to provide the product as a clear, viscous oil. The residual Me0H was removed through azeotroping with toluene (3 x 100 mL) to provide 5-chloro-N-((1S,2S)-1-hydroxy-1-phenylpropan-2-yl)-N-methylpentanamide (96.2 g, 339 mmol, 93%). LCMS (rn/z =266.0)

### Step 2. (R)-2-(3-Chloropropyl)-N-((1S,2S)-1-hydroxy-1-phenylpropan-2-yl)-N-methylpent-4-enamide

To a magnetically stirred suspension of LiCl (83 g, 1.96 mol) in THF (700 mL) at room temperature was added diisopropylamine (104 mL, 736 mmol) in one portion. n-BuLi (2.5M in hexane, 281 mL, 703 mmol) was added dropwise over 30 min using an addition funnel. The light yellow mixture stirred at −78° C. for 20 min and then was warmed to 0° C. for 15 min. The mixture was then cooled to −78° C. and 5-chloro-N-((1S,2S)-1-hydroxy-1-phenylpropan-2-yl)-N-methylpentanamide (92.8 g, 327 mmol) in THF (330 mL) was added dropwise over 30 min using an addition funnel. The mixture was stirred at −78° C. for 1 h and then was warmed to 0° C. for 25 min. Allylbromide (41.5 mL, 490 mmol) was then added slowly over 2 min via syringe and then the reaction was warmed to room temperature. The reaction stirred at room temperature for 50 min and was judged complete by LC/MS. The mixture was cooled to 0° C. and saturated aqueous NaHCO₃ (400 mL) and H₂O (200 mL) were added. EtOAc was added, the phases were separated and the aqueous phase was extracted with EtOAc (1500 mL total). The combined organic layers were washed with 1N HCl (4×150 mL), brine, dried over MgSO₄, filtered, and concentrated under reduced pressure to furnish (R)-2-(3-chloropropyl)-N-((1S,2S)-1-hydroxy-1-phenylpropan-2-yl)-N-methylpent-4-enamide as an orange oil (101.2 g, 312 mmol, 95%). The crude material was carried on without further purification. LC/MS (m/z=306.0).

### Step 3. (R)-2-(3-Chloropropyl)pent-4-en-1-ol

A magnetically stirred solution of diisopropylamine (184 mL, 1.29 mol) in THF (600 mL) was cooled to −78° C. n-BuLi (2.5M in hexane, 482 mL, 1.21 mol) was added dropwise over 35 min using an addition funnel. The cloudy mixture stirred at −78° C. for 15 min and then was warmed to 0° C. for 15 min during which time the solution became clear and light yellow. Borane-ammonia complex (90%, 42 g, 1.24 mol) was added in four equal portions, one minute apart. (Caution: vigorous evolution of gas). The cloudy mixture was warmed to room temperature for 20 min and then was recooled to 0° C. (R)-2-(3-chloropropyl)-N-((1S,2S)-1-hydroxy-1-phenylpropan-2-yl)-N-methylpent-4-enamide (100.2 g, 309 mmol) in THF (300 mL) was added dropwise over 10 min using an addition funnel. The reaction was warmed to room temperature and stirred for 2.5 h. The reaction was cooled to −10° C. and was quenched with HCl (3M, 1500 mL). The phases were separated and the aqueous phase was extracted with Et₂O (2000 mL total). The combined organic layers were washed with 3N HCl, brine, dried over MgSO₄, filtered, and concentrated under reduced pressure to furnish the crude product as a yellow oil. The crude material was purified by flash chromatography (ISCO; 330 g column; Hexane to 30% EtOAc/Hexane) to provide (R)-2-(3-chloropropyl)pent-4-en-1-ol as a clear, viscous oil (32.6 g, 200 mmol, 65%); NMR (400 MHz. CDCl₃) δ 5.82 (m, 1H), 5.07 (m, 2H), 3.78 (m, 1H), 3.58 (d, J=8.0 Hz, 2H), 3.54 (t, J=8 Hz, 2H), 2.14 (m, 2H), 1.85 (m, 2H), 1.64 (m, 1H), 1.49 (m, 1H).

### Step 4. (R)-3-Allyl-tetrahydro-2H-pyran

DMF (350 mL) was added to a round bottom flask containing NaH (60% w/w, 15 g, 0.376 mmol) and a magnetic stir bar. The suspension was cooled to 5-10° C. in an ice bath and stirred for 5 min. A solution of (R)-2-(3-chloropropyl)pent-4-en-1-ol (30.6 g, 188 mmol) in DMF (350 mL) was added via addition funnel over 25 min. Caution: Gas evolution and exotherm. The resulting creamy suspension was stirred for 30 min. The reaction was warmed to room temperature and the resulting beige suspension was stirred for 2 h, at which time it was judged complete by TLC. The reaction mixture was cooled to 0° C. and quenched by addition of H₂O (250 mL) and HCl (3N, 250 mL). The phases were separated and the aqueous phase was extracted with petroleum ether (4×250 mL). The combined with organic layers were washed with H₂O, brine, dried over MgSO₄, filtered, and concentrated under reduced pressure to furnish the crude product as a yellow oil. The crude material was purified by flash chromatography (ISCO; 120 g column; Hexane to 30% EtOAc/Hexane) to provide (R)-3-allyl-tetrahydro-2H-pyran as a clear oil (19.8 g, 157 mmol, 83%); NMR (400 MHz. CDCl₃) δ 5.72-5.82 (m, 1H), 5.00-5.06 (m, 2H), 3.86-3.91 (m, 2H), 3.37 (m, 1H), 3.08 (t, J=12 Hz, 1H), 1.85-1.98 (m, 3H), 1.59-1.69 (m, 3H), 1.15-1.21 (m, 1H).

### Step 5. (R)-2-(Tetrahydro-2H-pyran-3-yl)acetaldehyde

To a magnetically stirred solution of (R)-3-allyl-tetrahydro-2H-pyran (18.7 g, 148 mmol) in acetonitrile (740 mL) at room temperature was added RuCl₃.2H₂O (1.43 g, 5.92 mmol) in one portion. The resulting dark brown solution was stirred at room temperature for 5 min and then NaIO₄ (69 g, 326 mmol) was added in one portion. H₂O was added in small portions (10×8 mL) at 5 min intervals. The reaction was stirred at room temperature for 30 min, at which time it was judged complete by TLC. The reaction mixture was quenched by addition of saturated aqueous Na₂S₂O₃ (250 mL) and H₂O (1000 mL). The phases were separated and the aqueous phase was extracted with Et₂O (4×400 mL). The combined with organic layers were washed with H₂O, brine, dried over MgSO₄, filtered, and concentrated under reduced pressure to furnish the crude product as a yellow oil. The crude material was purified by flash chromatography (ISCO; 120 g column; Hexane to 40% EtOAc/Hexane) to provide (R)-2-(tetrahydro-2H-pyran-3-yl)acetaldehyde as a yellow oil (14.3 g, 111 mmol, 60%); ¹H NMR (400 MHz, CDCl₃) δ 9.78 (t, J=2, 1H), 3.84-3.88 (m, 2H), 3.40-3.47 (m, 1H), 3.17 (dd, J=11.2, 8.8 Hz, 1H), 2.31-2.41 (m, 2H), 2.21-2.28 (m, 1H), 1.88-1.93 (m, 1H), 1.61-1.72 (m, 2H), 1.29-1.33 (m, 1H).

### Step 6. (R,E)-N-(2-(Tetrahydro-2H-pyran-3-yl)ethylidene)methanamine

To a magnetically stirred solution of (R)-2-(tetrahydro-2H-pyran-3-yl)acetaldehyde (11 g, 85.8 mmol) in Et₂O (215 mL) at room temperature was added MeNH₂ (2M in THF, 215 mL, 429.2 mmol) and molecular sieves (4A, powdered, activated, 21.5 g). The reaction was stirred at room temperature for 1 h. The resulting mixture was then filtered and concentrated under reduced pressure to furnish (R,E)-N-(2-(tetrahydro-2H-pyran-3-yl)ethylidene)methanamine as a yellow oil (11.3 g, 80 mmol, 93%). The crude material was carried on without further purification. ¹H NMR (400 MHz, CDCl₃) δ 7.67 (m, 1H), 3.86-3.91 (m, 2H), 3.36-3.43 (m, 1H), 3.29 (s, 3H), 3.13 (dd, J=11.0, 9.8 Hz, 1H), 1.95-2.14 (m, 2H), 1.86-1.91 (m, 2H), 1.62-1.68 (m, 2H), 1.21-1.30 (m, 1H).

### Step 7. tent-Butyl (S)-1-cyano-2-((R)-tetrahydro-2H-pyran-3-yl)ethyl(methyl)-carbamate

A 2 L, round bottom flask was charged with toluene (400 mL), a magnetic stir bar, (R,E)-N-(2-(Tetrahydro-2H-pyran-3-yl)ethylidene)methanamine (11.3 g, 80.1 mmol) and 3-{(E)-[((1R,2R)-2-{[({(1S)-1-[(dimethylamino)carbonyl]-2,2-dimethylpropyl}amino)carbonothioyl]amino}cyclohexyl)imino]methyl}-5-(1,1-dimethylethyl)-4-hydroxyphenyl 2,2-dimethylpropanoate (J. Am. Chem. Soc., 2002, 124, 10012-10014) (0.9 g, 1.6 mmol). The mixture was cooled to −78° C. and trimethylsilanecarbonitrile (21.4 mL, 160.2 mmol) was added dropwise over 15 min using an addition funnel. Isopropyl alcohol (12.3 mL, 160.2 mmol) was then added dropwise over 10 min. The reaction stirred at −78° C. for 3 h and then was warmed to room temperature and stirred for 1 h. Bis(1,1-dimethylethyl) dicarbonate (35.0 g, 160.2 mmol) was then added and the resulting mixture was stirred at room temperature for 1 h. The reaction was quenched by the addition of saturated aqueous NaHCO₃ (400 mL) and EtOAc (300 mL). The layers were separated and the aqueous layer was washed with EtOAc (100 mL). The combined organic layers were dried over Na₂SO₄, filtered, and concentrated under reduced pressure to give the crude product. The crude material was divided into two parts and each was purified by flash chromatography (ISCO; 120 g column; 0% to 10% EtOAc/Hexane over 30 min, then 10% EtOAc/Hexane 47 min, then 10% to 20% EtOAc/Hexane over 2 min, then 20% EtOAc/Hexane for 11 min). The two purified batches were combined to provide tert-butyl (S)-1-cyano-2-((R)-tetrahydro-2H-pyran-3-yl)ethyl(methyl)carbamate (18.9 g, 70 mmol, 86%) as an orange oil. ¹H NMR (400 MHz, CDCl₃) δ 5.00 (brs, 1H), 3.83-3.90 (m, 2H), 3.42-3.48 (m, 1H), 3.19 (dd, J=11.3, 8.6, 1H), 2.92 (s, 3H), 1.85-1.95 (m, 1H), 1.60-1.82 (m, 5H), 1.50 (s, 9H), 1.28-1.33 (m, 1H).

### Step 8. tert-Butyl (S)-1-amino-3-((R)-tetrahydro-2H-pyran-3-yl)propan-2-yl(methyl)carbamate

tert-Butyl (S)-1-cyano-2-((R)-tetrahydro-2H-pyran-3-yl)ethyl(methyl)carbamate (397 mg, 4:1 mixture of diastereomers at the alpha-amino stereocenter) was dissolved in a solution of 4M NH₃ in MeOH (15 mL) and passed through a Raney-nickel cartridge (CatCart®, 50 mm) on an in-line hydrogenation apparatus (H-Cube) with the following settings: ambient temperature (14° C.), flow rate 1.0 mL/min, H₂ pressure 30 atm. The solution was recirculated so that the product solution was fed back into the apparatus. After thirty minutes, TLC analysis (1:9 MeOH/CH₂Cl₂, KMnO₄ stain) showed complete conversion of the starting material. After 60 min total reaction time, the solution was evaporated to yield 371 mg (92%) of tert-butyl (S)-1-amino-3-((R)-tetrahydro-2H-pyran-3-yl)propan-2-yl(methyl)carbamate as a clear, rose-colored oil. LC-MS (ELSD) m/z 273.6 (M+H)⁺.

### Intermediate Preparation 5

### 1,1-Dimethylethyl methyl {(1S)-2-({[(phenylmethyl)oxy]carbonyl}amino)-1-[(3R)-tetrahydro-2H-pyran-3-ylmethyl]ethyl}carbamate and 1,1-Dimethylethyl methyl {(1R)-2-({[(phenylmethyl)oxy]carbonyl}amino)-1-[(3R)-tetrahydro-2H-pyran-3-ylmethyl]ethyl}carbamate

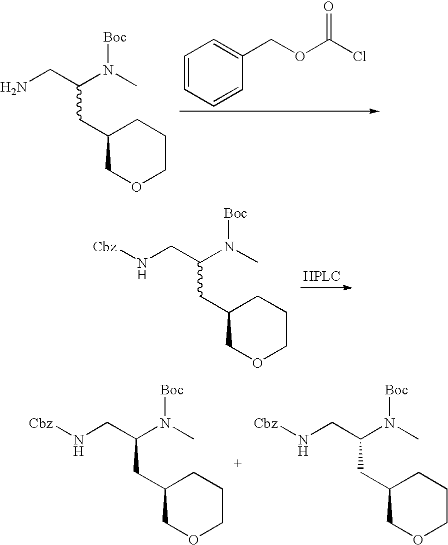

### Step 1. 1,1-Dimethylethyl methyl{2-({[(phenylmethyl)oxy]carbonyl}amino)-1-[(3R)-tetrahydro-2H-pyran-3-ylmethyl]ethyl}carbamate

To a 50 mL round-bottomed flask was added 1,1-dimethylethyl {2-amino-1-[(3R)-tetrahydro-2H-pyran-3-ylmethyl]ethyl}methylcarbamate (815 mg, 2.99 mmol) in dichloromethane (15 ml) to give a tan solution. The mixture was cooled to 0° C. (ice bath) and then N,N-diisopropylethylamine (1.045 ml, 5.98 mmol) and benzyl chloroformate (0.641 ml, 4.49 mmol) were added. After stirring for 3 hours at 0° C., the reaction was quenched with sat NH₄Cl (2 mL) and water (1 mL). The phases were separated and the organic layer was washed with sat NH₄Cl (2 mL). The aqueous layer was back extracted with CH₂Cl₂ (1×5 mL) and the combined organic layers and washed with saturated NaCl, dried over MgSO₄, filtered and concentrated to give 1.6 g of crude product as a reddish oil. The crude residue was purified by flash chromatography on silica gel {ISCO Combiflash, 40 g Analogix column, CH₂Cl₂/MeOH 0%→5%} and 1.10 g (dr 4:1) of 1,1-dimethylethyl methyl{2-({[(phenylmethypoxy]carbonyl}amino)-1-[(3R)-tetrahydro-2H-pyran-3-ylmethyl]ethyl}carbamate isolated as a reddish oil.

### Step 2. 1,1-Dimethylethyl methyl {(1S)-2-({[(phenylmethyl)oxy]carbonyl}amino)-1-[(3R)-tetrahydro-2H-pyran-3-ylmethyl]ethyl}carbamate and 1,1-Dimethylethyl methyl{(1R)-2-({[(phenylmethyl)oxy]carbonyl}amino)-1-[(3R)-tetrahydro-2H-pyran-3-ylmethyl]ethyl}carbamate

Purification via chiral HPLC [OD-H column (20×250 mm), 10/90 isopropanol/hexane with 0.1% diethylamine @ 10 mL/min] was necessary to separate the two diastereomers. The sample was dissolved in MeOH (10 mL), filtered and injected (16×). The combined fractions were collected and concentrated to give 1,1-dimethylethyl methyl{(1S)-2-({[(phenylmethyl)oxy]carbonyl}amino)-1-[(3R)-tetrahydro-2H-pyran-3-ylmethyl]ethyl}carbamate (732 mg, 1.765 mmol, 59.0% yield) (>99% de) as a pink oil (HPLC retention time of 7.46 min) and 95 mg of 1,1-dimethylethyl methyl{(1R)-2-({[(phenylmethyl)oxy]carbonyl}amino)-1-[(3S)-tetrahydro-2H-pyran-3-ylmethyl]ethyl}carbamate as a pink oil (HPLC retention time of 9.3 min). MS (m/z) 307.2 (M+H-Boc⁺).

### Intermediate Preparation 6

### 1,1-Dimethylethyl {(1S)-2-amino-1-[(3R)-tetrahydro-2H-pyran-3-ylmethyl]ethyl}methylcarbamate

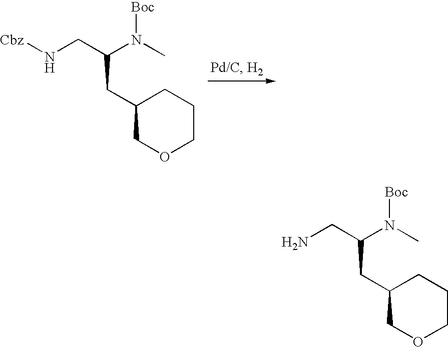

To a flask containing the 1,1-dimethylethyl methyl{(1S)-2-({[(phenylmethyl)oxy]carbonyl}amino)-1-[(3R)-tetrahydro-2H-pyran-3-ylmethyl]ethyl}carbamate and a large stirbar was added MeOH (10 mL). Palladium on carbon (0.093 g, 10% on carbon, 5 mol %) was added and a balloon of hydrogen affixed to the flask with a three-way valve. Very carefully, the contents of the flask were partially evacuated and refilled with N₂ several times while stirring, then partially evacuated and refilled with H₂ several times in such a way as to avoid bumping or excessive boiling. The hydrogenation was allowed to proceed at rt with vigorous stirring. After 1.5 h, TLC (5% MeOH/DCM) showed that the reaction was complete. The mixture was filtered through a pad of Celite and sand (cloudy, colorless), then through a 0.45 micron PTFE syringe filter (clear, colorless), and evaporated to yield 473.2 mg of 1,1-dimethylethyl {(1S)-2-amino-1-[(3R)-tetrahydro-2H-pyran-3-ylmethyl]ethyl}methylcarbamate as a clear slightly rose-colored heavy oil after drying in vacuo (100%).

### Intermediate Preparation 7

### 1,1-dimethylethyl {(1R)-2-amino-1-[(3R)-tetrahydro-2H-pyran-3-ylmethyl]ethyl}methylcarbamate

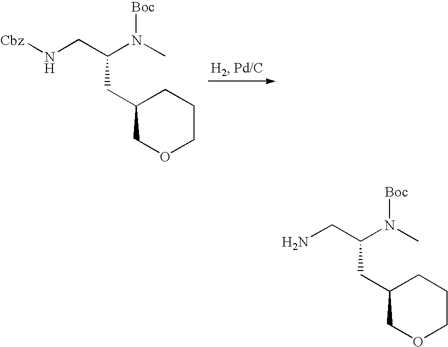

A solution of 1,1-dimethylethyl methyl{(1R)-2-({[(phenylmethyl)oxy]carbonyl}amino)-1-[(3R)-tetrahydro-2H-pyran-3-ylmethyl]ethyl}carbamate (0.175 g, 0.43 mmol) in 10 mL of MeOH was purged under nitrogen before it was charged with 10% Pd on carbon (0.023 g). The resulting mixture placed under a hydrogen balloon and was degassed three times and backfilled with hydrogen. The mixture was then maintained under hydrogen with stirring for 2 hours at room temperature. The crude material was filtered though a layer of celite under nitrogen and then though a 0.45 micron PTFE synringe filter to provide a clear solution which was concentrated to dryness to afford 1,1-dimethylethyl {(1R)-2-amino-1-[(3R)-tetrahydro-2H-pyran-3-ylmethyl]ethyl}methylcarbamate (0.08 g) as a colorless oil, which was used directly in the next reaction.

### Intermediate Preparation 8

### tert-butyl (S)-1-amino-3-((R)-tetrahydro-2H-pyran-3-yl)propan-2-yl(methyl)carbamate

Alternatively, tert-butyl (5)-1-amino-3-((R)-tetrahydro-2H-pyran-3-yl)propan-2-yl(methyl)carbamate may be prepared by the following procedures:

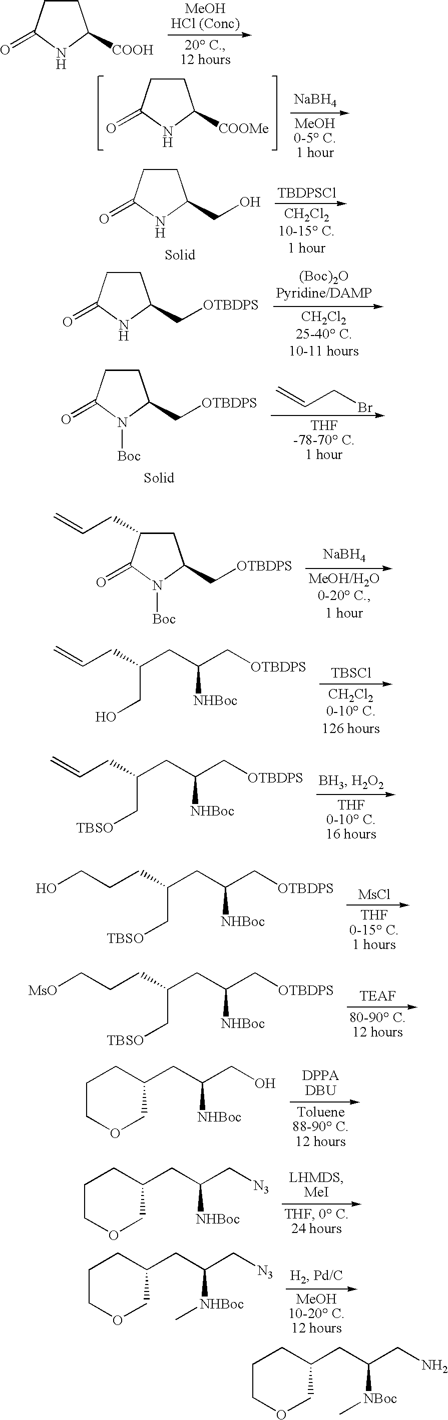

### Alternative Procedure:

Alternatively, tert-butyl (S)-1-amino-3-((R)-tetrahydro-2H-pyran-3-yl)propan-2-yl(methyl)carbamate may also be prepared by the following process where chiral hydrogenation catalysts may be used in a series of hydrogenation steps to provide enantiomerically enriched intermediates:

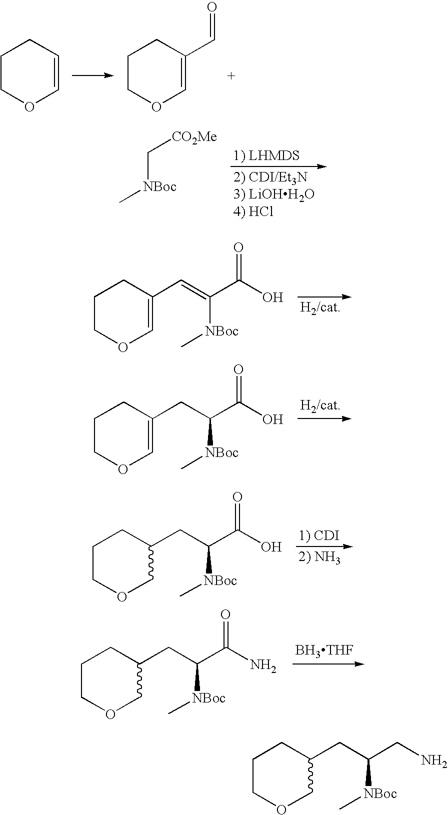

For example, hydrogenation of the dihydropyran-ene-amine to form the dihydropyran-amine may be accomplished in methanol, at 25° C., using about 88-110 psi hydrogen pressure, using 1-2 mol % of a catalyst generated from [Rh(nbd)₂]BF₄ and SL-M004-1 (SL-M004-1: (αR,αR)-2,2′-bis(α-N,N-dimethyl-aminophenylmethyl)-(S,S)-1,1′-bis[di(3,5-dimethyl-4-methoxyphenyl)phosphino]ferrocene, available from Solvias, Inc. Fort Lee, N.J.). Hydrogenation of the dihydropyran-amine to form the tetrahydropyran-amine may be accomplished at 50° C., using about 80 bar hydrogen pressure and 4 mol % catalyst loading of a catalyst generated from [Rh(COD)₂]O₃SCF₃ and SL-A109-2 (solvent: THF) or [Rh(nbd)₂]BF₄ and SL-A109-2 (solvent: methanol) (SL-A109-2: (S)-(6,6′-dimethoxybiphenyl-2,2′-diyl)-bis[bis(3,5-di-tert-butyl-4-methoxyphenyl)phosphine], available from Solvias, Inc. Fort Lee, N.J.).

### Intermediate Preparation 9

### 1,1-Dimethylethyl methyl{(1S)-2-({[(phenylmethyl)oxy]carbonyl}amino)-1-[(3S)-tetrahydro-2H-pyran-3-ylmethyl]ethyl}carbamate and 1,1-Dimethylethyl methyl{(1R)-2-({[(phenylmethyl)oxy]carbonyl}amino)-1-[(3S)-tetrahydro-2H-pyran-3-ylmethyl]ethyl}carbamate

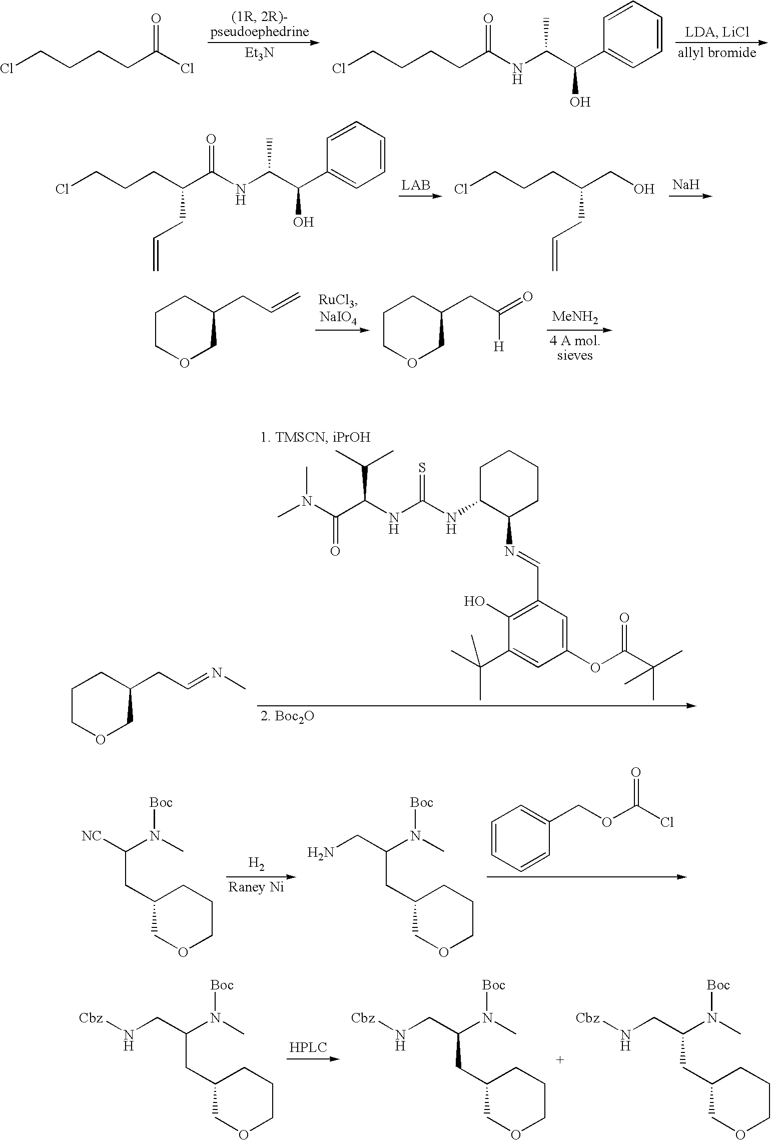

### Step 1. 5-Chloro-N-((1R,2R)-1-hydroxy-1-phenylpropan-2-yl)-N-methylpentanamide

5-Chloro-N-((1R,2R)-1-hydroxy-1-phenylpropan-2-yl)-N-methylpentanamide was prepared from 5-chloropentanoyl chloride (7.8 mL, 60.4 mmol) and (1R, 2R)-pseudoephedrine (9.9 g, 60.4 mmol) according to the method described in Intermediate Preparation 4, Step 1.

### Step 2. (S)-2-(3-Chloropropyl)-N-((1R,2R)-1-hydroxy-l-phenylpropan-2-yl)-N-methylpent-4-enamide

(5)-2-(3-Chloropropyl)-N-((1R,2R)-1-hydroxy-1-phenylpropan-2-yl)-N-methylpent-4-enamide was prepared from 5-chloro-N-((1R,2R)-1-hydroxy-1-phenylpropan-2-yl)-N-methylpentanamide (17.7 g, 60.2 mmol) according to the method described in Intermediate Preparation 4, Step 2.

### Step 3. (S)-2-(3-Chloropropyl)pent-4-en-1-ol

(S)-2-(3-Chloropropyl)pent-4-en-1-ol was prepared from (S)-2-(3-chloropropyl)-N-((1R,2R)-1-hydroxy-1-phenylpropan-2-yl)-N-methylpent-4-enamide (18.2 g, 56.2 mmol) according to the method described in Intermediate Preparation 4, Step 3.

### Step 4. (3S)-3-(2-propen-1-yl)tetrahydro-2H-pyran

(3S)-3-(2-propen-1-yl)tetrahydro-2H-pyran was prepared from (S)-2-(3-chloropropyl)pent-4-en-1-ol (0.951 g, 5.84 mmol) according to the method described in Intermediate Preparation 4, Step 4.

### Step 5. (3S)-tetrahydro-2H-pyran-3-ylacetaldehyde

(3S)-tetrahydro-2H-pyran-3-ylacetaldehyde was prepared from (3S)-3-(2-propen-1-yl)tetrahydro-2H-pyran (4.5 g, 35.6 mmol) according to the method described in Intermediate Preparation 4, Step 5.

### Step 6. N-{(1E)-2-[(3S)-tetrahydro-2H-pyran-3-yl]ethylidene}methanamine

N-{(1E)-2-[(3S)-tetrahydro-2H-pyran-3-yl]ethylidene}methanamine was prepared from (3S)-tetrahydro-2H-pyran-3-ylacetaldehyde (2.75 g, 21.5 mmol) according to the method described in Intermediate Preparation 4, Step 6.

### Step 7. 1,1-Dimethylethyl {1-cyano-2-[(3S)-tetrahydro-2H-pyran-3-yl]ethyl}methylcarbamate

1,1-Dimethylethyl {1-cyano-2-[(3S)-tetrahydro-2H-pyran-3-yl]ethyl}methylcarbamate was prepared as a 3:1 mixture of diastereomers from N-{(1E)-2-[(3S)-tetrahydro-2H-pyran-3-yl]ethylidene}methanamine (2.52 g, 17.8 mmol) according to the method described in Intermediate Preparation 4, Step 7.

### Step 8. 1,1-Dimethylethyl {2-amino-1-[(3S)-tetrahydro-2H-pyran-3-ylmethyl]ethyl}methylcarbamate

1,1-Dimethylethyl {2-amino-1-[(3S)-tetrahydro-2H-pyran-3-ylmethyl]ethyl}methylcarbamate was prepared from 1,1-dimethylethyl {1-cyano-2-[(3S)-tetrahydro-2H-pyran-3-yl]ethyl}methylcarbamate (3.75 g, 13.97 mmol) according to the method described in Intermediate Preparation 4, Step 8.

### Step 9. 1,1-Dimethylethyl methyl{2-({[(phenylmethyl)oxy]carbonyl}amino)-1-[(3S)-tetrahydro-2H-pyran-3-ylmethyl]ethyl}carbamate

1,1-Dimethylethyl methyl{2-({[(phenylmethyl)oxy]carbonyl}amino)-1-[(3S)-tetrahydro-2H-pyran-3-ylmethyl]ethyl}carbamate was prepared from 1,1-dimethylethyl {2-amino-1-[(3S)-tetrahydro-2H-pyran-3-ylmethyl]ethyl}methylcarbamate (3.71 g, 13.62 mmol) according to the method described in Intermediate Preparation 5, Step 1.

### Step 10. 1,1-Dimethylethyl methyl{(1S)-2-({[(phenylmethyl)oxy]carbonyl}amino)-1-[(3S)-tetrahydro-2H-pyran-3-ylmethyl]ethyl}carbamate and 1,1-Dimethylethyl methyl {(1R)-2-({[(phenylmethyl)oxy]carbonyl}amino)-1-[(3S)-tetrahydro-2H-pyran-3-ylmethyl]ethyl}carbamate

The diastereomers of 1,1-dimethylethyl methyl{2-({[(phenylmethyl)oxy]carbonyl}amino)-1-[(3S)-tetrahydro-2H-pyran-3-ylmethyl]ethyl}carbamate were separated via chiral, preparative HPLC (OD-H column (20×250 mm) 20/80 isopropanol/hexane w/0.1% DEA @ 12 mL/min, Runtime −22 min). A 730 mg sample was dissolved in 7.5 mL methanol and then filtered. Another second sample (870 mg) was also dissolved in 8 mL methanol and then filtered. Approximately 196 mg were injected onto the column in a total of 11 injections. The fractions corresponding to the first peak (retention time of 4.45 min) were combined and concentrated to afford 1,1-dimethylethyl methyl{(1S)-2-({[(phenylmethyl)oxy]carbonyl}amino)-1-[(3S)-tetrahydro-2H-pyran-3-ylmethyl]ethyl}carbamate (1.31 g). The fractions corresponding to the second peak (retention time of 8.74 min) were combined and concentrated to provide 1,1-dimethylethyl methyl {(1R)-2-({[(phenylmethyl)oxy]carbonyl}amino)-1-[(3S)-tetrahydro-2H-pyran-3-ylmethyl]ethyl}carbamate (0.176 g).

### Intermediate Preparation 10

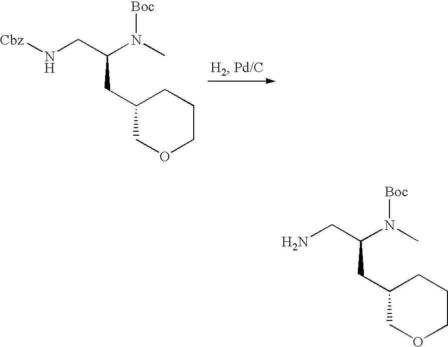

A solution of 1,1-dimethylethyl methyl{(1S)-2-({[(phenylmethyl)oxy]carbonyl}amino)-1-[(3S)-tetrahydro-2H-pyran-3-ylmethyl]ethyl}carbamate (1.31 g 3.22 mmol) in 25 ml of MeOH was purged under nitrogen and the flask charged with 10% Pd on carbon (0.171 g). The resulting mixture was fitted with a three-way adapter equipped with a hydrogen balloon. The flask was evacuated and backfilled three times with hydrogen and them maintained under a hydrogen atmosphere for 2 hours at room temperature. The crude material was filtered though a layer of celite under nitrogen and then though a 0.45 micron PTFE syringe filter and concentrated to afford 1,1-dimethylethyl {(1S)-2-amino-1-[(3S-tetrahydro-2H-pyran-3-ylmethyl]ethyl}methylcarbamate (0.876 g), which was used in the next step without further purification

### Intermediate Preparation 11

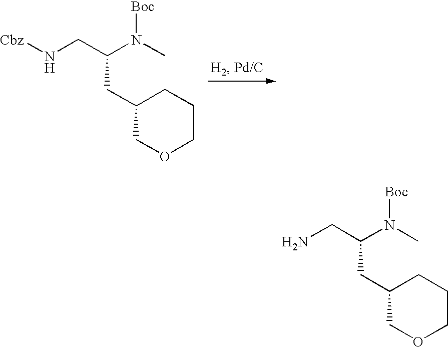

A solution of 1,1-dimethylethyl methyl{(1R)-2-({[(phenylmethyl)oxy]carbonyl}amino)-1-[(3S)-tetrahydro-2H-pyran-3-ylmethyl]ethyl}carbamate (0.176 g, 0.433 mmol) in 10 ml of MeOH was purged under nitrogen and the flask charged with 10% Pd on carbon (0.023 g). The resulting mixture was fitted with a three-way adapter equipped with a hydrogen balloon. The flask was evacuated and backfilled three times with hydrogen and them maintained under a hydrogen atmosphere for 2 hours at room temperature. The crude material was filtered though a layer of celite under nitrogen and then though a 0.45 micron PTFE syringe filter and concentrated to afford 1,1-dimethylethyl 1,1-dimethylethyl {(1R)-2-amino-1-[(3S)-tetrahydro-2H-pyran-3-ylmethyl]ethyl}methylcarbamate (0.120 g), which was used in the next step without further purification.

### Intermediate Preparation 12

### 1,1-Dimethylethyl [(1S)-2-azido-1-(cyclohexylmethyl)ethyl]methylcarbamate

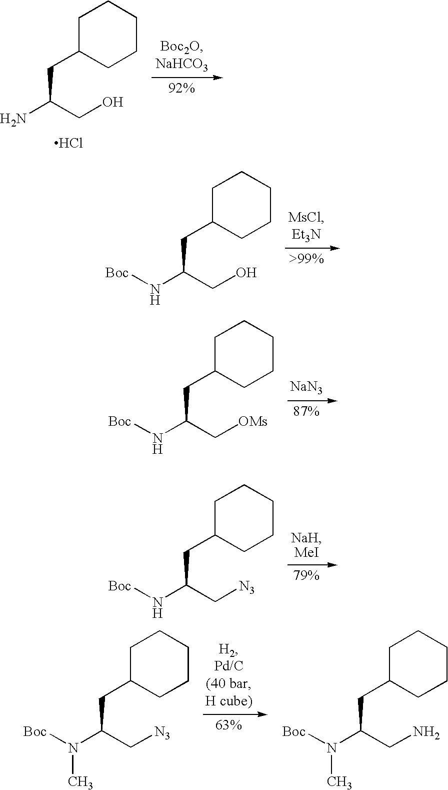

### Step 1. 1,1-dimethylethyl [(1S)-2-cyclohexyl-1-(hydroxymethyl)ethyl]carbamate

To a solution of (2S)-2-amino-3-cyclohexyl-1-propanol hydrochloride (5.0 g, 25.8 mmol) in dioxane (52 mL) and water (26 mL) at 0° C., sodium bicarbonate (2.16 g, 25.8 mmol) was added. Boc₂O then added in one portion. The resulting mixture was allowed to warm to room temperature and stir for 15 min before additional sodium bicarbonate (2.16 g, 25.8 mmol) was added. The mixture was then stirred overnight at room temperature. At this time the solvent was removed in vacuo and the residue taken up in ethyl acetate and water. The layers were separated and the aqueous layer extracted with ethyl acetate. The combined organics were then washed with brine, dried over Na₂SO₄, filtered, and concentrated in vacuo. The crude material was purified via column chromatography (ISCO, 40 g column, 0-20% ethyl acetate/methylene chloride) to give 6.08 g of 1,1-dimethylethyl [(1S)-2-cyclohexyl-1-(hydroxymethyl)ethyl]carbamate as a colorless oil (92%). MS (m/z) 258.6 (M+H⁺).

### Step 2. (2S)-3-Cyclohexyl-2-({[(1,1-dimethylethyl)oxy]carbonyl}amino)propyl methanesulfonate

To a solution of 1,1-dimethylethyl [(1S)-2-cyclohexyl-1-(hydroxymethyl)ethyl]carbamate (1.0 g, 3.89 mmol) and triethylamine (1.18 g, 11.7 mmol) in 16 mL of methylene chloride at 0° C., methanesulfonyl chloride (0.534 g, 4.66 mmol) was added. The resulting mixture was then warmed to rt and stirred for 50 min. The reaction mixture was washed the 0.1 N HCl, and the aqueous layer back-extracted with methylene chloride. The combined organic layers were then washed with saturated aqueous NaHCO₃, dried over Na₂SO₄, filtered and concentrated in vacuo to give 1.58 g (2S)-3-cyclohexyl-2-({[(1,1-dimethylethyl)oxy]carbonyl}amino)propyl methanesulfonate as a waxy yellow solid. The crude material was used in the next reaction without further purification. MS (m/z) 336.4 (M+H⁺).

### Step 3. 1,1-Dimethylethyl [(1S)-2-azido-1-(cyclohexylmethyl)ethyl]carbamate

To a solution of (2S)-3-cyclohexyl-2-({[(1,1-dimethylethyl)oxy]carbonyl}amino)propyl methanesulfonate (1.58 g, 3.89 mmol) in DMF (13 mL), sodium azide (1.26 g, 19.4 mmol) was added. The resulting mixture was then heated to 80° C. overnight. The mixture was then diluted was water and extracted with ether (3×). The combined organics were then washed with brine (3×), dried over Na₂SO₄, filtered and concentrated in vacuo. The crude product was purified via column chromatography (ISCO, 40 g cartride, 0-50% ethyl acetate/hexanes) to afford 0.945 g of 1,1-dimethylethyl [(1S)-2-azido-1-(cyclohexylmethyl)ethyl]carbamate as a colorless oil (87%). MS (m/z) 283.6 (M+H⁺).

### Step 4. 1,1-Dimethylethyl [(1S)-2-azido-1-(cyclohexylmethyl)ethyl]methylcarbamate

To a solution of 1,1-dimethylethyl [(1S)-2-azido-1-(cyclohexylmethyl)ethyl]carbamate (0.945 g, 3.35 mmol) in 17 mL of DMF at room temperature, sodium hydride (0.201 g, 5.02 mmol of a 60% dispersion in mineral oil) was added. Some gas evolution occurred and the solution turned yellow. Methyl iodide (0.312 mL, 5.02 mmol) then added and the resulting mixture stirred at room temperature for 1.5 h. The reaction mixture was quenched with 0.1 N HCl and partitioned between ether and water. The layers were separated and the aqueous layer backextracted with ether (2×). The combined organic layers were then washed with bring (3×), dried over Na₂SO₄, filtered and concentrated in vacuo. The crude product was purified via column chromatography (ISCO, 40 g cartridge, 0-50% ethyl acetate/hexanes), but still contained DMF. The crude material was then dissolved in ether, washed with brine (3×), dried over Na₂SO₄, filtered and concentrated in vacuo to provide 0.785 g of 1,1-dimethylethyl [(1S)-2-azido-1-(cyclohexylmethyl)ethyl]methylcarbamate as a colorless oil (79%). MS (m/z) 297.6 (M+H⁺).

The following diamine was prepared using procedures analogous to those described above substituting the indicated amino alcohol in Step 1:

| Diamine | Name | Amino Alcohol |
|---|---|---|
| 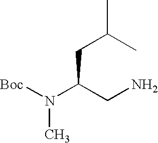 | 1,1-dimethylethyl [(1S)-1- (aminomethyl)-3- methylbutyl]methyl- carbamate | (2S)-2-amino-4- methyl-1-pentanol |

### Intermediate Preparation 13

### 1,1-dimethylethyl [(1S)-2-amino-1-(tetrahydro-2H-pyran-4-ylmethyl)ethyl]methylcarbamate

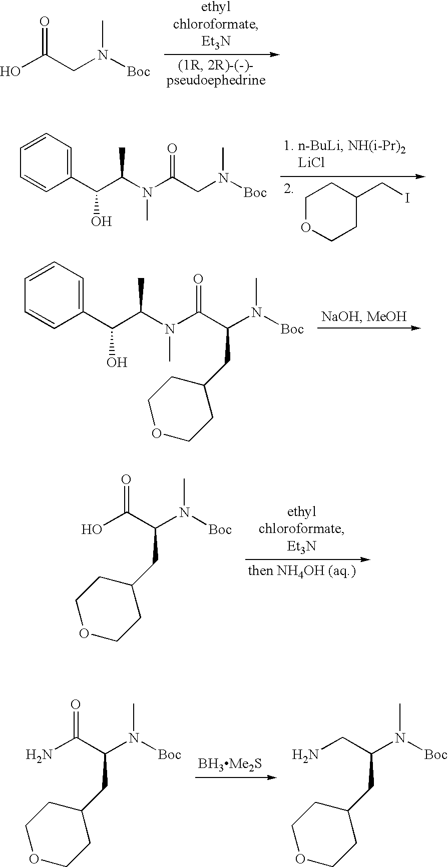

### Step 1. 1,1-dimethylethyl {2-[[(1R,2R)-2-hydroxy-1-methyl-2-phenylethyl](methyl)amino]-2-oxoethyl}methylcarbamate

To a solution of N-Boc-sarcosine (3.78 g, 20 mmol) and triethylamine (6.13 ml, 44 mmol) in THF (50 ml) at 0° C. was added ethyl chloroformate (1.91 mL, 20 mmol) to give a white suspension. The resulting suspension was stirred at 0° C. for 10 min and then warmed to rt for 2 h. The mixture was then recooled and (1R,2R)-(−)-pseudoephedrine (3.30 g, 20 mmol) was added and the resulting mixture was allowed to warm to room temperature and stir for 18 h. The reaction was concentrated and the residue dissolved in ethyl acetate and water (30 mL each). The layers were separated and the aqueous layer extracted with ethyl acetate (2×20 mL). The combined extracts were washed with HCl (1M, 20 mL), NaOH (1M, 20 mL), and brine, dried over MgSO₄, filtered and concentrated in vacuo, to give 5.07 g of crude material as a light amber oil. The product was purified via column chromatography (200 g silica gel 60, 230-400 mesh, 1-1.5% MeOH/CH₂Cl₂) to provide 1,1-dimethylethyl {2-[[(1R,2R)-2-hydroxy-1-methyl-2-phenylethyl](methyl)amino]-2-oxoethyl}methylcarbamate (2.77 g, 41.2%). MS (m/z) 337.0 (M+H^{±}).

### Step 2. 1,1-dimethylethyl [(1S)-2-[[(1R,2R)-2-hydroxy-1-methyl-2-phenylethyl](methyl)amino]-2-oxo-1-(tetrahydro-2H-pyran-4-ylmethyl)ethyl]methylcarbamate

To a solution of diisopropylamine (2.19 ml. 15.36 mmol) in THF (20 mL) at −78° C., was added n-butyl lithium (6.46 ml, 2.5M in hexane, 16.15 mmol) dropwise. The resulting mixture was stirred at −78° C. for 30 min and was then added to a mixture of 1,1-dimethylethyl {2-[[(1R,2R)-2-hydroxy-1-methyl-2-phenylethyl](methyl)amino]-2-oxoethyl}methylcarbamate (2.65 g, 7.88 mmol) and lithium chloride (2.0g, 47.3 mmol) via cannula at −23° C. The resulting mixture was stirred for 24 h and allowed to warm to room temperature before it was recooled in an ice bath and quenched with HCl (1M, 15.8 ml). The mixture was then extracted with EtOAc (3×20 ml) and the combined extracts washed with saturated NH₄Cl, brine, dried, filtered, and concentrated. This crude product was purified by column chromatography (160 g silica gel 60, 230-400 mesh, 25,30,40, then 50% EtOAc/hexanes) to provide 1,1-dimethylethyl [(1S)-2-[[(1R,2R)-2-hydroxy-1-methyl-2-phenylethyl](methyl)amino]-2-oxo-1-(tetrahydro-2H-pyran-4-ylmethyl)ethyl]methylcarbamate (510 mg, 95% pure and 1.2 g, 80% pure, 42% combined yield). MS (m/z) 435.2 (M+H⁺).

### Step 3. N-{[(1,1-dimethylethyl)oxy]carbonyl}-N-methyl-3-(tetrahydro-2H-pyran-4-yl)-L-alanine

To a solution of 1,1-dimethylethyl [(1S)-2-[[(1R,2R)-2-hydroxy-1-methyl-2-phenylethyl](methyl)amino]-2-oxo-1-(tetrahydro-2H-pyran-4-ylmethyl)ethyl]methylcarbamate (505 mg, 1.162 mmol) in methanol (20 mL), was added NaOH (5.81 ml, 1M). The resulting mixture was heated to reflux for 3 days. The reaction mixture was concentrated and the residue diluted with water (20 ml) and washed with ether (2×20 mL) and the combined ether washes were extracted with 0.5M NaOH (1×10 mL). The combined aqueous extracts were acidified with HCl (2M) to pH=1 and then extracted with EtOAc (2×50 ml). The combined organic extracts were washed with brine, dried, filtered and concentrated in vacuo to give N-{[(1,1-dimethylethyl)oxy]carbonyl}-N-methyl-3-(tetrahydro-2H-pyran-4-yl)-L-alanine (306 mg) as a clear oil, which was used in the next step without further purification. MS (m/z) 288.4 (M+H⁺).

### Step 4. 1,1-dimethylethyl [(1S)-2-amino-2-oxo-1-(tetrahydro-2H-pyran-4-ylmethyl)ethyl]methylcarbamate

To a solution of N-{[(1,1-dimethylethyl)oxy]carbonyl}-N-methyl-3-(tetrahydro-2H-pyran-4-yl)-L-alanine (296 mg, 1.03 mmol) and triethylamine (316 μl, 2.266 mmol) in THF (10 ml) at 0° C. was added ethyl chloroformate (98 μl, 1.03 mmol) to give a white suspension. The resulting suspension was stirred at 0° C. for 10 min and then warmed to rt for 2 h. The mixture was then recooled and ammonium hydroxide (0.5 ml) was added and the resulting mixture was allowed to warm to room temperature and stir for another 18 h. The reaction was concentrated and the residue diluted with ethyl acetate and water (10 mL each). The layers were separated and the aqueous layer extracted with ethyl acetate (2×10 mL). The combined organic extracts were washed with brine and dried over MgSO₄, filtered, and concentrated in vacuo to provide 1,1-dimethylethyl [(1S)-2-amino-2-oxo-1-(tetrahydro-2H-pyran-4-ylmethyl)ethyl]methylcarbamate (0.250 g), which was used in the next step without further purification. MS (m/z) 286.8 (M+H⁺).

### Step 5. 1,1-dimethylethyl [(1S)-2-amino-1-(tetrahydro-2H-pyran-4-ylmethyl)ethyl]methylcarbamate

To a refluxing solution of 1,1-dimethylethyl [(1S)-2-amino-2-oxo-1-(tetrahydro-2H-pyran-4-ylmethyl)ethyl]methylcarbamate (0.250 g, 0.873 mmol) in THF (10 ml) under argon was added borane dimethylsulfide complex (873 mL, 2M in THF, 1.75 mmol). The resulting mixture was heated at reflux for 2 h. After cooling to room temperature, the reaction mix was treated with KHSO₄ (600 mg) in water (6 ml), and the mixture was stirred at rt for 30 min. Excess NaOH (1N) was then added and the mixture extracted with ether. The ethereal extracts were washed with water, brine, dried, filtered, and concentrated. The crude material was purified via SCX column (loaded with methanol, washed with methanol and then eluted with 2M ammonia in methanol) to provide 1,1-dimethylethyl [(1S)-2-amino-1-(tetrahydro-2H-pyran-4-ylmethyl)ethyl]methylcarbamate (0.103 g, 43%). MS (m/z) 273.5 (M+H⁺).

### Intermediate Preparation 14

### 3-{(3-chlorophenyl)[(2-{[(methyloxy)carbonyl]amino}ethyl)oxy]methyl}benzoic acid

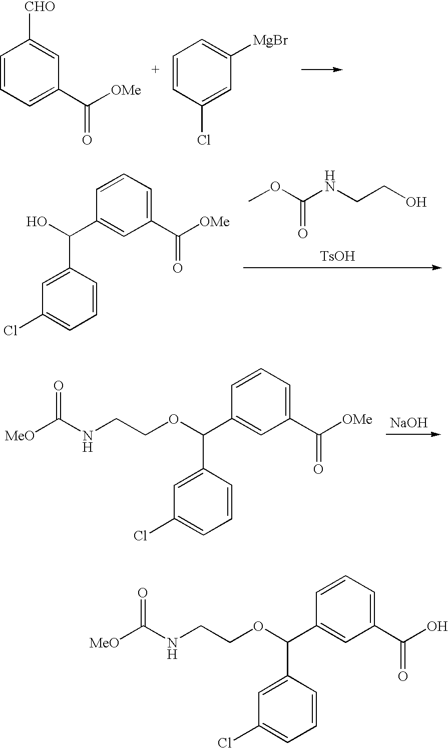

### Step 1. Methyl 3-[(3-chlorophenyl)(hydroxy)methyl]benzoate

To a solution of methyl-3-formylbenzoate (5 g, 30.5 mmol) in 70 mL of ether at 0° C., 3-chlorophenylmagnesium bromide (67 mL of a 0.5 M solution in THF, 33.5 mmol). After 1.5 h at 0° C., the reaction mixture was quenched by addition of saturated NaHCO₃ solution and water and the biphasic mixture was extracted with ethyl acetate. The combined organic layers were washed with brine, dried over MgSO₄, filtered and concentrated in vacuo to afford 9.2 g of a yellowish oil. This material was combined with 0.83 g of crude material (3.05 mmol of starting material) from a previous experiment and purified via column chromatography (ISCO; 10-100% ethyl acetate/hexanes) to afford 8.2 g of methyl 3-[(3-chlorophenyl)(hydroxy)methyl]benzoate (89% yield). MS (m/z) 277.3 (M+H⁺).

### Step 2. Methyl 3-{(3-chlorophenyl)[(2-{[(methyloxy)carbonyl]amino}ethyl)oxy]methyl}benzoate

To a solution of methyl 3-[(3-chlorophenyl)(hydroxy)methyl]benzoate (1.0 g, 3.6 mmol) and methyl (2-hydroxyethyl)carbamate (0.43 g, 3.6 mmol) in toluene p-toluenesulfonic acid (0.68 g, 3.6 mmol) was added. The resulting mixture was refluxed with a Dean-Stark trap for 1 h. The solvent was removed and the crude residue purified via column chromatography (ISCO, 5-100% ethyl acetate/hexanes) to give 0.270 g of methyl 3-{(3-chlorophenyl)[(2-{[(methyloxy)carbonyl]amino}ethyl)oxy]methyl}benzoate. MS (m/z) 378.4 (M+H⁺).

### Step 3. 3-{(3-chlorophenyl)[(2-{[(methyloxy)carbonyl]amino}ethyl)oxy]methyl}benzoic acid

To a solution of methyl 3-{(3-chlorophenyl)[(2-{[(methyloxy)carbonyl]amino}ethyl)oxy]methyl}benzoate (0.270 g, 0.7 mmol) in 3 mL of THF, sodium hydroxide (2.2 mL of a 2.5 N solution, 5.6 mmol) was added. The resulting mixture was stirred overnight at room temperature. The solvent was removed, the residue acidified with 1 N HCl,f and extracted with ethyl acetate. The combined organics were then dried over MgSO4, filtered and concentrated in vacuo. This material was combined with that from another experiment (0.92 mmol of starting material) and purified via column chromatography (ISCO, 50-100% ethyl acetate/hexanes) to give 0.300 g of 3-{(3-chlorophenyl)[(2-{[(methyloxy)carbonyl]amino}ethyl)oxy]methyl}benzoic acid as a white solid (51% yield). MS (m/z) 364.5 (M+H⁺).

The following benzoic acid intermediates were prepared using procedures analogous to those described above substituting the indicated aldehyde for methyl-3-formybenzoate in Step 1.

| Benzoic Acid | Name | Aldehyde |
|---|---|---|
| 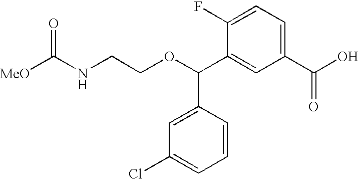 | 3-{(3-chlorophenyl)[(2- {[(methyloxy)carbonyl]amino} ethyl)oxy]methyl}-4- fluorobenzoic acid | methyl 4-fluoro-3- formylbenzoate |

### Intermediate Preparation 15

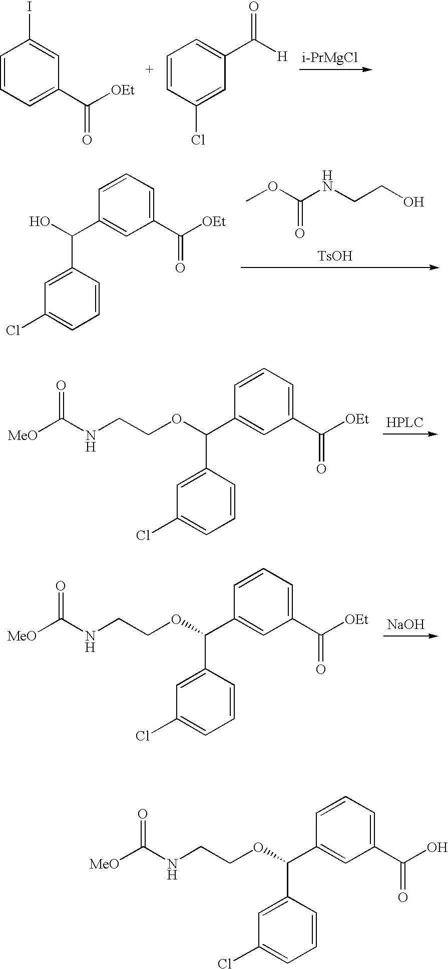

### Step 1. Ethyl 3-[(3-chlorophenyl)(hydroxy)methyl]benzoate

A 1 L 3-neck round bottom flask equipped with a 60 mL addition funnel was heated under vacuum with a heat gun. The vacuum line was replaced with a nitrogen line and a thermometer was added. Ethyl 3-iodobenzoate (18.29 ml, 109 mmol) was dissolved in tetrahydrofuran (THF) (362 ml). The mixture was cooled to −20 to −40° C. (dry ice/MeCN, monitored with internal thermometer) and isopropylmagnesium chloride in ether (59.8 ml, 120 mmol) was added dropwise using an addition funnel over 20 minutes. The reaction mixture was then stirred at −20 to −40° C. for 2.5 hours. 3-chlorobenzaldehyde (17.23 ml, 152 mmol) (dissolved in 40 mL of THF) was added over 20 minutes using a clean addition funnel. HPLC and TLC after one hour indicated that the iodide had been consumed. The mixture was warmed to 10° C. and 300 mL 1 N HCl was added carefully through an addition funnel followed by 200 mL of ethyl acetate. The layers were separated and the aqueous layer extracted with 50 mL EtOAc. The combined organic layers was dried over MgSO₄, filtered, and concentrated in vacuo. The crude oil was loaded directly onto a column and purified using silica gel chromatography (ISCO: 0-20% ethyl acetate/hexanes (30 min.), 20% (30 min.), 330 g silica) to afford 24.72 g of ethyl 3-[(3-chlorophenyl)(hydroxy)methyl]benzoate (-95% pure, 74% yield). MS (m/z) 290.8 (M+H^{±}).

### Step 2. Ethyl 3-{(3-chlorophenyl)[(2-{[(methyloxy)carbonyl]amino}ethyl)oxy]methyl}benzoate

Ethyl 3-[(3-chlorophenyl)(hydroxy)methyl]benzoate (1.63 g, 5.61 mmol), methyl (2-hydroxyethyl)carbamate (0.735 g, 6.17 mmol), and p-toluenesufonic acid monohydrate (1.173 g, 6.17 mmol) were dissolved in toluene (56.1 ml) and heated to reflux with a Dean-Stark trap for 2 hours. The mixture was then cooled to room temperature and sat. NaHCO₃ (50 mL) and EtOAc (50 mL) added. The layers were separated and the organic layer was dried (Na₂SO₄), filtered, and concentrated in vacuo. The compound was loaded onto florisil and purified via silica gel chromatography (ISCO: 0-20% ethyl acetate/hexanes (30 min.), 20% (20 min.), 40 g silica) to give 0.557 g of ethyl 3-{(3-chlorophenyl)[(2-{[(methyloxy)carbonyl]amino}ethyl)oxy]methyl}benzoate (24% yield). MS (m/z) 391.8 (M+H⁺).

### Step 3. Ethyl 3-{(3-chlorophenyl)[(2-{[(methyloxy)carbonyl]amino}ethyl)oxy]methyl}benzoate

The enantiomers of ethyl 3-{(3-chlorophenyl)[(2-{[(methyloxy)carbonyl]amino}ethyl)oxy]methyl}benzoate were separated using a Chiralpack IB-H column (20×250 mm) 20/90 isopropanol/hexane w/0.1% DEA @ 15 mL/min. The sample (545 mg) was dissolved in 6 mL methanol, filtered and injected (12 injections total). Both peaks were collected and checked by chiral HPLC. Peak #2 (retention time of 8.921 min) was concentrated in vacuo to give 0.224 g (41%) of the desired enantiomer, ethyl 3-{(R)-(3-chlorophenyl)[(2-{[(methyloxy)carbonyl]amino}ethyl)oxy]methyl}benzoate. MS (m/z) 392.5 (M+H⁺). Peak #1 (retention time of 6.663 min) was also concentrated in vacuo to give 0.185 g of the undesired enantiomer, ethyl 3-{(S)-(3-chlorophenyl)[(2-{[(methyloxy)carbonyl]amino}ethyl)oxy]methyl}benzoate. MS (m/z) 392.5 (M+H⁺).

### Step 4. 3-{(R)-(3-chlorophenyl)[(2-{[(methyloxy)carbonyl]amino}ethyl)oxy]methyl}benzoic acid

To a round bottom flask containing 430 mg of ethyl 3-{(R)-(3-chlorophenyl)[(2-{[(methyloxy)carbonyl]amino}ethyl)oxy]methyl }benzoate was added MeOH and THF and fully dissolved. Then the 2.5M NaOH was added (stirred rapidly at rt in air). The cloudy reaction turned clear over ca. half hour. LCMS analysis after 1.5 h indicated that the starting material had been consumed. The reaction mixture was then quenched by slow addition of 1.0 N HCl until pH 1 was achieved, then diluted with water (50 mL), and extracted with EtOAc (4×50 mL). The combined EtOAc layers were washed with brine (1×50 mL), dried over Na₂SO₄ (overnight), and concentrated in vacuo to yield 384.2 mg of 3-{(R)-(3-chlorophenyl)[(2-{[(methyloxy)carbonyl]amino}ethyl)oxy]methyl}benzoic acid as a clear heavy colorless oil (=100% yield). MS (m/z) 364.5 (M+H⁺).

The following benzoic acids were prepared using procedures to those analogous to those above substituting the indicated iodide for ethyl 3-iodobenzoate in Step 1. For racemic benzoic acids, Step 3 was omitted.

| Benzoic Acid | Name | Iodide |
|---|---|---|
| 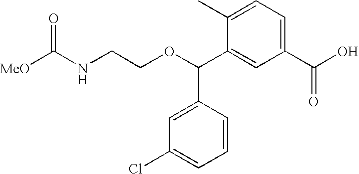 | 3-[(3-chlorophenyl)[(2- {[(methyloxy)carbonyl]amino}ethyl)oxy] methyl}-4-methylbenzoic acid | methyl 3-iodo-4- methylbenzoate |
| 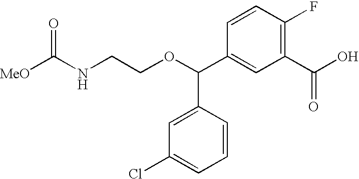 | 5-{(3-chlorophenyl)[(2- {[(methyloxy)carbonyl]amino}ethyl)oxy] methyl}-2-fluorobenzoic acid | methyl 2-fluoro-5- iodobenzoate |
| 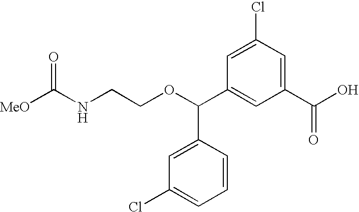 | 3-chloro-5-{(3-chlorophenyl)[(2- {[(methyloxy)carbonyl]amino}ethyl)oxy] methyl}benzoic acid | methyl 3-chloro- 5-iodobenzoate |

### Intermediate Preparation 16

### 3-{(S)-(3-chlorophenyl)[(2-{[(methyloxy)carbonyl]amino}ethyl)oxy]methyl}-4-methylbenzoic acid

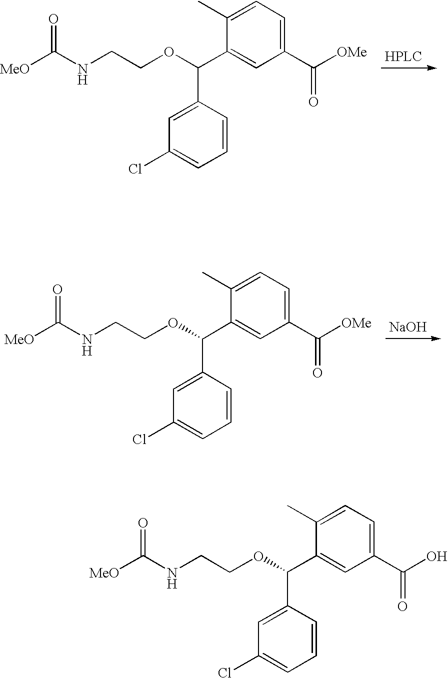

### Step 1. Methyl 3-{(5)-(3-chlorophenyl)[(2-{[(methyloxy)carbonyl]amino}ethyl)oxy]methyl}-4-methylbenzoate

The racemic sample, methyl 3-{(3-chlorophenyl)[(2-{[(methyloxy)carbonyl]amino}ethyl)oxy]methyl}-4-methylbenzoate, (0.433, 1.11 mmol) was dissolved in 4 mL, filtered and purified via preparative chiral HPLC (IB-H chiral column (20×250 mm), mobile phase 20% IPA/80% hexane with 0.1% diethylamine, 15 mL/min, 9 total injections). Fractions corresponding to the first peak (retention time of 6.592 min) were pooled and concentrated to afford methyl 3-{(S)-(3-chlorophenyl)[(2-{[(methyloxy)carbonyl]amino}ethyl)oxy]methyl}-4-methylbenzoate (0.173 g). Fractions corresponding to the second peak (retention time of 8.501 min) were combined and concentrated to give methyl 3-{(R)-(3-chlorophenyl)[(2-{[(methyloxy)carbonyl]amino}ethyl)oxy]methyl}-4-methylbenzoate (0.211 g).

### Step 2. 3-{(S)-(3-chlorophenyl)[(2-{[(methyloxy)carbonyl]amino}ethyl)oxy]methyl}-4-methylbenzoic acid

To a solution of methyl 3-{(S)-(3-chlorophenyl)[(2-{[(methyloxy)carbonyl]amino}ethyl)oxy]methyl}-4-methylbenzoate (0.17 g, 0.434 mmol) in methanol (4.34 mL) at room temperature, NaOH (1.735 mL, 1.735 mmol) was added. A white solid crashed out of solution, so THF (4.34 mL) was added to help solubility. The resulting mixture was stirred overnight at room temperature. The reaction was not complete, so an additional 2 equivalents of 1 N NaOH were added and the mixture stirred overnight. The methanol was removed in vacuo, the residue diluted with 5 mL of water, acidified to pH 3 with 1N HCl, and extracted with ethyl acetate (2×5 mL). The combined organic extracts were dried over MgSO₄, filtered, and concentrated. MS (m/z) 378.4 (M+H⁺).

### Intermediate Preparation 17

### 3-{(5-chloro-2-methylphenyl)[(2-{[(methyloxy)carbonyl]amino}ethyl)oxy]methyl}benzoic acid

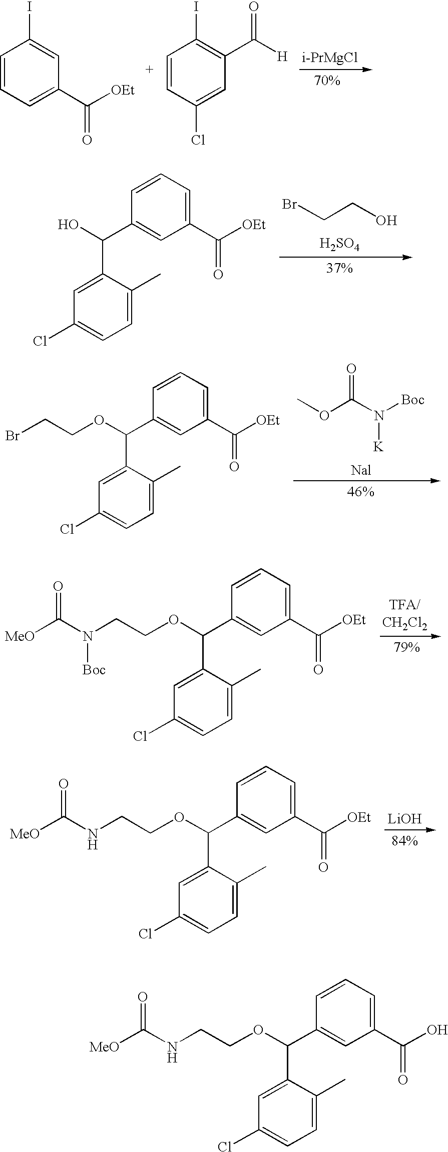

### Step 1. Ethyl 3-[(5-chloro-2-methylphenyl)(hydroxy)methyl]benzoate

To a solution of ethyl 3-iodobenzoate (13.16 g, 47.70 mmol) in THF at −30 to −40° C. isopropylmagnesium chloride (23.8 mL of a 2M solution, 47.70 mmol) was added dropwise. The resulting mixture was stirred for one hour before 5-chloro-2-methylbenzaldehyde (7.0 g, 45.3 mmol) was added. The reaction mixture was stirred at −30° C. for 30 min, then warmed to room temperature and stirred for an additional 10 min. Aqueous NH₄Cl and EtOAc were added and the layers separated. The organic layer was then washed with brine, dried over Na₂SO₄, filtered and concentrated in vacuo. The crude material was purified using silica gel chromatography to afford 10.2 g of ethyl 3-[(5-chloro-2-methylphenyl)(hydroxy)methyl]benzoate (70% yield).

### Step 2. Methyl 3-[[(2-bromoethyl)oxy](5-chloro-2-methylphenyl)methyl]benzoate

To 2-bromoethanol (33 mL, 469 mmol), was added methyl 3-[(5-chloro-2-methylphenyl)(hydroxy)methyl]benzoate (10.2 g, 33.5 mmol). After 5 min, sulfuric acid (10 drops) was added. The resulting mixture was then heated to 60 to 70° C. for 4 h before it was cooled to room temperature and diluted with ethyl acetate. The mixture was then washed with water, brine, dried over Na₂SO₄, filtered and concentrated in vacuo. The crude product was purified via column chromatography to give 5.1 g of methyl 3-[[(2-bromoethyl)oxy](5-chloro-2-methylphenyl)methyl]benzoate (37%).

### Step 3. Ethyl 3-{(5-chloro-2-methylphenyl)[(2-{{[(1,1-dimethylethyl)oxy]carbonyl}[(methyloxy)carbonyl]amino}ethyl)oxy]methyl}benzoate

To a solution of ethyl 3-[[(2-bromoethyl)oxy](5-chloro-2-methylphenyl)methyl]benzoate (5.1 g, 12.4 mmol) in acetone was added NaI (5.58 g, 37.2 mmol). The resulting mixture was then heated to 60° C. for 5 h before it was cooled to room temperature, filtered and washed with acetone. The acetone was removed and the residue diluted with ethyl acetate, washed with brine, dried over Na₂SO₄, filtered and concentrated in vacuo to afford the iodide intermediate. This material was dissolved in DMF and 1, 1-dimethylethyl methyl ester potassium salt (3.97 g, 18.6 mmol) added. The mixture was then heated to 50-60° C. overnight before it was cooled to room temperature, and quenched with aqueous NH₄Cl and ethyl acetate. The layers were separated and the organic phase washed with brine, dried over Na₂SO₄, filtered, and concentrated in vacuo. The crude material was purified via column chromatography to give 2.9 g of ethyl 3-{(5-chloro-2-methylphenyl)[(2-{{[(1,1-dimethylethyl)oxy]carbonyl}[(methyloxy)carbonyl]amino}ethyl)oxy]methyl}benzoate as a pale yellow oil (46%).

### Step 4. Ethyl 3-{(5-chloro-2-methylphenyl)[(2-{[(methyloxy)carbonyl]amino}ethyl)oxy]methyl}benzoate

To a solution of TFA/CH₂Cl₂ was added ethyl 3-{(5-chloro-2-methylphenyl)[(2-{{[(1,1-dimethylethyl)oxy]carbonyl}[(methyloxy)carbonyl]amino}ethyl)oxy]methyl}benzoate (0.60 g, 1.19 mmol). The mixture was then stirred at room temperature for 20 minutes before the solvent was removed. The residue was diluted with ethyl acetate and washed with aqueous NaHCO₃, brine, dried over Na₂SO₄, filtered and concentrated in vacuo to give 0.380 g of ethyl 3-{(5-chloro-2-methylphenyl)[(2-{[(methyloxy)carbonyl]amino}ethyl)oxy]methyl}benzoate as a pale yellow oil (79%).

### Step 5. 3-{(5-chloro-2-methylphenyl)[(2-{[(methyloxy)carbonyl]amino}ethyl)oxy]methyl}benzoic acid

To a solution of ethyl 3-{(5-chloro-2-methylphenyl)[(2-{[(methyloxy)carbonyl]amino}ethyl)oxy]methyl}benzoate (0.380 g, 0.938 mmol) in methanol was added lithium hydroxide (0.225 g, 3.75 mmol) and water. The resulting mixture was heated to 40-50° C. for 2 h before it was cooled to room temperature and the solvent removed. The residue was dissolved in ethyl acetate and acidified to pH 2-3. The organic layer was then washed with brine, dried over Na₂SO₄, filtered and concentrated in vacuo to afford 0.300 g of 3-{(5-chloro-2-methylphenyl)[(2-{[(methyloxy)carbonyl]amino}ethyl)oxy]methyl}benzoic acid as a pale yellow solid (84%).

### Intermediate Preparation 18

### 3-{(R)-(5-chloro-2-methylphenyl)[(2-{[(methyloxy)carbonyl]amino}ethyl)oxy]methyl}benzoic acid

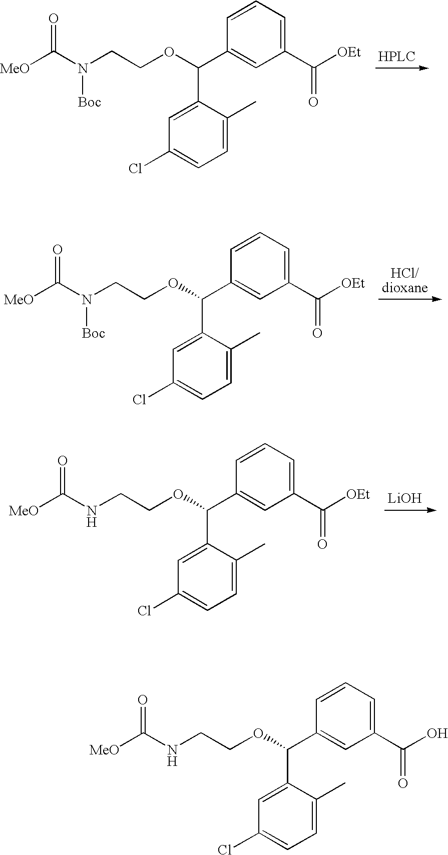

### Step 1. Ethyl 3-{(R)-(5-chloro-2-methylphenyl)[(2-{{[(1,1-dimethylethyl)oxy]carbonyl}[(methyloxy)carbonyl]amino}ethyl)oxy]methyl}benzoate

The mixture of ethyl 3-{(R)-(5-chloro-2-methylphenyl)[(2-{{[(1,1-dimethylethyl)oxy]carbonyl}[(methyloxy)carbonyl]amino}ethyl)oxy]methyl}benzoate and ethyl 3-{(S)-(5-chloro-2-methylphenyl)[(2-{{[(1,1-dimethylethyl)oxy]carbonyl}[methyloxy)carbonyl]amino}ethyl)oxy]methyl}benzoate was separated via preparative chiral HPLC (OJ-H column (20×250 mm) 100% methanol @ 10 mL/min, Runtime −15 min). The sample (833 mg) was dissolved in 20 mL methanol and then filtered and processed using 5 individual injections and 12 stack injections (approximately 62.5 mg per injection). Fractions corresponding to the first peak (retention time of 4.729 min) were pooled and concentrated to afford ethyl 3-{(R)-(5-chloro-2-methylphenyl)[(2-{{[(1,1-dimethylethyl)oxy]carbonyl}[(methyloxy)carbonyl]amino}ethyl)oxy]methyl}benzoate (0.287 g). Fractions corresponding to the second peak (retention time of 9.533 min) were also pooled and concentrated to provide ethyl 3-{(S)-(5-chloro-2-methylphenyl)[(2-{{[(1,1-dimethylethyl)oxy]carbonyl}[(methyloxy)carbonyl]amino}ethyl)oxy]methyl}benzoate (0.325 g).

### Step 2. Ethyl 3-{(R)-(5-chloro-2-methylphenyl)[(2-{[(methyloxy)carbonyl]amino}ethyl)oxy]methyl}benzoate

To a solution of ethyl 3-{(R)-(5-chloro-2-methylphenyl)[(2-{{[(1,1-dimethylethyl)oxy]carbonyl}[(methyloxy)carbonyl]amino}ethyl)oxy]methyl}benzoate (0.180 g, 0.36 mmol) in methylene chloride (10 mL), was added HCl (4M in dioxane, 3.56 mL). The resulting mixture was stirred at room temperature for 5 h. The solvent was removed in vacuo and the residue dissolved CH₂Cl₂ (10 mL) and washed with saturated NaHCO₃ (5 mL) and the aqueous was extracted with CH₂Cl₂ once more. The combined organic extracts were washed with brine, dried, filtered, and concentrated to afford ethyl 3-{(R)-(5-chloro-2-methylphenyl)[(2-{[(methyloxy)carbonyl]amino}ethyl)oxy]methyl}benzoate (0.140 g, 97%) as a clear oil, which was used in the next step without further purification. MS (m/z) 406.2 (M+H⁺).

### Step 3. 3-{(R)-(5-chloro-2-methylphenyl)[(2-{[(methyloxy)carbonyl]amino}ethyl)oxy]methyl}benzoic acid

To a solution of ethyl 3-{(R)-(5-chloro-2-methylphenyl)[(2-{[(methyloxy)carbonyl]amino}ethyl)oxy]methyl}benzoate (140 mg, 0.35 mmol) in THF (5 mL) and methanol (2 mL) was added LiOH (1M, 1.38 ml). The resulting mixture was stirred at rt for 16 h. The reaction was concentrated and the residue diluted with water (5 mL) and washed with EtOAc (5 mL). The aqueous layer was acidified with HCl (1N) to pH =2 and extracted with EtOAc (3×10 mL). The combined organic extracts were washed with brine, dried, filtered and concentrated in vacuo to give 3-{(R)-(5-chloro-2-methylphenyl)[(2-{[(methyloxy)carbonyl]amino}ethyl)oxy]methyl}benzoic acid (0.118 g, 91%) as a white solid. MS (m/z) 378.0 (M+H⁺).

### Intermediate Preparation 19

### 3-((3-chlorophenyl)(2-(methoxycarbonylamino)ethoxy)methyl)benzoic acid

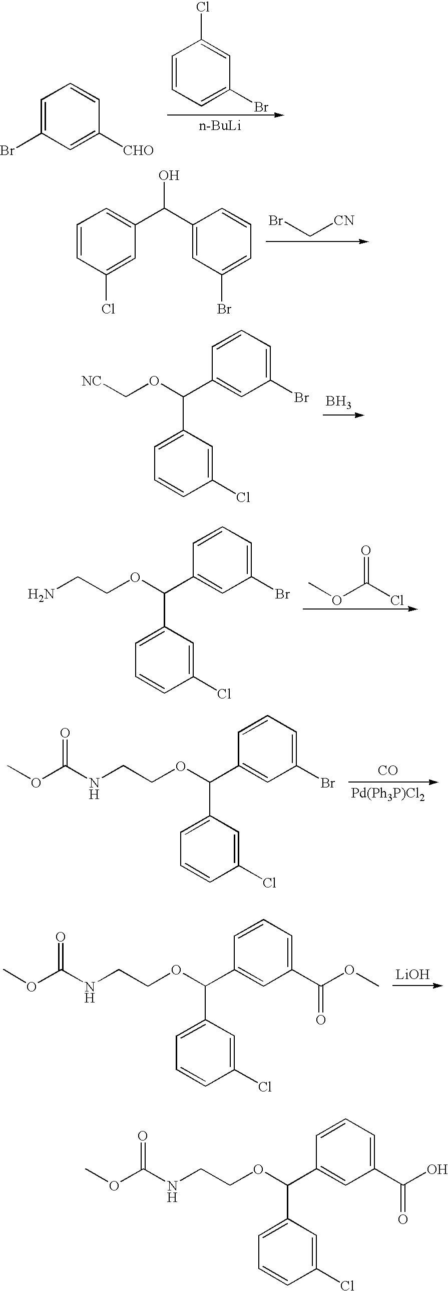

### Step 1. (3-bromophenyl)(3-chlorophenyl)methanol

A solution of 1-bromo-3-chlorobenzene (20 g, 104 mmol) in anhydrous THF (125 mL) under N₂ was cooled to −78° C., n-BuLi (2.5 M, 42 mL, 105 mmol) was then added drop wise over 20 min. After stirring an additional hour, 3-bromobenzaldehyde (12.5 mL, 104 mmol) was added drop wise and the reaction was stirred for another 10 min at −78° C. Sat. NH₄Cl (100 mL) was added and the reaction was warmed to room temperature and extracted with ether (300 mL). The ether layer was extracted with three 75 mL portions of 1 M sodium bisulfite, 75 mL of 1 M sodium hydroxide, 75 mL of water, and 75 mL of brine. The ether layer was dried over MgSO₄ and the solvent removed to give crude product, which was purified by chromatography on silica gel (16 g, 52%). ¹H NMR (CDCl₃) δ 2.27 (br, 1H), 5.75 (s, 1H), 7.14-7.54 (m, 8H).

### Step 2. 2-((3-bromophenyl)(3-chlorophenyl)methoxy)acetonitrile

To a solution of (3-bromophenyl)(3-chlorophenyl)methanol (32.5 g, 0.1 mol) in MeCN (325 mL), NaH (12 g, 0.3 mol) was added at 0° C. The mixture was stirred for 1 h at room temperature. The mixture was cooled to −40° C., and then bromoacetonitrile (35.7 g, 0.3 mol) was added in portions. The mixture was stirred for 0.5 h at −20° C. continually. The reaction was quenched with sat. NH₄Cl. The mixture was extracted with CH₂Cl₂. The organic layer was dried over Na₂SO₄, concentrated. The crude product was used without purification.

### Step 3. 2-((3-bromophenyl)(3-chlorophenyl)methoxy)ethanamine

2-((3-Bromophenyl)(3-chlorophenyl)methoxy)acetonitrile (23 g, 0.04 mol) was dissolved in anhydrous THF (300 mL), and the solution was heated to reflux under nitrogen. A solution of BH₃.Me₂S (12 mL, 0.12 mol) in THF was added dropwise, and stirring was continued under reflux overnight. The resulting solution was cooled to room temperature and MeOH was added drop wise to quench the reaction. After evaporation of the solution, the crude product was obtained and was used in the next step without further purification.

### Step 4. Methyl 2-((3-bromophenyl)(3-chlorophenyl)methoxy)ethylcarbamate

To a solution of 2-((3-bromophenyl)(3-chlorophenyl)methoxy)ethanamine (10.3 g, 30.4 mmol) and DMAP (1.9 g, 15.2 mmol) in anhydrous CH₂Cl₂ (150 mL), Et₃N (9.2 g, 91.2 mmol) was added. The resulting mixture was cooled to 0-5° C. under ice-water bath, a solution of methyl chloroformate (14.3 g, 152 mmol) in anhydrous CH₂Cl₂ (50 mL) was added drop wise. After addition, the reaction mixture was stirred for 1 h at 0° C. Upon completion of the reaction water was added. The aqueous layer was extracted with CH₂Cl₂. The combined organic layers were washed with 10% citric acid and brine, then dried over Na₂SO₄, filtered and concentrated to afford the crude product, which was purified by chromatography on silica gel (3 g, 25%). ¹H NMR (CDCl₃) δ 3.47 (m, 4H), 3.69 (s, 3H), 5.03 (brs, 1H), 5.26 (s, 1H), 7.12-7.50 (m, 8H).

### Step 5. Methyl 3-((3-chlorophenyl)(2-(methoxycarbonylamino)ethoxy)methyl)benzoate

A mixture of methyl 2-((3-bromophenyl)(3-chlorophenyl)methoxy)ethylcarbamate (600 mg, 1.5 mmol), Pd(Ph₃P)Cl₂ (106 mg, 0.15 mmol), Et₃N (305 mg, 3 mmol), MeOH (15 mL) was stirred in a sealed tube under 50 psi carbon monoxide atmosphere at 80° C. overnight. The reaction mixture was filtered. The solvent was removed in vacuo. The crude product was purified by preparative TLC (200 mg, 35%). ¹H NMR (CDCl₃) δ 3.42 (m, 2H), 3.53 (m, 2H), 3.67 (s, 3H), 3.91 (s, 3H), 5.05 (brs, 1H), 5.35 (s, 1H), 7.18-7.51 (m, 6H), 7.95 (m, 2H).

### Step 6. 3-((3-chlorophenyl)(2-(methoxycarbonylamino)ethoxy)methyl)benzoic acid

Methyl 3-((3-chlorophenyl)(2-(methoxycarbonylamino)ethoxy)methyl)benzoate (200 mg, 0.53 mmol) was dissolved in THF (10 mL) and treated with LiOH/H₂O (2 M, 10 mL) was added. The reaction mixture was stirred at room temperature for 2 days. A solution of 2 N HCl was added until pH 2 was reached, the aqueous layer was extracted with EtOAc three times. The combined organic layers were dried over Na₂SO₄, filtered, and then concentrated. The crude product was used without further purification (160 mg, 83%). ¹H NMR (CDCl₃) δ 3.43 (m, 2H), 3.54 (m, 2H), 3.69 (s, 3H), 5.06 (brs, 1H), 5.38 (s, 3H), 7.18-7.57 (m, 6H), 8.03 (m, 2H).

Specific conditions for synthesizing the disclosed aspartic protease inhibitor compounds according to the above schemes are provided below.

### Example 1

### Methyl 2-((R)-(3-chlorophenyl)(3-((S)-1-(methylamino)-3-(tetrahydro-2H-pyran-4-yl)propan-2-ylcarbamoyl)phenyl)methoxy)ethylcarbamate (I-1a)

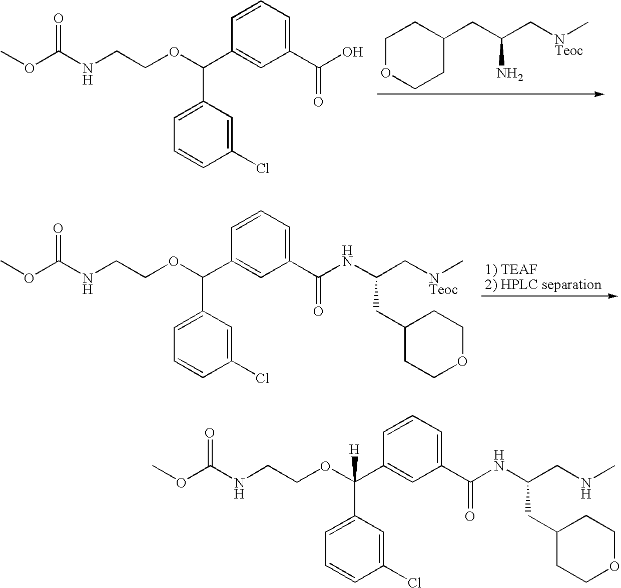

### Step 1. 2-(trimethylsilyl)ethyl (2S)-2-(3-((2-(methoxycarbonylamino)ethoxy)(3-chlorophenyl)methyl)benzamido)-3-(tetrahydro-2H-pyran-4-yl)propyl(methyl)carbamate

3-((3-chlorophenyl)(2-(methoxycarbonylamino)ethoxy)methyl)benzoic acid (60 mg, 0.165 mmol), (S)-2-(trimethylsilyl)ethyl 2-amino-3-(tetrahydro-2H-pyran-4-yl)propyl(methyl)carbamate (52 mg, 0.165 mmol) [prepared using procedures described in U.S. Provisional App. No. 60/736,564, filed on Nov. 14, 2005, and PCT App No. PCT/US2006/043920, filed November 13, 2006, the entire contents of which are hereby incorporated by reference], EDCI (79 mg, 0.413 mmol) and HOBt (56 mg, 0.413 mmol) was dissolved in CH₂Cl₂ (8 mL). The reaction mixture was stirred at room temperature overnight. The solvent was removed in vacuo. The crude product was purified by preparative TLC (89 mg, 82%).

### Step 2

### Methyl 2-((R)-(3-chlorophenyl)(3-((S)-1-(methylamino)-3-(tetrahydro-2H-pyran-4-yl)propan-2-ylcarbamoyl)phenyl)methoxy)ethylcarbamate

2-(trimethylsilyl)ethyl (2S)-2-(3-((2-(methoxycarbonylamino)ethoxy)(3-chlorophenyl)methyl)benzamido)-3-(tetrahydro-2H-pyran-4-yl)propyl(methyl)carbamate (89 mg, 0.135 mmol) and tetraethylammonium fluoride hydrate (44 mg, 0.296 mmol) were dissolved in MeCN (10 mL), the reaction mixture was refluxed for 1 h. The solvent was removed under reduced pressure to the residue, which was purified by preparative HPLC to produce methyl 2-((R)-(3-chlorophenyl)(3-((5)-1-(methylamino)-3-(tetrahydro-2H-pyran-4-yl)propan-2-ylcarbamoyl)phenyl)methoxy)ethylcarbamate (6 mg, 9%). Isomer 1: ¹H NMR (MeOD, 400 MH_{Z}) δ 1.28 (m, 3H), 2.70 (s, 3H), 3.16 (m, 3H), 3.50 (m, 2H), 3.60 (s, 3H), 3.90 (m, 2H), 4.53 (m, 1H), 5.50 (s, 1H), 7.26 (m, 3H), 7.40 (s, 1H), 7.50 (m, 1H), 7.60 (m, 1H), 7.78 (m, 1H), 7.90 (s, 1H); MS m/z: 518 (M⁺).

The following compounds were prepared following procedures analogous to those described above:
1) methyl 2-((S)-(3-chlorophenyl)(3-((S)-1-(methylamino)-3-((R)-tetrahydro-2H-pyran-3-yl)propan-2-ylcarbamoyl)phenyl)methoxy)ethylcarbamate (I-2a) and methyl 2-((R)-(3-chlorophenyl)(3-((S)-1-(methylamino)-3-((R)-tetrahydro-2H-pyran-3-yl)propan-2-ylcarbamoyl)phenyl)methoxy)ethylcarbamate (I-2b) using 2-(trimethylsilyl)ethyl (S)-2-amino-3-((R)-tetrahydro-2H-pyran-3-yl)propyl(methyl)carbamate, which was prepared using in Step 1, procedures described using is Step 1, procedures described in U.S. Provisional App. No. 60/736,564, filed on Nov. 14, 2005, and PCT Application No. PCT/US2006/043920, filed Nov. 13, 2006, the entire contents of which are hereby incorporated by reference. I-2a and I-2b were separated by preparative HPLC followed by chiral HPLC.
2) methyl 2-((R)-(3-chlorophenyl)(3-((S)-1-(methylamino)-3-((R)-oxepan-3-yl)propan-2-ylcarbamoyl)phenyl)methoxy)ethylcarbamate (I-4a) and methyl 2-((S)-(3-chlorophenyl)(3-((S)-1-(methylamino)-3-((R)-oxepan-3-yl)propan-2-ylcarbamoyl)phenyl)methoxy)ethylcarbamate (I-4b) using 2-(trimethylsilyl)ethyl (S)-2-amino-3-((R)-oxepan-3-yl)propyl(methyl)carbamate, which was prepared using is Step 1, procedures described in U.S. Provisional App. No. 60/736,564, filed on Nov. 14, 2005, and PCT App. No. PCT/US2006/043920, filed Nov. 13, 2006, the entire contents of which are hereby incorporated by reference. I-4a and I-4b were separated by preparative HPLC followed by chiral HPLC.

### Example 2

### Methyl 2-((R)-(3-chlorophenyl)(3-((S)-1-cyclohexyl-3-(methylamino)propan-2-ylcarbamoyl)phenyl)methoxy)ethylcarbamate (I-3a) and methyl 2-((5)-(3-chlorophenyl)(3-((5)-1-cyclohexyl-3-(methylamino)propan-2-ylcarbamoyl)phenyl)methoxy)ethylcarbamate (I-3b)

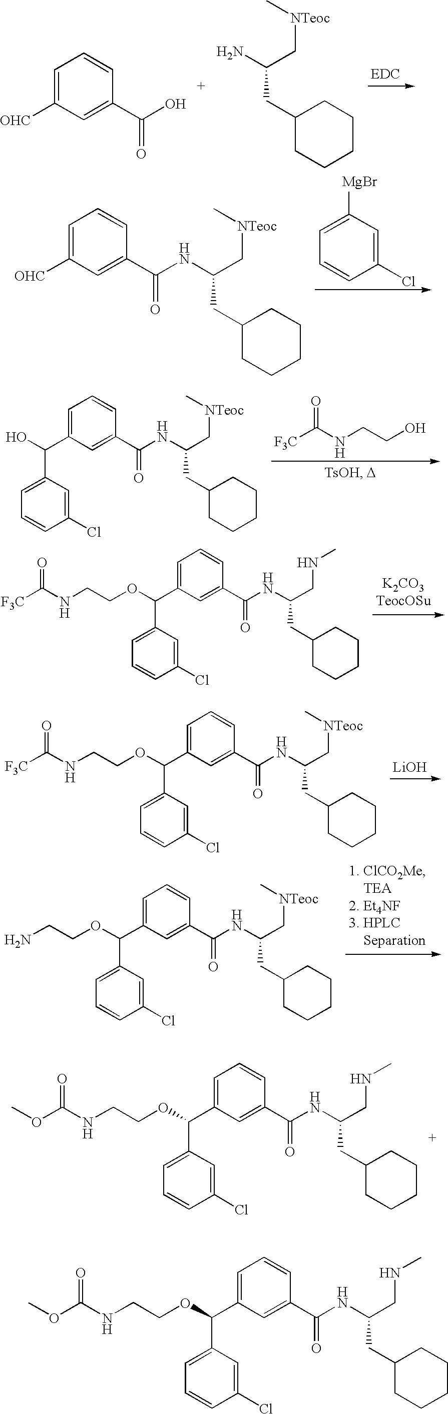

### Step 1. (S)-2-(trimethylsilyl)ethyl 3-cyclohexyl-2-(3-formylbenzamido)-propyl(methyl)carbamate

A mixture of 3-carboxybenzaldehyde (1.20 g, 8.01 mmol, 1.0 equiv), 2-(trimethylsilyl)ethyl (S)-2-amino-3-cyclohexylpropylmethylcarbamate, prepared using procedures described in PCT App No. 60/736,564, (2.57 g, 8.17 mmol, 1.02 equiv), EDC (2.86 g, 1.86 equiv), and DIEA (7 mL, 5 equiv) in CH₂Cl₂ (40 mL) was stirred at room temperature for 20 h. After evaporation of solvent, the residue was purified by chromatography on silica gel eluted with hexanes/EtOAc to afford (S)-2-(trimethylsilyl)ethyl 3-cyclohexyl-2-(3-formylbenzamido)-propyl(methyl)carbamate. LC-MS (3 min) m/z: 447 (M+H⁺).

### Step 2. 2-(trimethylsilyl)ethyl (2S)-2-(3-((3-chlorophenyl)(hydroxy)methyl)benzamido)-3-cyclohexylpropyl(methyl)carbamate

To a solution of (S)-2-(trimethylsilyl)ethyl 3-cyclohexyl-2-(3-formylbenzamido)-propyl(methyl)carbamate (0.61 g, 1.36 mmol, 1.0 equiv) in THF (15 mL) was added 8 mL of (3-chlorophenyl)magnesium bromide (0.50 M, 4.0 mmol, 2.9 equiv) at 0° C. under N₂. After 7 h, the reaction mixture was quenched with 10% Na₂CO₃ (3 mL), diluted with CH₂Cl₂, and dried over Na₂SO₄. After the solvent was removed in vacuo, the residue was purified by preparative HPLC (Phenomenex® Luna 5μ C18(2) 100A, 250×21.20 mm, 5 micron, 70%→90% CH₃CN/H₂O, 0.01% CF₃COOH over 8 min and then 90% CH₃CN/H₂O, 0.1% CF₃COOH over 7 min, flow rate 25 mL/min) to give 320 mg (42%) of 2-(trimethylsilyl)ethyl (2S)-2-(3-((3-chlorophenyl)(hydroxy)methyl)benzamido)-3-cyclohexylpropyl(methyl)carbamate. LC-MS (3 min) m/z: 559 (M+H⁺).

### Step 3. 3-((3-chlorophenyl)(2-(2,2,2-trifluoroacetamido)ethoxy)methyl)-N-((S)-1-cyclohexyl-3-(methylamino)propan-2-yl)benzamide

A mixture of 2-(trimethylsilyl)ethyl (2S)-2-(3-((3-chlorophenyl)(hydroxy)methyl)benzamido)-3-cyclohexylpropyl(methyl)carbamate (147 mg, 0.263 mmol, 1.0 equiv), 2,2,2-trifluoro-N-(2-hydroxyethyl)acetamide (1.31 g, 8.32 mmol, 32 equiv), and p-toluenesulfonic acid monohydrate (0.80 g, 4.20 mmol, 16 equiv) was dissolved in CH₃CN. After the solvent was removed, the residue was heated at 140° C. for 4.5 h. The mixture was cooled to room temperature and directly used in the next step without further purification. LC-MS (3 min) m/z: 554 (M+H⁺).

### Step 4. 2-(trimethylsilyl)ethyl (2S)-2-(3-((3-chlorophenyl)(2-(2,2,2-trifluoroacetamido)ethoxy)methyl)benzamido)-3-cyclohexylpropyl(methyl)carbamate

A mixture of 3-((3-chlorophenyl)(2-(2,2,2-trifluoroacetamido)ethoxy)methyl)-N-((S)-1-cyclohexyl-3-(methylamino)propan-2-yl)benzamide, obtained as described above, TeocOSu (295 mg, 1.14 mmol), and K₂CO₃ (2.14 g) in CH₂Cl₂ and H₂O was vigorously stirred at room temperature for 16 h. The mixture was diluted with saturated brine, extracted with CH₂Cl₂, and dried over Na₂SO₄. After the solvent was removed in vacuo, the crude product was purified by preparative HPLC (Phenomenex® Luna 5μ C18(2) 100A, 250×21.20 mm, 5 micron, 70%→90% CH₃CN/H₂O, 0.1% CF₃COOH over 8 min and then 90% CH₃CN/H₂O, 0.1% CF₃COOH over 10 min, flow rate 25 mL/min) to give 2-(trimethylsilyl)ethyl (2S)-2-(3-((3-chlorophenyl)(2-(2,2,2-trifluoroacetamido)ethoxy)methyl)benzamido)-3-cyclohexylpropyl(methyl)carbamate. LC-MS (3 min) m/z: 698 (M+H⁺).

### Step 5. 2-(trimethylsilyl)ethyl (2S)-2-(3-((2-aminoethoxy)(3-chlorophenyl)methyl)benzamido)-3-cyclohexylpropyl(methyl)carbamate

A mixture of 500 mg (0.0716 mmol) of 2-(trimethylsilyl)ethyl (2S)-2-(3-((3-chlorophenyl)(2-(2,2,2-trifluoroacetamido)ethoxy)methyl)benzamido)-3-cyclohexylpropyl(methyl)carbamate and 1.17 g of lithium hydroxide monohydrate in 25 mL of THF and 10 mL of water was vigorously stirred at room temperature for 5 h. After THF was removed in vacuo, the residue was extracted with CH₂Cl₂, dried over K₂CO₃. After the solvents were removed in vacuo, the crude product was used in the next step without further purification. LC-MS (3 min) m/z: 602 (M+H⁺).

### Step 6. 2-(trimethylsilyl)ethyl (2S)-2-(3-((2-(methoxycarbonyl)aminoethoxy)(3-chlorophenyl)methyl)benzamido)-3-cyclohexylpropyl(methyl)carbamate

A mixture of 2-(trimethylsilyl)ethyl (2S)-2-(3-((2-aminoethoxy)(3-chlorophenyl)methyl)benzamido)-3-cyclohexylpropyl(methyl)carbamate, obtained as described above, 105 mg (12 equiv) of 4-dimethylaminopyridine, 1 mL of triethylamine, and 288 mg (42 equiv) of methyl chloroformate in CH₂Cl₂ (18 mL) was stirred at room temperature for 16 h. After the solvents were removed in vacuo, the residue was purified by preparative HPLC (Phenomenex® Luna 5μ C18(2) 100A, 250×21.20 mm, 5 micron, 70%→90% CH₃CN/H₂O, 0.1% CF₃COOH over 8 min and then 90% CH₃CN/H₂O, 0.1% CF₃COOH over 7 min, flow rate 25 mL/min) to give 176 mg (37% in two steps) of 2-(trimethylsilyl)ethyl (2S)-2-(3-((2-(methoxycarbonyl)aminoethoxy)(3-chlorophenyl)methyl)benzamido)-3-cyclohexylpropyl(methyl)carbamate. LC-MS (3 min) m/z: 660 (M+H⁺).

### Step 7. methyl 2-((R)-(3-chlorophenyl)(3-((S)-1-cyclohexyl-3-(methylamino)propan-2-ylcarbamoyl)phenyl)methoxy)ethylcarbamate and methyl 2-((S)-(3-chlorophenyl)(3-((S)-1-cyclohexyl-3-(methylamino)propan-2-ylcarbamoyl)phenyl)methoxy)ethylcarbamate

A mixture of 2-(trimethylsilyl)ethyl (2S)-2-(3-((2-(methoxycarbonyl)aminoethoxy)(3-chlorophenyl)methyl)benzamido)-3-cyclohexylpropyl(methyl)carbamate (176 mg, 0.0266 mmol) and 8 mL of 0.5 M tetraethylammonium fluoride in acetonitrile was stirred at room temperature for 16 h and then purified by preparative HPLC (Phenomenex® Luna 5μ C18(2) 100A, 250×21.20 mm, 5 micron, 10%→90% CH₃CN/H₂O, 0.1% CF₃COOH over 13 min, flow rate 25 mL/min) to give 176 mg of TFA salt of the product as a mixture of diastereoisomers. LC-MS (3 min) m/z: 516 (M+H⁺).

The mixture was further separated by chiral HPLC (CHIRALCEL OD-H, 1 cmø×25 cm, 10% IPA in hexane with 0.025% diethylamine, flow rate 4 mL/min) to give two fractions in the ratio of 48:52, t_{R}=21.59 min and 32.57 min. Methyl 2-((R)-(3-chlorophenyl)(3-((S)-1-cyclohexyl-3-(methylamino)propan-2-ylcarbamoyl)phenyl)methoxy)ethylcarbamate (I-3a): ¹H NMR (400 MHz, CD₃OD) δ 7.80 (s, 1H), 7.68 (d, J=7.9 Hz, 1H), 7.48 (d, J=7.6 Hz, 1H), 7.39 (d, J=7.6 Hz, 1H), 7.35 (s, 1H), 7.24-7.17 (m, 3H), 5.43 (s, 1H), 4.39-4.32 (m, 1H), 3.55 (s, 3H), 3.45 (t, J=5.3 Hz, 2H), 3.28 (t, J=5.3 Hz, 2H), 2.72 (d, J=6.4 Hz, 2H), 2.41 (s, 3H), 1.84-0.80 (m, 15H). Methyl 2-((S)-(3-chlorophenyl)(3-((S)-1-cyclohexyl-3-(methylamino)propan-2-ylcarbamoyl)phenyl)methoxy)ethylcarbamate (I-3b): ¹H NMR (400 MHz, CD₃OD) δ 7.82-7.81 (m, 1H), 7.70 (d, J=7.6 Hz, 1H), 7.54 (d, J=7.6 Hz, 1H), 7.41 (t, J=7.6 Hz, 1H), 7.34 (s, 1H), 7.25-7.17 (m, 3H), 5.44 (s, 1H), 4.46-4.41 (m, 1H), 3.55 (s, 3H), 3.45 (t, J=5.3 Hz, 2H), 3.28 (t, J=5.7 Hz, 2H), 3.08 (dd, J=12.7, 3.4 Hz, 1H), 3.00-2.94 (m, 1H), 2.63 (s, 3H), 1.80-0.81 (m, 15H).

The following are compounds of the invention. Compound names were generated with the assistance of ChemDraw® versions 8.0 and 9.0 (CambridgeSoft Corporation, 100 CambridgePark Drive, Cambridge, Mass. 02140 USA). When the stereochemistry at a chiral center is not defined in the compound name this indicates that the sample prepared contained a mixture of isomers at this center.

Table of Compounds

| | | Synthetic | LC-MS | | | Selected |
|---|---|---|---|---|---|---|
| Cpd. | | Method | (3 min) | | | ¹H NMR |
| No. | Name | Example | t_{R} (min) | Mass observed | ¹H NMR Solvent | resonances |
| I-1a | methyl 2-((R)-(3- | 1 | 1.746 | 518 (M+) | CD₃OD | 1.28 (m, 3H), 2.70 (s, 3H), 3.16 (m, |
| | chlorophenyl)(3-((S)-1- | | | | | 3H), 3.50 (m, 2H), 3.60 (s, 3H), 3.90 |
| | (methylamino)-3- | | | | | (m, 2H), 4.53 (m, 1H), 5.50 (s, 1H), |
| | (tetrahydro-2H-pyran-4- | | | | | 7.26 (m, 3H), 7.40 (s, 1H), 7.50 (m, |
| | yl)propan-2- | | | | | 1H), 7.60 (m, 1H), 7.78 (m, 1H), |
| | ylcarbamoyl)phenyl)methoxy)ethylcarbamate | | | | | 7.90 (s, 1H) |
| I-2a | methyl 2-((S)-(3- | 1 | 1.26 | 518, | | ND |
| | chlorophenyl)(3-((S)-1- | | | 520 (M + 1) | | |
| | (methylamino)-3-((R)- | | | | | |
| | tetrahydro-2H-pyran-3- | | | | | |
| | yl)propan-2- | | | | | |
| | ylcarbamoyl)phenyl)methoxy)ethylcarbamate | | | | | |
| I-2b | methyl 2-((R)-(3- | 1 | 1.24 | 518, | CD₃OD | 7.80 (s, 1H), 7.67 (d, J = 7.9 Hz, |
| | chlorophenyl)(3-((S)-1- | | | 520 (M + 1) | | 1H), 7.45 (d, J = 7.9 Hz, 1H), 7.36 (d, |
| | (methylamino)-3-((R)- | | | | | J = 7.6 Hz, 1H), 7.34 (s, 1H), 7.22-7.15 |
| | tetrahydro-2H-pyran-3- | | | | | (m, 3H), 5.40 (s, 1H), 4.28-4.25 (m, |
| | yl)propan-2- | | | | | 1H), 3.89-3.85 (m, 1H), 3.73-3.69 (m, |
| | ylcarbamoyl)phenyl)methoxy)ethylcarbamate | | | | | 1H), 3.52 (s, 3H), 3.44-3.38 (m, 2H), |
| | | | | | | 3.30-3.24 (m, 2H), 3.09-3.04 (m, 2H), |
| | | | | | | 2.67 (d, J = 6.7 Hz, 2H), 2.36 (s, 3H), |
| | | | | | | 1.79-1.20 (m, 9H). |
| I-3a | methyl 2-((R)-(3- | 2 | 1.53 | 516, | CD₃OD | 7.80 (s, 1H), 7.68 (d, J = 7.9 Hz, 1H), |
| | chlorophenyl)(3-((S)-1- | | | 518 (M + 1) | | 7.48 (d, J = 7.6 Hz, 1H), 7.39 (d, |
| | cyclohexyl-3- | | | | | J = 7.6 Hz, 1H), 7.35 (s, 1H), 7.24-7.17 |
| | (methylamino)propan-2- | | | | | (m, 3H), 5.43 (s, 1H), 4.39-4.32 (m, |
| | ylcarbamoyl)phenyl)methoxy)ethylcarbamate | | | | | 1H), 3.55 (s, 3H), 3.45 (t, J = 5.3 Hz, |
| | | | | | | 2H), 3.28 (t, J = 5.3 Hz, 2H), 2.72 (d, |
| | | | | | | J = 6.4 Hz, 2H), 2.41 (s, 3H), 1.84-0.80 |
| | | | | | | (m, 15H). |
| I-3b | methyl 2-((S)-(3- | 2 | 1.53 | 516, | CD₃OD | 7.82-7.81 (m, 1H), 7.70 (d, J = 7.6 Hz, |
| | chlorophenyl)(3-((S)-1- | | | 518 (M + 1) | | 1H), 7.54 (d, J = 7.6 Hz, 1H), 7.41 (t, |
| | cyclohexyl-3- | | | | | J = 7.6 Hz, 1H), 7.34 (s, 1H), 7.25-7.17 |
| | (methylamino)propan-2- | | | | | (m, 3H), 5.44 (s, 1H), 4.46-4.41 (m, |
| | ylcarbamoyl)phenyl)methoxy)ethylcarbamate | | | | | 1H), 3.55 (s, 3H), 3.45 (t, J = 5.3 Hz, |
| | | | | | | 2H), 3.28 (t, J = 5.7 Hz, 2H), 3.08 (dd, |
| | | | | | | J = 12.7, 3.4 Hz, 1H), 3.00-2.94 (m, |
| | | | | | | 1H), 2.63 (s, 3H), 1.80-0.81 (m, 15H). |
| I-4a | methyl 2-((R)-(3- | 1 | 1.36 | 532, | CD₃OD | 7.79 (s, 1H), 7.66 (d, J = 7.6 Hz, 1H), |
| | chlorophenyl)(3-((S)-1- | | | 534 (M + 1) | | 7.45 (d, J = 7.9 Hz, 1H), 7.36 (d, |
| | (methylamino)-3-((R)- | | | | | J = 7.9 Hz, 1H), 7.33 (s, 1H), 7.22-7.14 |
| | oxepan-3-yl)propan-2- | | | | | (m, 3H), 5.40 (s, 1H), 4.24 (m, 1H), |
| | ylcarbamoyl)phenyl)methoxy)ethylcarbamate | | | | | 3.72 (dd, J = 12.3, 4.5 Hz, 1H), 3.64- |
| | | | | | | 3.54 (m, 2H), 3.52 (s, 3H), 3.44-3.36 |
| | | | | | | (m, 3H), 3.26 (t, J = 5.3 Hz, 2H), 2.64 |
| | | | | | | (d, J = 6.4 Hz, 2H), 2.33 (s, 3H), 1.72- |
| | | | | | | 1.30 (m, 11H). |
| I-4b | methyl 2-((S)-(3- | 1 | 1.34 | 532, | CD₃OD | 7.79 (s, 1H), 7.67 (d, J = 7.6 Hz, 1H), |
| | chlorophenyl)(3-((S)-1- | | | 534 (M + 1) | | 7.48 (d, J = 7.6 Hz, 1H), 7.36 (t, J = |
| | (methylamino)-3-((R)- | | | | | 7.6 Hz, 1H), 7.32 (s, 1H). 7.22-7.15 (m, |
| | oxepan-3-yl)propan-2- | | | | | 3H), 5.41 (s, 1H), 4.30-4.26 (m, 1H), |
| | ylcarbamoyl)phenyl)methoxy)ethylcarbamate | | | | | 3.70 (dd, J = 12.3, 4.1 Hz, 1H), 3.65- |
| | | | | | | 3.54 (m, 2H), 3.52 (s, 3H), 3.43-3.37 |
| | | | | | | (m, 3H), 3.26 (t, J = 5.9 Hz, 2H), |
| | | | | | | 2.82-2.77 (m, 2H), 2.44 (s, 3H), 1.72- |
| | | | | | | 1.30 (m, 11H). |

### Example 3

### Methyl {2-[((R)-(3-chlorophenyl){3-[({2S)-2-(methylamino)-3-[(3R)-tetrahydro-2H-pyran-3-yl]propyl}amino)carbonyl]phenyl}methyl)oxy]ethyl}carbamate hydrochloride

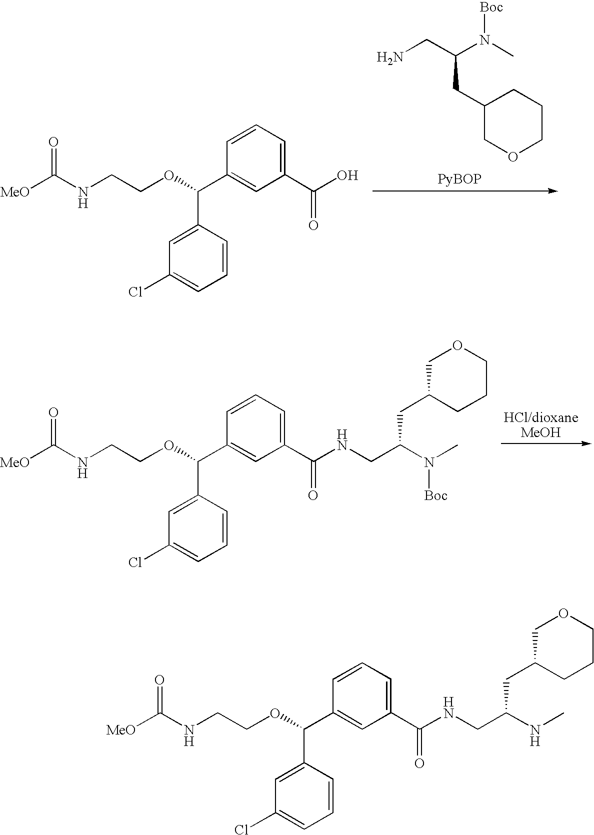

### Step 1. Methyl {2-[((R)-(3-chlorophenyl){3-[({(2S)-2-[{[(1,1-dimethylethyl)oxy]carbonyl}(methyl)amino]-3-[(3R)-tetrahydro-2H-pyran-3-yl]propyl}amino)carbonyl]phenyl}methyl)oxy]ethyl}carbamate

To a solution of 3-{(R)-(3-chlorophenyl)[(2-{[(methyloxy)carbonyl]amino}ethyl)oxy]methyl}benzoic acid (0.375 g, 1.031 mmol) in dichloromethane (10.31 ml) were added N,N-diisopropylethylamine (0.360 ml, 2.062 mmol), 1,1-dimethylethyl {(1S)-2-amino-1-[(3R)-tetrahydro-2H-pyran-3-ylmethyl]ethyl}methylcarbamate (0.309 g, 1.134 mmol), and PyBOP (0.590 g, 1.134 mmol). HPLC analysis after 1 hour indicated that the starting material had been consumed. The reaction mixture was concentrated, the crude material loaded onto florisil and purified using silica gel chromatography (ISCO: 30-75% ethyl acetate/hexanes (30 min.), 12 g silica) to give 0.68 g of methyl {2-[((R)-(3-chlorophenyl){3-[({(2S)-2-[{[(1,1-dimethylethyl)oxy]carbonyl}(methyl)amino]-3-[(3R)-tetrahydro-2H-pyran-3-yl]propyl}amino)carbonyl]phenyl}methyl)oxy]ethyl}carbamate that was 95% pure and contained a small amount of ethyl acetate by NMR (101% yield). MS (m/z) 618.6 (M+H⁺).

### Step 2. Methyl {2-[((R)-(3-chlorophenyl){3-[({(2S)-2-(methylamino)-3-[(3R)-tetrahydro-2H-pyran-3-yl]propyl}amino)carbonyl]phenyl}methyl)oxy]ethyl}carbamate hydrochloride

To a solution of methyl {2-[((R)-(3-chlorophenyl){3-[({(2S)-2-[{[(1,1-dimethylethyl)oxy]carbonyl}(methyl)amino]-3-[(3R)-tetrahydro-2H-pyran-3-yl]propyl}amino)carbonyl]phenyl}methyl)oxy]ethyl}carbamate (0.635 g, 1.027 mmol) in acetonitrile (10.27 ml) was added HCl in dioxane (1.284 ml, 5.14 mmol). The reaction mixture was concentrated and purified via HPLC (Agilent prep: 20-60% CH₃CN/H₂O, 0.1% TFA, 30×150 mm Sunfire C18, 25 mL/min, 15 min., 6 injections). The product fractions were concentrated on an EZ2 Genevac overnight. The product was then dissolved in EtOAc (30 mL) and 1 N NaOH (20 mL) added. The layers were separated and the aqueous layer extracted with EtOAc (2×10 mL). The combined organic layers were dried over MgSO₄, filtered, and concentrated to give 414 mg of methyl {2-[((R)-(3-chlorophenyl){3-[({(2S)-2-(methylamino)-3-[(3R)-tetrahydro-2H-pyran-3-yl]propyl}amino)carbonyl]phenyl}methyl)oxy]ethyl}carbamate (78% yield). The free base was then dissolved in 10 mL MeCN and added 0.4 mL of 4 N HCl/dioxane (2 eq. with respect to the 414 mg/0.8 mmol of free base) and concentrated. The material was azeotroped with additional acetonitrile and then MeOH and finally dissolved in 5 mL MeOH and filtered through Acrodisc CR 25 mm syringe filter with 0.2 um PTFE membrane to removed any particulate before it was concentrated to afford 0.570 g of methyl {2-[((R)-(3-chlorophenyl){3-[({(2S)-2-(methylamino)-3-[(3R)-tetrahydro-2H-pyran-3-yl]propyl}amino)carbonyl]phenyl}methyl)oxy]ethyl}carbamate hydrochloride as a white foam (71% yield). MS (m/z) 519.0 (M+H⁺). 1H NMR (400 MHz, DMSO-d6) δ 8.80 (t, J=5.7 Hz, 1H), 8.72 (s, 1H), 7.93 (s, 1H), 7.84 (d, J=7.9 Hz, 1H), 7.59 (d, J=7.5 Hz, 1H), 7.49 (s, 1H), 7.47 (t, J=7.6 Hz, 1H), 7.39-7.29 (m, 4H), 5.57 (s, 1H), 3.74 (dd, J=11.0, 3.5 Hz, 2H), 3.61 (dt, J=14.7, 4.4 Hz, 1H), 3.51 (s, 3H), 3.46 (dd, J=14.9, 6.1 Hz, 1H), 3.40 (t, J=5.9 Hz, 2H), 3.34 (s, 3H), 3.29 (dt, J=15.4, 5.3 Hz, 2H), 3.21 (q, J=5.8 Hz, 2H), 2.99 (dd, J=10.7, 9.2 Hz, 1H), 2.61 (s, 3H), 1.93-1.87 (m, 1H), 1.78-1.69 (m, 1H), 1.61-1.54 (m, 1H), 1.51-1.41 (m, 3H), 1.15 (ddd, J=23.3, 10.4, 3.5 Hz, 1H)

The compounds in the following Table 4 were prepared following procedures analogous to those described above, and optionally isolated as the designated salts.

TABLE 4

| Cpd. | | Mass |
|---|---|---|
| No. | Cpd Name | Observed |
| I′-1a | methyl {2-[((3-chlorophenyl){2-methyl-5-[({(2S)- | 532.5 |
| | 2-(methylamino)-3-[(3R)-tetrahydro-2H-pyran-3-yl]propyl}amino)carbonyl]phenyl}methyl)oxy]ethyl}carbamate | |
| I′-1b | methyl {2-[((S)-(3-chlorophenyl){2-methyl-5-[({(2S)-2- | 532.5 |
| | (methylamino)-3-[(3R)-tetrahydro-2H-pyran-3- | |
| | yl]propyl}amino)carbonyl]phenyl}methyl)oxy]ethyl}carbamate | |
| I′-2a | methyl {2-[((3-chlorophenyl){3-[({(2S)-2-(methylamino)- | 518.5 |
| | 3-[(3R)-tetrahydro-2H-pyran-3-yl]propyl}amino)carbonyl]phenyl}methyl)oxy]ethyl}carbamate | |
| I′-2b | methyl {2-[((R)-(3-chlorophenyl){3-[({(2S)-2- | 518.0 |
| | (methylamino)-3-[(3R)-tetrahydro-2H-pyran-3-yl]propyl}amino)carbonyl]phenyl} methyl)oxy]ethyl}carbamate | |
| I′-2c | methyl {2-[((R)-(3-chlorophenyl){3-[({(2R)-2- | 518.0 |
| | (methylamino)-3-[(3S)-tetrahydro-2H-pyran-3-yl]propyl}amino)carbonyl]phenyl}methyl)oxy]ethyl}carbamate | |
| I′-2d | methyl {2-[((R)-(3-chlorophenyl){3-[({(2S)-2- | 518.0 |
| | (methylamino)-3-[(3S)-tetrahydro-2H-pyran-3-yl]propyl}amino)carbonyl]phenyl}methyl)oxy]ethyl}carbamate | |
| I′-2e | methyl {2-[((R)-(3-chlorophenyl){3-[({(2R)-2- | 518.5 |
| | (methylamino)-3-[(3R)-tetrahydro-2H-pyran-3-yl]propyl}amino)carbonyl]phenyl}methyl)oxy]ethyl}carbamate | |
| I′-3a | methyl [2-({(3-chlorophenyl)[3-({[(2S)-3-cyclohexyl-2- | 516.7 |
| | (methylamino)propyl]amino}carbonyl)phenyl]methyl}oxy)ethyl] carbamate | |
| I′-4a | methyl [2-({(3-chlorophenyl)[3-({[(2S)-4-methyl-2- | 476.4 |
| | (methylamino) pentyl]amino}carbonyl)phenyl]methyl}oxy)ethyl]carbamate | |
| I′-5a | methyl [2-({(3-chlorophenyl)[3-({[(2S)-3-cyclohexyl-2- | 533.8 |
| | (methylamino)propyl]amino} carbonyl)-4- | |
| | fluorophenyl]methyl} oxy)ethyl] carbamate | |
| I′-6a | methyl {2-[((3-chlorophenyl){4-fluoro-3-[({(2S)-2- | 535.9 |
| | (methylamino)-3-[(3R)-tetrahydro-2H-pyran-3-yl]propyl}amino)carbonyl]phenyl}methyl)oxy]ethyl}carbamate | |
| I′-7a | methyl [2-({(3-chlorophenyl)[5-({[(2S)-3-cyclohexyl-2- | 530.5 |
| | (methylamino)propyl]amino} carbonyl)-2- | |
| | methylphenyl]methyl} oxy)ethyl] carbamate | |
| I′-8a | methyl (2-{[{3-chloro-5-[({(2S)-2-(methylamino)-3-[(3R)- | 552.5 |
| | tetrahydro-2H-pyran-3-yl]propyl}amino) carbonyl]phenyl}(3-chlorophenyl) methyl]oxy}ethyl)carbamate | |
| I′-9a | methyl (2-{[[3-chloro-5-({[(2S)-3-cyclohexyl-2- | 550.6 |
| | (methylamino)propyl] amino}carbonyl)phenyl](3- | |
| | chlorophenyl)methyl]oxy}ethyl) carbamate | |
| I′-10a | methyl [2-({(3-chlorophenyl)[5-({[(2S)-3-cyclohexyl-2- | 534.5 |
| | (methylamino)propyl]amino}carbonyl)-2- | |
| | fluorophenyl]methyl} oxy)ethyl] carbamate | |
| I′-11a | methyl [2-({(3-chlorophenyl)[3-({[(2S)-2-(methylamino)- | 518.5 |
| | 3-(tetrahydro-2H-pyran-4- | |
| | yl)propyl]amino}carbonyl)phenyl]methyl}oxy)ethyl]carbamate | |
| I′-12a | methyl {2-[((3-chlorophenyl){2-fluoro-5-[({(2S)-2- | 536.4 |
| | (methylamino)-3-[(3R)-tetrahydro-2H-pyran-3- | |
| | yl]propyl}amino)carbonyl]phenyl}methyl)oxy]ethyl}carbamate | |
| I′-13a | methyl {2-[((5-chloro-2-methylphenyl){3-[({(2S)-2- | 532.3 |
| | (methylamino)-3-[(3R)-tetrahydro-2H-pyran-3- | |
| | yl]propyl}amino) carbonyl] phenyl}methyl)oxy]ethyl}carbamate | |
| I′-13b | methyl {2-[((R)-(5-chloro-2-methylphenyl){3-[({(2S)-2- | 532.0 |
| | (methylamino)-3-[(3R)-tetrahydro-2H-pyran-3- | |
| | yl]propyl}amino) carbonyl]phenyl}methyl)oxy]ethyl}carbamate | |

### Example 4

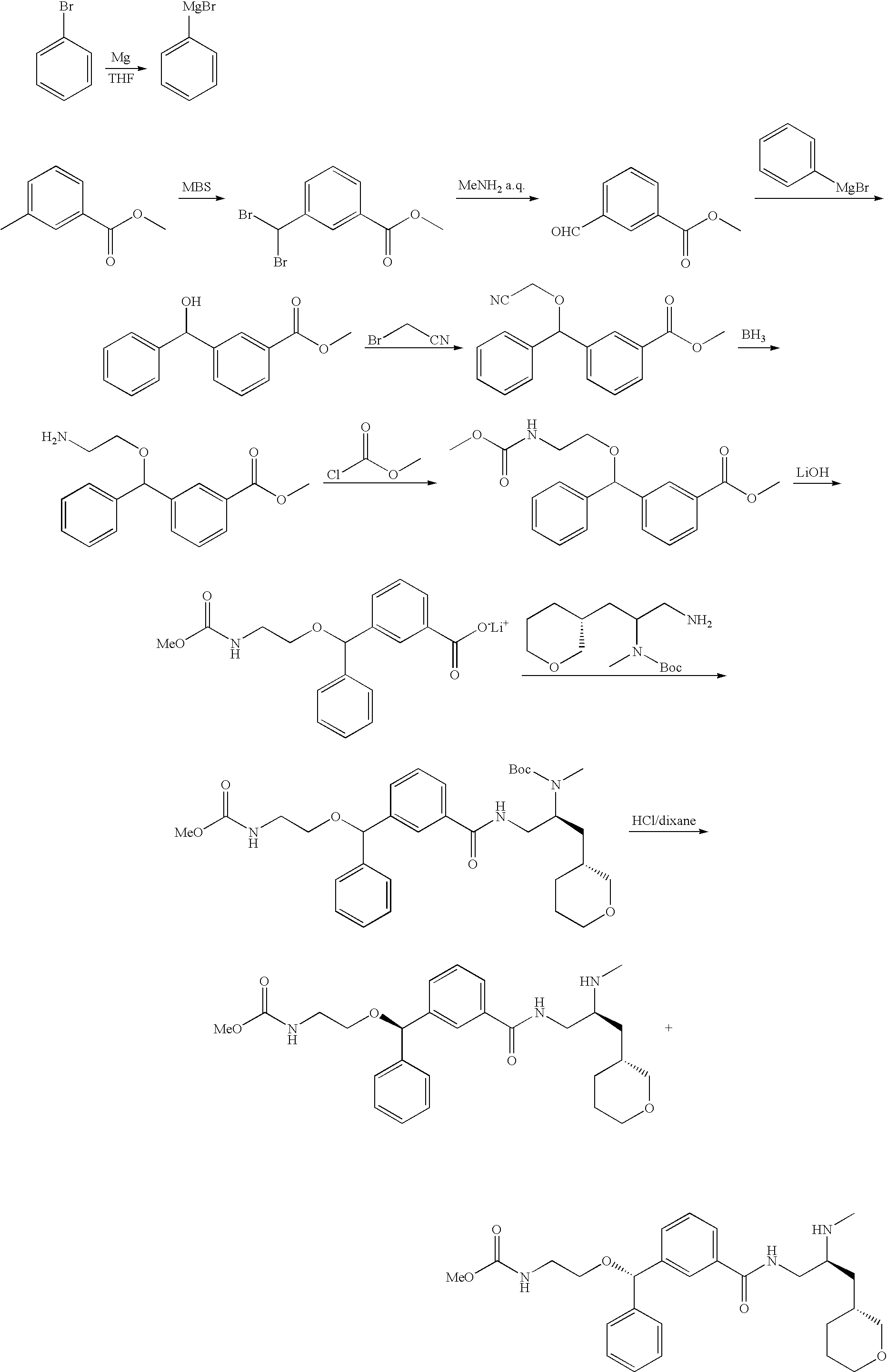

### Step 1. methyl 3-(dibromomethyl)benzoate

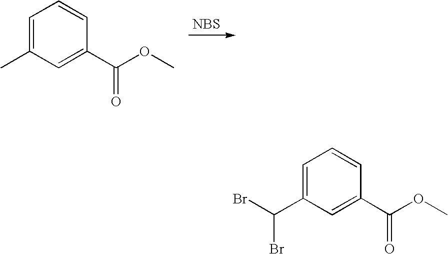

A mixture of methyl 3-methylbenzoate (30 g, 0.2 mol), NBS (78.3 g, 0.4 mol) and benzoic peroxyanhydride (24.2 g, 0.1 mol) in CCl₄ (500 mL) was refluxed for overnight. The mixture was cooled to room temperature and filtered off, and the filtrate was concentrated to give crude methyl 3-(dibromomethyl)benzoate (65 g, crude).

### Step 2 methyl 3-formylbenzoate

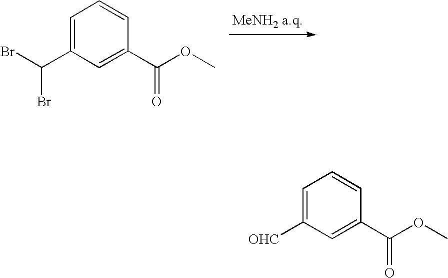

A solution MeNH₂ in water (33%, 300 mL) was added to a solution of methyl 3-(dibromomethyl)benzoate (65 g, crude) at ambient temperature. The above mixture was heated at 60° C. for 3 hours under N₂ The mixture was filtered, and the filtrate was concentrated until most MeOH was removed. The residue was extracted with diethyl ether, and the separated organic layer washed with water many times, dried over Na₂SO₄ and concentrated to give methyl 3-formylbenzoate (20.2 g). ¹H NMR (CDCl₃): 3.90 (s, 3H), 7.54 (t, 1H), 8.02 (d, 1H), 8.22 (d, 1H), 8.46 (s, 1H), 10.01 (s, 1H).

### Step 3. phenylmagnesium bromide

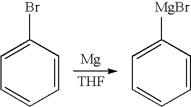

A 100 mL 3-neck flask, was placed under N₂ and equipped with a condenser and an addition funnel. Magnesium (2.9 g, 120 mmol) were added. Bromobenzene (15.7 g, 100 mmol) was taken up in dry THF (120 mL), and transferred to the addition funnel. The Grignard reaction was initiated with approximately 5 mL of the bromobenzene solution and iodine. The remained bromobenzene solution was added and the reaction was heated under reflux for 1 hour. The resulting solution was used for next step directly.

### Step 4. methyl 3-(hydroxy(phenyl)methyl)benzoate

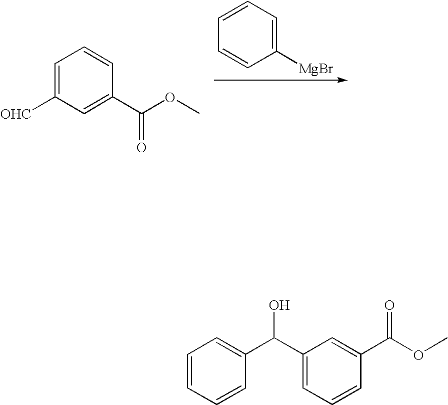

A solution of phenylmagnesium bromide in THF (0.8 M, 115 mL, 92 mmol) was added dropwise to a solution of methyl 3-formylbenzoate (10 g, 61 mmol) in THF (100 mL) at 0° C. The result mixture was stirred at 0° C. for 1 hour. The mixture was quenched with a solution of saturate NH₄Cl and extracted with ethyl acetate. The separated layer was dried over Na₂SO₄ and concentrated to give crude product, which was purified by silica gel chromatography to give 3-(hydroxy(phenyl)methyl)benzoic acid (3.03 g, 20%). ¹H NMR (CDCl₃): 2.25 (d, 1H), 3.85 (s, 3H), 5.23 (d, 1H), 7.19-7.38 (m, 6H), 7.52 (d, 1H), 7.87 (d, 1H), 8.02 (s, 1H).

### Step 5. methyl 3-((cyanomethoxy)(phenyl)methyl)benzoate

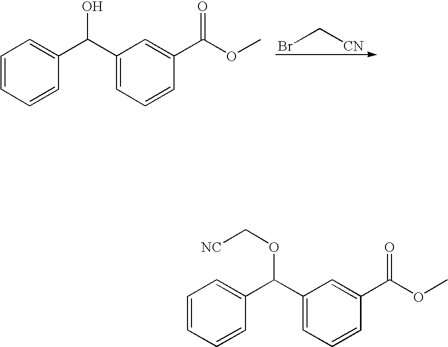

NaH (595 mg, 60%, 24.8 mmol) was added in portion to a mixture of methyl 3-(hydroxy(phenyl)methyl)benzoate (2 g, 8.26 mmol) in CH₃CN (30 mL) at 0° C. The mixture was stirred at 0° C. for 1 h. Then 2-bromoacetonitrile (3.0 g, 24.8 mmol) was added at 0° C. The resulting mixture was stirred for 2 hours at room temperature. The addition of the same of NaH and 2-bromoacetonitrile was repeated. The reaction mixture was quenched with sat. NH₄Cl. The mixture was extracted with dichloromethane. The separated organic layers was washed with brine, dried over Na₂SO₄ and concentrated to give to crude methyl 3-((cyanomethoxy)(phenyl)methyl)benzoate (0.7 g, 30%). ¹H NMR (CDCl₃): 3.91 (s, 3H), 4.24-4.30 (m, 2H), 5.67 (s, 1H), 7.30-7.40 (m, 5H), 7.44 (t, 1H), 7.53 (d, 1H), 7.97 (d, 1H), 8.04 (s, 1H).

### Step 6. methyl 3-((2-aminoethoxy)(phenyl)methyl)benzoate

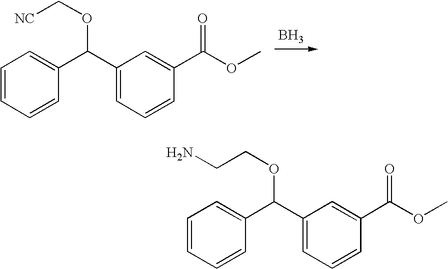

Borane-tetrahydrofuran complex (1M, 4.5 mL,4.5 mmol) was added to a solution of methyl 3-((cyanomethoxy)(phenyl)methyl)benzoate (500 mg, 1.8 mmol) in THF (5 mL) at 0° C. under nitrogen atmosphere. The above mixture was heated at 50° C. for 4 hours. The mixture was quenched with MeOH and concentrated to give crude methyl 3-((2-aminoethoxy)(phenyl)methyl)benzoate (480 mg), which was used for next step without purification.

### Step 7. methyl 3-((2-(methoxycarbonylamino)ethoxy)(phenyl)methyl)benzoate

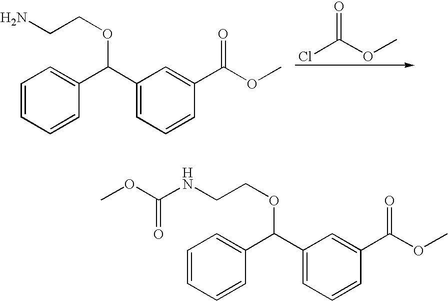

Methyl carbonochloridate (191 mg, 2.0 mmol) was added dropwise to a solution of methyl 3-((2-aminoethoxy)(phenyl)methyl)benzoate (480 mg, 1.7 mmol) and Et₃N (255 mg, 2.5 mmol) in THF (10 mL) at 0° C. The above mixture was stirred at room temperature for 0.5 h. The mixture was treated with water and CH₂Cl₂, and the separated organic layer was dried over Na₂SO₄ and concentrated to give crude product, which was purified by preparative TLC to give methyl 3-((2-(methoxycarbonylamino)ethoxy)(phenyl)methyl)benzoate (190 mg, 33%). ¹H NMR (CDCl₃): 3.37 (m, 2H), 3.47 (m, 2H), 3.60 (s, 3H), 3.83 (s, 3H), 5.02 (brs,1H), 5.34 (s, 1H), 7.19 (m, 2H), 7.23 (m, 3H), 7.34 (t, 1H), 7.45 (d, 1H), 7.88 (d, 1H), 7.96 (s, 1H).

### Step 8. lithium 3-((2-(methoxycarbonylamino)ethoxy)(phenyl)methyl)benzoate

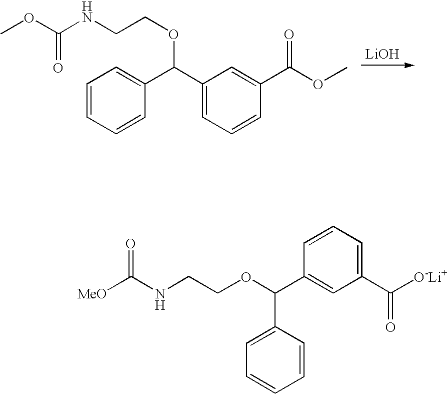

LiOH.H₂O (73 mg, 1.74 mmol) was added to a mixture of methyl 3-((2-(methoxycarbonylamino)ethoxy)(phenyl)methyl)benzoate (200 mg, 0.58 mmol) in MeOH (8 mL) and water (2 mL) at room temperature. The mixture was stirred at room temperature for overnight. The reaction mixture was concentrated to give crude lithium 3-((2-(methoxycarbonylamino)ethoxy)(phenyl)methyl)benzoate (270 mg).

### Step 9. [2-{3-[(2-methoxycarbonylamino-ethoxy)-phenyl-methyl]-benzoylamino}-1-(tetrahydro-pyran-3-ylmethyl)-ethyl]-methyl-carbamic acid tert-butyl ester

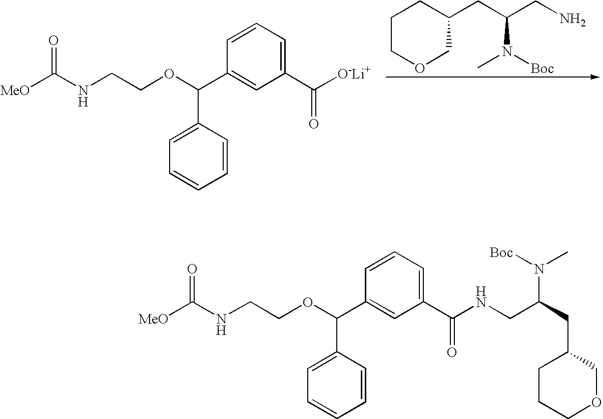

DIEA (299 mg, 2.3 mmol) was added dropwise to a mixture of lithium 3-((2-(methoxycarbonylamino)ethoxy)(phenyl)methyl)benzoate (194 mg, 0.6 mmol), HOBT (157 mg, 1.2 mmol), EDCI (230 mg, 1.2 mmol), and tert-butyl (S)-1-amino-3-((R)-tetrahydro-2H-pyran-3-yl)propan-2-yl(methyl)carbamate (174 mg, 0.6 mmol) in DMF (4 mL) at 0° C. The mixture was warmed to room temperature and stirred for overnight. After most DMF was removed, the residue was treated with water and ethyl acetate. The organic layers was dried over Na₂SO₄ and give crude [[2-{3-[(2-methoxycarbonylamino-ethoxy)-phenyl-methyl]-benzoylamino}-1-(tetrahydro-pyran-3-ylmethyl)-ethyl]-methyl-carbamic acid tent-butyl ester (351 mg, crude

### Step 10. methyl 2-((3-((S)-2-(methylamino)-3-((R)-tetrahydro-2H-pyran-3-yl)propylcarbamoyl)phenyl)(phenyl)methoxy)ethylcarbamate

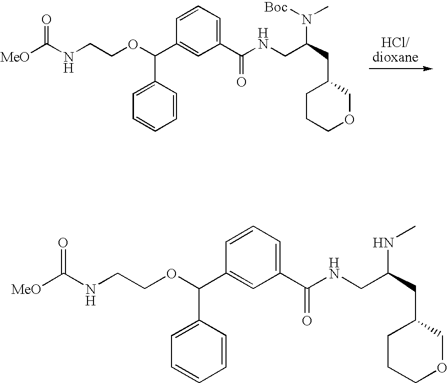

[2-{3-[(2-methoxycarbonylamino-ethoxy)-phenyl-methyl]-benzoylamino}-1-(tetrahydro-pyran-3-ylmethyl)-ethyl]-methyl-carbamic acid tert-butyl ester (350 mg, crude) was dissolved in a solution of HCl in dioxane (2N, 10 mL) at room temperature. The mixture was stirred at room temperature for overnight. The mixture was purified by preparative HPLC to give ethyl 2-((3-((S)-2-(methylamino)-3-((R)-tetrahydro-2H-pyran-3-yl)propylcarbamoyl)phenyl)(phenyl)methoxy)ethylcarbamate (65.8 mg, 23%). ¹H NMR (MeOH): 1.25 (m, 1H), 1.39-1.70 (m, 4H), 1.75 (m, 1H), 1.95 (d, 1H), 2.73 (s, 3H), 3.10 (t, 1H), 3.27 (t, 2H), 3.35 (m, 2H), 3.45 (t, 3H), 3.58 (m, 3H), 3.72 (s, 1H), 3.80 (m, 2H), 5.44 (s, 1H), 7.20 (d, 1H), 7.25 (t, 3H), 7.33 (d, 1H), 7.39 (t, 1H), 7.50 (s, 1H), 7.70 (d, 1H), 7.87 (s, 1H).

### Example 5

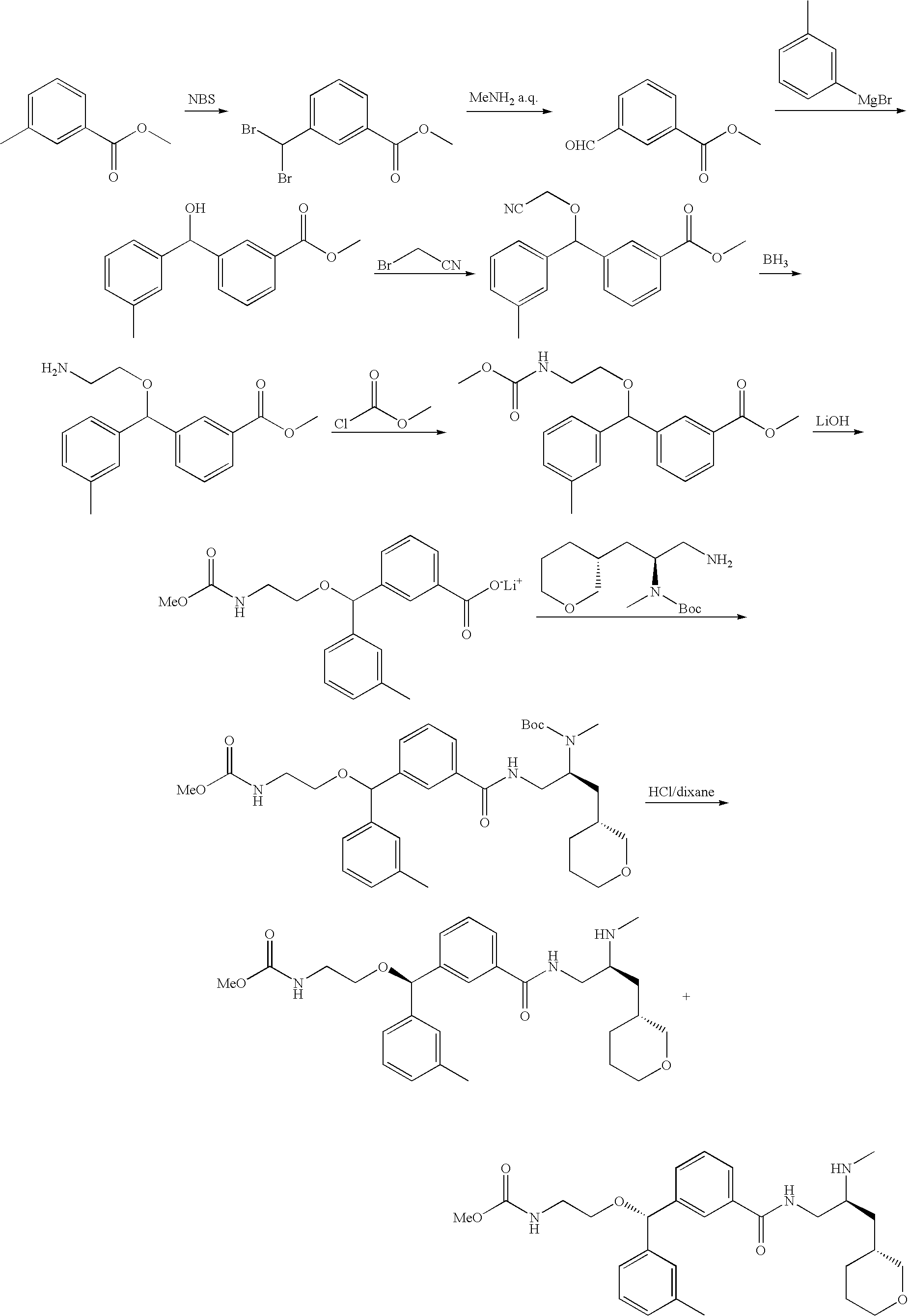

### Step 1. methyl 3-(dibromomethyl)benzoate

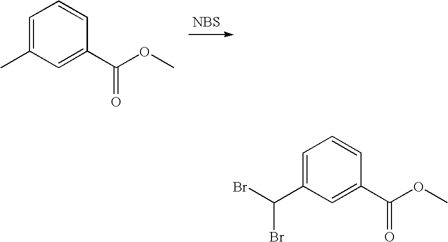

A mixture of methyl 3-methylbenzoate (30 g, 0.2 mol), NBS (78.3 g, 0.4 mol) and benzoic peroxyanhydride (24.2 g, 0.1 mol) in CCl₄ (500 mL) was heated under reflux overnight. The mixture was cooled to room temperature and filtered off, and the filtrate was concentrated to give crude methyl 3-(dibromomethyl)benzoate (65 g, crude).

### Step 2. methyl 3-formylbenzoate

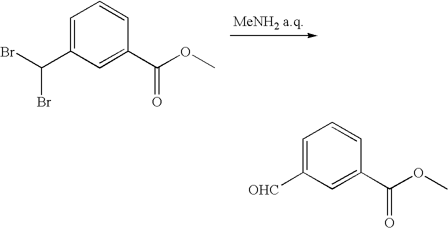

A solution MeNH₂ in water (33%, 300 mL) was added to a solution of methyl 3-(dibromomethyl)benzoate (65 g, crude) at ambient temperature. The above mixture was heated at 60° C. for 3 hours under N₂. The mixture was filtered, and the filtrate was concentrated until most MeOH was removed. The residue was extracted with diethyl ether, and the separated organic layer washed with water many times, dried over Na₂SO₄ and concentrated to give methyl 3-formylbenzoate (20.2 g). ¹H NMR (CDCl₃): 3.90 (s, 3H), 7.54 (t, 1H), 8.02 (d, 1H), 8.22 (d, 1H), 8.46 (s, 1H), 10.01 (s, 1H).

### Step 3. m-tolylmagnesium bromide

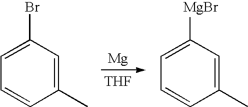

A 100 mL 3-neck flask, was placed under N₂ and equipped with a condenser and an addition funnel. Magnesium (4.2 g, 175 mmol) were added. 1-bromo-3-methylbenzene (25 g, 146 mmol) was taken up in dry THF (200 mL), and transferred to the addition funnel. The Grignard reaction was initiated with approximately 5 mL of 1-bromo-3-methylbenzene solution and iodine. The remaining 1-bromo-3-methylbenzene solution was added and the reaction was refluxed 1 hour. The resulting solution was used for next step directly.

### Step 4. methyl 3-(hydroxy(m-tolyl)methyl)benzoate

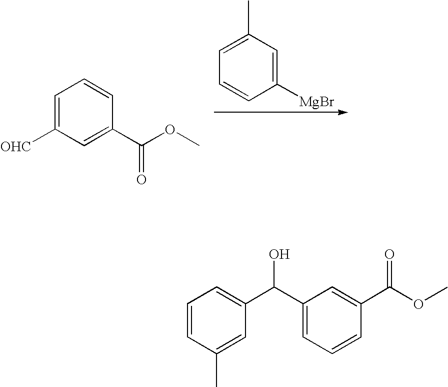

A solution of m-tolylmagnesium bromide in THF (0.73 M, 157 mL, 115 mmol) was added dropwise to a solution of methyl 3-formylbenzoate (15 g, 77 mmol) in THF (100 mL) at 0° C. The result mixture was stirred at 0° C. for 1 hour. The mixture was quenched with a solution of saturate NH₄Cl and extracted with ethyl acetate. The separated organic layer was dried over Na₂SO₄ and concentrated to give crude product, which was purified by silica gel chromatography to give methyl 3-(hydroxy(m-tolyl)methyl)benzoate (4 g, 20%). ¹H NMR (CDCl₃): 2.27 (brs, 1H), 2.35 (s, 3H), 3.92 (s, 3H), 5.87 (s, 1H), 7.10 (d, 1H), 7.17 (m, 2H), 7.26 (t, 1H), 7.42 (t, 1H), 7.59 (d, 1H), 7.96 (d, 1H), 8.11 (s, 1H).

### Step 5. methyl 3-((cyanomethoxy)(m-tolyl)methyl)benzoate

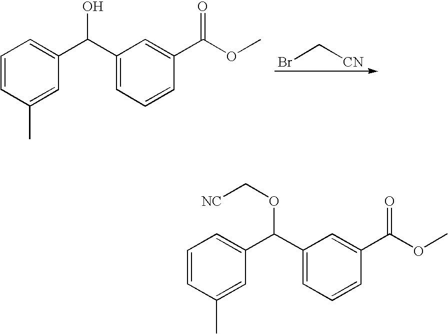

NaH (1.2 g, 60%, 30.3 mmol) was added in portion to a mixture of methyl 3-(hydroxy(m-tolyl)methyl)benzoate (2 g, 10.1 mmol) in CH₃CN (30 mL) at 0° C. The mixture was stirred at 0° C. for 1 h. Then 2-bromoacetonitrile (3.6 g, 30.3 mmol) was added at 0° C. The resulting mixture was stirred for 2 hours at room temperature. The addition of the same of NaH and 2-bromoacetonitrile was repeated. The reaction mixture was quenched with saturate NH₄Cl. The mixture was extracted with dichloromethane. The separated organic layers was washed with brine, dried over Na₂SO₄ and concentrated to give to crude methyl 3-((cyanomethoxy)(m-tolyl)methyl)benzoate (0.7 g, 30%). ¹H NMR (CDCl₃): 2.35 (s, 3H), 3.91 (s, 3H), 4.19-4.30 (m, 2H), 5.62 (s, 1H), 7.13 (m, 3H), 7.27 (m, 1H), 7.44 (t, 1H), 7.53 (d, 1H), 7.97 (d, 1H), 8.04 (s, 1H).

### Step 6. methyl 3-((2-aminoethoxy)(m-tolyl)methyl)benzoate

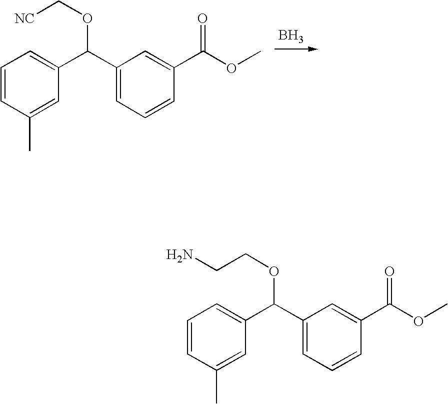

Borane-tetrahydrofuran complex (1M, 5.3 mmol) was added to a solution of methyl 3-((2-aminoethoxy)(phenyl)methyl)benzoate (500 mg, 2.1 mmol) in THF (5 mL) at 0° C. under nitrogen atmosphere. The above mixture was heated at 50° C. for 4 hours. The mixture was quenched with Me0H and concentrated to give crude methyl 3-((2-aminoethoxy)(m-tolyl)methyl)benzoate (480 mg, crude), which was used for next step without purification.

### Step 7. methyl 3((2-(methoxycarbonylamino)ethoxy)(m-tolyl)methyl)benzoate

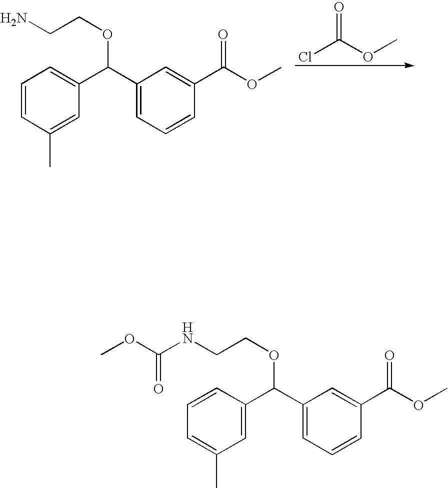

Methyl carbonochloridate (183 mg, 1.9 mmol) was added dropwise to a solution of methyl 3-((2-aminoethoxy)(m-tolyl)methyl)benzoate (480 mg, 1.6 mmol) and Et₃N (244 mg, 2.4 mmol) in THF (10 mL) at 0° C. The above mixture was stirred at room temperature for 0.5 h. The mixture was treated with water and CH₂Cl₂, and the separated organic layer was dried over Na₂SO₄ and concentrated to give crude product, which was purified by preparative TLC to give methyl 3-((2-(methoxycarbonylamino)ethoxy)(m-tolyl)methyl)benzoate (195 mg, 34%). ¹H NMR (CDCl₃): 2.33 (s, 3H), 3.38 (m, 2H), 3.52 (m, 2H), 3.91 (s, 3H), 5.07 (brs,1H), 5.36 (s, 1H), 7.07 (m, 3H), 7.20 (t, 3H), 7.38 (t, 1H), 7.52 (d, 1H), 7.93 (d, 1H), 8.02 (s, 1H).

### Step 8. lithium 3((2-(methoxycarbonylamino)ethoxy)(m-tolyl)methyl)benzoate

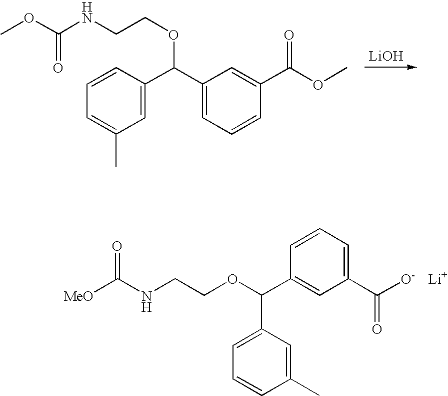

LiOH.H₂O (47 mg, 1.1 mmol) was added to a mixture of methyl 3-((2-(methoxycarbonylamino)ethoxy)(m-tolyl)methyl)benzoate (200 mg, 0.56 mmol) in MeOH (10 mL) and water (2 mL) at room temperature. The mixture was stirred at room temperature for overnight. The reaction mixture was concentrated to give crude lithium 3-((2-(methoxycarbonylamino)ethoxy)(phenyl)methyl)benzoate (250 mg, crude).

### Step 9. [2-{3-[(2-Methoxycarbonylamino-ethoxy)-m-tolyl-methyl]-benzoylamino}-1-(tetrahydro-pyran-3-ylmethyl)-ethyl]-methyl-carbamic acid tert-butyl ester

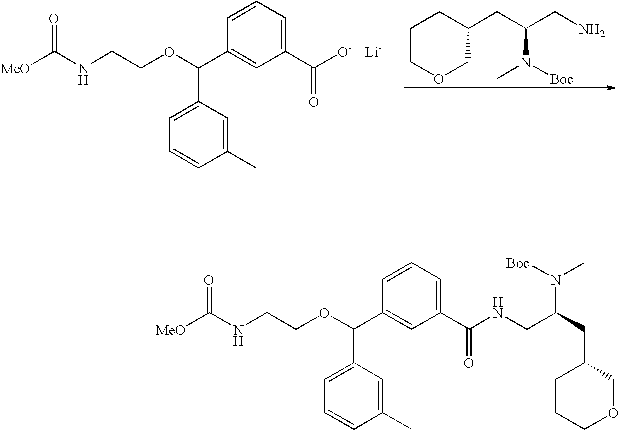

DIEA (222 mg, 1.7 mmol) was added dropwise to a mixture of lithium 3-((2-(methoxycarbonylamino)ethoxy)(m-tolyl)methyl)benzoate (150 mg, 0.4 mmol), HOBT (116 mg, 0.8 mmol), EDCI (170 mg, 0.8 mmol), and tert-butyl (S)-1-amino-3-((R)-tetrahydro-2H-pyran-3-yl)propan-2-yl(methyl)carbamate (129 mg, 0.5 mmol) in DMF (5 mL) at 0° C. The mixture was warmed to room temperature and stirred for overnight. After most DMF was removed, the residue was treated with water and ethyl acetate. The organic layers was dried over Na₂SO₄ and give crude [2-{3-[(2-methoxy carbonylamino-ethoxy)-m-tolyl-methyl]-benzoylamino}-1-(tetrahydro-pyran-3-ylmethyl)-ethyl]-methyl-carbamic acid tert-butyl ester (280 mg, crude).

### Step 10. methyl 2-((3-((S)-2-(methylamino)-3-((R)-tetrahydro-2H-pyran-3-yl)propylcarbamoyl)phenyl)(m-tolyl)methoxy)ethylcarbamate

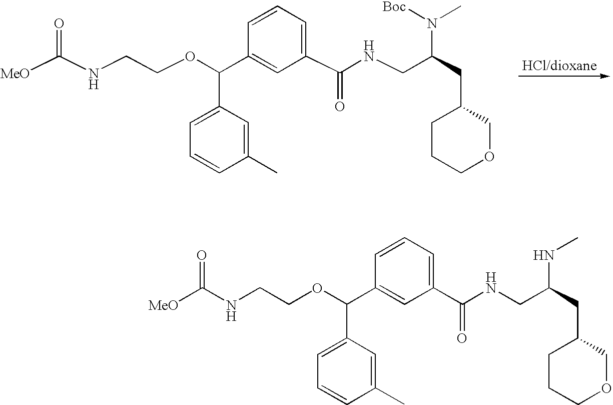

[2-{3-[(2-Methoxycarbonylamino-ethoxy)-m-tolyl-methyl]-benzoylamino}-1-(tetrahydro-pyran-3-ylmethyl)-ethyl]-methyl-carbamic acid tert-butyl ester (280 mg, 0.47 mmol) was dissolved in a solution of HCl in dioxane (2N, 8 mL) at room temperature. The mixture was stirred at room temperature for overnight. The mixture was purified by preparative HPLC and to give methyl 2-((3-((S)-2-(methylamino)-3-((R)-tetrahydro-2H-pyran-3-yl) propylcarbamoyl)phenyl)(m-tolyl)methoxy)ethylcarbamate (85.7 mg, 37%). ¹HNMR (MeOH): 1.25 (m, 1H), 1.39-1.68 (m, 4H), 1.75 (m, 1H), 1.95 (d, 1H), 2.22 (s, 1H), 2.73 (s, 3H), 3.08 (t, 1H), 3.27 (t, 2H), 3.35 (m, 2H), 3.45 (m, 3H), 3.58 (m, 31-1), 3.72 (s, 1H), 3.80 (m, 2H), 5.38 (s, 1H), 7.01 (d, 1H), 7.13 (m, 3H), 7.37 (t, 1H), 7.50 (s, 1H), 7.86 (s, 1H).

### Example 6

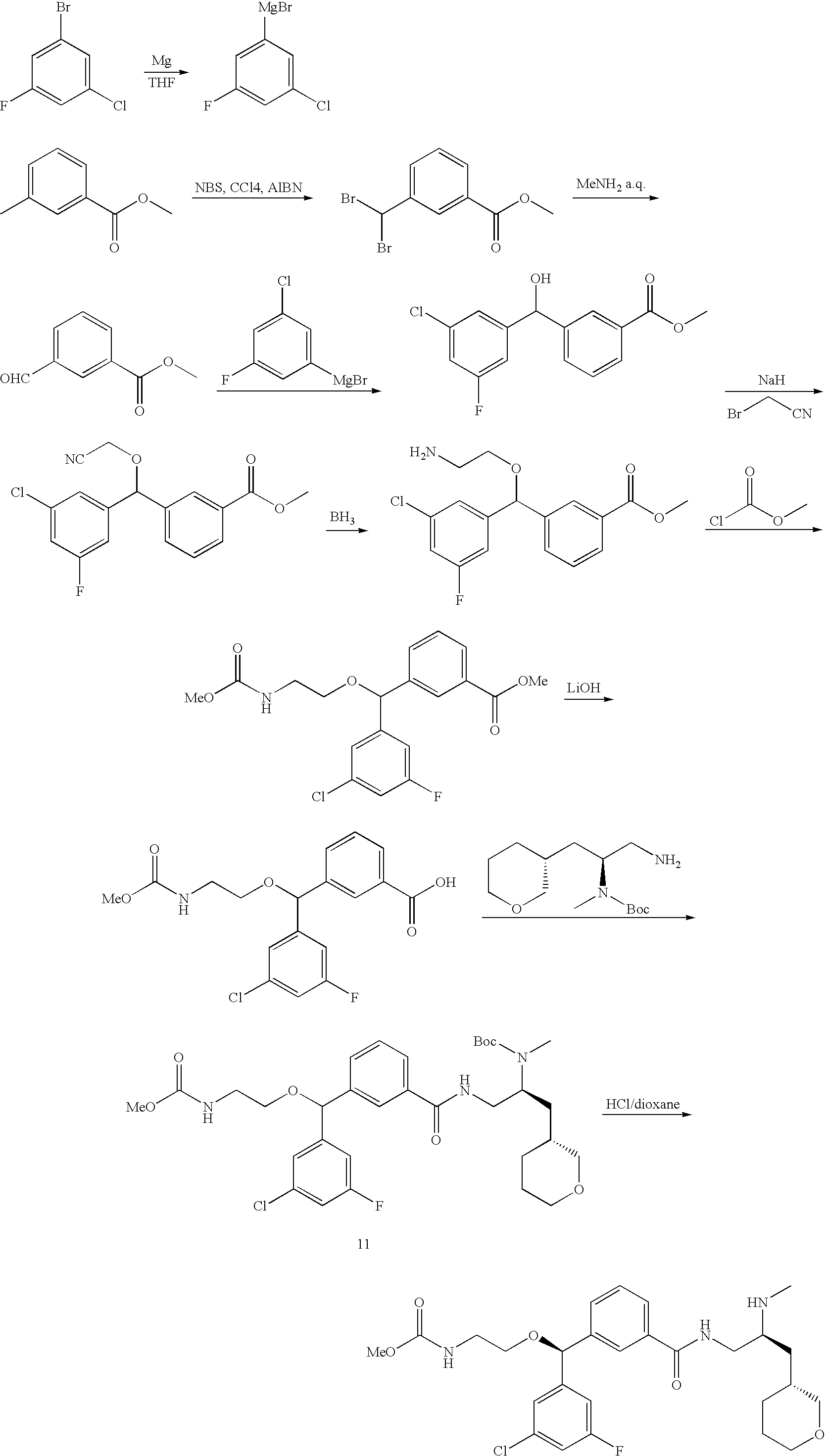

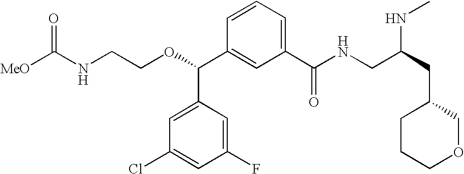

### Step 1. methyl 3-(dibromomethyl)benzoate

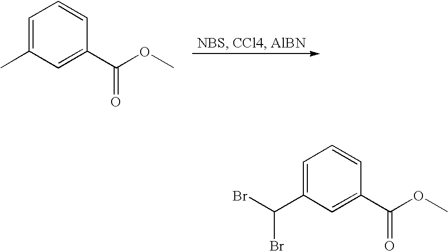

A mixture of methyl 3-methylbenzoate (150 g, 1 mol), NBS (407 g, 2.3 mmol), and benzoic peroxyanhydride (121 g, 0.5 mmol) in CCl₄ (1 L) was heated under reflux overnight. The mixture was filtered and the filtrate was concentrated to give methyl 3-(dibromomethyl)benzoate (300 g, 98%) ¹HNMR: (400 MHz, CDCl₃): δ=3.95 (s, 3H), 6.67 (s, 1H), 3.87 (s, 3H), 7.25 (m, 1H), 7.49 (m, 1H), 7.78 (m, 1H), 8.21 (s, 1H).

### Step 2. methyl 3-formylbenzoate

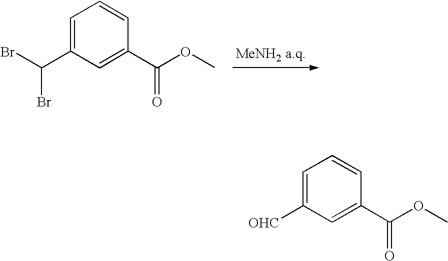

A mixture of methyl 3-(dibromomethyl)benzoate (200 g, 0.66 mol) in N(CH₃)₂ solution (33%) (500 ml) and MeOH (500 ml ) was heated to 60° C. for 2 hours. Then the mixture was concentrated and extracted with Et₂O, washed with water (200 mL) three times. The organic phase was dried over anhydrous Na₂SO₄, concentrated to give methyl 3-formylbenzoate (44 g, 41%). ¹HNMR: (400 MHz, CDCl₃): δ=3.88 (s, 3H), 7.57 (t, 1H), 8.02 (d, 1H), 8.16 (d, 1H), 8.46 (s, 1H), 10.00 (s, 1H).

### Step 3. (3-chloro-5-fluorophenyl)magnesium bromide

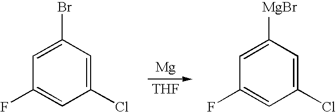

A 100 mL 3-neck flask was placed under N₂ and equipped with a condenser and an addition funnel. Magnesium (2.0 g, 83 mmol) were added. bromo-3-chloro-5-fluoro benzene (15 g, 72.5 mmol) was taken up in dry THF (70 mL), and transferred to the addition funnel. The Grignard reaction was initiated with approximately 2 mL of the bromo-3-chloro -5-fluorobenzene solution and iodine. The remaining bromo-3-chloro-5-fluorobenzene solution was added and the reaction was refluxed 1 hour.

### Step 4. methyl 3-((3-chloro-5-fluorophenyl)(hydroxy)methyl)benzoate

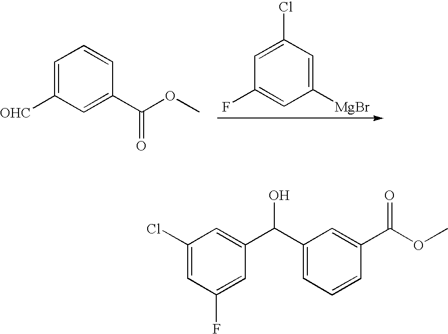

To a solution of methyl 3-formylbenzoate (7.9 g, 48.0 mmol) in THF (30 ml) was added above (3-chloro-5-fluorophenyl)magnesium bromide (72 mmol) at −78° C. under N₂. After addition, the mixture was allowed to warm to room temperature for 4 hours. The mixture was quenched by saturated NaHCO₃ solution, extracted with ethyl acetate, dried over anhydrous Na₂SO₄, concentrated to give methyl 3-((3-chloro-5-fluorophenyl)(hydroxy)methyl) benzoate which was purified by column (5.7 g, 40%). ¹HNMR: (400 MHz, CDCl₃): δ3.92 (s, 3H), 5.84 (s, 1H), 7.02 (m, 2H), 7.18 (s, 1H), 7.45 (t, 1H), 7.56 (d, 1H), 7.99 (d, 1H), 8.05 (s, 1H).

### Step 5. methyl 343-chloro-5-fluorophenyl)(hydroxy)methyl)benzoate

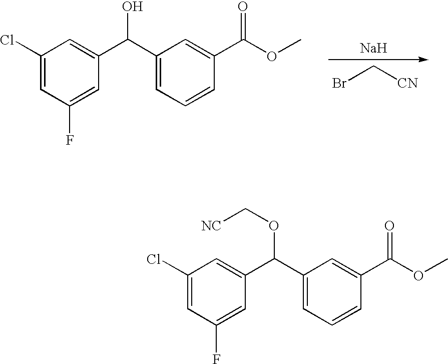

To a solution of methyl 3-((3-chloro-5-fluorophenyl)(hydroxy)methyl)benzoate (2.1 g, 7.12 mmol) in acetonitrile (20 mL) was added NaH (1.42 g, 35.6 mmol, 60% in oil) at 0° C. under N₂. After 1 hour later, then the mixture was cooled to −20° C., and 2-bromoacetonitrile was added dropwise, the mixture allowed to warmed to room temperature for 4 hours. The reaction was quenched with water. Acetonitrile was removed by reduced pressure, extracted with CH₂Cl₂, dried over Na₂SO₄ and concentrated in vacuo to give crude methyl 3-((3-chloro-5-fluorophenyl)(hydroxy)methyl)benzoate (2.3 g, 97%), which was used for the next step without further purification. ¹HNMR: (400 MHz, CDCl₃): δ=3.86 (s, 3H), 4.18 (q, 2H), 5.53 (s, 1H), 6.89 (d, 2H), 6.96 (d, 1H), 7.06 (s, 1H), 7.43 (m, 2H), 7.93 (s, 1H), 7.97 (d, 1H).

### Step 6. methyl 3-((2-aminoethoxy)(3-chloro-5-fluorophenyl)methyl)benzoate

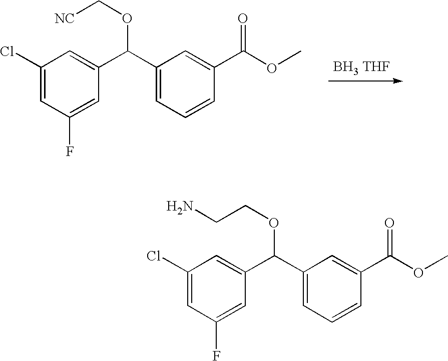

A mixture of methyl 3-((3-chloro-5-fluorophenyl)(hydroxy)methyl)benzoate (1.8 g, 5.4 mmol) in THF (30 mL) was heated to 60° C., then BH₃THF (1M/L, 15 mL) was added dropwise. After addition, the mixture stirred for 4 hours at 60° C. The mixture was quenched by Me0H, concentrated to give methyl 3-((2-aminoethoxy)(3-chloro-5-fluorophenyl)methyl)benzoate, which was used for the next step without further purification (1.72 g, crude).

### Step 7 methyl 3-((3-chloro-5-fluorophenyl)(2-(methoxycarbonylamino)ethoxy)methyl)-benzoate

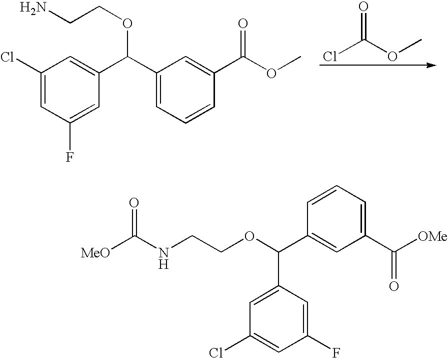

A solution of methyl 3-((2-aminoethoxy)(3-chloro-5-fluorophenyl)methyl)benzoate (1.0 g, 2.97 mmol) in dry CH₂Cl₂ (20 mL) and Et₃N (600 mg, 5.94 mmol) was cooled to 0° C. in ice-water bath, methyl carbonochloridate (420 mg, 4.46 mmol) was added dropwise. After addition, the reaction mixture was stirred for 30 min at room temperature. The mixture was concentrated to give the crude product, which was purified by preparative TLC to give methyl 3-((3-chloro-5-fluorophen-yl)(2-(methoxycarbonylamino)ethoxy)methyl)-benzoate (150 mg, 13%). ¹HNMR: (400 MHz, CDCl₃): δ=3.41 (m, 4H), 3.59 (s, 3H), 3.85 (s, 3H), 4.95 (s, 1H), 5.26 (s, 1H), 6.89 (m, 2H), 7.04 (s, 1H), 7.40 (m, 2H), 7.91 (m, 2H)

### Step 8 3-((3-chloro-5-fluorophenyl)(2-(methoxycarbonylamino)ethoxy)methyl)benzoic acid

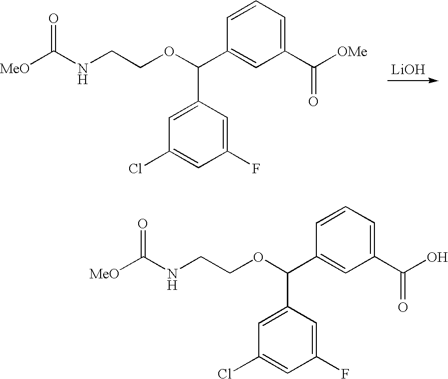

To a solution of methyl 3-((3-chloro-5-fluorophenyl)(2-(methoxycarbonylamino)ethoxy)methyl)-benzoate (180 mg, 0.379 mmol) in MeOH/H₂O (3:1, 30 mL) was added LiOH.H₂O (48 mg, 1.14 mmol) at room temperature. Then the mixture was heated to 60° C. for 3 hours. The mixture was concentrated, washed with 1N HCl solution, extracted with ethyl acetate, dried over anhydrous Na₂SO₄, concentrated to give 3-((3-chloro-5-fluorophenyl)(2-methoxycarbon-ylamino)ethoxy)methyl)benzoic acid, which was used for the next step without further purification (170 mg, 98%).

### Step 9 [2-{3-[(3-Chloro-5-fluoro-phenyl)-(2-methoxycarbonylamino-ethoxy)-methyl]-benzoylamino}-1-(tetrahydro-pyran-3-ylmethyl)-ethyl]-methyl-carbamic acid tert-butyl ester

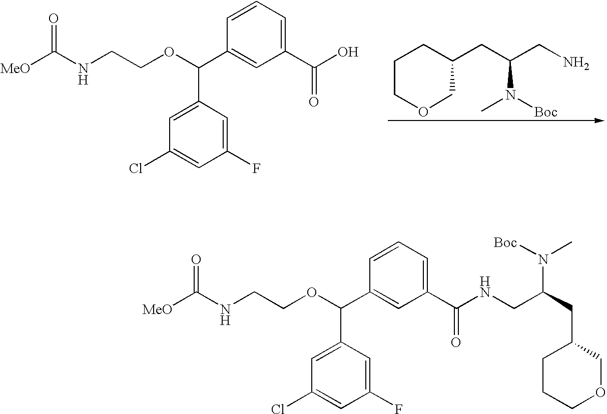

A mixture of 3-((3-chloro-5-fluorophenyl)(2-(methoxycarbon-ylamino)ethoxy)methyl)benzoic acid (170 mg, 0.45 mmol), tert-butyl (S)-1-amino-3-((R)-tetrahydro-2H-pyran-3-yl)propan-2-yl(methyl)carbamate (121 mg, 0.45 mmol), EDCI (175 mg, 0.9 mmol), HOBt (120 mg, 0.9 mmol) and DIEA (120 mg, 0.9 mmol) in DMF (10 mL) was stirred at room temperature for 3 hours. The mixture was concentrated and washed with water. The organic layer was concentrated to give crude [2-{3-[(3-Chloro-5-fluoro-phenyl)-(2-methoxy carbonylamino-ethoxy)-methyl]-benzoylamin-o}-1-(tetrahydro-pyran-3-ylmethyl)-ethyl]-methyl-carbamic acid tert-butyl ester, which was used for the next step without further purification (250 mg, 88%).

### Step 10 methyl 2-((S)-(3-chloro-5-fluorophenyl)(3-((S)-2-(methylamino)-3-((R)-tetrahydro-2H-pyran-3-yl)propylcarbamoyl)phenyl)methoxy)ethylcarbamate and methyl 2-((R)-(3-chloro-5-fluorophen-yl)(3-((S)-2-(methylamino)-3-((R)-tetrahydro-2H-pyran-3-yl)propylcarbamoyl)phenyl)methoxy)ethylcarbamate

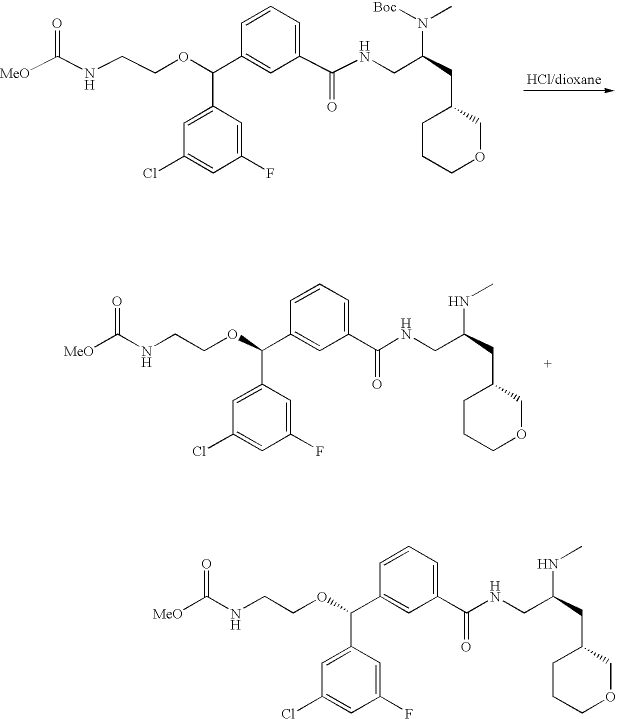

[2-{3-[(3-Chloro-5-fluoro-phenyl)-(2-methoxycarbonylamino-ethoxy)-methyl]-benzo-ylamino}-1-(tetrahydro-pyran-3-ylmethyl)-ethyl]-methyl-carbamic acid tert-butyl ester (250 mg, 0.394 mmol) was dissolved in HCl/dioxane (10 mL) at 0° C. The mixture was stirred for 1 hour, concentrated to give the crude product, which was purified by preparative HPLC and chiral HPLC to give methyl 2-((S)-(3-chloro-5-fluorophenyl)(3-((S)-2-(m-ethylamino)-3-((R)-tetrahydro-2H-pyran-3-yl)propylcarbamoyl)phenyl)methoxy)ethylcarbamate (12.3 mg, 6%) and methyl 2-((R)-(3-chloro-5-fluorophen-yl)(3-((S)-2-(methylamino)-3-((R)-tetrahydro-2H -pyran-3-yl)propyl carbamoyl)phenyl)methoxy)ethylcarbamate (14.5 mg, 7%). Methyl 2-((R)-(3-chloro-5-fluorophen-yl)(3-((S)-2-(methylamino)-3-((R)-tetrahydro-2H-pyran-3-yl)propylcarbamoyl)phenyl)methoxy)ethylcarbamate: ¹HNMR: (400 MHz, CDCl₃): δ=1.25 (m, 1H), 1.38 (m, 2H), 1.61 (m, 2H), 1.75 (m, 1H), 1.95 (d, 1H), 2.53 (s, 3H), 3.09 (t, 1H), 3.35 (m, 4H), 3.5 (m, 4H), 3.60 (s, 3H), 3.84 (m, 2H), 5.49 (s, 1H), 7.08 (q, 2H), 7.24 (s, 1H), 7.46 (m, 1H), 7.55 (m, 1H), 7.77 (d, 1H), 7.87 (s, 1H). Methyl 2-((S)-(3-chloro-5-fluorophenyl)(3-((S)-2-(methylamino)-3-((R)-tetrahydro-2H-pyran-3-yl)propylcarbamoyl)phenyl)methoxy)ethylcarbamate: ¹HNMR: (400 MHz, CDCl₃): δ=1.28 (m, 1H), 1.48-1.63 (m, 4H), 1.79 (m, 1H), 1.95 (d, 1H), 2.72 (s, 3H), 3.13 (t, 1H), 3.35 (m, 3H), 3.40 (m, 1H), 3.52 (m, 3H), 3.60 (s, 3H), 3.81 (d, 1H), 3.84 (d, 2H), 5.49 (s, 1H), 7.06 (q, 2H), 7.22 (s, 1H), 7.47 (t, 1H), 7.56 (d, 1H), 7.79 (d, 1H), 7.89 (s, 1H).

### Example 7

### In Vitro Activity Studies—IC₅₀ for Renin

The compounds of the invention have enzyme-inhibiting properties. In particular, they inhibit the action of the natural enzyme renin. The latter passes from the kidneys into the blood where it effects the cleavage of angiotensinogen, releasing the decapeptide angiotensin I, which is then cleaved in the blood, lungs, the kidneys and other organs by angiotensin converting enzyme to form the octapeptide angiotensin II. The octapeptide increases blood pressure both directly by binding to its receptor, causing arterial vasoconstriction, and indirectly by liberating from the adrenal glands the sodium-ion-retaining hormone aldosterone, accompanied by an increase in extracellular fluid volume. That increase can be attributed to the action of angiotensin II. Inhibitors of the enzymatic activity of renin bring about a reduction in the formation of angiotensin I. As a result, a smaller amount of angiotensin II is produced. The reduced concentration of that active peptide hormone is the direct cause of the hypotensive effect of renin inhibitors.

The action of renin inhibitors in vitro is demonstrated experimentally by means of a test that measures the increase in fluorescence of an internally quenched peptide substrate. The sequence of this peptide corresponds to the sequence of human angiotensinogen. The following test protocol is used: All reactions are carried out in a flat bottom white opaque microtiter plate. A 4 μL aliquot of 400 μM renin substrate (DABCYL-γ-Abu-Ile-His-Pro-Phe-His-Leu-Val-Ile-His-Thr-EDANS) in 192 μL assay buffer (50 mM BES, 150 mM NaCl, 0.25 mg/mL bovine serum albumin, pH7.0) is added to 4 μL of test compound in DMSO at various concentrations ranging from 10 μM to 1 nM final concentrations. Next, 100 μL of trypsin-activated recombinant human renin (final enzyme concentration of 0.2-2 nM) in assay buffer is added, and the solution is mixed by pipetting. The increase in fluorescence at 495 nm (excitation at 340 nm) is measured for 60-360 min at rt using a Perkin-Elmer Fusion microplate reader. The slope of a linear portion of the plot of fluorescence increases as a function of time is then determined, and the rate is used for calculating percent inhibition in relation to uninhibited control. The percent inhibition values are plotted as a function of inhibitor concentration, and the IC₅₀ is determined from a fit of this data to a four parameter equation. The IC₅₀ is defined as the concentration of a particular inhibitor that reduces the formation of product by 50% relative to a control sample containing no inhibitor. (Wang G. T. et al. Anal. Biochem. 1993, 210, 351; Nakamura, N. et al. J. Biochem. (Tokyo) 1991, 109, 741; Murakami, K. et al. Anal Biochem. 1981, 110, 232).

The IC₅₀ values of the disclosed compounds for renin listed in Table 2 were determined according to the protocols described in Example 3. In these in vitro systems, the compounds of the invention exhibit 50% inhibition at concentrations of from approximately 5000 nM to approximately 0.01 nM. Preferred compounds of the invention exhibit 50% inhibition at concentrations of from approximately 50 n M to approximately 0.01 nM. More preferred compounds of the invention exhibit 50% inhibition at concentrations of from approximately 5 nM to approximately 0.01 nM. Highly preferred compounds of the invention exhibit 50% inhibition at concentrations of from approximately 5 nM to approximately 0.01 nM and exhibit 50% inhibition at concentrations of from approximately 10 nM to approximately 0.01 nM in the in vitro assay in the presence of human plasma described below.

### Example 8

### In Vitro Activity Studies

Alternatively, the potency of renin inhibitors is measured using the following in vitro renin assay. In this assay, renin-catalyzed proteolysis of a fluorescently labeled peptide converts the peptide from a weakly fluorescent to a strongly fluorescent molecule. The following test protocol is used. Substrate solution (5 ul; 2 uM Arg-Glu-Lys(5-Fam)-Ile-His-Pro-Phe-His-Leu-Val-Ile-His-Thr-Lys(5,6 Tamra)-Arg-CONH₂ in 50 mM Hepes, 125 mM NaCl, 0.1% CHAPS, pH 7.4) then trypsin-activated recombinant human renin (Scott, Martin J. et. al. Protein Expression and Purification 2007, 52(1), 104-116; 5 uL; 600 pM renin in 50 mM Hepes, 125 mM NaCl, 0.1% CHAPS, pH 7.4) are added sequentially to a black Greiner low volume 384-well plate (cat. #784076) pre-stamped with a 100 nl DMSO solution of compound at the desired concentration. The assay plates are incubated at room temperature for 2 hours with a cover plate then quenched by the addition of a stop solution (2 uL; 5 uM of Bachem C-3195 in 50 mM Hepes, 125 mM NaCl, 0.1% CHAPS, pH 7.4, 10% DMSO). The assay plates are read on an LJL Acquest using a 485 nm excitation filter, a 530 nm emission filter, and a 505 nm dichroic filter. Compounds are initially prepared in neat DMSO at a concentration of 10 mM. For inhibition curves, compounds were diluted using a three fold serial dilution and tested at 11 concentrations (e.g. 50 μM-0.8 nM or 25 nM or 2.5 μM to 42 pM). Curves were analyzed using ActivityBase and XLfit, and results were expressed as pIC₅₀ values.

The in vitro enzyme activity studies were conducted for the compounds of Tables 3 and 4. Each of the compounds demonstrated an in vitro IC₅₀ of less than 1000 nM.

### Example 9

### In Vitro Activity of the Disclosed Compounds in Human Plasma

The action of renin inhibitors in vitro in human plasma can be demonstrated experimentally by the decrease in plasma renin activity (PRA) levels observed in the presence of the compounds. Incubations mixtures will contain in the final volume 250 μL 95.5 mM N,N-bis(2-hydroxyethyl)-2-aminoethanesulfonic acid, pH 7.0, 8 mM EDTA, 0.1 mM neomycin sulfate, 1 mg/ml sodium azide, 1 mM phenylmethanesulfonyl fluoride, 2% DMSO and 87.3% of pooled mixed-gender human plasma stabilized with EDTA. For plasma batches with low PRA (less than 1 ng/ml/hr) ˜2 pM of recombinant human renin will be added to achieve PRA of 3-4 ng/ml/hr. The cleavage of endogenous angiotensinogen in plasma will be carried out at 37° C. for 90 min and the product angiotensin I is measured by competitive radioimmunoassay using DiaSorin PRA kit. Uninhibited incubations containing 2% DMSO and fully inhibited controls with 2 μM of isovaleryl-Phe-Nle-Sta-Ala-Sta-OH will be used for deriving percent of inhibition for each concentration of inhibitors and fitting dose-response data into a four parametric model from which IC₅₀ values, defined as concentrations of inhibitors at which 50% inhibition occurs, will be determined.

### Example 10

### Efficacy of the Disclosed Inhibitors in a Transgenic Rat Model

The efficacy of the renin inhibitors may also be evaluated in vivo in double transgenic rats engineered to express human renin and human angiotensinogen (Bohlender J, Fukamizu A, Lippoldt A, Nomura T, Dietz R, Menard J, Murakami K, Luft F C, Ganten D. High human renin hypertension in transgenic rats. Hypertension 1997, 29, 428-434).

Experiments could be conducted in 5-10 week-old double transgenic rats (dTGRs). The model has been described in detail earlier. Briefly, the human renin construct that may be used to generate transgenic animals (hRen) is made up of the entire genomic human renin gene (10 exons and 9 introns), with 3.0 kB of the 5′-promoter region and 1.2 kB of 3′ additional sequences. A human angiotensinogen construct containing the entire human angiotensinogen gene (5 exons and 4 introns), with 1.3 kB of 5′-flanking and 2.4 kB of 3′-flanking sequences may be used to generate rats producing human angiotensinogen (hAogen). The hRen and hAogen rats may be rederived using embryo transfer from breeding pairs obtained under license from Ascencion Gmbh (Germany). The hAogen and hRen may then be crossed to produce the double transgenic dTGR) off-spring. The dTGr rats should be maintained on irradiated rodent chow (5VO2, Purina Mills Inc) and normal water. Radio telemetry transmitters (TA11PAC40, Data Sciences International) may be surgically implanted at 5-6 weeks of age. The telemetry system can provide 24-h recordings of systolic, mean, diastolic arterial pressure (SAP, MAP, DAP, respectively) and heart rate (HR). Prior to dosing, baseline hemodynamic measures should be obtained for 24 hours. Rats may then be dosed orally with vehicle or drug and monitored up to 48 hours post-dose.

### Example 11

### In Vivo Activity

The cardiac and systemic hemodynamic efficacy of selective renin inhibitors can be evaluated in vivo in sodium-depleted, normotensive cynomolgus monkeys and in sodium-depleted, normotensive beagle dogs following a single oral and intravenous administration of the test compound. Arterial blood pressure is monitored by telemetry in freely moving, conscious animals.

Cynomolgus Monkey: Six male naïve cynomolgus monkeys weighing between 2.5 and 3.5 kg can be used in the studies. At least 4 weeks before the experiment, the monkeys are anesthetized with ketamine hydrochloride (15 mg/kg, i.m.) and xylazine hydrochloride (0.7 mg/kg, i.m.), and are implanted into the abdominal cavity with a transmitter (Model #TL11M2-D70-PCT, Data Sciences, St. Paul, Minn.). The pressure catheter is inserted into the lower abdominal aorta via the femoral artery. The bipotential leads are placed in Lead II configuration. The animals are housed under constant temperature (19-25° C.), humidity (>40%) and lighting conditions (12 h light and dark cycle), are fed once daily, and are allowed free access to water. The animals are sodium depleted by placing them on a low sodium diet (0.026%, Expanded Primate Diet 829552 MP-VENaCl(P), Special Diet Services, Ltd., UK) 7 days before the experiment and furosemide (3 mg/kg, intramuscularly i.m., Aventis Pharmaceuticals) is administered at −40 h and −16 h prior to administration of test compound.

For oral dosing, the renin inhibitors are formulated in 0.5% methylcellulose at dose levels of 10 and 30 mg/kg (5 ml/kg) by infant feeding tubes. For intravenous delivery, a silastic catheter is implanted into posterior vena cava via a femoral vein. The catheter is attached to the delivery pump via a tether system and a swivel joint. Test compound (dose levels of 0.1 to 10 mg/kg, formulated at 5% dextrose) is administered by continuous infusion (1.67 mL/kg/h) or by bolus injection (3.33 mL/kg in 2 min).

Arterial blood pressures (systolic, diastolic and mean) and body temperature are recorded continuously at 500 Hz and 50 Hz, respectively, using the Dataquest™ A.R.T. (Advanced Research Technology) software. Heart rate is derived from the phasic blood pressure tracing. During the recording period, the monkeys are kept in a separate room without human presence to avoid pressure changes secondary to stress. All data are expressed as mean±SEM. Effects of the renin inhibitors on blood pressure are assessed by ANOVA, taking into account the factors dose and time compared with the vehicle group.

Beagle Dogs: Non-naive Beagle dogs (2 per sex) weighing between 9 and 11 kg can be used in the studies. Each animal is implanted subcutaneously with a telemetry transmitter (Data Sciences) and the blood pressure catheter is inserted into the left femoral artery. The electrocardiogram leads are also tunneled subcutaneously to the appropriate anatomical regions. The animals are housed under constant temperature and lighting conditions, are fed once daily, and are allowed free access to water. A sodium depleted state is produced by placing them on a low-sodium diet (<4 meq/day, a combination of canned Prescription Diet canine h/d, from Hill's Pet Products and dry pellets from Bio-Serv Inc., Frenchtown, N.J.) beginning 10 days before the experiment, and furosemide (3 mg/kg i.m.; Aventis Pharmaceuticals) is administered at −40 and −16 h prior to administration of test compound.

A renin inhibitor is orally administered by orogastric gavage to all overnight fasted animals at a dose level of 30 mg/kg (4 mL/kg formulated in 0.5% methylcellulose). Food is given 4 h postdose. In some experiments, the renin inhibitor is administered by bolus i.v. at increasing dose levels of 1, 3 and 6 mg/kg (2, 6 and 20 mg/mL formulated in sterile saline). Cardiovascular parameters are collected continuously at least 80 min predose and 3 h postdose, followed by every 10 min for 5 h and every 30 min for 16 h postdose. The Dataquest™ ART (version 2.2) software package from DSI (Data Sciences International) is used to collect telemetered cardiovascular data.

While this invention has been particularly shown and described with references to preferred embodiments thereof, it will be understood by those skilled in the art that various changes in form and details may be made therein without departing from the scope of the invention encompassed by the appended claims.

1. A compound represented by the following structural formula: 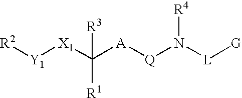
wherein:
X₁ is a covalent bond, —O—, —S—, —S(O)—, —S(O)₂—;
Y₁ is a covalent bond or C₁-C₁₀ alkylene, C₁-C₁₀ alkenylene or C₁-C₁₀ alkynylene, each optionally substituted at one or more substitutable carbon atom with halogen, cyano, nitro, hydroxy, (C₁-C₃)alkyl, (C₁-C₃)alkoxy or halo(C₁-C₃)alkoxy, provided that Y₁ is a covalent bond only when X₁ is a covalent bond;
A is a saturated or unsaturated 4-, 5-, 6-, or 7-membered ring which is optionally bridged by (CH₂)ₚ via bonds to two members of said ring, wherein said ring is composed of carbon atoms and 0-2 hetero atoms selected from the group consisting of 0, 1, or 2 nitrogen atoms, 0 or 1 oxygen atoms, and 0 or 1 sulfur atoms, said ring being optionally and independently substituted with zero to four halogen atoms, (C₁-C₆)alkyl groups, halo(C₁-C₆)alkyl groups or oxo groups such that when there is substitution with one oxo group on a carbon atom it forms a carbonyl group, and when there is substitution of one or two oxo groups on sulfur it forms sulfoxide or sulfone groups, respectively;
p is 1 to 3;
R¹ is (C₃-C₇) cycloalkyl, phenyl, heteroaryl, or bicyclic heteroaryl each optionally substituted with 1 to 3 groups independently selected from: fluorine, chlorine, bromine, cyano, nitro, hydroxy, (C₁-C₆)alkyl. (C₃-C₆)cycloalkyl, (C₄-C₇)cycloalkylalkyl, (C₂-C₆)alkenyl, (C₅-C₇)cycloalkylalkenyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₅-C₇)-cycloalkylalkynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy and (C₁-C₆)alkanesulfonyl: and phenyl, heteroaryl, phenoxy, heteroaryloxy, phenylthio, heteroarylthio, benzyl, heteroarylmethyl, benzyloxy and heteroarylmethoxy, each optionally substituted with 1 to 3 groups independently selected from: fluorine, chlorine, bromine, cyano, nitro, hydroxy, (C₁-C₃)alkyl, halo(C₁-C₃)alkyl, (C₁-C₃)-alkoxy, and halo(C₁-C₃)alkoxy, and aminocarbonyl;
R² is —NHC(═NR¹²)(NH₂), —NHC(═NR¹²)(NHR⁹), 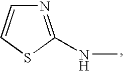
—OC(O)(NH₂), —OC(S)(NH₂), —SC(S)(NH₂), —SC(O)(NH₂), —OC(O)(NHR⁹), —OC(S)(NHR⁹), —SC(S)(NHR⁹), —SC(O)(NHR⁹), —NHC(O)OR⁹, —NHC(S)SR⁹, —NHC(S)OR⁹, —NHC(O)SR⁹, —C(O)R⁹, —C(S)R⁹, —C(O)(NH₂), —C(S)(NH₂), —C(O)(NHR⁹), —C(S)(NHR⁹) or —NHC(O)H, wherein R⁹ is a straight or branched C₁-C₅ alkyl, straight or branched C₁-C₅haloalkyl, (C₃-C₄)cycloalkyl or straight or branched C₁-C₅ alkoxyalkyl and R¹² is H, (C₁-C₆)alkyl, phenyl, heteroaryl, cyano, nitro, —S(O)R^{9,} —S(O₂)R⁹, —S(O₂)NHR⁹, —S(O₂)NR⁹R⁹, —C(O)R⁹, —C(S)R⁹, —C(O)OR⁹, —C(S)OR⁹, —C(O)(NH₂), —C(O)(NHR⁹);
R³ is —H, —F, C₁-C₅ alkyl, —NHC(O)R¹⁰, —OH or —OR¹⁰, wherein R¹⁰ is C₁-C₃ alkyl, provided that when R³ is —F or —OH, then X₁ is not —O—, —S—, —S(O)—, —S(O)₂— and R²—Y₁—X₁ is not —OC(O)(NH₂), —OC(S)(NH₂), —SC(S)(NH₂), —SC(O)(NH₂), —OC(O)(NHR⁹), —OC(S)(NHR⁹), —SC(S)(NHR⁹), —SC(O)(NHR⁹), —NHC(O)OR⁹, —NHC(S)OR⁹, —NHC(S)SR⁹, —NHC(O)SR⁹ or —NHC(O)H;
Q is Q1, Q2, Q3, Q4, Q5, or Q6: 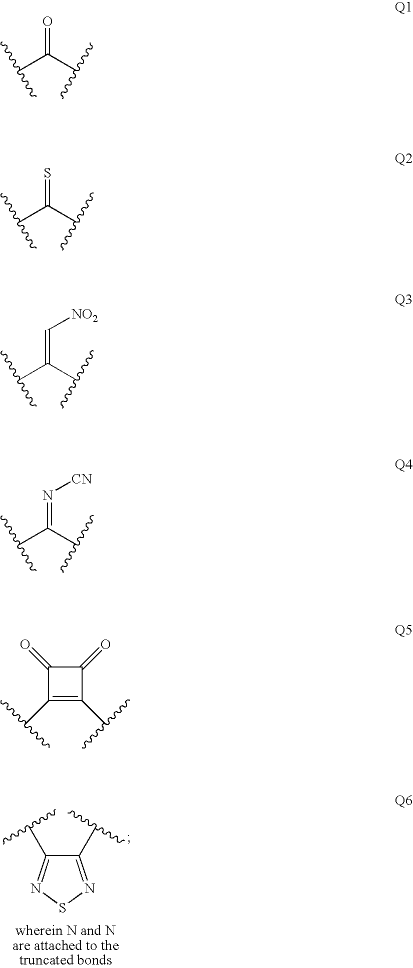
R⁴ is H, (C₁-C₆)alkyl, halo(C₁-C₆)alkyl, (C₁-C₃)alkoxy(C_{I}-C₃)alkyl, or cyano(C₁-C₆)alkyl;
G is OH, OR^{e}, NH₂, NHR^{e}, NR^{e}R^{f}, C(=NH)NH₂, C(═NH)NHR^{e}, NHC(═NH)NH₂, or NHC(═NH)NHR^{e};
L is 1) a linear (C₂-C₄)alkyl chain when G is OH, OR^{e}, NH₂, NHR^{e}, NR^{e}R^{f}, NHC(═NH)NH₂, or NHC(═NH)NHR^{e}, or 2) a linear (C₁-C₃)alkyl chain when G is C(═NH)NH₂ or C(═NH)NHR^{e};
L is optionally substituted by 1-4 groups independently selected from R⁵, R^{5a}, R⁶, and R^{6a}; one or more of the carbon atoms of L may be part of a 3-, 4-, 5-, 6-, or 7-membered saturated ring composed of carbon atoms, and 0-2 hetero atoms selected from 0 or 1 nitrogen atoms, 0 or 1 oxygen atoms, and 0 or 1 sulfur atoms; said saturated ring being optionally substituted with up to four groups selected from halogen, (C₁-C₆)alkyl, halo(C₃-C₆)alkyl, (C₃-C₆)cycloalkyl, halo(C₃-C₆)cycloalkyl, (C₄-C₇)cycloalkylalkyl, halo(C₄-C₇)cycloalkylalkyl, and oxo, such that when there is substitution with one oxo group on a carbon atom it forms a carbonyl group and when there is substitution of one or two oxo groups on sulfur it forms sulfoxide or sulfone groups, respectively;
R⁵, R^{5a}, R⁶, and R^{6a} is each independently selected from 1) H, (C₁-C₁₂)alkyl, halo(C₁-C₁₂)alkyl, hydroxy(C -C₁₂)alkyl, (C₃-C₁₀)cycloalkyl, (C₃-C₁₀)cycloalkyl, (C₃-C₁₀)cycloalkylalkyl, halo(C₃-C₁₀)cycloalkylalkyl, hydroxy(C₃-C₁₀)cycloalkylalkyl, (C₁-C₂)alkyl (C₃-C₁₀)cycloalkylalkyl, halo(C₁-C₂)alkyl (C₃-C₁₀)cycloalkylalkyl, di (C₁-C₂)alkyl (C₃-C₁₀)cycloalkylalkyl, hydroxy(C₁-C₂)alkyl (C₃-C₁₀)cycloalkylalkyl, hydroxy di (C₁-C₂)alkyl (C₃-C₁₀)cycloalkylalkyl, (C₂-C₁₂)alkenyl, (C₅-C₈)cycloalkyl(C₁-C₃)alkenyl, (C₂-C₁₂)alkynyl, (C₃-C₈)cycloalkyl(C₁-C₃)alkynyl, (C₄-C₁₂)bicycloalkyl(C₁-C₃)alkyl, (C₈-C₁₄)tricycloalkyl(C₁-C₃)alkyl, (C₁-C₆)alkoxy(C₁-C₆)alkyl, halo(C₁-C₆)alkoxy(C₁-C₆)alkyl, (C₃-C₈)cycloalkoxy(C₁-C₃)alkyl, (C₁-C₆)alkylthio(C₁-C₆)alkyl, halo(C₁-C₆)alkylthio(C₁-C₆)alkyl, (C₃-C₈)cycloalkylthio(C₁-C₃)alkyl, saturated heterocyclyl, and saturated heterocyclyl(C₁-C₃)alkyl wherein (a) hydrogen atoms in these groups are optionally substituted by 1 to 6 groups independently selected from halogen, cyano, nitro, hydroxy, (C₁-C₆)alkyl, (C₁-C₆)alkoxy, (C₃-C₆)cycloalkyl, (C₃-C₇)cycloalkylalkyl, halo(C₃-C₇)cycloalkylalkyl, (C₂-C₆)alkenyl, halo(C₂-C₆)alkenyl, (C₃-C₇)cycloalkylalkenyl, (C₂-C₆)alkynyl, halo(C₂-C₆)alkynyl, (C₃-C₇)cycloalkylalkoxy, halo(C₃-C₇)cycloalkylalkoxy, (C₃-C₇)cycloalkoxy, halo(C₁-C₆)alkyl, (C₃-C₇)cycloalkylalkynyl, halo(C₃-C₇)cycloalkylalkynyl, halo(C₁-C₆)alkoxy, halo(C₃-C₇)cycloalkyl, halo(C₃-C₇)cycloalkoxy, (C₁-C₆)alkylsulfonyl, aminocarbonyl and wherein (b) divalent sulfur atoms are optionally oxidized to sulfoxide or sulfone;
or 2) phenyl, naphthyl, heteroaryl, phenyl(C₁-C₃)alkyl, phenoxymethyl, naphthyl(C₁-C₃)alkyl, and heteroaryl(C₁-C₃)alkyl, each optionally substituted with 1 to 3 groups independently selected from: halogen, cyano, nitro, amino, hydroxy, carboxy, (C₁-C₆)alkyl, (C₃-C₆)cycloalkyl, (C₄-C₇)cycloalkylalkyl, (C₂-C₆)alkenyl, halo(C₂-C₆)alkenyl, (C₃-C₆)cycloalkylalkenyl, (C₂-C₆)alkynyl, halo(C₂-C₆)alkynyl, (C₃-C₆)cycloalkyl-(C₂-C₄)alkynyl, halo(C₃-C₇)cycloalkylalkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cyeloalkoxy, halo(C₄-C₇)cycloalkylalkoxy, (C₁-C₆)alkylthio, (C₃-C₆)cycloalkythio, (C₄-C₇)cycloalkylalkylthio, halo(C₁-C₆)alkylthio, halo(C₃-C₆)cycloalkythio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)-cycloalkylalkanesulfonyl, (C₁-C₆)alkylamino, di(C₁-C₆)alkylamino, (C₁-C₆)-alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio(C₁-C₆)alkyl, (C₃-C₈)cycloalkythio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkylalkylthio(C₁-C₆)alkyl, (C₁-C₈)alkanesulfinyl(C₁-C₆)alkyl, (C₃-C₈)-cycloalkanesulfinyl(C₁-C₆)alkyl, (C₄-C₈)cycloalkyl-alkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkane-sulfonyl(C₁-C₆)alkyl, (C₃-C₈)cycloalkanesulfonyl(C₁-C₆)alkyl, (C₄-C₈) cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkane-sulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylamino-carbonyl(C₁-C₆)alkyl, di(C₁-C₈)alkylaminocarbonyl(C₁-C₆)alkyl and (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylamino-carboxy(C₁-C₆)alkyl and di(C₁-C₈)alkylaminocarboxy(C₁-C₆)alkyl, phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl, wherein the aromatic and heteroaromatic groups are optionally substituted with 1 to 3 groups independently selected from fluorine, chlorine, cyano, (C₁-C₃)alkyl, halo(C₁-C₃)alkyl, (C₁-C₃)alkoxy, halo(C₁-C₃)-alkoxy, (C₁-C₃)alkanesulfonyl, and (C₁-C₃)alkoxycarbonyl;
R^{e} is a) (C₁-C₁₂)alkyl, (C₄-C₁₂)cycloalkylalkyl, halo(C₁-C₁₂)alkyl, halo(C₄-C₁₂)cycloalkylalkyl, (C₂-C₁₂)alkenyl, (C₅-C₁₂)cycloalkylalkenyl, halo(C₂-C₁₂)alkenyl, halo(C₅-C₁₂)cycloalkylalkenyl, (C₂-C₁₂)alkynyl, (C₅-C₁₂)cycloalkylalkynyl, halo(C₂-C₁₂)alkynyl, halo(C₅-C₁₂)cycloalkylalkynyl, (C₁-C₆)alkoxy(C₁-C₆)alkyl, halo(C₁-C₆)alkoxy(C₁-C₆)alkyl, (C₁-C₆)alkylthio(C₁-C₆)alkyl, halo(C₁-C₆)alkylthio(C₁-C₆)alkyl, (C₁-C₆)alkanesulfinyl(C₁-C₆)alkyl, halo(C₁-C₆)alkane-sulfinyl(C₁-C₆)alkyl, (C₁-C₆)alkanesulfonyl(C₁-C₆)alkyl, halo(C₁-C₆)alkanesulfonyl(C₁-C₆)alkyl, aminocarbonyl(C₁-C₆)alkyl, (C₁-C₆)alkylaminocarbonyl(C₁-C₆)alkyl, di(C₁-C₆)alkylamino-carbonyl(C₁-C₆)alkyl, cyano(C₁-C₆)alkyl, carboxy(C₁-C₆)alkyl, (C₁-C₆)alkoxycarbonyl(C₁-C₆)alkyl, saturated heterocyclyl, or saturated heterocyclyl(C₁-C₆)alkyl or b) phenyl, naphthyl, heteroaryl, phenyl(C₁-C₃)alkyl, naphthyl(C₁-C₃)alkyl, or heteroaryl(C₁-C₃)alkyl, each of a) and b) are optionally substituted by 1 to 3 groups independently selected from: 1) fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl-(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkythio, (C₄-C₇)cycloalkylalkylthio, halo(C₁-C₆)alkylthio, halo(C₃-C₆)cycloalkythio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)-cycloalkylalkanesulfonyl, (C₁-C₆)alkylamino, di(C₁-C₆)alkylamino, (C₁-C₆)alkoxy-(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)-cycloalkylalkoxy(C₁-C₆)alkyl, (C₁-C₈)alkylthio(C₁-C₆)alkyl, (C₃-C₈)cycloalkythio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkylalkylthio(C₁-C₆)alkyl, (C₁-C₈)alkanesulfinyl(C₁-C₆)alkyl, (C₃-C₈)-cycloalkanesulfinyl(C₁-C₆)alkyl, (C₄-C₈)cycloalkyl-alkanesulfinyl(C₁-C₆)alkyl halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkane-sulfonyl(C₁-C₆)alkyl, (C₃-C_{g})cycloalkanesulfonyl(C₁-C₆)alkyl, (C₄-C₈) cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkane-sulfonyl (C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl (C₁-C₆)alkyl, (C₁-C₈)alkylamino-carbonyl(C₁-C₆)alkyl, di(C₁-C₈)alkylaminocarbonyl(C₁-C₆)alkyl (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylamino-carboxy(C₁-C₆)alkyl and di(C₁-C₈)alkylaminocarboxy(C₁-C₆)alkyl; or 2) phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenyl sulfonyl, naphthylsulfonyl, heteroarylsulfonyl, bicyclic heteroarylsulfonyl, phenyl (C₁-C₃)alkyl, naphthyl(C₁-C₃)alkyl, heteroaryl(C₁-C₃)alkyl, and bicyclic heteroaryl(C₁-C₃)alkyl, each optionally substituted with 1 to 3 groups independently selected from fluorine, chlorine, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)-alkoxycarbonyl; or
b) R^{e} is a saturated divalent radical composed of carbon atoms, and 0, 1 or 2 hetero atoms selected from 0 or 1 nitrogen atoms, 0 or 1 oxygen atoms, and 0 or 1 sulfur atoms that is attached to any core carbon atom on L to form a saturated 3-, 4-, 5-, 6-, or 7-membered L-G ring; said L-G ring being optionally substituted with 1 to 4 groups selected from halogen, fluorine, (C₁-C₈)alkyl, halo(C₁-C₈)alkyl, (C₃-C₈)cycloalkyl, halo(C₃-C₈)cycloalkyl, hydroxy(C₃-C₈)cycloalkyl, (C₃-C₈)cycloalkyl(C₁-C₃)alkyl, halo(C₃-C₈)cycloalkyl(C₁-C₃)alkyl, hydroxy (C₃-C₈)cycloalkyl(C₁-C₃)alkyl, (C₁-C₈)alkoxy, halo(C₁-C₈)alkoxy, (C₃-C₈)cycloalkoxy, halo(C₃-C₈)cycloalkoxy, hydroxy(C₃-C₈)cycloalkoxy, (C₁-C₈)alkoxy(C₁-C₃)alkyl, halo(C₁-C₈)alkoxy(C₁-C₃)alkyl, (C₃-C₈)cycloalkoxy(C₁-C₃)alkyl, halo(C₃-C₈)cycloalkoxy(C₁-C₃)alkyl, hydroxy(C₃-C₈)cycloalkoxy(C₁-C₃)alkyl, (C₃-C₈)cycloalkyl(C₁-C₃)alkoxy(C₁-C₃)alkyl, halo(C₃-C₈)cycloalkyl(C₁-C₃)alkoxy(C₁-C₃)alkyl, hydroxy(C₃-C₈)cycloalkyl(C₁-C₃)alkoxy(C₁-C₃)alkyl, (C₁-C₈)alkylthio, halo(C₁-C₈)alkylthio, (C₃-C₈)cycloalkylthio, halo(C₃-C₈)cycloalkylthio, hydroxy(C₃-C₈)cycloalkylthio, (C₃-C₈)cycloalkyl(C₁-C₃)alkylthio, halo(C₃-C₈)cycloalkyl(C₁-C₃)alkylthio, hydroxy(C₃-C₈)cycloalkyl(C₁-C₃)alkylthio, (C₁-C₈)alkylthio(C₁-C₃)alkyl, halo(C₁-C₈)alkylthio(C₁-C₃)alkyl, (C₃-C₈)cycloalkylthio(C₁-C₃)alkyl, halo(C₃-C₈)cycloalkylthio(C₁-C₃)alkyl, hydroxy(C₃-C₈)cycloalkylthio(C₁-C₃)alkyl, (C₃-C₈)cycloalkyl(C₁-C₃)alkylthio(C₁-C₃)alkyl, halo(C₃-C₈)cycloalkyl (C₁-C₃)alkylthio(C₁-C₃)alkyl, hydroxy(C₃-C₈)cycloalkyl(C₁-C₃)alkylthio(C₁-C₃)alkyl, heterocyclyl, and oxo;
R^{f} is (C₁-C₆)alkyl or halo(C₁-C₆)alkyl;
or an enantiomer, diastereomer, or a pharmaceutically acceptable salt thereof; provided that
A is not 2,4-morpholine or 1,3-piperidine 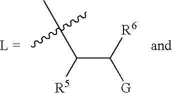
R² is —NHC(═NR₁₂)(NH₂), —NHC(═NR¹²)(NHR⁹), 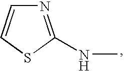
—OC(O)(NH₂), —OC(S)(NH₂), —SC(S)(NH₂), —SC(O)(NH₂), —OC(O)(NHR⁹), —OC(S)(NHR⁹), —SC(S)(NHR⁹), —SC(O)(NHR⁹), —NHC(O)OR⁹, —NHC(S)SR⁹, —NHC(S)OR⁹, —NHC(O)SR⁹, —C(O)R⁹, —C(S)R⁹, —C(O)(NH₂), —C(S)(NH₂), —C(O)(NHR⁹), —C(S)(NHR⁹) or —NHC(O)H, wherein R⁹ is a straight or branched C₁-C₅ alkyl, straight or branched C₁-C₅haloalkyl, (C₃-C₄)cycloalkyl or straight or branched C₁-C₅ alkoxyalkyl and R¹² is H, (C₁-C₆)alkyl, phenyl, heteroaryl, cyano, nitro, —S(O)R^{9,} —S(O₂)R⁹, —S(O₂)NHR⁹, —S(O₂)NR⁹R⁹, —C(O)R⁹, —C(S)R⁹, —C(O)OR⁹, —C(S)OR⁹, —C(O)(NH₂), —C(O)(NHR⁹).

2. The compound of claim 1, wherein
R² is —NHC(═NR₁₂)(NH₂), —NHC(═NR¹²)(NHR⁹), 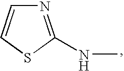
—OC(O)(NH₂), —OC(S)(NH₂), —OC(O)(NHR⁹), —OC(S)(NHR⁹), —NHC(O)OR⁹, —NHC(S)SR⁹, —NHC(S)OR⁹, —NHC(O)SR⁹, —C(O)R⁹, —C(S)R⁹, —C(O)(NH₂), —C(S)(NH₂), —C(O)(NHR⁹), —C(S)(NHR⁹) or —NHC(O)H, wherein R⁹ is a straight or branched C₁-C₅ alkyl, straight or branched C₁-C₅ haloalkyl, (C₃-C₄)cycloalkyl or straight or branched C₁-C₅ alkoxyalkyl and R¹² is H, (C₁-C₆)alkyl, phenyl, heteroaryl, cyano, nitro, —S(O)R^{9,} —S(O₂)R⁹, —S(O₂)NHR⁹, —S(O₂)NR⁹R⁹, —C(O)R⁹, —C(S)R⁹, —C(O)OR⁹, —C(S)OR⁹, —C(O)(NH₂), —C(O)(NHR⁹).

3. The compound of claim 2, wherein
A is a saturated or unsaturated 4-, 5-, 6-, or 7-membered ring which is optionally bridged by (CH₂)ₚ via bonds to two members of said ring, wherein said ring is composed of carbon atoms, said ring being optionally and independently substituted with zero to four halogen atoms, (C₁-C₆)alkyl groups, halo(C₁-C₆)alkyl groups or oxo groups such that when there is substitution with one oxo group on a carbon atom it forms a carbonyl group;
p is 1 to 3;
or an enantiomer, diastereomer, or a pharmaceutically acceptable salt thereof.

4. The compound of claim 3, wherein the compound is represented by the following structural formula: 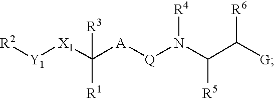
or an enantiomer, diastereomer, or a pharmaceutically acceptable salt thereof.

5. The compound of claim 4, wherein:
G is OH, NH₂ or NHR^{e}; and
R^{e} is a) (C₁-C₆)alkyl, halo(C₁-C₆)alkyl, (C₄-C₁₀)cycloalkylalkyl, (C₁-C₅)alkoxy(C₁-C₅)alkyl, or aminocarbonyl(C₁-C₆)alkyl or b) phenyl(C₁-C₂)alkyl optionally substituted with 1 to 3 groups independently selected from: fluorine, chlorine, cyano, (C₁-C₃)alkyl, halo(C₁-C₃)alkyl, (C₁-C₃)alkoxy, and halo(C₁-C₃)alkoxy; or c) R⁵ and R^{e} together are —CH₂—, —(CH₂)₂—, —(CH₂)₃—, or —(CH₂)₄—, optionally substituted with 1 or 2 groups independently selected from fluorine, (C₁-C₈)alkyl, halo(C₁-C₈)alkyl, (C₃-C₆)cycloalkyl, halo(C₃-C₆)cycloalkyl, hydroxy(C₃-C₆)cycloalkyl, (C₃-C₆)cycloalkyl(C₁-C₂)alkyl, halo(C₃-C₆)cycloalkyl(C₁-C₂)alkyl, hydroxy(C₃-C₆)cycloalkyl(C₁-C₂)alkyl, (C₁-C₈)alkoxy, halo(C₁-C₈)alkoxy, (C₃-C₆)cycloalkoxy, halo(C₃-C₆)cycloalkoxy, and heterocyclyl;
or an enantiomer, diastereomer, or a pharmaceutically acceptable salt thereof.

6. The compound of claim 5, wherein the compound is represented by the following structural formula: 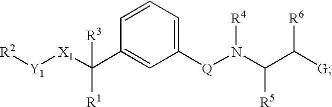
or an enantiomer, diastereomer, or a pharmaceutically acceptable salt thereof.

7. The compound of claim 6, wherein the compound is represented by the following structural formula: 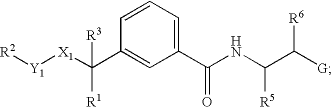
or an enantiomer, diastereomer, or a pharmaceutically acceptable salt thereof.

8. The compound of claim 7, wherein one of R⁵ and R⁶ is —H or methyl and the other is a) H, (C₁-C₁₀)alkyl, (C₄-C₁₀)cycloalkylalkyl, halo(C₄-C₁₀)alkyl, hydroxy(C₁-C₁₀)alkyl, halo(C₄-C₁₀)cycloalkylalkyl, hydroxy(C₄-C₁₀)cycloalkylalkyl, (C₁-C₂)alkyl(C₄-C₁₀)cycloalkylalkyl, halo(C₁-C₂)alkyl(C₄-C₁₀)cycloalkylalkyl, di(C₁-C₂)alkyl(C₄-C₁₀)cycloalkylalkyl, hydroxy(C₁-C₂)alkyl(C₄-C₁₀)cycloalkylalkyl, hydroxy di(C₁-C₂)alkyl(C₄-C₁₀)cycloalkylalkyl, (C₄-C₁₀)bicycloalkyl(C₁-C₃)alkyl, (C₈-C₁₂)tricycloalkyl(C₁-C₃)alkyl, (C₁-C₅)alkoxy(C₁-C₅)alkyl, halo(C₁-C₅)alkoxy(C₁-C₅)alkyl, (C₁-C₅)alkylthio(C₁-C₅)alkyl, halo(C₁-C₅)alkylthio(C₁-C₅)alkyl, or saturated heterocyclyl(C₁-C₃)alkyl; or b) phenyl(C₁-C₂)alkyl, phenoxymethyl or heteroaryl(C₁-C₂)alkyl each optionally substituted with 1 to 3 groups independently selected from fluorine, chlorine, cyano, (C₁-C₃)alkyl, halo(C₁-C₃)alkyl, (C₁-C₃)alkoxy, and halo(C₁-C₃)alkoxy,

9. The compound of claim 8, wherein R⁶ is —H or methyl.

10. The compound of claim 8, wherein R⁵ is —H or methyl.

11. The compound of claim 8, wherein the compound is represented by the following structural formula: 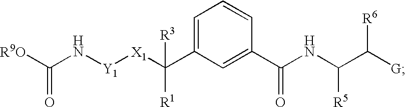
or an enantiomer, diastereomer, or a pharmaceutically acceptable salt thereof.

12. The compound of claim 11, wherein the compound is represented by the following structural formula: 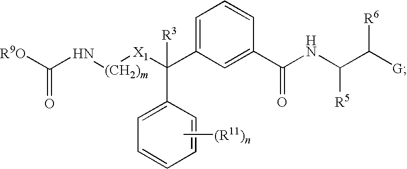
or an enantiomer, diastereomer, or a pharmaceutically acceptable salt thereof, wherein:
R¹¹ is fluorine, chlorine, bromine, cyano, nitro, hydroxy, (C₁-C₆)alkyl, (C₃-C₆)cycloalkyl, (C₄-C₇)cycloalkylalkyl, (C₂-C₆)alkenyl, (C₅-C₇)cycloalkylalkenyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₅-C₇)-cycloalkylalkynyl, (C₁-C₆)alkoxy, (C₃-C₆)cycloalkoxy, (C₄-C₂)cycloalkylalkoxy, halo(C₁-C₆)alkoxy, halo(C₃-C₆)cycloalkoxy, halo(C₄-C₇)cycloalkylalkoxy and (C₁-C₆)alkanesulfonyl; and phenyl, heteroaryl, phenoxy, heteroaryloxy, phenylthio, heteroarylthio, benzyl, heteroarylmethyl, benzyloxy and heteroarylmethoxy, each optionally substituted with 1 to 3 groups independently selected from: fluorine, chlorine, bromine, cyano, nitro, hydroxy, (C₁-C₃)alkyl, halo(C₁-C₃)alkyl, (C₁-C₃)-alkoxy, and halo(C₁-C₃)alkoxy, and aminocarbonyl;
n is 0, 1, 2 or 3; and
m is 2 or 3.

13. The compound of claim 12, wherein:
R⁵ is (C₁-C₇)alkyl, halo(C₁-C₇)alkyl, hydroxy(C₁-C₇)alkyl, cyclohexylmethyl, halocyclohexylmethyl, hydroxy cyclohexylmethyl, 2-(cyclohexyl)ethyl, (C₁-C₂)alkyl cyclohexylmethyl, di(C₁-C₂)alkyl cyclohexylmethyl, hydroxy (C₁-C₂)alkyl cyclohexylmethyl, hydroxy di(C₁-C₂)alkylcyclohexylmethyl, (3-noradamantyl)methyl, (tetrahydropyranyl)methyl, or oxepanyl methyl;
R⁶ is —H or methyl;
G is NH₂ or NHR^{e};
R^{e} is methyl or R⁵ and R⁶ together are —(CH₂)₃— optionally substituted with C₁-C₄ alkyl or cyclohexyl.

14. The compound of claim 12, wherein:
R⁶ is (C₁-C₇)alkyl, halo(C₁-C₇)alkyl, hydroxy(C₁-C₇)alkyl, cyclohexylmethyl, halocyclohexylmethyl, hydroxy cyclohexylmethyl, 2-(cyclohexyl)ethyl, (C₁-C₂)alkyl cyclohexylmethyl, di(C₁-C₂)alkyl cyclohexylmethyl, hydroxy (C₁-C₂)alkyl cyclohexylmethyl, hydroxy di(C₁-C₂)alkylcyclohexylmethyl, (3-noradamantyl)methyl, (tetrahydropyranyl)methyl, or oxepanyl methyl;
R⁵ is —H or methyl;
G is NH₂ or NHR^{e};
R^{e} is methyl or R⁶ and R^{e} together are —(CH₂)₃— optionally substituted with C₁-C₄ alkyl or cyclohexyl.

15. The compound of claim 13, wherein:
R⁹ is methyl or ethyl; and
R¹¹ is chloro, fluoro or methyl.

16. The compound of claim 14, wherein:
R⁹ is methyl or ethyl; and
R¹¹ is chloro, fluoro or methyl.

17. The compound of claim 12, wherein the compound is represented by the following structural formula: 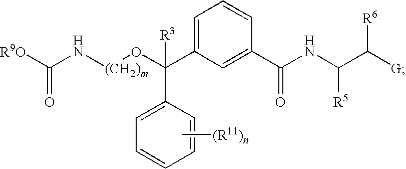
or an enantiomer, diastereomer, or a pharmaceutically acceptable salt thereof, wherein:
R⁵ is (C₁-C₇)alkyl, halo(C₁-C₇)alkyl, hydroxy(C₁-C₇)alkyl, cyclohexylmethyl, halocyclohexylmethyl, hydroxy cyclohexylmethyl, 2-(cyclohexyl)ethyl, (C₁-C₂)alkyl cyclohexylmethyl, di(C₁-C₂)alkyl cyclohexylmethyl, hydroxy (C₁-C₂)alkyl cyclohexylmethyl, hydroxy di(C₁-C₂)alkylcyclohexylmethyl, (3-noradamantyl)methyl, tetrahydropyranyl)methyl, or oxepanyl methyl;
R⁶ is —H or methyl;
G is NH₂ or NHR^{e};
R^{e} is methyl or R⁵ and R^{e} together are —(CH₂)₃— optionally substituted with C₁-C₄ alkyl or cyclohexyl.

18. The compound of claim 17, wherein:
R⁹ is methyl or ethyl; and
R¹¹ is chloro, fluoro or methyl.

19. The compound of claim 17, wherein the compound is represented by the following structural formula: 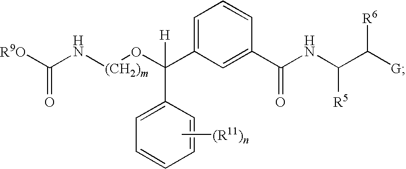
or an enantiomer, diastereomer, or a pharmaceutically acceptable salt thereof.

20. The compound of claim 17, wherein the compound is represented by a structural formula selected from: 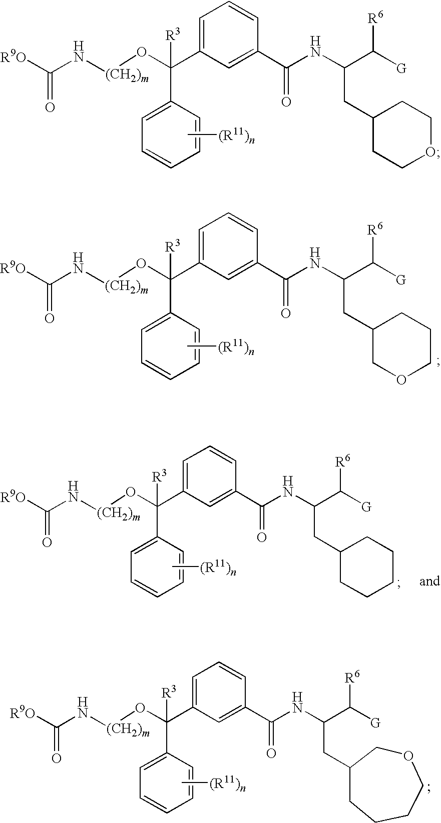
or an enantiomer, diastereomer, or a pharmaceutically acceptable salt thereof.

21. The compound of claim 1 selected from the group consisting of: methyl 2-((3-chlorophenyl)(3-(1-(methylamino)-3-(tetrahydro-2H-pyran-4-yl)propan-2-ylcarbamoyl)phenyl)methoxy)ethylcarbamate; methyl 2-((3-chlorophenyl)(3-(1-(methylamino)-3-(tetrahydro-2H-pyran-3-yl)propan-2-ylcarbamoyl)phenyl)methoxy)ethylcarbamate; methyl 2-((3-chlorophenyl)(3-(1-cyclohexyl-3-(methylamino)propan-2-ylcarbamoyl)phenyl)methoxy)ethylcarbamate; methyl 2-((3-chlorophenyl)(3-(1-(methylamino)-3-(oxepan-3-yl)propan-2-ylcarbamoyl)phenyl)methoxy)ethylcarbamate and pharmaceutically acceptable salts of any of the above.

22. The compound of claim 1 selected from the group consisting of: methyl 2-((R)-(3-chlorophenyl)(3-((S)-1-(methylamino)-3-(tetrahydro-2H-pyran-4-yl)propan-2-ylcarbamoyl)phenyl)methoxy)ethylcarbamate; methyl 2-((S)-(3-chlorophenyl)(3-((S)-1-(methylamino)-3-((R)-tetrahydro-2H-pyran-3-yl)propan-2-ylcarbamoyl)phenyl)methoxy)ethylcarbamate; methyl 2-((R)-(3-chlorophenyl)(3-((S)-1-(methylamino)-3-((R)-tetrahydro-2H-pyran-3-yl)propan-2-ylcarbamoyl)phenyl)methoxy)ethylcarbamate; methyl 2-((R)-(3-chlorophenyl)(3-((S)-1-cyclohexyl-3-(methylamino)propan-2-ylcarbamoyl)phenyl)methoxy)ethylcarbamate; methyl 2-((S)-(3-chlorophenyl)(3-((S)-1-cyclohexyl-3-(methylamino)propan-2-ylcarbamoyl)phenyl)methoxy)ethylcarbamate; methyl 2-((R)-(3-chlorophenyl)(3-((S)-1-(methylamino)-3-((R)-oxepan-3-yl)propan-2-ylcarbamoyl)phenyl)methoxy)ethylcarbamate; methyl 2-((S)-(3-chlorophenyl)(3-((S)-1-(methylamino)-3-((R)-oxepan-3-yl)propan-2-ylcarbamoyl)phenyl)methoxy)ethylcarbamate and pharmaceutically acceptable salts of any of the above.

23. The compound of claim 8, wherein the compound is represented by the following structural formula: 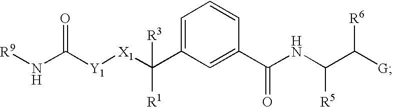
or an enantiomer, diastereomer, or a pharmaceutically acceptable salt thereof.

24. The compound of claim 23, wherein the compound is represented by the following structural formula: 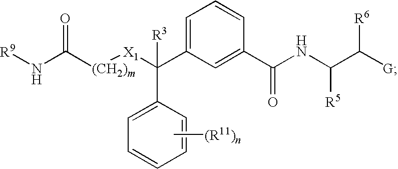
or an enantiomer, diastereomer, or a pharmaceutically acceptable salt thereof, wherein:
R¹¹ is fluorine, chlorine, bromine, cyano, nitro, hydroxy, (C₁-C₆)alkyl, (C₃-C₆)cycloalkyl, (C₄-C₇)cycloalkylalkyl, (C₂-C₆)alkenyl, (C₅-C₇)cycloalkylalkenyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₅-C₇)-cycloalkylalkynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy and (C₁-C₆)alkanesulfonyl; and phenyl, heteroaryl, phenoxy, heteroaryloxy, phenylthio, heteroarylthio, benzyl, heteroarylmethyl, benzyloxy and heteroarylmethoxy, each optionally substituted with 1 to 3 groups independently selected from: fluorine, chlorine, bromine, cyano, nitro, hydroxy, (C₁-C₃)alkyl, halo(C₁-C₃)alkyl, (C₁-C₃)-alkoxy, and halo(C₁-C₃)alkoxy, and aminocarbonyl;
n is 0, 1, 2 or 3; and
m is 2 or 3.

25. The compound of claim 24, wherein:
R⁵ is (C₁-C₇)alkyl, halo(C₁-C₇)alkyl, hydroxy(C₁-C₇)alkyl, cyclohexylmethyl, halocyclohexylmethyl, hydroxy cyclohexylmethyl, 2-(cyclohexyl)ethyl, (C₁-C₂)alkyl cyclohexylmethyl, di(C₁-C₂)alkyl cyclohexylmethyl, hydroxy (C₁-C₂)alkyl cyclohexylmethyl, hydroxy di(C₁-C₂)alkylcyclohexylmethyl, (3-noradamantyl)methyl, (tetrahydropyranyl)methyl, or oxepanyl methyl;
R⁶ is —H or methyl;
G is NH₂ or NHR^{e};
R^{e} is methyl or R⁵ and R^{e} together are —(CH₂)₃— optionally substituted with C₁-C₄ alkyl or cyclohexyl.

26. The compound of claim 25, wherein:
R⁹ is methyl or ethyl; and
R¹¹ is chloro, fluoro or methyl.

27. The compound of claim 24, wherein the compound is represented by the following structural formula: 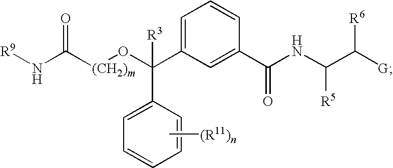
or an enantiomer, diastereomer, or a pharmaceutically acceptable salt thereof, wherein:
R⁵ is (C₁-C₇)alkyl, halo(C₁-C₇)alkyl, hydroxy(C₁-C₇)alkyl, cyclohexylmethyl, halocyclohexylmethyl, hydroxy cyclohexylmethyl, 2-(cyclohexyl)ethyl, (C₁-C₂)alkyl cyclohexylmethyl, di(C₁-C₂)alkyl cyclohexylmethyl, hydroxy (C₁-C₂)alkyl cyclohexylmethyl, hydroxy di(C₁-C₂)alkylcyclohexylmethyl, (3-noradamantyl)methyl, (tetrahydropyranyl)methyl, or oxepanyl methyl;
R⁶ is —H or methyl;
G is NH₂ or NHR^{e};
R^{e} is methyl or R⁵ and R^{e} together are —(CH₂)₃— optionally substituted with C₁-C₄ alkyl or cyclohexyl.

28. The compound of claim 27, wherein:
R⁹ is methyl or ethyl; and
R¹¹ is chloro, fluoro or methyl.

29. The compound of claim 28, wherein the compound is represented by the following structural formula: 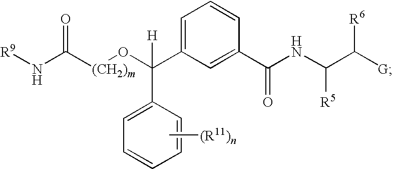
or an enantiomer, diastereomer, or a pharmaceutically acceptable salt thereof.

30. The compound of claim 8, wherein the compound is represented by the following structural formula: 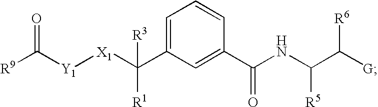
or an enantiomer, diastereomer, or a pharmaceutically acceptable salt thereof.

31. The compound of claim 30, wherein the compound is represented by the following structural formula: 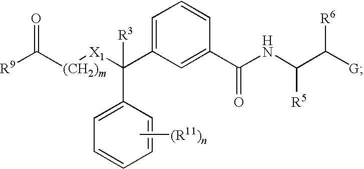
or an enantiomer, diastereomer, or a pharmaceutically acceptable salt thereof, wherein:
R¹ is fluorine, chlorine, bromine, cyano, nitro, hydroxy, (C₁-C₆)alkyl, (C₃-C₆)cycloalkyl, (C₄-C₇)cycloalkylalkyl, (C₂-C₆)alkenyl, (C₅-C₇)cycloalkylalkenyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₅-C₇)cycloalkylalkynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy and (C₁-C₆)alkanesulfonyl; and phenyl, heteroaryl, phenoxy, heteroaryloxy, phenylthio, heteroarylthio, benzyl, heteroarylmethyl, benzyloxy and heteroarylmethoxy, each optionally substituted with 1 to 3 groups independently selected from: fluorine, chlorine, bromine, cyano, nitro, hydroxy, (C₁-C₃)alkyl, halo(C₁-C₃)alkyl, (C₁-C₃)-alkoxy, and halo(C₁-C₃)alkoxy, and aminocarbonyl;
n is 0, 1, 2 or 3; and
m is 2, 3 or 4.

32. The compound of claim 31, wherein:
R⁵ i s (C₁-C₇)alkyl, halo(C₁-C₇)alkyl, hydroxy(C₁-C₇)alkyl, cyclohexylmethyl, halocyclohexylmethyl, hydroxy cyclohexylmethyl, 2-(cyclohexyl)ethyl, (C₁-C₂)alkyl cyclohexylmethyl, di(C₁-C₂)alkyl cyclohexylmethyl, hydroxy (C₁-C₂)alkyl cyclohexylmethyl, hydroxy di(C₁-C₂)alkylcyclohexylmethyl, (3-noradamantyl)methyl, (tetrahydropyranyl)methyl, or oxepanyl methyl;
R⁶ is —H or methyl;
G is NH₂ or NHR^{e};
R^{e} is methyl or R⁵ and R^{e} together are —(CH₂)₃— optionally substituted with C₁-C₄ alkyl or cyclohexyl.

33. The compound of claim 32, wherein:
R⁹ is methyl or ethyl; and
R¹¹ is chloro, fluoro or methyl.

34. The compound of claim 31, wherein the compound is represented by the following structural formula: 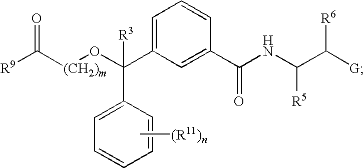
or an enantiomer, diastereomer, or a pharmaceutically acceptable salt thereof, wherein:
R⁵ is (C₁-C₇)alkyl, halo(C₁-C₇)alkyl, hydroxy(C₁-C₇)alkyl, cyclohexylmethyl, halocyclohexylmethyl, hydroxy cyclohexylmethyl, 2-(cyclohexyl)ethyl, (C₁-C₂)alkyl cyclohexylmethyl, di(C₁-C₂)alkyl cyclohexylmethyl, hydroxy(C₁-C₂)alkyl cyclohexylmethyl, hydroxy di(C₁-C₂)alkylcyclohexylmethyl, (3-noradamantyl)methyl, (tetrahydropyranyl)methyl, or oxepanyl methyl;
R⁶ is —H methyl;
G is NH₂ or NHR^{e};
R^{e} is methyl or R⁵ and R^{e} together are —(CH₂)₃— optionally substituted with C₁-C₄ alkyl or cyclohexyl.

35. The compound of claim 34, wherein:
R⁹ is methyl or ethyl; and
R¹¹ is chloro, fluoro or methyl.

36. The compound of claim 35, wherein the compound is represented by a structural formula selected from: 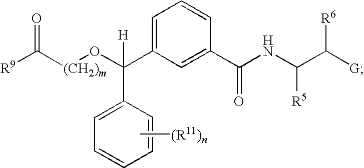
or an enantiomer, diastereomer, or a pharmaceutically acceptable salt thereof.

37. The compound of claim 8, wherein the compound is represented by the following structural formula: 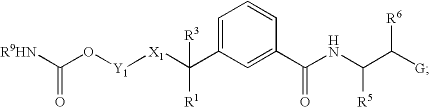
or an enantiomer, diastereomer, or a pharmaceutically acceptable salt thereof.

38. The compound of claim 37, wherein the compound is represented by the following structural formula: 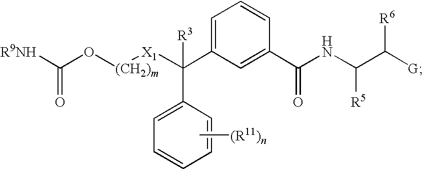
or an enantiomer, diastereomer, or a pharmaceutically acceptable salt thereof, wherein:
R¹¹ is fluorine, chlorine, bromine, cyano, nitro, hydroxy, (C₁-C₆)alkyl, (C₃-C₆)cycloalkyl, (C₄-C₇)cycloalkylalkyl, (C₂-C₆)alkenyl, (C₅-C₇)cycloalkylalkenyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₅-C₇)cycloalkylalkynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy and (C₁-C₆)alkanesulfonyl; and phenyl, heteroaryl, phenoxy, heteroaryloxy, phenylthio, heteroarylthio, benzyl, heteroarylmethyl, benzyloxy and heteroarylmethoxy, each optionally substituted with 1 to 3 groups independently selected from: fluorine, chlorine, bromine, cyano, nitro, hydroxy, (C₁-C₃)alkyl, halo(C₁-C₃)alkyl, (C₁-C₃)-alkoxy, and halo(C₁-C₃)alkoxy, and aminocarbonyl;
n is 0, 1, 2 or 3; and
m is 2 or 3.

39. The compound of claim 38, wherein:
R⁵ is (C₁-C₇)alkyl, halo(C₁-C₇)alkyl, hydroxy(C₁-C₇)alkyl, cyclohexylmethyl, halocyclohexylmethyl, hydroxy cyclohexylmethyl, 2-(cyclohexyl)ethyl, (C₁-C₂)alkyl cyclohexylmethyl, di(C₁-C₂)alkyl cyclohexylmethyl, hydroxy (C₁-C₂)alkyl cyclohexylmethyl, hydroxy di(C₁-C₂)alkylcyclohexylmethyl, (3-noradamantyl)methyl, (tetrahydropyranyl)methyl, or oxepanyl methyl;
R⁶ is H or methyl;
G is NH₂ or NHR^{e};
R^{e} is methyl or R⁵ and R^{e} together are —(CH₂)₃— optionally substituted with C₁-C₄ alkyl or cyclohexyl.

40. The compound of claim 39, wherein:
R⁹ is methyl or ethyl; and
R¹¹ is chloro, fluoro or methyl.

41. The compound of claim 38, wherein the compound is represented by the following structural formula: 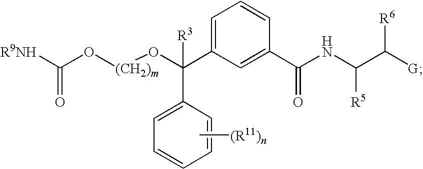
or an enantiomer, diastereomer, or a pharmaceutically acceptable salt thereof, wherein:
R⁵ is (C₁-C₇)alkyl, halo(C₁-C₇)alkyl, hydroxy(C₁-C₇)alkyl, cyclohexylmethyl, halocyclohexylmethyl, hydroxy cyclohexylmethyl, 2-(cyclohexyl)ethyl, (C₁-C₂)alkyl cyclohexylmethyl, di(C₁-C₂)alkyl cyclohexylmethyl, hydroxy (C₁-C₂)alkyl cyclohexylmethyl, hydroxy di(C₁-C₂)alkylcyclohexylmethyl, (3-noradamantyl)methyl, (tetrahydropyranyl)methyl, or oxepanyl methyl;
R⁶ is —H or methyl;
G is NH₂ or NHR^{e};
R^{e} is methyl or R⁵ and R^{e} together are —(CH₂)₃— optionally substituted with C₁-C₄ alkyl or cyclohexyl.

42. The compound of claim 41, wherein:
R⁹ is methyl or ethyl; and
R^{H} is chloro, fluoro or methyl.

43. The compound of claim 42, wherein the compound is represented by the following structural formula: 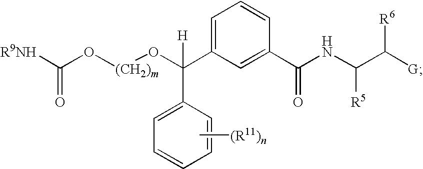
or an enantiomer, diastereomer, or a pharmaceutically acceptable salt thereof.

44. The compound of claim 8, wherein the compound is represented by the following structural formula: 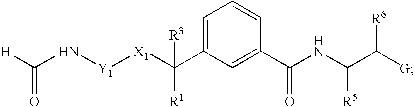
or an enantiomer, diastereomer, or a pharmaceutically acceptable salt thereof.

45. The compound of claim 44, wherein the compound is represented by the following structural formula: 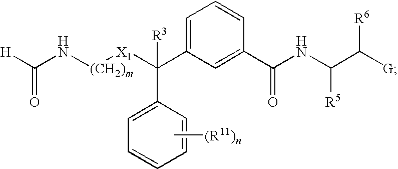
or an enantiomer, diastereomer, or a pharmaceutically acceptable salt thereof, wherein:
R¹¹ is fluorine, chlorine, bromine, cyano, nitro, hydroxy, (C₁-C₆)alkyl, (C₃-C₆)cycloalkyl, (C₄-C₇)cycloalkylalkyl, (C₂-C₆)alkenyl, (C₅-C₇)cycloalkylalkenyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₅-C₇)cycloalkylalkynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy and (C₁-C₆)alkanesulfonyl; and phenyl, heteroaryl, phenoxy, heteroaryloxy, phenylthio, heteroarylthio, benzyl, heteroarylmethyl, benzyloxy and heteroarylmethoxy, each optionally substituted with 1 to 3 groups independently selected from: fluorine, chlorine, bromine, cyano, nitro, hydroxy, (C₁-C₃)alkyl, halo(C₁-C₃)alkyl, (C₁-C₃)-alkoxy, and halo(C₃-C₃)alkoxy, and aminocarbonyl;
n is 0, 1, 2 or 3; and
m is 2, 3 or 4.

46. The compound of claim 45, wherein:
R⁵ is (C₁-C₇)alkyl, halo(C₁-C₇)alkyl, hydroxy(C₁-C₇)alkyl, cyclohexylmethyl, halocyclohexylmethyl, hydroxy cyclohexylmethyl, 2-(cyclohexyl)ethyl, (C₁-C₂)alkyl cyclohexylmethyl, di(C₁-C₂)alkyl cyclohexylmethyl, hydroxy (C₁-C₂)alkyl cyclohexylmethyl, hydroxy di(C₁-C₂)alkylcyclohexylmethyl, (3-noradamantyl)methyl, (tetrahydropyranyl)methyl, or oxepanyl methyl;
R⁶ is or methyl;
G is NH₂ or NHR^{e};
R^{e} is methyl or R⁵ and R^{e} together are —(CH₂)₃— optionally substituted with C₁-C₄ alkyl or cyclohexyl.

47. The compound of claim 46, wherein:
R¹¹ is chloro, fluoro or methyl.

48. The compound of claim 45, wherein the compound is represented by the following structural formula: 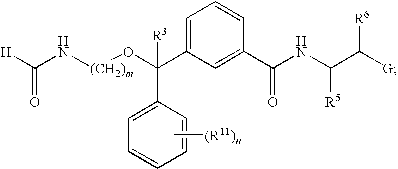
or an enantiomer, diastereomer, or a pharmaceutically acceptable salt thereof, wherein:
R⁵ is (C₁-C₇)alkyl, halo(C₁-C₇)alkyl, hydroxy(C₁-C₇)alkyl, cyclohexylmethyl, halocyclohexylmethyl, hydroxy cyclohexylmethyl, 2-(cyclohexyl)ethyl, (C₁-C₂)alkyl cyclohexylmethyl, di(C₁-C₂)alkyl cyclohexylmethyl, hydroxy(C₁-C₂)alkyl cyclohexylmethyl, hydroxy di(C₁-C₂)alkylcyclohexylmethyl, (3-noradamantyl)methyl, (tetrahydropyranyl)methyl, or oxepanyl methyl;
R⁶ is —H or methyl;
G is NH₂ or NHR^{e};
R^{e} is methyl or R⁵ and R^{e} together are —(CH₂)₃— optionally substituted with C₁-C₄ alkyl or cyclohexyl.

49. The compound of claim 48, wherein:
R¹¹ is chloro, fluoro or methyl.

50. The compound of claim 49, wherein the compound is represented by the following structural formula: 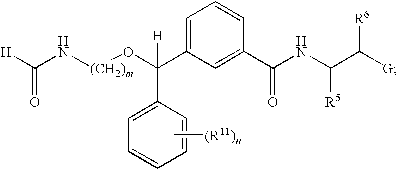
or an enantiomer, diastereomer, or a pharmaceutically acceptable salt thereof.

51. The compound of claim 16, wherein compound is represented by the following structural formula: 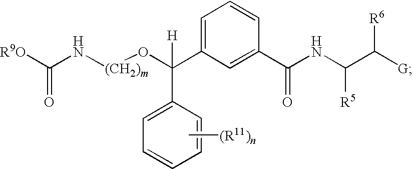
or an enantiomer, diastereomer, or a pharmaceutically acceptable salt thereof.

52. The compound of claim 51, wherein R⁶ is (tetrahydropyranyl)methyl.

53. A pharmaceutical composition comprising a pharmaceutically acceptable carrier or diluent and the compound of claim 1 or a pharmaceutically acceptable salt thereof.

54. The pharmaceutical composition of claim 53 further comprising a α-blocker, β-blocker, calcium channel blocker, diuretic, natriuretic, saluretic, centrally acting antiphypertensive, angiotensin converting enzyme (ACE) inhibitor, dual ACE and neutral endopeptidase (NEP) inhibitor, angiotensin-receptor blocker (ARB), aldosterone synthase inhibitor, aldosterone-receptor antagonist, or endothelin receptor antagonist.

55.-62. (canceled)
